(19) United States
(12) Patent Application Publication
(10) Pub. No.: US 2023/0126157 A1
(43) Pub. Date: Apr. 27, 2023

(54) MIRNA-485 INHIBITOR FOR GENE UPREGULATION

(71) Applicant: BIORCHESTRA CO., LTD., Daejeon (KR)

(72) Inventors: Jin-Hyeob RYU, Daejeon (KR); Han Seok KOH, Daejeon (KR); Dae Hoon KIM, Daejeon (KR); Hyun Su MIN, Daejeon (KR); Yu Na LIM, Daejeon (KR)

(73) Assignee: BIORCHESTRA CO., LTD., Daejeon (KR)

(21) Appl. No.: 17/760,343

(22) PCT Filed: Feb. 5, 2021
(86) PCT No.: PCT/IB2021/050974

§ 371 Date: Aug. 8, 2022

Related U.S. Application Data
(60) Provisional application No. 62/971,767, filed on Feb. 7, 2020.
(60) Provisional application No. 62/989,486, filed on Mar. 13, 2020.
(60) Provisional application No. 63/047,155, filed on Jul. 1, 2020.
(60) Provisional application No. 63/064,314, filed on Aug. 11, 2020.

(51) Int. Cl. C12N 15/113; A61K 47/64; A61K 47/55; A61K 47/54; A61K 47/60

(52) U.S. Cl. CPC C12N 15/1138; C12N 15/1137; A61K 47/6455; A61K 47/55; A61K 47/542; A61K 47/60; C12N 2310/141; C12N 2310/3181; C12N 2310/314; C12N 2310/313; C12N 2310/3231

(57) ABSTRACT

The present disclosure includes the use of a miRNA inhibitor for treating a disease or condition associated with a decreased level of SIRT1, PGC-1α, CD36, LRRK2, NRG1, STMN2, VLDLR, NRXN1, GRIA4, NXPH1, PSD-95, and/or synaptophysin protein or SIRT1, PGC-1α, CD36, LRRK2, NRG1, STMN2, VLDLR, NRXN1, GRIA4, NXPH1, PSD-95, and/or synaptophysin gene expression. In some aspects, the miRNA inhibitor can be used to treat a disease or condition associated with an increased level of caspase-3 protein or gene expression. The miRNA inhibitor useful for the present disclosure can inhibit miR-485 expression and/or activity, which in turn can increase the level of SIRT1, PGC-1α, CD36, LRRK2, NRG1, STMN2, VLDLR, NRXN1, GRIA4, NXPH1, PSD-95, and/or synaptophysin protein or gene expression; and/or can decrease the level of caspase 3 protein or gene expression.

### CROSS-REFERENCE TO RELATED APPLICATIONS

This PCT application claims the priority benefit of U.S. Provisional Application No. 62/971,767, filed Feb. 7, 2020; 62/989,486, filed Mar. 13, 2020; 63/047,155, filed Jul. 1, 2020; and 63/064,314, filed Aug. 11, 2020; each of which is incorporated herein by reference in its entirety.

### REFERENCE TO SEQUENCE LISTING SUBMITTED ELECTRONICALLY VIA EFS-WEB

The content of the electronically submitted sequence listing in ASCII text file (Name: 4366_014PC04_Seqlisting_ST25.txt; Size: 264,023 bytes; and Date of Creation: Feb. 5, 2021) filed with the application is herein incorporated by reference in its entirety.

### FIELD OF THE DISCLOSURE

The present disclosure provides the use of a miR-485 inhibitor (e.g., polynucleotide encoding a nucleotide molecule comprising at least one miR-485 binding site) for the treatment of diseases and disorders associated with reduced SIRT1 expression (e.g., neurodegenerative diseases and disorders, e.g., Alzheimer's disease).

### BACKGROUND OF THE DISCLOSURE

Sirtulin 1 (also known as NAD-dependent deacetylase sirtuin-1) is an enzyme that in humans is encoded by the SIRT1 gene. It belongs to a family of nicotinamide adenine dinucleotide (NAD)-dependent histone deacetylases and can deacetylate a variety of substrates. Rahman, S., et al., Cell Communication and Signaling 9:11 (2011). Accordingly, sirtulin 1 has been described as playing a role in a broad range of physiological functions, including control of gene expression, metabolism, and aging. And, abnormal sirtulin activity has been associated with certain human diseases. For instance, subjects with neurodegenerative disorders have been described as exhibiting low levels of sirtulin 1 activity.

Neurodegenerative disorders, such as Alzheimer's disease (AD) and Parkinson's disease, are common and growing cause of mortality and morbidity worldwide. It is estimated that by 2050, more than 100 million people worldwide will be affected by AD. Gaugler et al., Alzheimer's Dement 12(4): 459-509 (2016); Pan et al., Sci Adv 5(2) (2019). The costs of AD are estimated at more than 800 billion USD globally. Over the past two decades, investigators have been trying to develop compounds and antibodies that can inhibit Aβ production and aggregation, or, promote amyloid beta clearance. Unfortunately, these attempts have not achieved successful clinical benefits in large clinical trials with mild AD patients. Panza et al., Nat Rev Neurol 15(2): 73-88 (2019).

Currently, there are no known cures for neurodegenerative disorders. Available treatment options are generally limited to alleviating the various symptoms, as opposed to addressing the underlying causes of the disorders. Therefore, new and more effective approaches to treating neurodegenerative disorders are highly desirable.

### BRIEF SUMMARY OF THE DISCLOSURE

Provided herein is a method of increasing a level of a SIRT1 protein and/or a SIRT1 gene in a subject in need thereof comprising administering to the subject a compound that inhibits miR-485 (miRNA inhibitor). In some aspects, the subject has a disease or a condition associated with a decreased level of a SIRT1 protein and/or a SIRT1 gene. In some aspects, the miRNA inhibitor induces autophagy and/or treats or prevents inflammation.

Also provided herein is a method of increasing a level of a CD36 protein and/or a CD36 gene in a subject in need thereof comprising administering to the subject a compound that inhibits miR-485 (miRNA inhibitor). In some aspects, the subject has a disease or a condition associated with a decreased level of a CD36 protein and/or a CD36 gene.

Present disclosure further provides a method of increasing a level of a PGC-1α protein and/or a PGC-1α gene in a subject in need thereof comprising administering to the subject a compound that inhibits miR-485 (miRNA inhibitor). In some aspects, the subject has a disease or a condition associated with a decreased level of a PGC-1α protein and/or a PGC-1α gene.

Provided herein is a method of increasing a level of a LRRK2 protein and/or a LRRK2 gene in a subject in need thereof comprising administering to the subject a compound that inhibits miR-485 (miRNA inhibitor). In some aspects, the subject has a disease or a condition associated with a decreased level of a LRRK2 protein and/or a LRRK2 gene.

Also provided herein is a method of increasing a level of a NRG1 protein and/or a NRG1 gene in a subject in need thereof comprising administering to the subject a compound that inhibits miR-485 (miRNA inhibitor). In some aspects, the subject has a disease or a condition associated with a decreased level of a NRG1 protein and/or a NRG1 gene.

Provided herein is a method of increasing a level of a STMN2 protein and/or a STMN2 gene in a subject in need thereof comprising administering to the subject a compound that inhibits miR-485 (miRNA inhibitor). In some aspects, the subject has a disease or a condition associated with a decreased level of a STMN2 protein and/or a STMN2 gene.

Provided herein is a method of increasing a level of a VLDLR protein and/or a VLDLR gene in a subject in need thereof comprising administering to the subject a compound that inhibits miR-485 (miRNA inhibitor). In some aspects, the subject has a disease or a condition associated with a decreased level of a VLDLR protein and/or a VLDLR gene.

Provided herein is a method of increasing a level of a NRXN1 protein and/or a NRXN1 gene in a subject in need thereof comprising administering to the subject a compound that inhibits miR-485 (miRNA inhibitor). In some aspects, the subject has a disease or a condition associated with a decreased level of a NRXN1 protein and/or a NRXN1 gene.

Provided herein is a method of increasing a level of a GRIA4 protein and/or a GRIA4 gene in a subject in need thereof comprising administering to the subject a compound that inhibits miR-485 (miRNA inhibitor). In some aspects, the subject has a disease or a condition associated with a decreased level of a GRIA4 protein and/or a GRIA4 gene.

Provided herein is a method of increasing a level of a NXPH1 protein and/or a NXPH1 gene in a subject in need thereof comprising administering to the subject a compound that inhibits miR-485 (miRNA inhibitor). In some aspects, the subject has a disease or a condition associated with a decreased level of a NXPH1 protein and/or a NXPH1 gene.

Provided herein is a method of increasing a level of a PSD-95 protein and/or a PSD-95 gene in a subject in need thereof comprising administering to the subject a compound that inhibits miR-485 (miRNA inhibitor). In some aspects, the subject has a disease or a condition associated with a decreased level of a PSD-95 protein and/or a PSD-95 gene.

Provided herein is a method of increasing a level of a synaptophysin protein and/or a synaptophysin gene in a subject in need thereof comprising administering to the subject a compound that inhibits miR-485 (miRNA inhibitor). In some aspects, the subject has a disease or a condition associated with a decreased level of a synaptophysin protein and/or a synaptophysin gene.

Provided herein is a method of decreasing a level of a caspase-3 protein and/or a caspase-3 gene in a subject in need thereof comprising administering to the subject a compound that inhibits miR-485 (miRNA inhibitor). In some aspects, the subject has a disease or a condition associated with an increased level of a caspase-3 protein and/or a caspase-3 gene.

In some aspects, a miR-485 inhibitor that can be used in the above methods induces neurogenesis. In certain aspects, inducing neurogenesis comprises an increased proliferation, differentiation, migration, and/or survival of neural stem cells and/or progenitor cells. In some aspects, inducing neurogenesis comprises an increased number of neural stem cells and/or progenitor cells. In some aspects, inducing neurogenesis comprises an increased axon, dendrite, and/or synapse development. In some aspects, a miR-485 inhibitor induces phagocytosis.

Also provided herein is a method of treating a disease or condition associated with an abnormal level of a SIRT1 protein and/or a SIRT1 gene in a subject in need thereof comprising administering to the subject a compound that inhibits miR-485 (miRNA inhibitor), wherein the miRNA inhibitor increases the level of the SIRT1 protein and/or SIRT1 gene. Also provided herein is a method of treating a disease or condition associated with an abnormal level of a CD36 protein and/or a CD36 gene in a subject in need thereof comprising administering to the subject a compound that inhibits miR-485 (miRNA inhibitor), wherein the miRNA inhibitor increases the level of the CD36 protein and/or CD36 gene. Also provided herein is a method of treating a disease or condition associated with an abnormal level of a PGC-1α protein and/or a PGC-1α gene in a subject in need thereof comprising administering to the subject a compound that inhibits miR-485 (miRNA inhibitor), wherein the miRNA inhibitor increases the level of the PGC-1α protein and/or PGC-1α gene. Also provided herein is a method of treating a disease or condition associated with an abnormal level of a LRRK2 protein and/or a LRRK2 gene in a subject in need thereof comprising administering to the subject a compound that inhibits miR-485 (miRNA inhibitor), wherein the miRNA inhibitor increases the level of the LRRK2 protein and/or LRRK2 gene. Also provided herein is a method of treating a disease or condition associated with an abnormal level of a NRG1 protein and/or a NRG1 gene in a subject in need thereof comprising administering to the subject a compound that inhibits miR-485 (miRNA inhibitor), wherein the miRNA inhibitor increases the level of the NRG1 protein and/or NRG1 gene. Also provided herein is a method of treating a disease or condition associated with an abnormal level of a STMN2 protein and/or a STMN2 gene in a subject in need thereof comprising administering to the subject a compound that inhibits miR-485 (miRNA inhibitor), wherein the miRNA inhibitor increases the level of the STMN2 protein and/or STMN2 gene. Also provided herein is a method of treating a disease or condition associated with an abnormal level of a VLDLR protein and/or a VLDLR gene in a subject in need thereof comprising administering to the subject a compound that inhibits miR-485 (miRNA inhibitor), wherein the miRNA inhibitor increases the level of the VLDLR protein and/or VLDLR gene. Also provided herein is a method of treating a disease or condition associated with an abnormal level of a NRXN1 protein and/or a NRXN1 gene in a subject in need thereof comprising administering to the subject a compound that inhibits miR-485 (miRNA inhibitor), wherein the miRNA inhibitor increases the level of the NRXN1 protein and/or NRXN1 gene. Also provided herein is a method of treating a disease or condition associated with an abnormal level of a GRIA4 protein and/or a GRIA4 gene in a subject in need thereof comprising administering to the subject a compound that inhibits miR-485 (miRNA inhibitor), wherein the miRNA inhibitor increases the level of the GRIA4 protein and/or GRIA4 gene. Also provided herein is a method of treating a disease or condition associated with an abnormal level of a NXPH1 protein and/or a NXPH1 gene in a subject in need thereof comprising administering to the subject a compound that inhibits miR-485 (miRNA inhibitor), wherein the miRNA inhibitor increases the level of the NXPH1 protein and/or NXPH1 gene. Also provided herein is a method of treating a disease or condition associated with an abnormal level of a PSD-95 protein and/or a PSD-95 gene in a subject in need thereof comprising administering to the subject a compound that inhibits miR-485 (miRNA inhibitor), wherein the miRNA inhibitor increases the level of the PSD-95 protein and/or PSD-95 gene. Also provided herein is a method of treating a disease or condition associated with an abnormal level of a synaptophysin protein and/or a synaptophysin gene in a subject in need thereof comprising administering to the subject a compound that inhibits miR-485 (miRNA inhibitor), wherein the miRNA inhibitor increases the level of the synaptophysin protein and/or synaptophysin gene. Also provided herein is a method of treating a disease or condition associated with an abnormal level of a caspase-3 protein and/or a caspase-3 gene in a subject in need thereof comprising administering to the subject a compound that inhibits miR-485 (miRNA inhibitor), wherein the miRNA inhibitor decreases the level of the caspase-3 protein and/or caspase-3 gene.

In some aspects, the miRNA inhibitor inhibits miR485-3p. In some aspects, the miR485-3p comprises 5′-GUCAUACACGGCUCUCCUCUCU-3′ (SEQ ID NO: 1). In some aspects, the miRNA inhibitor comprises a nucleotide sequence comprising 5′-UGUAUGA-3′ (SEQ ID NO: 2) and wherein the miRNA inhibitor comprises about 6 to about 30 nucleotides in length.

In some aspects, the miRNA inhibitor increases transcription of an SIRT1 gene and/or expression of a SIRT1 protein; increases transcription of a CD36 gene and/or expression of a CD36 protein; increases transcription of a PGC1 gene and/or expression of a PGC1 protein; increases transcription of a LRRK2 gene and/or expression of a LRRK2 protein; increases transcription of a NRG1 gene and/or expression of a NRG1 protein; increases transcription of a STMN2 gene and/or expression of a STMN2 protein; increases transcription of a VLDLR gene and/or expression of a VLDLR protein; increases transcription of a NRXN1 gene and/or expression of a NRXN1 protein; increases transcription of a GRIA4 gene and/or expression of a GRIA4 protein; increases transcription of a NXPH1 gene and/or expression of a NXPH1 protein; increases transcription of a PSD-95 gene and/or expression of a PSD-95 protein; increases transcription of a synaptophysin gene and/or expression of a synaptophysin protein; decreases transcription of a caspase-3 gene and/or expression of a caspase-3 protein; or any combination thereof.

In some aspects, the miRNA inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 5′ of the nucleotide sequence. In some aspects, the miRNA inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 3′ of the nucleotide sequence.

In some aspects, the miRNA inhibitor has a sequence selected from the group consisting of: 5′-UGUAUGA-3′ (SEQ ID NO: 2), 5′-GUGUAUGA-3′ (SEQ ID NO: 3), 5′-CGUGUAUGA-3′ (SEQ ID NO: 4), 5′-CCGUGUAUGA-3′ (SEQ ID NO: 5), 5′-GCCGUGUAUGA-3′ (SEQ ID NO: 6), 5′-AGCCGUGUAUGA-3′ (SEQ ID NO: 7), 5′-GAGCCGUGUAUGA-3′ (SEQ ID NO: 8), 5′-AGAGCCGUGUAUGA-3′ (SEQ ID NO: 9), 5′-GAGAGCCGUGUAUGA-3′ (SEQ ID NO: 10), 5′-GGAGAGCCGUGUAUGA-3′ (SEQ ID NO: 11), 5′-AGGAGAGCCGUGUAUGA-3′ (SEQ ID NO: 12), 5′-GAGGAGAGCCGUGUAUGA-3′ (SEQ ID NO: 13), 5′-AGAGGAGAGCCGUGUAUGA-3′ (SEQ ID NO: 14), and 5′-GAGAGGAGAGCCGUGUAUGA-3′ (SEQ ID NO: 15).

In some aspects, the miRNA inhibitor has a sequence selected from the group consisting of: 5′-UGUAUGAC-3′ (SEQ ID NO: 16), 5′-GUGUAUGAC-3′ (SEQ ID NO: 17), 5′-CGUGUAUGAC-3′ (SEQ ID NO: 18), 5′-CCGUGUAUGAC-3′ (SEQ ID NO: 19), 5′-GCCGUGUAUGAC-3′ (SEQ ID NO: 20), 5′-AGCCGUGUAUGAC-3′ (SEQ ID NO: 21), 5′-GAGCCGUGUAUGAC-3′ (SEQ ID NO: 22), 5′-AGAGCCGUGUAUGAC-3′ (SEQ ID NO: 23), 5′-GAGAGCCGUGUAUGAC-3′ (SEQ ID NO: 24), 5′-GGAGAGCCGUGUAUGAC-3′ (SEQ ID NO: 25), 5′-AGGAGAGCCGUGUAUGAC-3′ (SEQ ID NO: 26), 5′-GAGGAGAGCCGUGUAUGAC-3′ (SEQ ID NO: 27), 5′-AGAGGAGAGCCGUGUAUGAC-3′ (SEQ ID NO: 28), 5′-GAGAGGAGAGCCGUGUAUGAC-3′ (SEQ ID NO: 29), and AGAGAGGAGAGCCGUGUAUGAC (SEQ ID NO: 30).

In some aspects, the miRNA inhibitor has a sequence selected from the group consisting of: 5′-TGTATGA-3′ (SEQ ID NO: 62), 5′-GTGTATGA-3′ (SEQ ID NO: 63), 5′-CGTGTATGA-3′ (SEQ ID NO: 64), 5′-CCGTGTATGA-3′ (SEQ ID NO: 65), 5′-GCCGTGTATGA-3′ (SEQ ID NO: 66), 5′-AGCCGTGTATGA-3′ (SEQ ID NO: 67), 5′-GAGCCGTGTATGA-3′ (SEQ ID NO: 68), 5′-AGAGCCGTGTATGA-3′ (SEQ ID NO: 69), 5′-GAGAGCCGTGTATGA-3′ (SEQ ID NO: 70), 5′-GGAGAGCCGTGTATGA-3′ (SEQ ID NO: 71), 5′-AGGAGAGCCGTGTATGA-3′ (SEQ ID NO: 72), 5′-GAGGAGAGCCGTGTATGA-3′ (SEQ ID NO: 73), 5′-AGAGGAGAGCCGTGTATGA-3′ (SEQ ID NO: 74), 5′-GAGAGGAGAGCCGTGTATGA-3′ (SEQ ID NO: 75); 5′-TGTATGAC-3′ (SEQ ID NO: 76), 5′-GTGTATGAC-3′ (SEQ ID NO: 77), 5′-CGTGTATGAC-3′ (SEQ ID NO: 78), 5′-CCGTGTATGAC-3′ (SEQ ID NO: 79), 5′-GCCGTGTATGAC-3′ (SEQ ID NO: 80), 5′-AGCCGTGTATGAC-3′ (SEQ ID NO: 81), 5′-GAGCCGTGTATGAC-3′ (SEQ ID NO: 82), 5′-AGAGCCGTGTATGAC-3′ (SEQ ID NO: 83), 5′-GAGAGCCGTGTATGAC-3′ (SEQ ID NO: 84), 5′-GGAGAGCCGTGTATGAC-3′ (SEQ ID NO: 85), 5′-AGGAGAGCCGTGTATGAC-3′ (SEQ ID NO: 86), 5′-GAGGAGAGCCGTGTATGAC-3′ (SEQ ID NO: 87), 5′-AGAGGAGAGCCGTGTATGAC-3′ (SEQ ID NO: 88), 5′-GAGAGGAGAGCCGTGTATGAC-3′ (SEQ ID NO: 89), and 5′-AGAGAGGAGAGCCGTGTATGAC-3′ (SEQ ID NO: 90).

In some aspects, the sequence of the miRNA inhibitor is at least about 50%, at least about 55%, at least about 60%, at least about 65%, at least about 70%, at least about 75%, at least about 80%, at least about 85%, at least about 90%, or at least about 95% sequence identity to 5′-AGAGAGGAGAGCCGUGUAUGAC-3′ (SEQ ID NO: 30) or 5′-AGAGAGGAGAGCCGTGTATGAC-3′ (SEQ ID NO: 90). In certain aspects, the miRNA inhibitor has a sequence that has at least 90% similarity to 5′-AGAGAGGAGAGCCGUGUAUGAC-3′ (SEQ ID NO: 30) or 5′-AGAGAGGAGAGCCGTGTATGAC-3′ (SEQ ID NO: 90). In some aspects, the miRNA inhibitor comprises the nucleotide sequence 5′-AGAGAGGAGAGCCGUGUAUGAC-3′ (SEQ ID NO: 30) or 5′-AGAGAGGAGAGCCGTGTATGAC-3′ (SEQ ID NO: 90) with one substitution or two substitutions. In some aspects, the miRNA inhibitor comprises the nucleotide sequence 5′-AGAGAGGAGAGCCGUGUAUGAC-3′ (SEQ ID NO: 30) or 5′-AGAGAGGAGAGCCGTGTATGAC-3′ (SEQ ID NO: 90). In some aspects, the miRNA inhibitor comprises the nucleotide sequence 5′-AGAGAGGAGAGCCGUGUAUGAC-3′ (SEQ ID NO: 30).

In some aspects, the miRNA inhibitor comprises at least one modified nucleotide. In certain aspects, the at least one modified nucleotide is a locked nucleic acid (LNA), an unlocked nucleic acid (UNA), an arabino nucleic acid (ABA), a bridged nucleic acid (BNA), and/or a peptide nucleic acid (PNA).

In some aspects, the miRNA inhibitor comprises a backbone modification. In certain aspects, the backbone modification is a phosphorodiamidate morpholino oligomer (PMO) and/or phosphorothioate (PS) modification.

In some aspects, the miRNA inhibitor is delivered in a delivery agent. In certain aspects, the delivery agent is a micelle, an exosome, a lipid nanoparticle, an extracellular vesicle, or a synthetic vesicle.

In some aspects, the miRNA inhibitor is delivered by a viral vector. In certain aspects, the viral vector is an AAV, an adenovirus, a retrovirus, or a lentivirus. In some aspects, the viral vector is an AAV that has a serotype of AAV2, AAV3, AAV4, AAV5, AAV6, AAV7, AAV8, AAV9, AAV10, or any combination thereof.

In some aspects, the miRNA inhibitor is delivered with a delivery agent. In certain aspects, the delivery agent comprises a lipidoid, a liposome, a lipoplex, a lipid nanoparticle, a polymeric compound, a peptide, a protein, a cell, a nanoparticle mimic, a nanotube, or a conjugate.

In some aspects, the delivery agent comprises a cationic carrier unit comprising

[WP]-L1-[CC]-L2-[AM] (formula I)

or

[WP]-L1-[AM]-L2-[CC] (formula II)
wherein
WP is a water-soluble biopolymer moiety;
CC is a positively charged carrier moiety;
AM is an adjuvant moiety; and,
L1 and L2 are independently optional linkers, and
wherein when mixed with a nucleic acid at an ionic ratio of about 1:1, the cationic carrier unit forms a micelle.

In some aspects, the miRNA inhibitor interacts with the cationic carrier unit via an ionic bond. In some aspects, the water-soluble polymer comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (“POZ”) poly(N-acryloylmorpholine), or any combinations thereof. In other aspects, the water-soluble polymer comprises polyethylene glycol (“PEG”), polyglycerol, or poly(propylene glycol) (“PPG”).

In some aspects, the water-soluble polymer comprises:

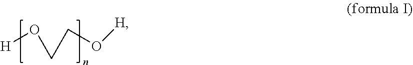

In some aspects, n is 1-1000. In certain aspects, the n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141. In further aspects, the n is about 80 to about 90, about 90 to about 100, about 100 to about 110, about 110 to about 120, about 120 to about 130, about 140 to about 150, about 150 to about 160.

In some aspects, the water-soluble polymer is linear, branched, or dendritic.

In some aspects, the cationic carrier moiety comprises one or more basic amino acids. In certain aspects, the cationic carrier moiety comprises at least three, at least four, at least five, at least six, at least seven, at least eight, at least nine, at least ten, at least 11, at least 12, at least 13, at least 14, at last 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 basic amino acids. In certain aspects, the cationic carrier moiety comprises about 30 to about 50 basic amino acids.

In some aspects, the basic amino acid comprises arginine, lysine, histidine, or any combination thereof. In some aspects, the cationic carrier moiety comprises about 40 lysine monomers.

In some aspects, the adjuvant moiety is capable of modulating an immune response, an inflammatory response, and/or a tissue microenvironment. In certain aspects, the adjuvant moiety comprises an imidazole derivative, an amino acid, a vitamin, or any combination thereof.

In some aspects, the adjuvant moiety comprises:

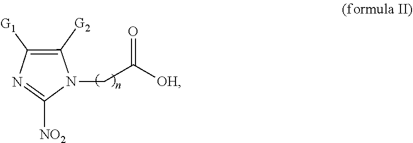

wherein each of G1 and G2 is H, an aromatic ring, or 1-10 alkyl, or G1 and G2 together form an aromatic ring, and wherein n is 1-10.

In some aspects, the adjuvant moiety comprises nitroimidazole. In certain aspects, the adjuvant moiety comprises metronidazole, tinidazole, nimorazole, dimetridazole, pretomanid, ornidazole, megazol, azanidazole, benznidazole, or any combination thereof.

In some aspects, the adjuvant moiety comprises an amino acid.

In some aspects, the adjuvant moiety comprises

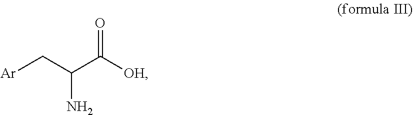

wherein Ar is

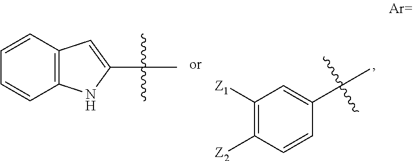

and

wherein each of Z1 and Z2 is H or OH.

In some aspects, the adjuvant moiety comprises a vitamin. In certain aspects, the vitamin comprises a cyclic ring or cyclic hetero atom ring and a carboxyl group or hydroxyl group.

In some aspects, the vitamin comprises:

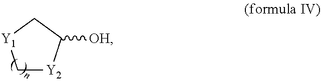

wherein each of Y1 and Y2 is C, N, O, or S, and wherein n is 1 or 2.

In some aspects, the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof. For example, the vitamin can be vitamin B3.

In some aspects, the adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, or at least about 20 vitamin B3. In certain aspects, the adjuvant moiety comprises about 10 vitamin B3.

In some aspects, the delivery agent comprises about a water-soluble biopolymer moiety with about 120 to about 130 PEG units, a cationic carrier moiety comprising a poly-lysine with about 30 to about 40 lysines, and an adjuvant moiety with about 5 to about 10 vitamin B3.

In some aspects, the delivery agent is associated with the miRNA inhibitor, thereby forming a micelle. For example, the association can be a covalent bond, a non-covalent bond, or an ionic bond.

In some aspects, the cationic carrier unit and the miRNA inhibitor in the micelle is mixed in a solution so that the ionic ratio of the positive charges of the cationic carrier unit and the negative charges of the miRNA inhibitor is about 1: 1. In some aspects, the cationic carrier unit is capable of protecting the miRNA inhibitor from enzymatic degradation.

In some aspects, a disease or a condition that can be treated with the present disclosure comprises Alzheimer's disease. In certain aspects, the disease or condition comprises autism spectrum disorder, mental retardation, seizure, stroke, Parkinson's disease, spinal cord injury, or any combination thereof. In certain aspects, the disease or condition is Parkinson's disease.

In some aspects, the delivery agent is a micelle. In some aspects, the micelle comprises (i) about 100 to about 200 PEG units, (ii) about 30 to about 40 lysines, each with an amine group, (iii) about 15 to about 20 lysines, each with a thiol group, and (iv) about 30 to about 40 lysines, each linked to vitamin B3. In some aspects, the micelle comprises (i) about 120 to about 130 PEG units, (ii) about 32 lysines, each with an amine group, (iii) about 16 lysines, each with a thiol group, and (iv) about 32 lysines, each linked to vitamin B3.

In some aspects, a targeting moiety is further linked to the PEG units. In some aspects, the targeting moiety is a LAT 1 targeting ligand. In some aspects, the targeting moiety is pennyl alanine.

### BRIEF DESCRIPTION OF THE DRAWINGS/FIGURES

FIG. 1 shows an exemplary architecture of a carrier unit of the present disclosure. The example presented includes a cationic carrier moiety, which can interact electrostatically with anionic payloads, e.g., nucleic acids such as antisense oligonucleotides targeting a gene, e.g., miRNA (antimirs). In some aspects, AM can be located between WP and CC. The CC and AM components are portrayed in a linear arrangement for simplicity. However, as exemplified in FIG. 4, CC and AM can be arranged in a scaffold fashion.

FIGS. 2A, 2B, 2C, and 2D shows that SIRT1 expression is decreased in Alzheimer's disease subjects. FIG. 2A provides a comparison of representative SIRT1 protein expression in precentral gyrus tissues from normal (i.e., subjects without AD) and AD patients (n=6 for each group). FIG. 2B provides a quantitative comparison of the results shown in FIG. 2A. SIRT1 bands were analyzed by densitometry and normalized to β-actin. Relative levels of SIRT1 protein are shown from control (n=6) or AD precentral gyrus (n=6) tissues. FIG. 2C provides a comparison of SIRT1 mRNA expression in in 6 mo-old wild-type (WT) (n=4), 6 mo-old 5×FAD (n=3), 11 mo-old wild-type (WT), and 11 mo-old 5×FAD mice (n=3). Comparative analyzes were performed for mice at the same ages. FIG. 2D provides a comparison of SIRT1 mRNA expression in 5×FAD mice by age. Each age group's 5×FAD expression was normalized to WT. In FIGS. 2B, 2C, and 2D, the bars represent mean±SD.

FIGS. 3A and 3B provide comparison of miR485-3p and miR485-5p expression in normal (i.e., subjects without AD) and AD patients, respectively.

FIG. 4 provides a comparison of relative levels of mouse miR485-3p expression in primary cortical neurons transfected with either the control oligonucleotide or the miR485 inhibitor. The graph on the left shows miR485-3p expression after treatment with miR485-3p ASO (also referred to herein as “miRNA inhibitor” or “miR-485 inhibitor”) for 3 hours. The graph on the right shows expression after treatment with miR485-3p ASO for 6 hours. In each of the graphs, the left bar represents the control group and the right bar represents the miR-485 inhibitor transfected group.

FIGS. 5A and 5B show that miR-485 inhibitors can increase SIRT1 and PGC-1α expression. FIG. 5A provides western blot results showing SIRT1 and PGC-1α protein expression in mouse primary cortical neurons transfected with miR-control, miR485-3p (“miR485-3p mimic”), or miR-485 inhibitor (“miR485-3p ASO”). FIG. 5B provides a quantitative comparison of the results shown in FIG. 5A.

FIGS. 6A, 6B, and 6C show that miR-485 inhibitor functionally binds to the 3′ UTR of SIRT1. FIG. 6A is a schematic representation of the wild type (WT) or mutant form (MT) in SIRT1 3′-UTR showing the putative miR-485-3p target site. FIG. 6B provides a comparison of the relative luciferase activity in HEK293T cells co-transfected with SIRT1 3′-UTR WT or MT reporter constructs and miR-control, miR-485-3p for 48 hours. At least three independent experiments were performed. FIG. 6C provides a comparison of the relative binding of miR485-3p onto 3′ UTR of SIRT1 harboring mutant seed region compared to WT 3′ UTR of SIRT1.

FIGS. 7A, 7B, 7C, 7D, 7E, 7F, and 7G show that the miR-485 inhibitor reduces Aβ deposition and alters APP processing. FIG. 7A provides the schedule of miR-485 inhibitor ICV injections in 10 mo-old 5×FAD mice. FIG. 7B provides representative images of immunohistochemistry staining for Aβ (6E10) in the cortex and hippocampal DG region from control (n=5) and miR-485 inhibitor (“miR485-3p ASO”) (n=5) injected 5×FAD mice. FIG. 7C provides a quantitative comparison (mean number of Aβ plaques per mm²) of the results shown in FIG. 7B. FIG. 7D provides immunoblot for insoluble Aβ fractions in control (n=3) or miR-485 inhibitor (“miR485-3p ASO”) (n=3) injected 10 mo-old 5×FAD mice. FIG. 7E provides a quantitative comparison of the data shown in FIG. 7D. The left bar represents the control group and the right bar represents the miR-485 inhibitor groups. FIG. 7F provides western blot showing APP, sAPPβ, sAPPα, β-CTFs, BACE1, Adam10, SIRT1 and PGC-1α protein expression in control (n=3) or miR-485 inhibitor (“miR485-3p ASO”) (n=3) injected 10 mo-old 5×FAD mice. FIG. 7G provides a quantitative comparison (i.e., relative levels) of the data shown in FIG. 7F. In each of the graphs shown in FIG. 7G, the left bar represents the control and the right bar represents the miR-485 inhibitor group.

FIGS. 8A, 8B, 8C, 8D, 8E, 8F, 8G, and 8H show that miR-485 inhibitor enhances phagocytosis of Aβ both in vitro and in vivo by increasing CD36 expression. FIG. 8A provides an immunohistochemistry analysis of Iba1 (microglia) and β-amyloid 1-16 (6E10, to detect Aβ plaque) on coronal sections of control (n=11 images from five mice) or miR485-3p ASO (“miR485-3p ASO”) (n=11 images from five mice) injected 5×FAD mice. FIG. 8B provides a quantitative comparison (mean number of Iba1⁺Aβ⁺ cells per mm²) of the data shown in FIG. 8A. FIG. 8C provides representative images of ThS staining to Aβ plaque in hippocampus and cortex of control (n=7 images from three mice) or miR-485 inhibitor (“miR485-3p ASO”) (n=7 images from three mice) administrated mice. FIG. 8D provides a quantitative comparison of the data shown in FIG. 8C. FIG. 8E provides an immunohistochemistry analysis showing the uptake of Aβ plaques (Aβ 1-42) by the primary glial cells (Iba1+) in mouse primary mixed glial cells transfected and/or treated with one of the following: (i) transfected with control oligonucleotide, (ii) treated with fAβ(1-42) (1 μM), or (iii) transfected with miR-485 inhibitor (“miR485-3p ASO”) and treated with fAβ(1-42) (1 μM). FIG. 8F provides immunohistochemistry analysis of histological brain sections from control (n=6) or miR-485 inhibitor (“miR485-3p ASO”) (n=6) injected 5×FAD mice using anti-Iba1, anti-CD68 (phagosome) and anti-β-amyloid 1-16 (6E10). FIG. 8G provides a quantitative comparison (mean number of Iba1⁺Aβ⁺ CD68⁺ cells per mm²) of the results shown in FIG. 8F. FIG. 8H provides a comparison of Aβ levels in supernatant of BV2 microglia cells transfected with either control oligonucleotide or miR-485 inhibitor (“miR485-3p ASO”) and further treated with fAβ(1-42) (1 μM). Supernatant was collected after 4 hours of treatment and analyzed using ELISA.

FIGS. 9A, 9B, 9C, 9D, and 9E show that miR-485 inhibitor can increase CD36 expression. FIG. 9A provides a comparison of the relative levels of CD36 protein expression in control (n=3) or miR-485 inhibitor (“miR485-3p ASO”) (n=3) injected 10 mo-old 5×FAD mice. FIG. 9B provides a quantitative comparison of the results shown in FIG. 9A. FIG. 9C provides an immunohistochemistry analysis of on histological brain sections from the control or miR-485 inhibitor (“miR485-3p ASO”) treated 5×FAD mice using anti-Iba1 and anti-β-amyloid 1-16 (6E10). FIG. 9D provides cell surface expression of CD36 as measured by flow cytometry using Alexa488-conjugated anti-CD36 antibody in control (n=3), miR485-3p mimic, or miR-485 inhibitor (“miR485-3p ASO”) (n=3) transfected primary mixed glial cells. FIG. 9E provides a quantitative comparison (relative mean fluorescence intensity) of the results shown in FIG. 9D.

FIG. 10 shows that miR-485 inhibitor can functionally bind to the 3′ UTR of CD36. Relative luciferase activity was measured in HEK293T cells co-transfected with CD36 3′-UTR WT or MT reporter constructs and miR-control or miR-485 inhibitor for 48 h.

FIG. 11 shows that miR-485 inhibitor can promote increased Aβ phagocytosis through CD36 regulation. Aβ levels in supernatant of BV2 microglia cells transfected with either control oligonucleotide or miR-485 inhibitor (“miR485-3p ASO”) and further treated with fAβ(1-42) (1 μM). Where indicated, a blocking anti-CD36 antibody was also added. Supernatant was collected after 4 hours of treatment and analyzed using ELISA.

FIGS. 12A, 12B, 12C, 12D, 12E, 12F, 12H, 12I, and 12J show that miR-485 inhibitor can reduce neuroinflammation in glial cells. FIG. 12A provides western blot analysis showing SIRT1, NF-κB (p65), TNF-α and IL-1β protein expression in control or miR-485 inhibitor (“miR485-3p ASO”) transfected primary mixed glial cells treated with fAβ(1-42) (1 μM) for 3 or 6 hours. “(1)” corresponds to cells transfected with the control oligonucleotide alone. “(2)” corresponds to cells treated with fAβ(1-42) alone. “(3)” corresponds to cells transfected with the miR-485 inhibitor and treated with fAβ(1-42). FIG. 12B provides a quantitative comparison of the results provided in FIG. 12A. In each of the graphs shown in FIG. 12B, the left bar represents the control, the middle bar represents cells treated with fAβ(1-42) alone, and the right bar represents the cells transfected with the miR-485 inhibitor and treated with fAβ(1-42). FIG. 12C provides immunoblot detection of Iba1, NF-κB (p65), TNF-α and IL-1b protein in control (n=3) or miR-485 inhibitor (“miR485-3p ASO”) (n=3) injected 10 mo-old 5×FAD mice. Results from two independent experiments are shown (i.e., Set #1 and Set #2). FIG. 12D provides a quantitative comparison of the results shown in FIG. 12C. In each of the graphs shown in FIG. 12D, the left bar represents the control and the right bar represents the miR-485 inhibitor group. FIG. 12E provides an immunohistochemistry analysis of Iba1 and TNF-α expression in the control (n=11 images from five mice) or miR-485 inhibitor (“miR485-3p ASO”) (n=11 images from five mice) injected 5×FAD mice. FIG. 12F provides a quantitative comparison (mean number of Iba1 and TNF-α-stained cells per mm²) of the results shown in FIG. 12E. FIG. 12G provides an immunohistochemistry analysis of Iba1 and IL-1β expression in the control (n=11 images from five mice) or miR-485 inhibitor (“miR485-3p ASO”) (n=11 images from five mice) injected 5×FAD mice. FIG. 12H provides a quantitative comparison (mean number of Iba1 and IL-1β-stained cells per mm²) of the results shown in FIG. 12G. FIGS. 12I and 12J provide comparison of the amount of TNF-α (FIG. 12I) and IL-1β (FIG. 12J) observed in the supernatant of primary mixed glial cells treated with mPFF (mouse alpha synuclein aggregation form; 1 μg/mL) and varying concentrations of miR-485 inhibitor (50 and 100 nM).

FIGS. 13A, 13B, 13C, 13D, 13E, 13F, and 13G show that miR-485 inhibitor ameliorates neuronal loss, promotes neurogenesis, and increases post-synapse. FIG. 13A provides immunoblot showing NeuN and cleaved caspase 3 protein expression in the hippocampus (left) and cortex (right) of control (n=3) or miR-485 inhibitor (“miR485-3p ASO”) (n=5) injected 10 mo-old 5×FAD mice. FIG. 13B provides a quantitative comparison of the results provided in FIG. 13A. FIG. 13C provides an immunohistochemistry analysis showing NeuN and cleaved caspase-3 expression in coronal brain sections from control (n=4) or miR-485 inhibitor (“miR485-3p ASO”) (n=5) injected 10 mo-old 5×FAD mice. FIG. 13D provides a quantitative comparison (mean number of NeuN and cleaved caspase-3-stained cells per mm²) of the results shown in FIG. 13C. FIG. 13E provides immunoblot analysis of PSD-95 protein expression in control (n=3) or miR-485 inhibitor (“miR485-3p ASO”) (n=5) injected 10 mo-old 5×FAD mice. Results from two independent experiments are shown (i.e., Set #1 and Set #2). FIG. 13F provides a quantitative comparison of the results shown in FIG. 13E. FIG. 13G provides a comparison of doublecortin (DCX)-positive cells in the brain tissue of control mice or 5×FAD mice treated with the miR-485 inhibitor.

FIGS. 14A and 14B show that miR-485 inhibitor improves cognitive decline in 5×FAD mice. FIGS. 14A and 14B provides the results from the Y-maze and passive avoidance tests, respectively for mice (10 mo-old 5×FAD mice) treated with either the control oligonucleotide or the miR-485 inhibitor (“miR485-3p ASO”). Average alternation (%) for control or miR485-3p injected 5×FAD mice and total entry number into each arm on Y-maze. Average step through latency and time in dark compartment in seconds for control or miR485-3p injected 5×FAD mice on passive avoidance test.

FIG. 15 provides a schematic diagram of possible non-limiting different means by which a miR-485 inhibitor can treat Alzheimer's disease as demonstrated through 5×FAD mice. miR-485 inhibitor in 5×FAD can increase SIRT1 expression in neurons. SIRT1 in turn can reduce amyloid beta production through regulation of amyloid production enzymes. Also, miR-485 inhibitor can enhance CD36 expression and phagocytosis of Aβ plaque in glial cells. At the same time, miR-485 inhibitor can induce SIRT1 expression and reduce neuroinflammation and neuronal damage.

FIGS. 16A, 16B, and 16C show that the expression of SIRT1 and PGC-1α increases in mouse brain cortex after a single intraventricular administration of a miR-485 inhibitor. FIG. 16A provides the expression level of SIRT1 (left graph) and PGC-1α (right graph) at 6, 24, 48, and 72 hours after administration of the miR-485 inhibitor (100 μg/mouse). FIGS. 16B and 16C show the positive correlation between SIRT1 and PGC-1α expression, respectively, and time over a course of about 50 hours. In each of FIGS. 16A, 16B, and 16C, SIRT1 and PGC-1α expression level are shown normalized to the control (i.e., expression level in mice not treated with the miR-485 inhibitor). The percent values provided in FIG. 16A represent the average percent increase in SIRT1 and PGC-1α expression over the control at 48 hours post miR-485 inhibitor administration. In FIG. 16A, the p values provided represent the p value oft test. In FIGS. 16B and 16C, the p values provided represent the p value of Pearson's correlation. “C.C” represents the correlation coefficient of Pearson's correlation.

FIGS. 17A, 17B, and 17C show that the expression of SIRT1 and PGC-1α increases in the hippocampus of mouse brain after a single intravenous administration of a miR-485 inhibitor. FIG. 17A provides the expression level of SIRT1 (left graph) and PGC-1α (right graph) at 6, 24, 48, and 72 hours after administration of the miR-485 inhibitor (100 μg/mouse). FIGS. 17B and 17C show the positive correlation between SIRT1 and PGC-1α expression, respectively, and time over a course of about 24 hours. In each of FIGS. 17A, 17B, and 17C, SIRT1 and PGC-1α expression level are shown normalized to the control (i.e., expression level in mice not treated with the miR-485 inhibitor). The percent values provided in FIG. 17A represent the average percent increase in SIRT1 and PGC-1α expression over the control at 24 hours post miR-485 inhibitor administration. In FIG. 17A, the p values provided represent the p value oft test. In FIGS. 17B and 17C, the p values provided represent the p value of Pearson's correlation. “C.C” represents the correlation coefficient of Pearson's correlation.

FIGS. 18A and 18B show that the expression of CD36 increases in mouse brain after a single after a single intravenous administration of a miR-485 inhibitor (100 μg/mouse). FIG. 18A provides the expression level of CD36 at 24, 48, 72, and 120 hours after administration of the miR-485 inhibitor (100 μg/mouse). FIG. 18B shows the positive correlation between CD36 expression and time over a course of about 80 hours. In each of FIGS. 18A and 18B, CD36 expression is shown normalized to the control (i.e., expression level in mice not treated with the miR-485 inhibitor). The percent value provided in FIG. 18A represents the average percent increase in CD36 expression over the control at 48 hours post miR-485 inhibitor administration. In FIG. 18A, the p values provided represent the p value of t test. In FIG. 18B, the p value provided represents the p value of Pearson's correlation. “C.C” represents the correlation coefficient of Pearson's correlation.

FIGS. 19A and 19B show that the administration of a miR-485 inhibitor has no observable effect on body weight of male and female rats, respectively. As shown, male and female rats received one of the following doses of the miR-485 inhibitor: (i) 0 mg/kg (G1), (ii) 3.75 mg/kg (G2), (iii) 7.5 mg/kg (G3), and (iv) 15 mg/kg (G4). Body weight was measured at days 0, 3, 7, and 14 post miR-485 inhibitor administration.

FIGS. 20A and 20B show that the administration of miR-485 inhibitor has no effect on mortality in male and female rats, respectively. As shown, male and female rats received one of the following doses of the miR-485 inhibitor: (i) 0 mg/kg (G1), (ii) 3.75 mg/kg (G2), (iii) 7.5 mg/kg (G3), and (iv) 15 mg/kg (G4). Mortality of the animals was measured daily from days 0 to 14 days post miR-485 inhibitor administration.

FIGS. 21A and 21B show that the administration of a miR-485 inhibitor has no lasting clinical adverse effects when administered to male and female rats, respectively. As shown, male and female rats received one of the following doses of the miR-485 inhibitor: (i) 0 mg/kg (G1), (ii) 3.75 mg/kg (G2), (iii) 7.5 mg/kg (G3), and (iv) 15 mg/kg (G4). The adverse effects measured included the following: (i) NOA (no observable abnormalities), (ii) congestion (tail), (iii) edema (face), (iv) edema (forelimb), and (v) edema (hind limb). Adverse effects were measured at 0 hour, 0.5 hour, 1 hour, 2 hours, 4 hours, 6 hours, 1 day, 3 days, 5 days, 8 days, 11 days, and 14 days post miR-485 inhibitor administration.

FIGS. 22A and 22B show that the administration of a miR-485 inhibitor has no observable pathological abnormalities in male and female rats, respectively. As shown, male and female rats received one of the following doses of the miR-485 inhibitor: (i) 0 mg/kg (G1), (ii) 3.75 mg/kg (G2), (iii) 7.5 mg/kg (G3), and (iv) 15 mg/kg (G4).

FIGS. 23A, 23B, 23C, 23D, 23E, 23F, 23G, 23H, 23I, 23J, and 23K show the therapeutic effects of miR-485 inhibitor administration in a Parkinson's disease mouse model (i.e., 6-OHDA mice). FIG. 23A provides a schematic of the experimental design. FIG. 23B provides a comparison of rotarod latency (time it took the animals to fall off the Rotarod-treadmill as described in the Examples) for 6-OHDA mice treated with PBS or miR-485 inhibitor. FIG. 23C provides a comparison of the latency to when the animals fall from the wired cage for 6-OHDA mice treated with PBS or miR-485 inhibitor. FIG. 23D provides a comparison of the time it takes to climb down the pole for 6-OHDA mice treated with PBS or miR-485 inhibitor. FIG. 23E provides a comparison of the number of foot slips (left graph) and the time it took to cross the length of the beam for 6-OHDA mice treated with PBS or miR-485 inhibitor. FIGS. 23F and 23H provide western blot analysis showing tyrosine hydroxylase (TH) expression in the substantia nigra (SN) and striatum (STR), respectively, of mice from the following groups: (i) wild-type mice treated with PBS (Con+PBS; 1^{st} and 3^{rd} columns from the left); (ii) 6-OHDA mice treated with PBS (Exp+PBS; 2^{nd} and 4^{th} columns from the left); (iii) wild-type mice treated with miR-485 inhibitor (Con+miR-485; 5^{th} and 7^{th} columns from the left); and (iv) 6-OHDA mice treated with miR-485 inhibitor (Exp+miR-485; 6^{th} and 8^{th} columns from the left). FIGS. 23G and 23I provide a quantitative comparison (relative TH expression) of the results shown in FIGS. 23F and 23H, respectively. FIG. 23J provide western blot analysis showing the expression of the following proteins as measured in the substantia nigra of mice from the different treatment groups: TNF-α, IL-1β, Iba1, GFAP, and β-actin (control). The different treatment groups were as follows: (i) wild-type mice treated with PBS (Con+PBS; 1^{st} and 3^{rd} columns from the left); (ii) 6-OHDA mice treated with PBS (Exp+PBS; 2^{nd} and 4^{th} columns from the left); (iii) wild-type mice treated with miR-485 inhibitor (Con+miR-485; 5^{th} and 7^{th} columns from the left); and (iv) 6-OHDA mice treated with miR-485 inhibitor (Exp+miR-485; 6^{th} and 8^{th} columns from the left). FIG. 23K provides a quantitative comparison of the IL-1β expression shown in FIG. 23J.

FIGS. 24A and 24B show the effect of miR-485 inhibitor on autophagy in primary cortical neurons and primary mixed glial cells, respectively. FIG. 24A provides western blot results comparing the expression of p62 and LC3B in primary cortical neurons treated with mPFF mouse alpha synuclein aggregation form; 1 μg/mL) and increasing concentrations of miR-485 inhibitor. In FIG. 24A, the gel on the left shows the results after 24-hour treatment, and the gel on the right shows the results after 48-hour treatment. FIG. 24B provides western blot results comparing the expression of p62 and LC3B in primary mixed glial cells treated with mPFF mouse alpha synuclein aggregation form; 1 μg/mL) and increasing concentrations of miR-485 inhibitor. In each of FIGS. 24A and 24B, the first column (from left) represents untreated cells (i.e., no mPFF and no miR-485 inhibitor), and the second column (from left) represents cells treated only the mPFF.

FIGS. 25A and 25B show viral vector injection sites and lentivirus induced miR-485-3p overexpression in the mouse hippocampus, respectively. FIG. 25A shows target bilateral viral vector injection sites (i.e., dentate gyrus (DG) and CA1 in posterior hippocampus). FIG. 25B shows green fluorescent protein (GFP) expression in posterior and anterior hippocampus DG and CA1.

FIG. 26 shows a scheme of rodent behavioral tests for cognition and memory. OFT (open field test), Y-MAZE, NORT (novel object recognition test), and PAT (passive avoidance test) were conducted on the days indicated.

FIGS. 27A and 27B show the results from the open field test (OFT) for either the lenti-control vector (n=8) (black bar) or lenti-miR485-3p vector (n=7) (gray bar) injected mice. FIG. 27A provides the total distance (cm) traveled for 30 minutes for control or lenti-miR485-3p vector injected mice. FIG. 27B provides the center zone activity (%) for control or lenti-miR485-3p vector injected mice. An error bar represents mean±standard error of the mean the mean (SEM). Statistical significance was determined by unpaired t-test, followed by Bonferroni post hoc statistic test.

FIGS. 28A and 28B show the results from the Y-maze test for either the lenti-control vector (n=8) (black bar) or lenti-miR485-3p vector (n=7) (gray bar) injected mice. FIG. 28A shows the total entry number into each arm on Y-maze and FIG. 28B shows average alternation (%) for control or lenti-miR485-3p injected mice. P value=0.795. An error bar represents mean±SEM. Statistical significance was determined by unpaired t-test, followed by Bonferroni post hoc statistic test.

FIGS. 29A, 29B, 29C, 29D, 29E, 29F, and 29G show the results from the novel object recognition test (NORT) for either the lenti-control vector or lenti-miR485-3p vector injected mice. FIG. 29A shows the novel object recognition test experimental scheme. Example 19 (under “novel objection recognition test”) provides a detailed description of the experimental scheme. FIG. 29B shows the preference of the animals from the different treatment groups for either the novel or familiar objects after the short-term memory test (object x miR485-3p interaction p value=0.1288; object p=0.5287, F(1.22)=0.4098; virus p=0.0143, F(1.22)=7.075; lenti-control n=11, lenti-mir485-3p n=9). FIG. 29C shows the preference of the animals from the different treatment groups for either the novel or familiar objects at day 3 after being placed in the chamber (or next day after object recognition training) (long-term memory test2) (object x mir485-3p interaction p value<0.0001, F(1.26)=33.75, object p=0.0459, F(1.26)=43.18; lenti-control n=8, lenti-mir485-3p n=7). FIG. 29D shows the preference of the animals from the different treatment groups for either the novel or familiar objects at day 24 after being placed in the chamber (or day 22 after objection recognition training) (long-term memory test3) (object x mir485-3p interaction p value=0.0169, F(1.26)=6.523, object p<0.0001, F(1.26)=37.29; lenti-control n=8, lenti-mir485-3p n=7). FIGS. 29E, 29F, and 29G provide the discrimination index (the ability to distinguish between new and familiar objects), based on the results provided in FIGS. 29B, 29C, and 29D, respectively. An error bar represents mean±SEM. Statistical significance was determined by a two-way Anova and unpaired t-test, followed by Bonferroni post hoc statistic test. N. S.=not significant.

FIG. 30 shows the results from the passive avoidance test (PAT), i.e., entry latency time (sec), for either the lenti-control vector (n=8) (black) or lenti-miR485-3p vector (n=7) (gray) injected mice. P value=0.18, An error bar represents mean±SEM. Statistical significance was determined by unpaired t-test, followed by Bonferroni post hoc statistic test.

FIGS. 31A and 31B show experimental design and results from testing amyloid beta (Aβ) production and neuron to neuron spreading of Aβ. FIG. 31A shows the experimental design as described in Example 19. FIG. 31B shows immunocytochemistry results for the lenti-control vector or lenti-miR485-3p transduced cells testing virus expression (3^{rd} and 6^{th} images, respectively) and amyloid beta (2^{nd} and 5^{th} images, respectively).

FIG. 32 shows results from testing cleaved tau (C3) production and neuron to neuron spreading of cleaved tau. It shows immunocytochemistry results for the lenti-control vector or lenti-miR485-3p transduced cells testing virus expression (3^{rd} and 6^{th} images, respectively) and cleaved tau production (C3) (2^{nd} and 5^{th} images, respectively).

FIGS. 33A and 33B show results from testing PSD-95 and synaptophysin protein expression, respectively. FIG. 33A shows immunocytochemistry results for the lenti-control vector or lenti-miR485-3p transduced cells testing virus expression (3rd and 6th images, respectively) and PSD-95 protein expression (2nd and 5th images, respectively). FIG. 33B shows immunocytochemistry results for the lenti-control vector or lenti-miR485-3p transduced cells testing virus expression (3rd and 6th images, respectively) and synaptophysin protein expression (2nd and 5th images, respectively).

FIG. 34 shows results from testing cleaved caspase 3 protein expression. It shows immunocytochemistry results for the lenti-control vector or lenti-miR485-3p transduced cells testing virus expression (3^{rd} and 6^{th} images, respectively) and cleaved caspase 3 expression (2^{nd} and 5^{th} images, respectively).

FIGS. 35A, 35B, and 35C show experimental design (FIG. 35A) and results from testing microglia cell specific marker (ionized calcium-binding adaptor protein-1 (Iba-1) (FIG. 35B)), and cleaved caspase 3 protein expression in mouse primary microglia cells (FIG. 35C). FIG. 35A shows the experimental design as described in Example 19. FIG. 35B shows immunocytochemistry results for the lenti-control vector or lenti-miR485-3p transduced cells testing virus expression (3^{rd} and 6^{th} images, respectively) and Iba-1 expression (2^{nd} and 5^{th} images, respectively). FIG. 35C shows immunocytochemistry results for the lenti-control vector or lenti-miR485-3p transduced cells testing virus expression (3^{rd} and 6^{th} images, respectively) and cleaved caspase 3 expression in mouse primary microglia cells (2^{nd} and 5^{th} images, respectively).

FIGS. 36A and 36B show results from testing astrocyte specific marker, glial fibrillary acidic protein (GFAP) and cleaved caspase 3 protein expression in mouse primary astrocytes, respectively. FIG. 36A shows immunocytochemistry results for the lenti-control vector or lenti-miR485-3p transduced cells testing virus expression (3rd and 6th images, respectively) and GFAP expression in mouse primary astrocytes (2nd and 5th images, respectively). FIG. 36B shows immunocytochemistry results for the lenti-control vector or lenti-miR485-3p transduced cells testing virus expression (3rd and 6th images, respectively) and cleaved caspase 3 protein expression in mouse primary astrocytes (2nd and 5th images, respectively).

FIGS. 37A, 37B, and 37C show experimental design (FIG. 37A) and results from testing microglia cell specific marker (ionized calcium-binding adaptor protein-1 (Iba-1) (FIG. 37B)), and cleaved caspase 3 protein expression in human microglia cells (FIG. 37C). FIG. 37A shows the experimental design as described in Example 19. FIG. 37B shows immunocytochemistry results for the lenti-control vector or lenti-miR485-3p transduced cells testing virus expression (3^{rd} and 6^{th} images, respectively) and Iba-1 expression (2^{nd} and 5^{th} images, respectively) in human microglia cells. FIG. 37C shows immunocytochemistry results for the lenti-control vector or lenti-miR485-3p transduced cells testing virus expression (3^{rd} and 6^{th} images, respectively) and cleaved caspase 3 expression in in human microglia cells (2^{nd} and 5^{th} images, respectively).

FIGS. 38A and 38B show results from testing astrocyte specific marker, glial fibrillary acidic protein (GFAP) and cleaved caspase 3 protein expression in human astrocytes, respectively. FIG. 38A shows immunocytochemistry results for the lenti-control vector or lenti-miR485-3p transduced cells testing virus expression (3rd and 6th images, respectively) and GFAP expression in human astrocytes (2nd and 5th images, respectively). FIG. 38B shows immunocytochemistry results for the lenti-control vector or lenti-miR485-3p transduced cells testing virus expression (3rd and 6th images, respectively) and cleaved caspase 3 protein expression in human astrocytes (2nd and 5th images, respectively).

FIGS. 39A, 39B, 39C, and 39D show the therapeutic effects of two different doses (2 mg/kg or 5 mg/kg) of miR-485 inhibitors in a Parkinson's disease mouse model (i.e., 6-OHDA). Healthy animals and 6-OHDA mice treated with PBS were used as controls. FIG. 39A provides a comparison of rotarod latency (time it took the animals to fall off the Rotarod-treadmill as described in the Examples). FIG. 39B provides a comparison of the time it takes to climb down the pole. FIG. 39C provides a comparison of the latency to when the animals fall from the wired cage. FIG. 39D provides a comparison of the number of foot slips that occurred in crossing the length of the beam. In each of the figures, “(1)” corresponds to the healthy control group; “(2)” corresponds to 6-OHDA mice treated with PBS; “(3)” corresponds to 6-OHDA mice treated with 2.5 mg/kg of miR-485 inhibitor; and “(4)” corresponds to 6-OHDA mice treated with 5 mg/kg of miR-485 inhibitor.

FIGS. 40A, 40B, 40C, and 40D show the effect of miR-485 inhibitor on autophagy in BV2 microglial cells. FIG. 40A provides western blot results comparing the expression of FOXO3a, LC3-I, and LC3-II proteins in the BV2 cells treated with fibrillar amyloid beta (oAβ) and transfected with varying doses of the miR-485 inhibitor (0 nM, 50 nM, 100 nM, or 300 nM). Cells that were neither treated with oAβ nor transfected with the miR-485 inhibitor were used as control. FIGS. 40B, 40C, and 40D provide quantitative comparison of the expression of FOXO3, p62, and LC3-II proteins, respectively, in BV2 cells from the treatment groups. In each of the figures, “(1)” corresponds to the control cells (i.e., neither treated with oAβ nor transfected with the miR-485 inhibitor); “(2)” corresponds to BV2 cells treated with oAβ alone; “(3)” corresponds to BV2 cells treated with oAβ and 50 nM of the miR-485 inhibitor; “(4)” corresponds to BV2 cells treated with oAβ and 100 nM of the miR-485 inhibitor; and “(5)” corresponds to BV2 cells treated with oAβ and 300 nM of the miR-485 inhibitor.

### DETAILED DESCRIPTION OF THE DISCLOSURE

The present disclosure is directed to the use of a miR-485 inhibitor, comprising a nucleotide sequence encoding a nucleotide molecule that comprises at least one miR-485 binding site, wherein the nucleotide molecule does not encode a protein. In some aspects, the miRNA binding site or sites can bind to endogenous miR-485, which inhibits and/or reduces the expression level of an endogenous SIRT1 protein and/or a SIRT1 gene. In some aspects, the binding of endogenous miR-485 to the miRNA binding site or sites can inhibit and/or reduce the expression level of an endogenous CD36 protein and/or a CD36 gene. Similarly, in some aspects, the binding of endogenous miR-485 to the miRNA binding site or sites can inhibit and/or reduce the expression level of an endogenous PGC-1α. In some aspects, the binding of endogenous miR-485 to the miRNA binding site or sites can inhibit and/or reduce the expression level of an endogenous LRRK2 protein and/or a LRRK2 gene. In some aspects, the binding of endogenous miR-485 to the miRNA binding site or sites can inhibit and/or reduce the expression level of an endogenous NRG1 protein and/or a NRG1 gene. In some aspects, the binding of endogenous miR-485 to the miRNA binding site or sites can inhibit and/or reduce the expression level of an endogenous STMN2 protein and/or a STMN2 gene. In some aspects, the binding of endogenous miR-485 to the miRNA binding site or sites can inhibit and/or reduce the expression level of an endogenous VLDLR protein and/or a VLDLR gene. In some aspects, the binding of endogenous miR-485 to the miRNA binding site or sites can inhibit and/or reduce the expression level of an endogenous NRXN1 protein and/or a NRXN1 gene. In some aspects, the binding of endogenous miR-485 to the miRNA binding site or sites can inhibit and/or reduce the expression level of an endogenous GRIA4 protein and/or a GRIA4 gene. In some aspects, the binding of endogenous miR-485 to the miRNA binding site or sites can inhibit and/or reduce the expression level of an endogenous NXPH1 protein and/or a NXPH1 gene. In some aspects, the binding of endogenous miR-485 to the miRNA binding site or sites can inhibit and/or reduce the expression level of an endogenous PSD-95 protein and/or a PSD-95 gene. In some aspects, the binding of endogenous miR-485 to the miRNA binding site or sites can inhibit and/or reduce the expression level of an endogenous synaptophysin protein and/or a synaptophysin gene. In some aspects, the binding of endogenous miR-485 to the miRNA binding site or sites can promote and/or increase the expression level of an endogenous caspase-3 protein and/or a caspase-3 gene.

Accordingly, in some aspects, the present disclosure is directed to a method of increasing a level of a SIRT1 protein and/or SIRT1 gene in a subject in need thereof comprising administering an miR-485 inhibitor to the subject. In further aspects, increasing the level of a SIRT1 protein and/or SIRT1 gene in a subject can be useful in treating a disease or condition associated with reduced levels of a SIRT1 protein and/or a SIRT1 gene (e.g., neurodegenerative diseases and disorders). In some aspects, the present disclosure is directed to a method of increasing a level of a CD36 protein and/or CD36 gene in a subject in need thereof comprising administering an miR-485 inhibitor to the subject. In further aspects, increasing the level of a CD36 protein and/or CD36 gene in a subject can be useful in treating a disease or condition associated with reduced levels of a CD36 protein and/or a CD36 gene (e.g., neurodegenerative diseases and disorders). In some aspects, the present disclosure is directed to a method of increasing a level of a PGC-1α protein and/or PGC-1α gene in a subject in need thereof comprising administering an miR-485 inhibitor to the subject. In further aspects, increasing the level of a PGC-1α protein and/or PGC-1α gene in a subject can be useful in treating a disease or condition associated with reduced levels of a PGC-1α protein and/or a PGC-1α gene (e.g., neurodegenerative diseases and disorders). In some aspects, the present disclosure is directed to a method of increasing a level of a LRRK2 protein and/or LRRK2 gene in a subject in need thereof comprising administering an miR-485 inhibitor to the subject. In further aspects, increasing the level of a LRRK2 protein and/or LRRK2 gene in a subject can be useful in treating a disease or condition associated with reduced levels of a LRRK2 protein and/or a LRRK2 gene (e.g., neurodegenerative diseases and disorders). In some aspects, the present disclosure is directed to a method of increasing a level of a NRG1 protein and/or NRG1 gene in a subject in need thereof comprising administering an miR-485 inhibitor to the subject. In further aspects, increasing the level of a NRG1 protein and/or NRG1 gene in a subject can be useful in treating a disease or condition associated with reduced levels of a NRG1 protein and/or a NRG1 gene (e.g., neurodegenerative diseases and disorders). In some aspects, the present disclosure is directed to a method of increasing a level of a STMN2 protein and/or STMN2 gene in a subject in need thereof comprising administering an miR-485 inhibitor to the subject. In further aspects, increasing the level of a STMN2 protein and/or STMN2 gene in a subject can be useful in treating a disease or condition associated with reduced levels of a STMN2 protein and/or a STMN2 gene (e.g., neurodegenerative diseases and disorders). In some aspects, the present disclosure is directed to a method of increasing a level of a VLDLR protein and/or VLDLR gene in a subject in need thereof comprising administering an miR-485 inhibitor to the subject. In further aspects, increasing the level of a VLDLR protein and/or VLDLR gene in a subject can be useful in treating a disease or condition associated with reduced levels of a VLDLR protein and/or a VLDLR gene (e.g., neurodegenerative diseases and disorders). In some aspects, the present disclosure is directed to a method of increasing a level of a NRXN1 protein and/or NRXN1 gene in a subject in need thereof comprising administering an miR-485 inhibitor to the subject. In further aspects, increasing the level of a NRXN1 protein and/or NRXN1 gene in a subject can be useful in treating a disease or condition associated with reduced levels of a NRXN1 protein and/or a NRXN1 gene (e.g., neurodegenerative diseases and disorders). In some aspects, the present disclosure is directed to a method of increasing a level of a GRIA4 protein and/or GRIA4 gene in a subject in need thereof comprising administering an miR-485 inhibitor to the subject. In further aspects, increasing the level of a GRIA4 protein and/or GRIA4 gene in a subject can be useful in treating a disease or condition associated with reduced levels of a GRIA4 protein and/or a GRIA4 gene (e.g., neurodegenerative diseases and disorders). In some aspects, the present disclosure is directed to a method of increasing a level of a NXPH1 protein and/or NXPH1 gene in a subject in need thereof comprising administering an miR-485 inhibitor to the subject. In further aspects, increasing the level of a NXPH1 protein and/or NXPH1 gene in a subject can be useful in treating a disease or condition associated with reduced levels of a NXPH1 protein and/or a NXPH1 gene (e.g., neurodegenerative diseases and disorders). In some aspects, the present disclosure is directed to a method of increasing a level of a PSD-95 protein and/or PSD-95 gene in a subject in need thereof comprising administering an miR-485 inhibitor to the subject. In further aspects, increasing the level of a PSD-95 protein and/or PSD-95 gene in a subject can be useful in treating a disease or condition associated with reduced levels of a PSD-95 protein and/or a PSD-95 gene (e.g., neurodegenerative diseases and disorders). In some aspects, the present disclosure is directed to a method of increasing a level of a synaptophysin protein and/or synaptophysin gene in a subject in need thereof comprising administering an miR-485 inhibitor to the subject. In further aspects, increasing the level of a synaptophysin protein and/or synaptophysin gene in a subject can be useful in treating a disease or condition associated with reduced levels of a synaptophysin protein and/or a synaptophysin gene (e.g., neurodegenerative diseases and disorders). In some aspects, the present disclosure is directed to a method of decreasing a level of a caspase-3 protein and/or caspase-3 gene in a subject in need thereof comprising administering an miR-485 inhibitor to the subject. In further aspects, decreasing the level of a caspase-3 protein and/or caspase-3 gene in a subject can be useful in treating a disease or condition associated with increased levels of a caspase-3 protein and/or a caspase-3 gene (e.g., neurodegenerative diseases and disorders).

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, ‘a’ represents adenine, ‘c’ represents cytosine, ‘g’ represents guanine, ‘t’ represents thymine, and ‘u’ represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). In some aspects, an “AAV” includes a derivative of a known AAV. In some aspects, an “AAV” includes a modified or an artificial AAV.

The terms “administration,” “administering,” and grammatical variants thereof refer to introducing a composition, such as a miRNA inhibitor of the present disclosure, into a subject via a pharmaceutically acceptable route. The introduction of a composition, such as a micelle comprising a miRNA inhibitor of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “associated with” refers to a close relationship between two or more entities or properties. For instance, when used to describe a disease or condition that can be treated with the present disclosure (e.g., disease or condition associated with an abnormal level of a SIRT1 protein and/or SIRT1 gene), the term “associated with” refers to an increased likelihood that a subject suffers from the disease or condition when the subject exhibits an abnormal expression of the protein and/or gene. In some aspects, the abnormal expression of the protein and/or gene causes the disease or condition. In some aspects, the abnormal expression does not necessarily cause but is correlated with the disease or condition. Non-limiting examples of suitable methods that can be used to determine whether a subject exhibits an abnormal expression of a protein and/or gene associated with a disease or condition are provided elsewherein the present disclosure.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95%, about 98%, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence can apply to the entire length of a polynucleotide or polypeptide or can apply to a portion, region or feature thereof.

The term “derived from,” as used herein, refers to a component that is isolated from or made using a specified molecule or organism, or information (e.g., amino acid or nucleic acid sequence) from the specified molecule or organism. For example, a nucleic acid sequence that is derived from a second nucleic acid sequence can include a nucleotide sequence that is identical or substantially similar to the nucleotide sequence of the second nucleic acid sequence. In the case of nucleotides or polypeptides, the derived species can be obtained by, for example, naturally occurring mutagenesis, artificial directed mutagenesis or artificial random mutagenesis. The mutagenesis used to derive nucleotides or polypeptides can be intentionally directed or intentionally random, or a mixture of each. The mutagenesis of a nucleotide or polypeptide to create a different nucleotide or polypeptide derived from the first can be a random event (e.g., caused by polymerase infidelity) and the identification of the derived nucleotide or polypeptide can be made by appropriate screening methods, e.g., as discussed herein. In some aspects, a nucleotide or amino acid sequence that is derived from a second nucleotide or amino acid sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% to the second nucleotide or amino acid sequence, respectively, wherein the first nucleotide or amino acid sequence retains the biological activity of the second nucleotide or amino acid sequence.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide.

The terms “complementary” and “complementarity” refer to two or more oligomers (i.e., each comprising a nucleobase sequence), or between an oligomer and a target gene, that are related with one another by Watson-Crick base-pairing rules. For example, the nucleobase sequence “T-G-A (5′→3′),” is complementary to the nucleobase sequence “A-C-T (3′→5′).” Complementarity can be “partial,” in which less than all of the nucleobases of a given nucleobase sequence are matched to the other nucleobase sequence according to base pairing rules. For example, in some aspects, complementarity between a given nucleobase sequence and the other nucleobase sequence can be about 70%, about 75%, about 80%, about 85%, about 90%, or about 95%. Accordingly, in certain aspects, the term “complementary” refers to at least about 80%, at least about 85%, at least about 90%, at least about 91%, at least about 92%, at least about 93%, at least about 94%, at least about 95%, at least about 96%, at least about 97%, at least about 98%, or at least about 99% match or complementarity to a target nucleic acid sequence (e.g., miR-485 nucleic acid sequence). Or, there can be “complete” or “perfect” (100%) complementarity between a given nucleobase sequence and the other nucleobase sequence to continue the example. In some aspects, the degree of complementarity between nucleobase sequences has significant effects on the efficiency and strength of hybridization between the sequences.

The term “downstream” refers to a nucleotide sequence that is located 3′ to a reference nucleotide sequence. In certain aspects, downstream nucleotide sequences relate to sequences that follow the starting point of transcription. For example, the translation initiation codon of a gene is located downstream of the start site of transcription.

The terms “excipient” and “carrier” are used interchangeably and refer to an inert substance added to a pharmaceutical composition to further facilitate administration of a compound, e.g., a miRNA inhibitor of the present disclosure.

The term “expression,” as used herein, refers to a process by which a polynucleotide produces a gene product, e.g., RNA or a polypeptide. It includes without limitation transcription of the polynucleotide into micro RNA binding site, small hairpin RNA (shRNA), small interfering RNA (siRNA), or any other RNA product. It includes, without limitation, transcription of the polynucleotide into messenger RNA (mRNA), and the translation of mRNA into a polypeptide. Expression produces a “gene product.” As used herein, a gene product can be, e.g., a nucleic acid, such as an RNA produced by transcription of a gene. As used herein, a gene product can be either a nucleic acid, RNA or miRNA produced by the transcription of a gene, or a polypeptide which is translated from a transcript. Gene products described herein further include nucleic acids with post transcriptional modifications, e.g., polyadenylation or splicing, or polypeptides with post translational modifications, e.g., phosphorylation, methylation, glycosylation, the addition of lipids, association with other protein subunits, or proteolytic cleavage.

As used herein, the term “homology” refers to the overall relatedness between polymeric molecules, e.g. between nucleic acid molecules. Generally, the term “homology” implies an evolutionary relationship between two molecules. Thus, two molecules that are homologous will have a common evolutionary ancestor. In the context of the present disclosure, the term homology encompasses both to identity and similarity.

In some aspects, polymeric molecules are considered to be “homologous” to one another if at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the monomers in the molecule are identical (exactly the same monomer) or are similar (conservative substitutions). The term “homologous” necessarily refers to a comparison between at least two sequences (e.g., polynucleotide sequences).

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

As used herein, the term “identity” refers to the overall monomer conservation between polymeric molecules, e.g., between polynucleotide molecules. The term “identical” without any additional qualifiers, e.g., polynucleotide A is identical to polynucleotide B, implies the polynucleotide sequences are 100% identical (100% sequence identity). Describing two sequences as, e.g.,“70% identical,” is equivalent to describing them as having, e.g., “70% sequence identity.”

Calculation of the percent identity of two polypeptide or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions, or bases in the case of polynucleotides, are then compared.

When a position in the first sequence is occupied by the same amino acid or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs that can be used to align different sequences (e.g., polynucleotide sequences) are available from various sources. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at worldwideweb.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (%ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at worldwideweb.tcoffee.org, and alternatively available, e.g., from the EBI. It will also be appreciated that the final alignment used to calculate percent sequence identity can be curated either automatically or manually.

As used herein, the terms “isolated,” “purified,” “extracted,” and grammatical variants thereof are used interchangeably and refer to the state of a preparation of desired composition of the present disclosure, e.g., a miRNA inhibitor of the present disclosure, that has undergone one or more processes of purification. In some aspects, isolating or purifying as used herein is the process of removing, partially removing (e.g., a fraction) of a composition of the present disclosure, e.g., a miRNA inhibitor of the present disclosure from a sample containing contaminants.

In some aspects, an isolated composition has no detectable undesired activity or, alternatively, the level or amount of the undesired activity is at or below an acceptable level or amount. In other aspects, an isolated composition has an amount and/or concentration of desired composition of the present disclosure, at or above an acceptable amount and/or concentration and/or activity. In other aspects, the isolated composition is enriched as compared to the starting material from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material.

In some aspects, isolated preparations are substantially free of residual biological products. In some aspects, the isolated preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites.

The term “linked” as used herein refers to a first amino acid sequence or polynucleotide sequence covalently or non-covalently joined to a second amino acid sequence or polynucleotide sequence, respectively. The first amino acid or polynucleotide sequence can be directly joined or juxtaposed to the second amino acid or polynucleotide sequence or alternatively an intervening sequence can covalently join the first sequence to the second sequence. The term “linked” means not only a fusion of a first polynucleotide sequence to a second polynucleotide sequence at the 5′-end or the 3′-end, but also includes insertion of the whole first polynucleotide sequence (or the second polynucleotide sequence) into any two nucleotides in the second polynucleotide sequence (or the first polynucleotide sequence, respectively). The first polynucleotide sequence can be linked to a second polynucleotide sequence by a phosphodiester bond or a linker. The linker can be, e.g., a polynucleotide.

A “miRNA inhibitor,” as used herein, refers to a compound that can decrease, alter, and/or modulate miRNA expression, function, and/or activity. The miRNA inhibitor can be a polynucleotide sequence that is at least partially complementary to the target miRNA nucleic acid sequence, such that the miRNA inhibitor hybridizes to the target miRNA sequence. For instance, in some aspects, a miR-485 inhibitor of the present disclosure comprises a nucleotide sequence encoding a nucleotide molecule that is at least partially complementary to the target miR-485 nucleic acid sequence, such that the miR-485 inhibitor hybridizes to the miR-485 sequence. In further aspects, the hybridization of the miR-485 to the miR-485 sequence decreases, alters, and/or modulates the expression, function, and/or activity of miR-485 (e.g., hybridization results in an increase in the expression of SIRT1 protein and/or SIRT1 gene).

The terms “miRNA,” “miR,” and “microRNA” are used interchangeably and refer to a microRNA molecule found in eukaryotes that is involved in RNA-based gene regulation. The term will be used to refer to the single-stranded RNA molecule processed from a precursor. In some aspects, the term “antisense oligomers” can also be used to describe the microRNA molecules of the present disclosure. Names of miRNAs and their sequences related to the present disclosure are provided herein. MicroRNAs recognize and bind to target mRNAs through imperfect base pairing leading to destabilization or translational inhibition of the target mRNA and thereby downregulate target gene expression. Conversely, targeting miRNAs via molecules comprising a miRNA binding site (generally a molecule comprising a sequence complementary to the seed region of the miRNA) can reduce or inhibit the miRNA-induced translational inhibition leading to an upregulation of the target gene.

The terms “mismatch” or “mismatches” refer to one or more nucleobases (whether contiguous or separate) in an oligomer nucleobase sequence (e.g., miR-485 inhibitor) that are not matched to a target nucleic acid sequence (e.g., miR-485) according to base pairing rules. While perfect complementarity is often desired, in some aspects, one or more (e.g., 6, 5, 4, 3, 2, or 1 mismatches) can occur with respect to the target nucleic acid sequence. Variations at any location within the oligomer are included. In certain aspects, antisense oligomers of the disclosure (e.g., miR-485 inhibitor) include variations in nucleobase sequence near the termini, variations in the interior, and if present are typically within about 6, 5, 4, 3, 2, or 1 subunit of the 5′ and/or 3′ terminus. In some aspects, one, two, or three nucleobases can be removed and still provide on-target binding.

As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances, a modulator can increase and/or decrease a certain concentration, level, activity or function relative to a control, or relative to the average level of activity that would generally be expected or relative to a control level of activity. In some aspects, a miRNA inhibitor disclosed herein, e.g., a miR-485 inhibitor, can modulate (e.g., decrease, alter, or abolish) miR-485 expression, function, and/or activity, and thereby, modulate SIRT1 protein or gene expression and/or activity.

“Nucleic acid,” “nucleic acid molecule,” “nucleotide sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein.

The terms “pharmaceutically acceptable carrier,” “pharmaceutically acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “pharmaceutical composition” refers to one or more of the compounds described herein, such as, e.g., a miRNA inhibitor of the present disclosure, mixed or intermingled with, or suspended in one or more other chemical components, such as pharmaceutically acceptable carriers and excipients. One purpose of a pharmaceutical composition is to facilitate administration of preparations comprising a miRNA inhibitor of the present disclosure to a subject.

The term “polynucleotide,” as used herein, refers to polymers of nucleotides of any length, including ribonucleotides, deoxyribonucleotides, analogs thereof, or mixtures thereof.

In some aspects, the term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide.

In some aspects, the term “polynucleotide” includes polydeoxyribonucleotides (containing 2-deoxy-D-ribose), polyribonucleotides (containing D-ribose), including tRNA, rRNA, shRNA, siRNA, miRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

In some aspects of the present disclosure, a polynucleotide can be, e.g., an oligonucleotide, such as an antisense oligonucleotide. In some aspects, the oligonucleotide is an RNA. In some aspects, the RNA is a synthetic RNA. In some aspects, the synthetic RNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine).

The terms “polypeptide,” “peptide,” and “protein” are used interchangeably herein to refer to polymers of amino acids of any length, e.g., that are encoded by the SIRT 1 gene. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. The term “polypeptide,” as used herein, refers to proteins, polypeptides, and peptides of any size, structure, or function.

Polypeptides include gene products, naturally occurring polypeptides, synthetic polypeptides, homologs, orthologs, paralogs, fragments and other equivalents, variants, and analogs of the foregoing.

A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to about 50 amino acids long, e.g., about 5, about 10, about 15, about 20, about 25, about 30, about 35, about 40, about 45, or about 50 amino acids long.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

As used herein, the terms “promoter” and “promoter sequence” are interchangeable and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. In some aspects, a promoter that can be used with the present disclosure includes a tissue specific promoter.

As used herein, “prophylactic” refers to a therapeutic or course of action used to prevent the onset of a disease or condition, or to prevent or delay a symptom associated with a disease or condition.

As used herein, a “prophylaxis” refers to a measure taken to maintain health and prevent the onset of a disease or condition, or to prevent or delay a symptom associated with a disease or condition.

As used herein, the term “gene regulatory region” or “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites, or stem-loop structures. If a coding region is intended for expression in a eukaryotic cell, a polyadenylation signal and transcription termination sequence will usually be located 3′ to the coding sequence.

In some aspects, a miR-485 inhibitor disclosed herein (e.g., a polynucleotide encoding a RNA comprising one or more miR-485 binding site) can include a promoter and/or other expression (e.g., transcription) control elements operably associated with one or more coding regions. In an operable association a coding region for a gene product is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other expression control elements, besides a promoter, for example enhancers, operators, repressors, and transcription termination signals, can also be operably associated with a coding region to direct gene product expression.

As used herein, the term “similarity” refers to the overall relatedness between polymeric molecules, e.g. between polynucleotide molecules (e.g. miRNA molecules). Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the nucleic acids are compared, e.g., according to their evolutionary proximity, charge, volume, flexibility, polarity, hydrophobicity, aromaticity, isoelectric point, antigenicity, or combinations thereof.

The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications.

As used herein, the phrase “subject in need thereof” includes subjects, such as mammalian subjects, that would benefit from administration of a miRNA inhibitor of the disclosure (e.g., miR-485 inhibitor), e.g., to increase the expression level of SIRT1 protein and/or SIRT1 gene.

As used herein, the term “therapeutically effective amount” is the amount of reagent or pharmaceutical compound comprising a miRNA inhibitor of the present disclosure that is sufficient to a produce a desired therapeutic effect, pharmacologic and/or physiologic effect on a subject in need thereof. A therapeutically effective amount can be a “prophylactically effective amount” as prophylaxis can be considered therapy.

The terms “treat,” “treatment,” or “treating,” as used herein refers to, e.g., the reduction in severity of a disease or condition; the reduction in the duration of a disease course; the amelioration or elimination of one or more symptoms associated with a disease or condition (e.g., diabetes); the provision of beneficial effects to a subject with a disease or condition, without necessarily curing the disease or condition. The term also includes prophylaxis or prevention of a disease or condition or its symptoms thereof.

The term “upstream” refers to a nucleotide sequence that is located 5′ to a reference nucleotide sequence.

A “vector” refers to any vehicle for the cloning of and/or transfer of a nucleic acid into a host cell. A vector can be a replicon to which another nucleic acid segment can be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters.

### II. Methods of Use

In some aspects, miR-485 inhibitors of the present disclosure can exert therapeutic effects (e.g., in a subject suffering from a neurodegenerative disease) by regulating the expression and/or activity of one or more genes. As described herein, in some aspects, miR-485 inhibitors disclosed herein are capable of regulating the expression and/or activity of a gene selected from SIRT1, CD36, PGC1, LRRK2, NRG1, STMN2, VLDLR, NRXN1, GRIA4, NXPH1, DLG4 (also referred to herein as “PSD-95 gene”), SYP (also referred to herein as “synaptophysin gene”), CASP3 (also referred to herein as “caspase-3 gene”), or combinations thereof. Not to be bound by any one theory, through such regulation, the miR-485 inhibitors can affect many biological processes, including, but not limited to, cellular homeostasis (e.g., CD36, SIRT1, PGC1α), protein homeostasis (e.g., LRRK2 and SIRT1), those associated with the autophagy-lysosomal pathway (e.g., SIRT1 and CD36), phagocytosis (e.g., CD36), glial biology (e.g., CD36 and SIRT1), neurogenesis/synaptogenesis (e.g., SIRT1, PGC1α, STMN2, and NRG1) neuroinflammation (e.g., CD36 and SIRT1), those associated with the mitochondria (e.g., PGC1α), and combinations thereof (e.g., SIRT1 and PGC1α).

SIRT1 Regulation

In some aspects, the present disclosure provides a method of increasing an expression of a SIRT1 protein and/or a SIRT1 gene in a subject in need thereof, comprising administering to the subject a compound that inhibits miR-485 activity (i.e., miR-485 inhibitor). In certain aspects, inhibiting miR-485 activity increases the expression of a SIRT1 protein and/or SIRT1 gene in the subject.

Sirtuin 1 (SIRT1), also known as NAD-dependent deacetylase sirtuin-1, is a protein that in humans is encoded by the SIRT1 gene. The SIRT1 gene is located on chromosome 10 in humans (nucleotides 67,884,656 to 67,918,390 of GenBank Accession Number NC_000010.11, plus strand orientation). Synonyms of the SIRT1 gene, and the encoded protein thereof, are known and include “regulatory protein SIR2 homolog 1,” “silent mating-type information regulation 2 homolog 1,” “SIR2,” “SIR2-Like Protein 1,” “SIR2L1,” “SIR2alpha,” “Sirtuin Type 1,” “hSIRT1,” or “hSIR2.”

There are at least two known isoforms of human SIRT1 protein, resulting from alternative splicing. SIRT1 isoform 1 (UniProt identifier: Q96EB6-1) consists of 747 amino acids and has been chosen as the canonical sequence (SEQ ID NO: 31). SIRT1 isoform 2 (also known as “delta-exon8) (UniProt identifier: Q96EB6-2) consists of 561 amino acids and differs from the canonical sequence as follows: 454-639: missing (SEQ ID NO: 32). Table 1 below provides the sequences for the two SIRT1 isoforms.

TABLE 1
SIRT1 Protein Isoforms

| | |
|---|---|
| Isoform 1 | MADEAALALQPGGSPSAAGADREAASSPAGEPLRKRPRRDGPGLERSPGEPGGAAPEREV |
| (UniProt: | PAAARGCPGAAAAALWREAEAEAAAAGGEQEAQATAAAGEGDNGPGLQGPSREPPLADNL |
| Q96EB6-1) | YDEDDDDEGEEEEEAAAAAIGYRDNLLFGDEIITNGFHSCESDEEDRASHASSSDWTPRP |
| (SEQ ID NO: 31) | RIGPYTFVQQHLMIGTDPRTILKDLLPETIPPPELDDMTLWQIVINILSEPPKRKKRKDI |
| | NTIEDAVKLLQECKKIIVLTGAGVSVSCGIPDFRSRDGIYARLAVDFPDLPDPQAMFDIE |
| | YFRKDPRPFFKFAKEIYPGQFQPSLCHKFIALSDKEGKLLRNYTQNIDTLEQVAGIQRII |
| | QCHGSFATASCLICKYKVDCEAVRGDIFNQVVPRCPRCPADEPLAIMKPEIVFFGENLPE |
| | QFHRAMKYDKDEVDLLIVIGSSLKVRPVALIPSSIPHEVPQILINREPLPHLHFDVELLG |
| | DCDVIINELCHRLGGEYAKLCCNPVKLSEITEKPPRTQKELAYLSELPPTPLHVSEDSSS |
| | PERTSPPDSSVIVTLLDQAAKSNDDLDVSESKGCMEEKPQEVQTSRNVESIAEQMENPDL |
| | KNVGSSTGEKNERTSVAGTVRKCWPNRVAKEQISRRLDGNQYLFLPPNRYIFHGAEVYSD |
| | SEDDVLSSSSCGSNSDSGTCQSPSLEEPMEDESEIEEFYNGLEDEPDVPERAGGAGFGTD |
| | GDDQEAINEAISVKQEVTDMNYPSNKS |
| Isoform 2 | MADEAALALQPGGSPSAAGADREAASSPAGEPLRKRPRRDGPGLERSPGEPGGAAPEREV |
| (UniProt: | PAAARGCPGAAAAALWREAEAEAAAAGGEQEAQATAAAGEGDNGPGLQGPSREPPLADNL |
| Q96EB6-2) | YDEDDDDEGEEEEEAAAAAIGYRDNLLFGDEIITNGFHSCESDEEDRASHASSSDWTPRP |
| (SEQ ID NO: 32) | RIGPYTFVQQHLMIGTDPRTILKDLLPETIPPPELDDMTLWQIVINILSEPPKRKKRKDI |
| | NTIEDAVKLLQECKKIIVLTGAGVSVSCGIPDFRSRDGIYARLAVDFPDLPDPQAMFDIE |
| | YFRKDPRPFFKFAKEIYPGQFQPSLCHKFIALSDKEGKLLRNYTQNIDTLEQVAGIQRII |
| | QCHGSFATASCLICKYKVDCEAVRGDIFNQVVPRCPRCPADEPLAIMKPEIVFFGENLPE |
| | QFHRAMKYDKDEVDLLIVIGSSLKVRPVALIPSNQYLFLPPNRYIFHGAEVYSDSEDDVL |
| | SSSSCGSNSDSGTCQSPSLEEPMEDESEIEEFYNGLEDEPDVPERAGGAGFGTDGDDQEA |
| | INEAISVKQEVTDMNYPSNKS |

As used herein, the term “SIRT1” (including its synonyms) includes any variants or isoforms of SIRT1 which are naturally expressed by cells. Accordingly, in some aspects, a miR-485 inhibitor disclosed herein can increase the expression of SIRT1 isoform 1. In some aspects, a miR-485 inhibitor disclosed herein can increase the expression of SIRT1 isoform 2. In further aspects, a miR-485 inhibitor disclosed herein can increase the expression of both SIRT1 isoform 1 and isoform 2. Unless indicated otherwise, both isoform 1 and isoform 2 are collectively referred to herein as “SIRT1.”

In some aspects, a miR-485 inhibitor of the present disclosure increases the expression of SIRT1 protein and/or SIRT1 gene by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% compared to a reference (e.g., expression of SIRT1 protein and/or SIRT1 gene in a corresponding subject that did not receive an administration of the miR-485 inhibitor).

Not to be bound by any one theory, in some aspects, a miR-485 inhibitor disclosed herein increases the expression of SIRT1 protein and/or SIRT1 gene by reducing the expression and/or activity of miR-485, e.g., miR-485-3p. In some aspects, a miR-485 inhibitor of the present disclosure can reduce the expression and/or activity of miR-485-3p.

In some aspects, a miR-485 inhibitor disclosed herein decreases the expression and/or activity of miR-485-3p by at least about 5%, at least about 10%, at least about 20%, at least about 30%, at least about 40%, at least about 50%, at least about 60%, at least about 70%, at least about 80%, at least about 90%, or about 100% compared to a reference (e.g., miR-485-3p expression in a corresponding subject that did not receive an administration of the miR-485 inhibitor). In certain aspects, a miR-485 inhibitor disclosed herein decreases the expression and/or activity of miR-485-5p by at least about 5%, at least about 10%, at least about 20%, at least about 30%, at least about 40%, at least about 50%, at least about 60%, at least about 70%, at least about 80%, at least about 90%, or about 100% compared to a reference (e.g., miR-485-5p expression in a corresponding subject that did not receive an administration of the miR-485 inhibitor). In further aspects, a miR-485 inhibitor disclosed herein decreases the expression and/or activity of both miR-485-3p and miR-485-5p by at least about 5%, at least about 10%, at least about 20%, at least about 30%, at least about 40%, at least about 50%, at least about 60%, at least about 70%, at least about 80%, at least about 90%, or about 100% compared to a reference (e.g., miR-485-3p and miR-485-5p expression in a corresponding subject that did not receive an administration of the miR-485 inhibitor). In some aspects, the expression of miR-485-3p and/or miR-485-5p is completely inhibited after the administration of the miR-485 inhibitor.

As described herein, a miR-485 inhibitor of the present disclosure can increase the expression of SIRT1 protein and/or SIRT1 gene when administered to a subject. Accordingly, in some aspects, the present disclosure provides a method of treating a disease or condition associated with an abnormal (e.g., reduced) level of a SIRT1 protein and/or SIRT1 gene in a subject in need thereof. In certain aspects, the method comprises administering to the subject a compound that inhibits miR-485 activity (i.e., miR-485 inhibitor), wherein the miR-485 inhibitor increases the level of the SIRT1 protein and/or SIRT1 gene.

CD36 Regulation

As described herein, Applicant has identified that the human CD36 3′-UTR comprises a target site for miR-485-3p and that the binding of miR-485-3p can decrease CD36 expression (see, e.g., Examples 7 and 8). Accordingly, in some aspects, the present disclosure provides a method of increasing an expression of a CD36 protein and/or a CD36 gene in a subject in need thereof, comprising administering to the subject a compound that inhibits miR-485 activity (i.e., miR-485 inhibitor). In certain aspects, inhibiting miR-485 activity increases the expression of a CD36 protein and/or CD36 gene in the subject.

Cluster determinant 36 (CD36) is also known as platelet glycoprotein 4, is a protein that in humans is encoded by the CD36 gene. The CD36 gene is located on chromosome 7 (nucleotides 80,602,656 to 80,679,277 of GenBank Accession Number NC_000007.14, plus strand orientation). Synonyms of the CD36 gene, and the encoded protein thereof, are known and include “platelet glycoprotein IV,” “fatty acid translocase,” “scavenger receptor class B member 3,” “glycoprotein 88,” “glycoprotein IIIb,” “glycoprotein IV,” “thrombospondin receptor,” “GPIIIB,” “PAS IV,” “GP3B,” “GPIV,” “FAT,” “GP4,” “BDPLT10,” “SCARB3,” “CHDS7,” “PASIV,” or “PAS-4.”

There are at least four known isoform of human CD36 protein, resulting from alternative splicing. CD36 isoform 1 (UniProt identifier: P16671-1) consists of 472 amino acids and has been chosen as the canonical sequence (SEQ ID NO: 36). CD36 isoform 2 (also known as “ex8-del”) (UniProt identifier: P16671-2) consists of 288 amino acids and differs from the canonical sequence as follows: 274-288: SIYAVFESDVNLKGI→ETCVHFTSSFSVCKS; and 289-472: missing (SEQ ID NO: 37). CD36 Isoform 3 (also known as “ex6-7-del”) (UniProt identifier: P16671-3) consists of 433 amino acids and differs from the canonical sequence as follows: 234-272: missing (SEQ ID NO: 38). CD36 isoform 4 (also known as “ex4-del” (UniProt identifier: P16671-4) consists of 412 amino acids and differs from the canonical sequence as follows: 144-203: missing (SEQ ID NO: 39). Table 2 below provides the sequences for the four CD36 isoforms.

TABLE 2
CD36 Protein Isoforms

| | |
|---|---|
| Isoform 1 | MGCDRNCGLIAGAVIGAVLAVFGGILMPVGDLLIQKTIKKQVVLEEGTIAFKNWVKTGTE |
| (UniProt: P16671-1) | VYRQFWIFDVQNPQEVMMNSSNIQVKQRGPYTYRVRFLAKENVTQDAEDNTVSFLQPNGA |
| (SEQ ID NO: 36) | IFEPSLSVGTEADNFTVLNLAVAAASHIYQNQFVQMILNSLINKSKSSMFQVRTLRELLW |
| 36) | GYRDPFLSLVPYPVTTTVGLFYPYNNTADGVYKVFNGKDNISKVAIIDTYKGKRNLSYWE |
| | SHCDMINGTDAASFPPFVEKSQVLQFFSSDICRSIYAVFESDVNLKGIPVYRFVLPSKAF |
| | ASPVENPDNYCFCTEKIISKNCTSYGVLDISKCKEGRPVYISLPHFLYASPDVSEPIDGL |
| | NPNEEEHRTYLDIEPITGFTLQFAKRLQVNLLVKPSEKIQVLKNLKRNYIVPILWLNETG |
| | TIGDEKANMFRSQVTGKINLLGLIEMILLSVGVVMFVAFMISYCACRSKTIK |
| Isoform 2 | MGCDRNCGLIAGAVIGAVLAVFGGILMPVGDLLIQKTIKKQVVLEEGTIAFKNWVKTGTE |
| (UniProt: P16671-2) | VYRQFWIFDVQNPQEVMMNSSN1QVKQRGPYTYRVRFLAKENVTQDAEDNTVSFLQPNGA |
| (SEQ ID NO: 37) | IFEPSLSVGTEADNFTVLNLAVAAASHIYQNQFVQMILNSLINKSKSSMFQVRTLRELLW |
| | GYRDPFLSLVPYPVTTTVGLFYPYNNTADGVYKVFNGKDNISKVAIIDTYKGKRNLSYWE |
| | SHCDMINGTDAASFPPFVEKSQVLQFFSSDICRETCVHFTSSFSVCKS |
| Isoform 3 | MGCDRNCGLIAGAVIGAVLAVFGGILMPVGDLLIQKTIKKQVVLEEGTIAFKNWVKTGTE |
| (UniProt: P16671-3) | VYRQFWIFDVQNPQEVMMNSSNIQVKQRGPYTYRVRFLAKENVTQDAEDNTVSFLQPNGA |
| (SEQ ID NO: 38) | IFEPSLSVGTEADNFTVLNLAVAAASHIYQNQFVQMILNSLINKSKSSMFQVRTLRELLW |
| | GYRDPFLSLVPYPVTTTVGLFYPYNNTADGVYKVFNGKDNISKVAIIDTYKGKRSIYAVF |
| | ESDVNLKGIPVYRFVLPSKAFASPVENPDNYCFCTEKIISKNCTSYGVLDISKCKEGRPV |
| | YISLPHFLYASPDVSEPIDGLNPNEEEHRTYLDIEPITGFTLQFAKRLQVNLLVKPSEKI |
| | QVLKNLKRNYIVPILWLNETGTIGDEKANMFRSQVTGKINLLGLIEMILLSVGVVMFVAF |
| | MISYCACRSKTIK |
| Isoform 4 | MGCDRNCGLIAGAVIGAVLAVFGGILMPVGDLLIQKTIKKQVVLEEGTIAFKNWVKTGTE |
| (UniProt: P16671-4) | VYRQFWIFDVQNPQEVMMNSSNIQVKQRGPYTYRVRFLAKENVTQDAEDNTVSFLQPNGA |
| (SEQ ID NO: 39) | IFEPSLSVGTEADNFTVLNLAVAYNNTADGVYKVFNGKDNISKVAIIDTYKGKRNLSYWE |
| | SHCDMINGTDAASFPPFVEKSQVLQFFSSDICRSIYAVFESDVNLKGIPVYRFVLPSKAF |
| | ASPVENPDNYCFCTEKIISKNCTSYGVLDISKCKEGRPVYISLPHFLYASPDVSEPIDGL |
| | NPNEEEHRTYLDIEPITGFTLQFAKRLQVNLLVKPSEKIQVLKNLKRNYIVPILWLNETG |
| | TIGDEKANMFRSQVTGKINLLGLIEMILLSVGVVMFVAFMISYCACRSKTIK |

As used herein, the term “CD36” (including its synonyms) includes any variants or isoforms of CD36 which are naturally expressed by cells. Accordingly, in some aspects, a miR-485 inhibitor disclosed herein can increase the expression of CD36 isoform 1. In some aspects, a miR-485 inhibitor disclosed herein can increase the expression of CD36 isoform 2. In some aspect, a miR-485 inhibitor disclosed herein can increase the expression of CD36 isoform 3. In some aspects, a miR-485 inhibitor disclosed herein can increase the expression of CD36 isoform 4. In further aspects, a miR-485 inhibitor disclosed herein can increase the expression of both CD36 isoform 1 and isoform 2, and/or isoform 3 and isoform 4, and/or isoform 1 and isoform 4, and/or isoform 2 and isoform 3. In some aspects, a miR-485 inhibitor disclosed herein can increase the expression of all CD36 isoforms. Unless indicated otherwise, isoform 1, isoform 2, isoform 3, and isoform 4 are collectively referred to herein as “CD36.”

In some aspects, a miR-485 inhibitor of the present disclosure increases the expression of CD36 protein and/or CD36 gene by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% compared to a reference (e.g., expression of CD36 protein and/or CD36 gene in a corresponding subject that did not receive an administration of the miR-485 inhibitor).

Not to be bound by any one theory, in some aspects, a miR-485 inhibitor disclosed herein increases the expression of CD36 protein and/or CD36 gene by reducing the expression and/or activity of miR-485. There are two known mature forms of miR-485: miR-485-3p and miR-485-5p. As disclosed herein, in some aspects, a miR-485 inhibitor of the present disclosure can reduce the expression and/or activity of miR-485-3p. In some aspects, a miR-485 inhibitor can reduce the expression and/or activity of miR-485-5p. In further aspects, a miR-485 inhibitor disclosed herein can reduce the expression and/or activity of both miR-485-3p and miR-485-5p.

PGC1 Regulation

The disclosures provided herein demonstrates that the miR-485 inhibitors of the present disclosure can further regulate the expression of PGC-1α, e.g., in a subject suffering from a disease or disorder disclosed herein (see, e.g., Example 3). Therefore, in some aspects, the present disclosure provides a method of increasing an expression of a PGC-1α protein and/or a PGC-1α gene in a subject in need thereof, comprising administering to the subject a compound that inhibits miR-485 activity (i.e., miR-485 inhibitor). In certain aspects, inhibiting miR-485 activity increases the expression of a PGC-1α protein and/or PGC-1α gene in the subject.

Peroxisome proliferator-activated receptor gamma coactivator 1-alpha (PGC1-βalso known as PPARG Coactivator 1 Alpha or Ligand Effect Modulator-6, is a protein that in humans is encoded by the PPARGC1A gene. The PGC1-α gene is located on chromosome 4 in humans (nucleotides 23,792,021 to 24,472,905 of GenBank Accession Number NC_000004.12, plus strand orientation). Synonyms of the PGC1-α gene, and the encoded protein thereof, are known and include “PPARGC1A,” “LEM6,” “PGC1,” “PGC1A,” “PGC-1v,” “PPARGC1, “PGC1alpha, ” or “PGC-1(alpha).”

There are at least nine known isoforms of human PGC1-α protein, resulting from alternative splicing. PGC1-α isoform 1 (UniProt identifier: Q9UBK2-1) consists of 798 amino acids and has been chosen as the canonical sequence (SEQ ID NO: 40). PGC1-α isoform 2 (also known as “Isoform NT-7a”) (UniProt identifier: Q9UBK2-2) consists of 271 amino acids and differs from the canonical sequence as follows: 269-271: DPK→LFL; 272-798: Missing (SEQ ID NO: 41). PGC1-α isoform 3 (also known as “Isoform B5”) (UniProt identifier: Q9UBK2-3) consists of 803 amino acids and differs from the canonical sequence as follows: 1-18: MAWDMCNQDSESVWSDIE→MDETSPRLEEDWKKVLQREAGWQ (SEQ ID NO: 42). PGC1-α isoform 4 (also known as “Isoform B4”) (UniProt identifier: Q9UBK2-4) consists of 786 amino acids and differs from the canonical sequence as follows: 1-18: MAWDMCNQDSESVWSDIE→MDEGYF (SEQ ID NO: 43). PGC1-α isoform 5 (also known as “Isoform B4-8a”) (UniProt identifier: Q9UBK2-5) consists of 289 amino acids and differs from the canonical sequence as follows: 1-18: MAWDMCNQDSESVWSDIE→MDEGYF; 294-301: LTPPTTPP→VKTNLISK; 302-798: Missing (SEQ ID NO: 44). PGC1-α isoform 6 (also known as “Isoform B5-NT”) (UniProt identifier: Q9UBK2-6) consists of 276 amino acids and differs from the canonical sequence as follows: 1-18: MAWDMCNQDSESVWSDIE→MDETSPRLEEDWKKVLQREAGWQ; 269-271: DPK→LFL; 272-798: Missing (SEQ ID NO: 45). PGC1-α isoform 7 (also known as “B4-3ext”) (UniProt identifier: Q9UBK2-7) consists of 138 amino acids and differs from the canonical sequence as follows: 1-18: MAWDMCNQDSESVWSDIE→MDEGYF; 144-150: LKKLLLA→VRTLPTV; 151-798: Missing (SEQ ID NO: 46). PGC1-α isoform 8 (also known as “Isoform 8a”) (UniProt identifier: Q9UBK2-8) consists of 301 amino acids and differs from the canonical sequence as follows: 294-301: LTPPTTPP VKTNLISK; 302-798: Missing (SEQ ID NO: 47). PGC1-α isoform 9 (also known as “Isoform 9” or “L-PGG-1alpha”) (UniProt identifier: Q9UBK2-9) consists of 671 amino acids and differs from the canonical sequence as follows: 1-127: Missing (SEQ ID NO: 48). Table 3 below provides the sequences for the nine PGC1-α isoforms.

TABLE 3
PGC1-α Protein Isoforms

| | |
|---|---|
| Isoform 1 | MAWDMCNQDSESVWSDIECAALVGEDQPLCPDLPELDLSELDVNDLDTDSFLGGLKWCSD |
| (UniProt: | QSEIISNQYNNEPSNIFEKIDEENEANLLAVLTETLDSLPVDEDGLPSFDALTDGDVTTD |
| Q9UBK2-1) | NEASPSSMPDGTPPPQEAEEPSLLKKLLLAPANTQLSYNECSGLSTQNHANHNHRIRTNP |
| (SEQ ID NO: | AIVKTENSWSNKAKSICQQQKPQRRPCSELLKYLTTNDDPPHTKPTENRNSSRDKCTSKK |
| 40) | KSHTQSQSQHLQAKPTTLSLPLTPESPNDPKGSPFENKTIERTLSVELSGTAGLTPPTTP |
| | PHKANQDNPFRASPKLKSSCKTVVPPPSKKPRYSESSGTQGNNSTKKGPEQSELYAQLSK |
| | SSVLTGGHEERKTKRPSLRLFGDHDYCQSINSKTEILINISQELQDSRQLENKDVSSDWQ |
| | GQICSSTDSDQCYLRETLEASKQVSPCSTRKQLQDQEIRAELNKHFGHPSQAVFDDEADK |
| | TGELRDSDFSNEQFSKLPMFINSGLAMDGLFDDSEDESDKLSYPWDGTQSYSLFNVSPSC |
| | SSFNSPCRDSVSPPKSLFSQRPQRMRSRSRSFSRHRSCSRSPYSRSRSRSPGSRSSSRSC |
| | YYYESSHYRHRTHRNSPLYVRSRSRSPYSRRPRYDSYEEYQHERLKREEYRREYEKRESE |
| | RAKQRERQRQKAIEERRVIYVGKIRPDTTRTELRDRFEVFGEIEECTVNLRDDGDSYGFI |
| | TYRYTCDAFAALENGYTLRRSNETDFELYFCGRKQFFKSNYADLDSNSDDFDPASTKSKY |
| | DSLDFDSLLKEAQRSLRR |
| Isoform 2 | MAWDMCNQDSESVWSDIECAALVGEDQPLCPDLPELDLSELDVNDLDTDSFLGGLKWCSD |
| (UniProt: | QSEIISNQYNNEPSNIFEKIDEENEANLLAVLTETLDSLPVDEDGLPSFDALTDGDVTTD |
| Q9UBK2-2) | NEASPSSMPDGTPPPQEAEEPSLLKKLLLAPANTQLSYNECSGLSTQNHANHNHRIRTNP |
| (SEQ ID NO: | AIVKTENSWSNKAKSICQQQKPQRRPCSELLKYLTTNDDPPHTKPTENRNSSRDKCTSKK |
| 41) | KSHTQSQSQHLQAKPTTLSLPLTPESPNLFL |
| Isoform 3 | MDETSPRLEEDWKKVLQREAGWQCAALVGEDQPLCPDLPELDLSELDVNDLDTDSFLGGL |
| (UniProt: | KWCSDQSEIISNQYNNEPSNIFEKIDEENEANLLAVLTETLDSLPVDEDGLPSFDALTDG |
| Q9UBK2-3) | DVTTDNEASPSSMPDGTPPPQEAEEPSLLKKLLLAPANTQLSYNECSGLSTQNHANHNHR |
| (SEQ ID NO: | IRTNPAIVKTENSWSNKAKSICQQQKPQRRPCSELLKYLTTNDDPPHTKPTENRNSSRDK |
| 42) | CTSKKKSHTQSQSQHLQAKPTTLSLPLTPESPNDPKGSPFENKTIERTLSVELSGTAGLT |
| | PPTTPPHKANQDNPFRASPKLKSSCKTVVPPPSKKPRYSESSGTQGNNSTKKGPEQSELY |
| | AQLSKSSVLTGGHEERKTKRPSLRLFGDHDYCQSINSKTEILINISQELQDSRQLENKDV |
| | SSDWQGQICSSTDSDQCYLRETLEASKQVSPCSTRKQLQDQEIRAELNKHFGHPSQAVFD |
| | DEADKTGELRDSDFSNEQFSKLPMFINSGLAMDGLFDDSEDESDKLSYPWDGTQSYSLFN |
| | VSPSCSSFNSPCRDSVSPPKSLFSQRPQRMRSRSRSFSRHRSCSRSPYSRSRSRSPGSRS |
| | SSRSCYYYESSHYRHRTHRNSPLYVRSRSRSPYSRRPRYDSYEEYQHERLKREEYRREYE |
| | KRESERAKQRERQRQKAIEERRVIYVGKIRPDTTRTELRDRFEVFGEIEECTVNLRDDGD |
| | SYGFITYRYTCDAFAALENGYTLRRSNETDFELYFCGRKQFFKSNYADLDSNSDDFDPAS |
| | TKSKYDSLDFDSLLKEAQRSLRR |
| Isoform 4 | MDEGYFCAALVGEDQPLCPDLPELDLSELDVNDLDTDSFLGGLKWCSDQSEIISNQYNNE |
| (UniProt: | PSNIFEKIDEENEANLLAVLTETLDSLPVDEDGLPSFDALTDGDVTTDNEASPSSMPDGT |
| Q9UBK2-4) | PPPQEAEEPSLLKKLLLAPANTQLSYNECSGLSTQNHANHNHRIRTNPAIVKTENSWSNK |
| (SEQ ID NO: | AKSICQQQKPQRRPCSELLKYLTTNDDPPHTKPTENRNSSRDKCTSKKKSHTQSQSQHLQ |
| 43) | AKPTTLSLPLTPESPNDPKGSPFENKTIERTLSVELSGTAGLTPPTTPPHKANQDNPFRA |
| | SPKLKSSCKTVVPPPSKKPRYSESSGTQGNNSTKKGPEQSELYAQLSKSSVLTGGHEERK |
| | TKRPSLRLFGDHDYCQSINSKTEILINISQELQDSRQLENKDVSSDWQGQICSSTDSDQC |
| | YLRETLEASKQVSPCSTRKQLQDQEIRAELNKHFGHPSQAVFDDEADKTGELRDSDFSNE |
| | QFSKLPMFINSGLAMDGLFDDSEDESDKLSYPWDGTQSYSLFNVSPSCSSFNSPCRDSVS |
| | PPKSLFSQRPQRMRSRSRSFSRHRSCSRSPYSRSRSRSPGSRSSSRSCYYYESSHYRHRT |
| | HRNSPLYVRSRSRSPYSRRPRYDSYEEYQHERLKREEYRREYEKRESERAKQRERQRQKA |
| | IEERRVIYVGKIRPDTTRTELRDRFEVFGEIEECTVNLRDDGDSYGFITYRYTCDAFAAL |
| | ENGYTLRRSNETDFELYFCGRKQFFKSNYADLDSNSDDFDPASTKSKYDSLDFDSLLKEA |
| | QRSLRR |
| Isoform 5 | MDEGYFCAALVGEDQPLCPDLPELDLSELDVNDLDTDSFLGGLKWCSDQSEIISNQYNNE |
| (UniProt: | PSNIFEKIDEENEANLLAVLTETLDSLPVDEDGLPSFDALTDGDVTTDNEASPSSMPDGT |
| Q9UBK2-5) | PPPQEAEEPSLLKKLLLAPANTQLSYNECSGLSTQNHANHNHRIRTNPAIVKTENSWSNK |
| (SEQ ID NO: | AKSICQQQKPQRRPCSELLKYLTTNDDPPHTKPTENRNSSRDKCTSKKKSHTQSQSQHLQ |
| 44) | AKPTTLSLPLTPESPNDPKGSPFENKTIERTLSVELSGTAGVKTNLISK |
| Isoform 6 | MDETSPRLEEDWKKVLQREAGWQCAALVGEDQPLCPDLPELDLSELDVNDLDTDSFLGGL |
| (UniProt: | KWCSDQSEIISNQYNNEPSNIFEKIDEENEANLLAVLTETLDSLPVDEDGLPSFDALTDG |
| Q9UBK2-6) | DVTTDNEASPSSMPDGTPPPQEAEEPSLLKKLLLAPANTQLSYNECSGLSTQNHANHNHR |
| (SEQ ID NO: | IRTNPAIVKTENSWSNKAKSICQQQKPQRRPCSELLKYLTTNDDPPHTKPTENRNSSRDK |
| 45) | CTSKKKSHTQSQSQHLQAKPTTLSLPLTPESPNLFL |
| Isoform 7 | MDEGYFCAALVGEDQPLCPDLPELDLSELDVNDLDTDSFLGGLKWCSDQSEIISNQYNNE |
| (UniProt: | PSNIFEKIDEENEANLLAVLTETLDSLPVDEDGLPSFDALTDGDVTTDNEASPSSMPDGT |
| Q9UBK2-7) | PPPQEAEEPSLVRTLPTV |
| (SEQ ID NO: | |
| 46) | |
| Isoform 8 | MAWDMCNQDSESVWSDIECAALVGEDQPLCPDLPELDLSELDVNDLDTDSFLGGLKWCSD |
| (UniProt: | QSEIISNQYNNEPSNIFEKIDEENEANLLAVLTETLDSLPVDEDGLPSFDALTDGDVTTD |
| Q9UBK2-8) | NEASPSSMPDGTPPPQEAEEPSLLKKLLLAPANTQLSYNECSGLSTQNHANHNHRIRTNP |
| (SEQ ID NO: | AIVKTENSWSNKAKSICQQQKPQRRPCSELLKYLTTNDDPPHTKPTENRNSSRDKCTSKK |
| 47) | KSHTQSQSQHLQAKPTTLSLPLTPESPNDPKGSPFENKTIERTLSVELSGTAGVKTNLIS |
| | K |
| Isoform 9 | MPDGTPPPQEAEEPSLLKKLLLAPANTQLSYNECSGLSTQNHANHNHRIRTNPAIVKTEN |
| (UniProt: | SWSNKAKSICQQQKPQRRPCSELLKYLTTNDDPPHTKPTENRNSSRDKCTSKKKSHTQSQ |
| Q9UBK2-9) | SQHLQAKPTTLSLPLTPESPNDPKGSPFENKTIERTLSVELSGTAGLTPPTTPPHKANQD |
| (SEQ ID NO: | NPFRASPKLKSSCKTVVPPPSKKPRYSESSGTQGNNSTKKGPEQSELYAQLSKSSVLTGG |
| 48) | HEERKTKRPSLRLFGDHDYCQSINSKTEILINISQELQDSRQLENKDVSSDWQGQICSST |
| | DSDQCYLRETLEASKQVSPCSTRKQLQDQEIRAELNKHFGHPSQAVFDDEADKTGELRDS |
| | DFSNEQFSKLPMFINSGLAMDGLFDDSEDESDKLSYPWDGTQSYSLFNVSPSCSSFNSPC |
| | RDSVSPPKSLFSQRPQRMRSRSRSFSRHRSCSRSPYSRSRSRSPGSRSSSRSCYYYESSH |
| | YRHRTHRNSPLYVRSRSRSPYSRRPRYDSYEEYQHERLKREEYRREYEKRESERAKQRER |
| | QRQKAIEERRVIYVGKIRPDTTRTELRDRFEVFGEIEECTVNLRDDGDSYGFITYRYTCD |
| | AFAALENGYTLRRSNETDFELYFCGRKQFFKSNYADLDSNSDDFDPASTKSKYDSLDFDS |
| | LLKEAQRSLRR |

As used herein, the term “PGC1-α” (including its synonyms) includes any variants or isoforms of PGC1-α which are naturally expressed by cells. Accordingly, in some aspects, a miR-485 inhibitor disclosed herein can increase the expression of PGC1-α isoform 1. In some aspects, a miR-485 inhibitor disclosed herein can increase the expression of PGC1-α isoform 2. Accordingly, in some aspects, a miR-485 inhibitor disclosed herein can increase the expression of PGC1-α isoform 1. In some aspects, a miR-485 inhibitor disclosed herein can increase the expression of PGC1-α isoform 2. Accordingly, in some aspects, a miR-485 inhibitor disclosed herein can increase the expression of PGC1-α isoform 3. In some aspects, a miR-485 inhibitor disclosed herein can increase the expression of PGC1-α isoform 4. Accordingly, in some aspects, a miR-485 inhibitor disclosed herein can increase the expression of PGC1-α isoform 5. In some aspects, a miR-485 inhibitor disclosed herein can increase the expression of PGC1-α isoform 6. Accordingly, in some aspects, a miR-485 inhibitor disclosed herein can increase the expression of PGC1-α isoform 7. In some aspects, a miR-485 inhibitor disclosed herein can increase the expression of PGC1-α isoform 8. Accordingly, in some aspects, a miR-485 inhibitor disclosed herein can increase the expression of PGC1-α isoform 9. In further aspects, a miR-485 inhibitor disclosed herein can increase the expression of PGC1-α isoform 1, isoform 2, isoform 3, isoform 4, isoform 5, isoform 6, isoform 7, isoform 8, and isoform 9. Unless indicated otherwise, both isoform 1 and isoform 2 are collectively referred to herein as “PGC1-α.”

In some aspects, a miR-485 inhibitor of the present disclosure increases the expression of PGC1-α protein and/or PGC1-α gene by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% compared to a reference (e.g., expression of PGC1-α protein and/or PGC1-α gene in a corresponding subject that did not receive an administration of the miR-485 inhibitor).

Not to be bound by any one theory, in some aspects, a miR-485 inhibitor disclosed herein increases the expression of PGC1-α protein and/or PGC1-α gene by reducing the expression and/or activity of miR-485. There are two known mature forms of miR-485: miR-485-3p and miR-485-5p. In some aspects, a miR-485 inhibitor of the present disclosure can reduce the expression and/or activity of miR-485-3p. In some aspects, a miR-485 inhibitor can reduce the expression and/or activity of miR-485-5p. In further aspects, a miR-485 inhibitor disclosed herein can reduce the expression and/or activity of both miR-485-3p and miR-485-5p.

LRRK2 Regulation

In some aspects, the disclosures provided herein demonstrates that the miR-485 inhibitors of the present disclosure can regulate the expression of LRRK2, e.g., in a subject suffering from a disease or disorder disclosed herein (e.g., Parkinson's disease). Therefore, in some aspects, the present disclosure provides a method of increasing an expression of a LRRK2 protein and/or a LRRK2 gene in a subject in need thereof, comprising administering to the subject a compound that inhibits miR-485 activity (i.e., miR-485 inhibitor). In certain aspects, inhibiting miR-485 activity increases the expression of a LRRK2 protein and/or LRRK2 gene in the subject.

Leucine-rich repeat kinase 2 (LRRK2) is a kinase enzyme that in humans is encoded by the LRRK2 gene. The LRRK2 gene is located on chromosome 12 in humans (nucleotides 40,224,890 to 40,369,285 of GenBank Accession Number NC_000012.12, plus strand orientation). Synonyms of the LRRK2 gene, and the encoded protein thereof, are known and include PARK8, RIPK7, ROCO2, AURA17, and DARDARIN.

Table 4 below provides the amino acid sequence for the LRRK2 protein.

TABLE 4
LRRK2 Protein Sequence

| | |
|---|---|
| LRRK2 | MASGSCQGCEEDEETLKKLIVRLNNVQEGKQIETLVQILEDLLVFTYSERASKLFQGKNIHVPLLIVLDS |
| (UniProt: | YMRVASVQQVGWSLLCKLIEVCPGTMQSLMGPQDVGNDWEVLGVHQLILKMLTVHNASVNLSVIGLKTLD |
| Q5S007.2) | LLLTSGKITLLILDEESDIFMLIFDAMHSFPANDEVQKLGCKALHVLFERVSEEQLTEFVENKDYMILLS |
| (SEQ ID | ALTNFKDEEEIVLHVLHCLHSLAIPCNNVEVLMSGNVRCYNIVVEAMKAFPMSERIQEVSCCLLHRLTLG |
| NO: 110) | NFFNILVLNEVHEFVVKAVQQYPENAALQISALSCLALLTETIFLNQDLEEKNENQENDDEGEEDKLFWL |
| | EACYKALTWHRKNKHVQEAACWALNNLLMYQNSLHEKIGDEDGHFPAHREVMLSMLMHSSSKEVFQASAN |
| | ALSTLLEQNVNFRKILLSKGIHLNVLELMQKHIHSPEVAESGCKMLNHLFEGSNTSLDIMAAVVPKILTV |
| | MKRHETSLPVQLEALRAILHFIVPGMPEESREDTEFHHKLNMVKKQCFKNDIHKLVLAALNRFIGNPGIQ |
| | KCGLKVISSIVHFPDALEMLSLEGAMDSVLHTLQMYPDDQEIQCLGLSLIGYLITKKNVFIGTGHLLAKI |
| | LVSSLYRFKDVAEIQTKGFQTILAILKLSASFSKLLVHHSFDLVIFHQMSSNIMEQKDQQFLNLCCKCFA |
| | KVAMDDYLKNVMLERACDQNNSIMVECLLLLGADANQAKEGSSLICQVCEKESSPKLVELLLNSGSREQD |
| | VRKALTISIGKGDSQIISLLLRRLALDVANNSICLGGFCIGKVEPSWLGPLFPDKTSNLRKQTNIASTLA |
| | RMVIRYQMKSAVEEGTASGSDGNFSEDVLSKFDEWTFIPDSSMDSVFAQSDDLDSEGSEGSFLVKKKSNS |
| | ISVGEFYRDAVLQRCSPNLQRHSNSLGPIFDHEDLLKRKRKILSSDDSLRSSKLQSHMRHSDSISSLASE |
| | REYITSLDLSANELRDIDALSQKCCISVHLEHLEKLELHQNALTSFPQQLCETLKSLTHLDLHSNKFTSF |
| | PSYLLKMSCIANLDVSRNDIGPSVVLDPTVKCPTLKQFNLSYNQLSFVPENLTDVVEKLEQLILEGNKIS |
| | GICSPLRLKELKILNLSKNHISSLSENFLEACPKVESFSARMNFLAAMPFLPPSMTILKLSQNKFSCIPE |
| | AILNLPHLRSLDMSSNDIQYLPGPAHWKSLNLRELLFSHNQISILDLSEKAYLWSRVEKLHLSHNKLKEI |
| | PPEIGCLENLTSLDVSYNLELRSFPNEMGKLSKIWDLPLDELHLNFDFKHIGCKAKDIIRFLQQRLKKAV |
| | PYNRMKLMIVGNTGSGKTTLLQQLMKTKKSDLGMQSATVGIDVKDWPIQIRDKRKRDLVLNVWDFAGREE |
| | FYSTHPHFMTQRALYLAVYDLSKGQAEVDAMKPWLFNIKARASSSPVILVGTHLDVSDEKQRKACMSKIT |
| | KELLNKRGFPAIRDYHFVNATEESDALAKLRKTIINESLNFKIRDQLVVGQLIPDCYVELEKIILSERKN |
| | VPIEFPVIDRKRLLQLVRENQLQLDENELPHAVHFLNESGVLLHFQDPALQLSDLYFVEPKWLCKIMAQI |
| | LTVKVEGCPKHPKGIISRRDVEKFLSKKRKFPKNYMSQYFKLLEKFQIALPIGEEYLLVPSSLSDHRPVI |
| | ELPHCENSEIIIRLYEMPYFPMGFWSRLINRLLEISPYMLSGRERALRPNRMYWRQGIYLNWSPEAYCLV |
| | GSEVLDNHPESFLKITVPSCRKGCILLGQVVDHIDSLMEEWFPGLLEIDICGEGETLLKKWALYSFNDGE |
| | EHQKILLDDLMKKAEEGDLLVNPDQPRLTIPISQIAPDLILADLPRNIMLNNDELEFEQAPEFLLGDGSF |
| | GSVYRAAYEGEEVAVKIFNKHTSLRLLRQELVVLCHLHHPSLISLLAAGIRPRMLVMELASKGSLDRLLQ |
| | QDKASLTRTLQHRIALHVADGLRYLHSAMIIYRDLKPHNVLLFTLYPNAAIIAKIADYGIAQYCCRMGIK |
| | TSEGTPGFRAPEVARGNVIYNQQADVYSFGLLLYDILTTGGRIVEGLKFPNEFDELEIQGKLPDPVKEYG |
| | CAPWPMVEKLIKQCLKENPQERPTSAQVFDILNSAELVCLTRRILLPKNVIVECMVATHHNSRNASIWLG |
| | CGHTDRGQLSFLDLNTEGYTSEEVADSRILCLALVHLPVEKESWIVSGTQSGTLLVINTEDGKKRHTLEK |
| | MTDSVTCLYCNSFSKQSKQKNFLLVGTADGKLAIFEDKTVKLKGAAPLKILNIGNVSTPLMCLSESTNST |
| | ERNVMWGGCGTKIFSFSNDFTIQKLIETRTSQLFSYAAFSDSNIITVVVDTALYIAKQNSPVVEVWDKKT |
| | EKLCGLIDCVHFLREVMVKENKESKHKMSYSGRVKTLCLQKNTALWIGTGGGHILLLDLSTRRLIRVIYN |
| | FCNSVRVMMTAQLGSLKNVMLVLGYNRKNTEGTQKQKEIQSCLTVWDINLPHEVQNLEKHIEVRKELAEK |
| | MRRTSVE |

As used herein, the term “LRRK2” (including its synonyms) includes any variants or isoforms of LRRK2 which are naturally expressed by cells.

In some aspects, a miR-485 inhibitor of the present disclosure increases the expression of LRRK2 protein and/or LRRK2 gene by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% compared to a reference (e.g., expression of LRRK2 protein and/or LRRK2 gene in a corresponding subject that did not receive an administration of the miR-485 inhibitor).

Not to be bound by any one theory, in some aspects, a miR-485 inhibitor disclosed herein increases the expression of LRRK2 protein and/or LRRK2 gene by reducing the expression and/or activity of miR-485, e.g., miR-485-3p.

NRG1 Regulation

The disclosures provided herein demonstrates that the miR-485 inhibitors of the present disclosure can further regulate the expression of NRG1, e.g., in a subject suffering from a disease or disorder disclosed herein (see, e.g., Parkinson's disease). Therefore, in some aspects, the present disclosure provides a method of increasing an expression of a NRG1 protein and/or a NRG1 gene in a subject in need thereof, comprising administering to the subject a compound that inhibits miR-485 activity (i.e., miR-485 inhibitor). In certain aspects, inhibiting miR-485 activity increases the expression of a NRG1 protein and/or NRG1 gene in the subject.

Neuregulin 1 is a cell adhesion molecule that in humans is encoded by the NRG1 gene. NRG1 is one of four proteins in the neuregulin family that act on the EGFR family of receptors. The NRG1 gene is located on chromosome 8 in humans (nucleotides 31,639,245 to 32,774,046 of GenBank Accession Number NC_000008.11). Synonyms of the NRG1 gene, and the encoded protein thereof, are known and include “GGF,” “HGL,” “HRG,” “NDF,” “ARIA,” “GGF2,” “HRG1,” “HRGA,” “SMDF,” “MST131,” “MSTP131,” and “NRG1-IT2.”

There are at least 11 known isoforms of human NRG1 protein, resulting from alternative splicing. NRG1 isoform 1 (also known as “Alpha”) (UniProt identifier: Q02297-1) is 640 amino acids in length and has been chosen as the canonical sequence (SEQ ID NO: 91). NRG1 isoform 2 (also known as “Alpha1A”) (UniProt identifier: Q02297-2) is 648 amino acids long and differs from the canonical sequence as follows: 234-234: K→KHLGIEFIE (SEQ ID NO: 92). NRG1 isoform 3 (also known as “Alpha2B”) (UniProt identifier: Q02297-3) is 462 amino acids long and differs from the canonical sequence as follows: (i) 424-462: YVSAMTTPAR . . . SPPVSSMTVS→HNLIAELRRN . . . SSIPHLGFIL; and (ii) 463-640: Missing (SEQ ID NO: 93). NRG1 isoform 4 (also known as “Alpha3”) (UniProt identifier: Q02297-4) consists of 247 amino acids and differs from the canonical sequence as follows: (i) 234-247: KAEELYQKRVLTIT→SAQMSLLVIAAKTT; and (ii) 248-260: Missing (SEQ ID NO: 94). NRG1 isoform 6 (also known as “Beta1” and “Beta1A”) (UniProt identifier: Q02297-6) is 645 amino acids in length and differs from the canonical sequence as follows: 213-234: QPGFTGARCTENVPMKVQNQEK→PNEFTGDRCQNYVMASFYKHLGIEFME (SEQ ID NO: 95). NRG1 isoform 7 (also known as “Beta2”) (UniProt identifier: Q02297-7) consists of 647 amino acids and differs from the canonical sequence as follows: 213-233: QPGFTGARCTENVPMKVQNQE→PNEFTGDRCQNYVMASFY (SEQ ID NO: 96). NRG1 isoform 8 (also known as “Beta3” and “GGFHFB1”) (UniProt identifier: Q02297-8) is made up of 241 amino acids and differs from the canonical sequence as follows: (i) 213-241: QPGFTGARCTENVPMKVQNQEKAEELYQK→PNEFTGDRCQNYVMASFYSTSTPFLSLPE; and (ii) 242-640: Missing (SEQ ID NO: 97). NRG1 isoform 9 (also known as “GGF2” and “GGFHPP2”) (UniProt identifier: Q02297-9) is 422 amino acids in length and differs from the canonical sequence as follows: 1-33: MSERKEGRGKGKGKKKERGSGKKPESAAGSQSP→MRWRRAPRRS . . . EVSRVLCKRC; (2) 134-168: EIITGMPASTEGAYVSSESPIRISVSTEGANTSSS→A; (3) 213-241: QPGFTGARCTENVPMKVQNQEKAEELYQK→PNEFTGDRCQNYVMASFYSTSTPFLSLPE; and (iv) 242-640: Missing (SEQ ID NO: 98). NRG1 isoform 10 (also known as “SMDF”) (UniProt identifier: Q02297-10) is 296 amino acids long and differs from the canonical sequence as follows: (i) 1-166: Missing; (ii) 167-167: S→MEIYSPDMSE . . . ETNLQTAPKL; (iii) 213-241: QPGFTGARCTENVPMKVQNQEKAEELYQK→PNEFTGDRCQNYVMASFYSTSTPFLSLPE; and (iv) 242-640: Missing (SEQ ID NO: 99). NRG1 isoform 11 (also known as “Type IV-beta1a”) (UniProt identifier: Q02297-11) is 590 amino acids long and differs from the canonical sequence as follows: (i) 1-21: Missing; (ii) 22-33: KKPESAAGSQSP→MGKGRAGRVGTT; (iii) 134-168: EIITGMPASTEGAYVSSESPIRISVSTEGANTSSS→A; and (iv) 213-234: QPGF TGARCTENVPMKVQNQEK→PNEFTGDRCQNYVMASFYKHLGIEFME (SEQ ID NO: 100). NRG1 isoform 12 (UniProt identifier: Q02297-12) consists of 420 amino acids and differs from the canonical sequence as follows: (i) 213-233: QPGFTGARCTENVPMKVQNQE→PNEFTGDRCQNYVMASFY; and (ii) 424-640: Missing (SEQ ID NO: 101).

Table 5 below provides the amino acid sequences for the NRG1 protein, including known isoforms.

TABLE 5
NRG1 Protein Sequence

| | |
|---|---|
| Isoform 1 | MSERKEGRGKGKGKKKERGSGKKPESAAGSQSPALPPRLKEMKSQESAAGSKLVLRCETS |
| (UniProt: | SEYSSLRFKWFKNGNELNRKNKPQNIKIQKKPGKSELRINKASLADSGEYMCKVISKLGN |
| Q02297-1) | DSASANITIVESNEIITGMPASTEGAYVSSESPIRISVSTEGANTSSSTSTSTTGTSHLV |
| (SEQ ID NO: | KCAEKEKTFCVNGGECFMVKDLSNPSRYLCKCQPGFTGARCTENVPMKVQNQEKAEELYQ |
| 91) | KRVLTITGICIALLVVGIMCVVAYCKTKKQRKKLHDRLRQSLRSERNNMMNIANGPHHPN |
| | PPPENVQLVNQYVSKNVISSEHIVEREAETSFSTSHYTSTAHHSTTVTQTPSHSWSNGHT |
| | ESILSESHSVIVMSSVENSRHSSPTGGPRGRLNGTGGPRECNSFLRHARETPDSYRDSPH |
| | SERYVSAMTTPARMSPVDFHTPSSPKSPPSEMSPPVSSMTVSMPSMAVSPFMEEERPLLL |
| | VTPPRLREKKFDHHPQQFSSFHHNPAHDSNSLPASPLRIVEDEEYETTQEYEPAQEPVKK |
| | LANSRRAKRTKPNGHIANRLEVDSNTSSQSSNSESETEDERVGEDTPFLGIQNPLAASLE |
| | ATPAFRLADSRTNPAGRFSTQEEIQARLSSVIANQDPIAV |
| Isoform 2 | MSERKEGRGKGKGKKKERGSGKKPESAAGSQSPALPPRLKEMKSQESAAGSKLVLRCETS |
| (UniProt: | SEYSSLRFKWFKNGNELNRKNKPQNIKIQKKPGKSELRINKASLADSGEYMCKVISKLGN |
| Q02297-2) | DSASANITIVESNEIITGMPASTEGAYVSSESPIRISVSTEGANTSSSTSTSTTGTSHLV |
| (SEQ ID NO: | KCAEKEKTFCVNGGECFMVKDLSNPSRYLCKCQPGFTGARCTENVPMKVQNQEKHLGIEF |
| 92) | IEAEELYQKRVLTITGICIALLVVGIMCVVAYCKTKKQRKKLHDRLRQSLRSERNNMMNI |
| | ANGPHHPNPPPENVQLVNQYVSKNVISSEHIVEREAETSFSTSHYTSTAHHSTTVTQTPS |
| | HSWSNGHTESILSESHSVIVMSSVENSRHSSPTGGPRGRLNGTGGPRECNSFLRHARETP |
| | DSYRDSPHSERYVSAMTTPARMSPVDFHTPSSPKSPPSEMSPPVSSMTVSMPSMAVSPFM |
| | EEERPLLLVTPPRLREKKFDHHPQQFSSFHHNPAHDSNSLPASPLRIVEDEEYETTQEYE |
| | PAQEPVKKLANSRRAKRTKPNGHIANRLEVDSNTSSQSSNSESETEDERVGEDTPFLGIQ |
| | NPLAASLEATPAFRLADSRTNPAGRFSTQEEIQARLSSVIANQDPIAV |
| Isoform 3 | MSERKEGRGKGKGKKKERGSGKKPESAAGSQSPALPPRLKEMKSQESAAGSKLVLRCETS |
| (UniProt: | SEYSSLRFKWFKNGNELNRKNKPQNIKIQKKPGKSELRINKASLADSGEYMCKVISKLGN |
| Q02297-3) | DSASANITIVESNEIITGMPASTEGAYVSSESPIRISVSTEGANTSSSTSTSTTGTSHLV |
| (SEQ ID NO: | KCAEKEKTFCVNGGECFMVKDLSNPSRYLCKCQPGFTGARCTENVPMKVQNQEKAEELYQ |
| 93) | KRVLTITGICIALLVVGIMCVVAYCKTKKQRKKLHDRLRQSLRSERNNMMNIANGPHHPN |
| | PPPENVQLVNQYVSKNVISSEHIVEREAETSFSTSHYTSTAHHSTTVTQTPSHSWSNGHT |
| | ESILSESHSVIVMSSVENSRHSSPTGGPRGRLNGTGGPRECNSFLRHARETPDSYRDSPH |
| | SERHNLIAELRRNKAHRSKCMQIQLSATHLRSSSIPHLGFIL |
| Isoform 4 | MSERKEGRGKGKGKKKERGSGKKPESAAGSQSPALPPRLKEMKSQESAAGSKLVLRCETS |
| (UniProt: | SEYSSLRFKWFKNGNELNRKNKPQNIKIQKKPGKSELRINKASLADSGEYMCKVISKLGN |
| Q02297-4) | DSASANITIVESNEIITGMPASTEGAYVSSESPIRISVSTEGANTSSSTSTSTTGTSHLV |
| (SEQ ID NO: | KCAEKEKTFCVNGGECFMVKDLSNPSRYLCKCQPGFTGARCTENVPMKVQNQESAQMSLL |
| 94) | VIAAKTT |
| Isoform 6 | MSERKEGRGKGKGKKKERGSGKKPESAAGSQSPALPPRLKEMKSQESAAGSKLVLRCETS |
| (UniProt: | SEYSSLRFKWFKNGNELNRKNKPQNIKIQKKPGKSELRINKASLADSGEYMCKVISKLGN |
| Q02297-6) | DSASANITIVESNEIITGMPASTEGAYVSSESPIRISVSTEGANTSSSTSTSTTGTSHLV |
| (SEQ ID NO: | KCAEKEKTFCVNGGECFMVKDLSNPSRYLCKCPNEFTGDRCQNYVMASFYKHLGIEFMEA |
| 95) | EELYQKRVLTITGICIALLVVGIMCVVAYCKTKKQRKKLHDRLRQSLRSERNNMMNIANG |
| | PHHPNPPPENVQLVNQYVSKNVISSEHIVEREAETSFSTSHYTSTAHHSTTVTQTPSHSW |
| | SNGHTESILSESHSVIVMSSVENSRHSSPTGGPRGRLNGTGGPRECNSFLRHARETPDSY |
| | RDSPHSERYVSAMTTPARMSPVDFHTPSSPKSPPSEMSPPVSSMTVSMPSMAVSPFMEEE |
| | RPLLLVTPPRLREKKFDHHPQQFSSFHHNPAHDSNSLPASPLRIVEDEEYETTQEYEPAQ |
| | EPVKKLANSRRAKRTKPNGHIANRLEVDSNTSSQSSNSESETEDERVGEDTPFLGIQNPL |
| | AASLEATPAFRLADSRTNPAGRFSTQEEIQARLSSVIANQDPIAV |
| Isoform 7 | MSERKEGRGKGKGKKKERGSGKKPESAAGSQSPALPPRLKEMKSQESAAGSKLVLRCETS |
| (UniProt: | SEYSSLRFKWFKNGNELNRKNKPQNIKIQKKPGKSELRINKASLADSGEYMCKVISKLGN |
| Q02297-7) | DSASANITIVESNEIITGMPASTEGAYVSSESPIRISVSTEGANTSSSTSTSTTGTSHLV |
| (SEQ ID NO: | KCAEKEKTFCVNGGECFMVKDLSNPSRYLCKCPNEFTGDRCQNYVMASFYKAEELYQKRV |
| 96) | LTITGICIALLVVGIMCVVAYCKTKKQRKKLHDRLRQSLRSERNNMMNIANGPHHPNPPP |
| | ENVQLVNQYVSKNVISSEHIVEREAETSFSTSHYTSTAHHSTTVTQTPSHSWSNGHTESI |
| | LSESHSVIVMSSVENSRHSSPTGGPRGRLNGTGGPRECNSFLRHARETPDSYRDSPHSER |
| | YVSAMTTPARMSPVDFHTPSSPKSPPSEMSPPVSSMTVSMPSMAVSPFMEEERPLLLVTP |
| | PRLREKKFDHHPQQFSSFHHNPAHDSNSLPASPLRIVEDEEYETTQEYEPAQEPVKKLAN |
| | SRRAKRTKPNGHIANRLEVDSNTSSQSSNSESETEDERVGEDTPFLGIQNPLAASLEATP |
| | AFRLADSRTNPAGRFSTQEEIQARLSSVIANQDPIAV |
| Isoform 8 | MSERKEGRGKGKGKKKERGSGKKPESAAGSQSPALPPRLKEMKSQESAAGSKLVLRCETS |
| (UniProt: | SEYSSLRFKWFKNGNELNRKNKPQNIKIQKKPGKSELRINKASLADSGEYMCKVISKLGN |
| Q02297-8) | DSASANITIVESNEIITGMPASTEGAYVSSESPIRISVSTEGANTSSSTSTSTTGTSHLV |
| (SEQ ID NO: | KCAEKEKTFCVNGGECFMVKDLSNPSRYLCKCPNEFTGDRCQNYVMASFYSTSTPFLSLP |
| 97) | E |
| Isoform 9 | MRWRRAPRRSGRPGPRAQRPGSAARSSPPLPLLPLLLLLGTAALAPGAAAGNEAAPAGAS |
| (UniProt: | VCYSSPPSVGSVQELAQRAAVVIEGKVHPQRRQQGALDRKAAAAAGEAGAWGGDREPPAA |
| Q02297-9) | GPRALGPPAEEPLLAANGTVPSWPTAPVPSAGEPGEEAPYLVKVHQVWAVKAGGLKKDSL |
| (SEQ ID NO: | LTVRLGTWGHPAFPSCGRLKEDSRYIFFMEPDANSTSRAPAAFRASFPPLETGRNLKKEV |
| 98) | SRVLCKRCALPPRLKEMKSQESAAGSKLVLRCETSSEYSSLRFKWFKNGNELNRKNKPQN |
| | IKIQKKPGKSELRINKASLADSGEYMCKVISKLGNDSASANITIVESNATSTSTTGTSHL |
| | VKCAEKEKTFCVNGGECFMVKDLSNPSRYLCKCPNEFTGDRCQNYVMASFYSTSTPFLSL |
| | PE |
| Isoform 10 | MEIYSPDMSEVAAERSSSPSTQLSADPSLDGLPAAEDMPEPQTEDGRTPGLVGLAVPCCA |
| (UniProt: | CLEAERLRGCLNSEKICIVPILACLVSLCLCIAGLKWVFVDKIFEYDSPTHLDPGGLGQD |
| Q02297-10) | PIISLDATAASAVWVSSEAYTSPVSRAQSESEVQVTVQGDKAVVSFEPSAAPTPKNRIFA |
| (SEQ ID NO: | FSFLPSTAPSFPSPTRNPEVRTPKSATQPQTTETNLQTAPKLSTSTSTTGTSHLVKCAEK |
| 99) | EKTFCVNGGECFMVKDLSNPSRYLCKCPNEFTGDRCQNYVMASFYSTSTPFLSLPE |
| Isoform 11 | MGKGRAGRVGTTALPPRLKEMKSQESAAGSKLVLRCETSSEYSSLRFKWFKNGNELNRKN |
| (UniProt: | KPQNIKIQKKPGKSELRINKASLADSGEYMCKVISKLGNDSASANITIVESNATSTSTTG |
| Q02297-11) | TSHLVKCAEKEKTFCVNGGECFMVKDLSNPSRYLCKCPNEFTGDRCQNYVMASFYKHLGI |
| (SEQ ID NO: | EFMEAEELYQKRVLTITGICIALLVVGIMCVVAYCKTKKQRKKLHDRLRQSLRSERNNMM |
| 100) | NIANGPHHPNPPPENVQLVNQYVSKNVISSEHIVEREAETSFSTSHYTSTAHHSTTVTQT |
| | PSHSWSNGHTESILSESHSVIVMSSVENSRHSSPTGGPRGRLNGTGGPRECNSFLRHARE |
| | TPDSYRDSPHSERYVSAMTTPARMSPVDFHTPSSPKSPPSEMSPPVSSMTVSMPSMAVSP |
| | FMEEERPLLLVTPPRLREKKFDHHPQQFSSFHHNPAHDSNSLPASPLRIVEDEEYETTQE |
| | YEPAQEPVKKLANSRRAKRTKPNGHIANRLEVDSNTSSQSSNSESETEDERVGEDTPFLG |
| | IQNPLAASLEATPAFRLADSRTNPAGRFSTQEEIQARLSSVIANQDPIAV |
| Isoform 12 | MSERKEGRGKGKGKKKERGSGKKPESAAGSQSPALPPRLKEMKSQESAAGSKLVLRCETS |
| (UniProt: | SEYSSLRFKWFKNGNELNRKNKPQNIKIQKKPGKSELRINKASLADSGEYMCKVISKLGN |
| Q02297-12) | DSASANITIVESNEIITGMPASTEGAYVSSESPIRISVSTEGANTSSSTSTSTTGTSHLV |
| (SEQ ID NO: | KCAEKEKTFCVNGGECFMVKDLSNPSRYLCKCPNEFTGDRCQNYVMASFYKAEELYQKRV |
| 101) | LTITGICIALLVVGIMCVVAYCKTKKQRKKLHDRLRQSLRSERNNMMNIANGPHHPNPPP |
| | ENVQLVNQYVSKNVISSEHIVEREAETSFSTSHYTSTAHHSTTVTQTPSHSWSNGHTESI |
| | LSESHSVIVMSSVENSRHSSPTGGPRGRLNGTGGPRECNSFLRHARETPDSYRDSPHSER |

As used herein, the term “NRG1” (including its synonyms) includes any variants or isoforms of NRG1 which are naturally expressed by cells. Accordingly, in some aspects, a miR-485 inhibitor disclosed herein can increase the expression of NRG1 isoform 1 (i.e., canonical sequence). In some aspects, a miR-485 inhibitor disclosed herein can increase the expression of NRG1 isoform 2. In some aspects, a miR-485 inhibitor disclosed herein can increase the expression of NRG1 isoform 3. In some aspects, a miR-485 inhibitor disclosed herein can increase the expression of NRG1 isoform 4. In some aspects, a miR-485 inhibitor disclosed herein can increase the expression of NRG1 isoform 6. In some aspects, a miR-485 inhibitor disclosed herein can increase the expression of NRG1 isoform 7. In some aspects, a miR-485 inhibitor disclosed herein can increase the expression of NRG1 isoform 8. In some aspects, a miR-485 inhibitor disclosed herein can increase the expression of NRG1 isoform 9. In some aspects, a miR-485 inhibitor disclosed herein can increase the expression of NRG1 isoform 10. In some aspects, a miR-485 inhibitor disclosed herein can increase the expression of NRG1 isoform 11. In some aspects, a miR-485 inhibitor disclosed herein can increase the expression of NRG1 isoform 12. In some aspects, a miR-485 inhibitor disclosed herein can increase the expression of NRG1 isoform 1, NRG1 isoform 2, NRG1 isoform 3, NRG1 isoform 4, NRG1 isoform 6, NRG1 isoform 7, NRG1 isoform 8, NRG1 isoform 9, NRG1 isoform 10, NRG1 isoform 11, and NRG1 isoform 12. Unless indicated otherwise, the above-described isoforms of NRG1 are collectively referred to herein as “NRG1.”

In some aspects, a miR-485 inhibitor of the present disclosure increases the expression of NRG1 protein and/or NRG1 gene by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% compared to a reference (e.g., expression of NRG1 protein and/or NRG1 gene in a corresponding subject that did not receive an administration of the miR-485 inhibitor).

Not to be bound by any one theory, in some aspects, a miR-485 inhibitor disclosed herein increases the expression of NRG1 protein and/or NRG1 gene by reducing the expression and/or activity of miR-485, e.g., miR-485-3p.

STMN2 Regulation

The disclosures provided herein demonstrates that the miR-485 inhibitors of the present disclosure can further regulate the expression of STMN2, e.g., in a subject suffering from a disease or disorder disclosed herein (see, e.g., Parkinson's disease). Therefore, in some aspects, the present disclosure provides a method of increasing an expression of a STMN2 protein and/or a STMN2 gene in a subject in need thereof, comprising administering to the subject a compound that inhibits miR-485 activity (i.e., miR-485 inhibitor). In certain aspects, inhibiting miR-485 activity increases the expression of a STMN2 protein and/or STMN2 gene in the subject.

Stathmin-2 is a member of the stathmin family of phosphoproteins and in humans is encoded by the STMN2 gene. Stathmin proteins function in microtubule dynamics and signal transduction. The encoded protein plays a regulatory role in neuronal growth and is also thought to be involved in osteogenesis. The STMN2 gene is located on chromosome 8 in humans (nucleotides 79,611,117 to 79, 666,162 of NC_000008.11). Synonyms of the STMN2 gene, and the encoded protein thereof, are known and include “SCG10” and “SCGN10.”

There are at least 2 known isoforms of human STMN2 protein, resulting from alternative splicing. STMN2 isoform 1 (UniProt identifier: Q93045-1) is 179 amino acids in length and has been chosen as the canonical sequence (SEQ ID NO: 102). STMN2 isoform 2 (UniProt identifier: Q93045-2) is 187 amino acids in length and differs from the canonical sequence as follows: 161-179: ERHAAEVRRNKELQVELSG→LVKFISSELKESIESQFLELQREGEKQ (SEQ ID NO: 103).

Table 6 below provides the amino acid sequences for the STMN2 protein.

TABLE 6
STMN2 Protein Sequence

| Isoform 1 | MAKTAMAYKEKMKELSMLSLICSCFYPEPRNINIYTYDDMEVKQINKRASGQAFELILKP |
|---|---|
| (UniProt: | PSPISEAPRTLASPKKKDLSLEEIQKKLEAAEERRKSQEAQVLKQLAEKREHEREVLQKA |
| Q93045-1) | LEENNNFSKMAEEKLILKMEQIKENREANLAAIIERLQEKERHAAEVRRNKELQVELSG |
| (SEQ ID NO: | |
| 102) | |
| Isoform 2 | MAKTAMAYKEKMKELSMLSLICSCFYPEPRNINIYTYDDMEVKQINKRASGQAFELILKP |
| (UniProt: | PSPISEAPRTLASPKKKDLSLEEIQKKLEAAEERRKSQEAQVLKQLAEKREHEREVLQKA |
| Q93045-2) | LEENNNFSKMAEEKLILKMEQIKENREANLAAIIERLQEKLVKFISSELKESIESQFLEL |
| (SEQ ID NO: | QREGEKQ |
| 103) | |

As used herein, the term “STMN2” (including its synonyms) includes any variants or isoforms of STMN2 which are naturally expressed by cells. Accordingly, in some aspects, a miR-485 inhibitor disclosed herein can increase the expression of STMN2 isoform 1 (i.e., canonical sequence). In some aspects, a miR-485 inhibitor disclosed herein can increase the expression of STMN2 isoform 2. In some aspects, a miR-485 inhibitor disclosed herein can increase the expression of STMN2 isoform 1 and STMN2 isoform 2. Unless indicated otherwise, the above-described isoforms of STMN2 are collectively referred to herein as “ STMN2.”

In some aspects, a miR-485 inhibitor of the present disclosure increases the expression of STMN2 protein and/or STMN2 gene by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% compared to a reference (e.g., expression of STMN2 protein and/or STMN2 gene in a corresponding subject that did not receive an administration of the miR-485 inhibitor).

Not to be bound by any one theory, in some aspects, a miR-485 inhibitor disclosed herein increases the expression of STMN2 protein and/or STMN2 gene by reducing the expression and/or activity of miR-485, e.g., miR-485-3p.

VLDLR Regulation

The disclosures provided herein demonstrates that the miR-485 inhibitors of the present disclosure can further regulate the expression of VLDLR, e.g., in a subject suffering from a disease or disorder disclosed herein (see, e.g., Alzheimer's disease). Therefore, in some aspects, the present disclosure provides a method of increasing an expression of a VLDLR protein and/or a VLDLR gene in a subject in need thereof, comprising administering to the subject a compound that inhibits miR-485 activity (i.e., miR-485 inhibitor). In certain aspects, inhibiting miR-485 activity increases the expression of a VLDLR protein and/or VLDLR gene in the subject.

Very-low-density-lipoprotein receptor (VLDLR) is a transmembrane lipoprotein receptor of the low-density-lipoprotein (LDL) receptor family. VLDLR is expressed in many tissues and plays an important role in various biological processes, including neuronal migration in the developing brain. In humans, VLDLR is encoded by the VLDLR gene, which is located on chromosome 9 (nucleotides 2,621,786 to 2,660,056 of NC_000009.12). Synonyms of the VLDLR gene, and the encoded protein thereof, are known and include “ CAMRQ 1,” “CARMQ1,” “CHRMQ1,” “VLDLRCH,” and “VLDL-R.”

There are at least 2 known isoforms of human VLDLR protein, resulting from alternative splicing. VLDLR isoform long (Uniprot identifier: P98155-1) is 873 amino acids in length and has been chosen as the canonical sequence (SEQ ID NO: 111). VLDLR isoform short (Uniprot identifier: P98155-2) is 845 amino acids long and differs from the canonical sequence as follows: 751-779: STATTVTYSETKDTNTTEISATSGLVPGG →R. (SEQ ID NO: 112). Table 7 (below) provides the amino acid sequences for the VLDLR proteins.

TABLE 7
VLDLR Protein Sequence

| | |
|---|---|
| Isoform Long | MGTSALWALWLLLALCWAPRESGATGTGRKAKCEPSQFQCTNGRCITLLWKCDGDEDCVD |
| (UniProt: | GSDEKNCVKKTCAESDFVCNNGQCVPSRWKCDGDPDCEDGSDESPEQCHMRTCRIHEISC |
| P98155-1) | GAHSTQCIPVSWRCDGENDCDSGEDEENCGNITCSPDEFTCSSGRCISRNFVCNGQDDCS |
| (SEQ ID NO: | DGSDELDCAPPTCGAHEFQCSTSSCIPISWVCDDDADCSDQSDESLEQCGRQPVIHTKCP |
| 111) | ASEIQCGSGECIHKKWRCDGDPDCKDGSDEVNCPSRTCRPDQFECEDGSCIHGSRQCNGI |
| | RDCVDGSDEVNCKNVNQCLGPGKFKCRSGECIDISKVCNQEQDCRDWSDEPLKECHINEC |
| | LVNNGGCSHICKDLVIGYECDCAAGFELIDRKTCGDIDECQNPGICSQICINLKGGYKCE |
| | CSRGYQMDLATGVCKAVGKEPSLIFTNRRDIRKIGLERKEYIQLVEQLRNTVALDADIAA |
| | QKLFWADLSQKAIFSASIDDKVGRHVKMIDNVYNPAAIAVDWVYKTIYWTDAASKTISVA |
| | TLDGTKRKFLFNSDLREPASIAVDPLSGFVYWSDWGEPAKIEKAGMNGFDRRPLVTADIQ |
| | WPNGITLDLIKSRLYWLDSKLHMLSSVDLNGQDRRIVLKSLEFLAHPLALTIFEDRVYWI |
| | DGENEAVYGANKFTGSELATLVNNLNDAQDIIVYHELVQPSGKNWCEEDMENGGCEYLCL |
| | PAPQINDHSPKYTCSCPSGYNVEENGRDCQSTATTVTYSETKDTNTTEISATSGLVPGGI |
| | NVTTAVSEVSVPPKGTSAAWAILPLLLLVMAAVGGYLMWRNWQHKNMKSMNFDNPVYLKT |
| | TEEDLSIDIGRHSASVGHTYPAISVVSTDDDLA |
| Isoform Short | MGTSALWALWLLLALCWAPRESGATGTGRKAKCEPSQFQCTNGRCITLLWKCDGDEDCVD |
| (UniProt: | GSDEKNCVKKTCAESDFVCNNGQCVPSRWKCDGDPDCEDGSDESPEQCHMRTCRIHEISC |
| P98155-2) | GAHSTQCIPVSWRCDGENDCDSGEDEENCGNITCSPDEFTCSSGRCISRNFVCNGQDDCS |
| (SEQ ID NO: | DGSDELDCAPPTCGAHEFQCSTSSCIPISWVCDDDADCSDQSDESLEQCGRQPVIHTKCP |
| 112) | ASEIQCGSGECIHKKWRCDGDPDCKDGSDEVNCPSRTCRPDQFECEDGSCIHGSRQCNGI |
| | RDCVDGSDEVNCKNVNQCLGPGKFKCRSGECIDISKVCNQEQDCRDWSDEPLKECHINEC |
| | LVNNGGCSHICKDLVIGYECDCAAGFELIDRKTCGDIDECQNPGICSQICINLKGGYKCE |
| | CSRGYQMDLATGVCKAVGKEPSLIFTNRRDIRKIGLERKEYIQLVEQLRNTVALDADIAA |
| | QKLFWADLSQKAIFSASIDDKVGRHVKMIDNVYNPAAIAVDWVYKTIYWTDAASKTISVA |
| | TLDGTKRKFLFNSDLREPASIAVDPLSGFVYWSDWGEPAKIEKAGMNGFDRRPLVTADIQ |
| | WPNGITLDLIKSRLYWLDSKLHMLSSVDLNGQDRRIVLKSLEFLAHPLALTIFEDRVYWI |
| | DGENEAVYGANKFTGSELATLVNNLNDAQDIIVYHELVQPSGKNWCEEDMENGGCEYLCL |
| | PAPQINDHSPKYTCSCPSGYNVEENGRDCQRINVTTAVSEVSVPPKGTSAAWAILPLLLL |
| | VMAAVGGYLMWRNWQHKNMKSMNFDNPVYLKTTEEDLSIDIGRHSASVGHTYPAISVVST |
| | DDDLA |

As used herein, the term “VLDLR” (including its synonyms) includes any variants or isoforms of VLDLR which are naturally expressed by cells. Accordingly, in some aspects, a miR-485 inhibitor disclosed herein can increase the expression of VLDLR isoform long (i.e., canonical sequence). In some aspects, a miR-485 inhibitor disclosed herein can increase the expression of VLDLR isoform short. In some aspects, a miR-485 inhibitor disclosed herein can increase the expression of VLDLR isoform long and VLDLR isoform short. Unless indicated otherwise, the above-described isoforms of VLDLR are collectively referred to herein as “ VLDLR.”

In some aspects, a miR-485 inhibitor of the present disclosure increases the expression of VLDLR protein and/or VLDLR gene by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% compared to a reference (e.g., expression of VLDLR protein and/or VLDLR gene in a corresponding subject that did not receive an administration of the miR-485 inhibitor).

Not to be bound by any one theory, in some aspects, a miR-485 inhibitor disclosed herein increases the expression of VLDLR protein and/or VLDLR gene by reducing the expression and/or activity of miR-485, e.g., miR-485-3p.

NRXN1 Regulation

The disclosures provided herein demonstrates that the miR-485 inhibitors of the present disclosure can further regulate the expression of NRXN1, e.g., in a subject suffering from a disease or disorder disclosed herein (see, e.g., Alzheimer's disease). Therefore, in some aspects, the present disclosure provides a method of increasing an expression of a NRXN1 protein and/or a NRXN1 gene in a subject in need thereof, comprising administering to the subject a compound that inhibits miR-485 activity (i.e., miR-485 inhibitor). In certain aspects, inhibiting miR-485 activity increases the expression of a NRXN1 protein and/or NRXN1 gene in the subject.

Neurexin 1 (NRXN1) is a protein that in humans is encoded by the NRXN1 gene. The NRXN1 gene is located on chromosome 2 in humans (nucleotides 49,918,503 to 51,032,536 of NC_000003.12). Synonyms of the NRXN1 gene, and the encoded protein thereof, are known and include “PTHSL2,” “SCZD17,” and “Hs.22998.”

There are at least six known isoforms of human NRXN1 protein, resulting from alternative promoter usage and alternative splicing. NRXN1 isoform 1a (UniProt identifier: Q9ULB1-1) consists of 1,477 amino acids and has been chosen as the canonical sequence (SEQ ID NO: 104). NRXN1 isoform 2a (UniProt identifier: Q9ULB1-2) consists of 1,496 amino acids and differs from the isoform 1a canonical sequence as follows: 379-386: missing; 1239-1239: A→AGNNDNERLAIARQRIPYRLGRVVDEWLLDK; 1373-1375: missing (SEQ ID NO: 105). NRXN1 isoform 3a (UniProt identifier: Q9ULB1-3) consists of 1,547 amino acids and differs from the isoform 1a canonical sequence as follows: 258-258: →EIKFGLQCVLPVLLHDNDQGKYCCINTAKPLTEK; 386-386: M→MVNKLHCS; 1239-1239: A→AGNNDNERLAIARQRIPYRLGRVVDEWLLDK (SEQ ID NO: 106). NRXN1-beta isoform 4 (UniProt identifier: Q9ULB1-4) consists of 139 amino acids and differs from the isoform 1a canonical sequence as follows: 1-1335: missing; 1336-1344: GKPPTKEPI→MDMRWHCEN; 1373-1375: missing (SEQ ID NO: 107). NRXN1 isoform 1b (UniProt identifier: P58400-2) consists of 472 amino acids and has been chosen as the canonical sequence for NRXN1-beta (SEQ ID NO: 108). NRXN1-beta isoform 3b (UniProt identifier: P58400-1) consists of 442 amino acids and differs from the isoform 1b canonical sequence as follows: 205-234: missing (SEQ ID NO: 109).

Table 8 below provides the sequence for the six NRXN1 isoforms.

TABLE 8
NRXN1 Protein Isoforms

| | |
|---|---|
| Isoform 1a | MGTALLQRGGCFLLCLSLLLLGCWAELGSGLEFPGAEGQWTRFPKWNACCESEMSFQLKT |
| (UniProt: | RSARGLVLYFDDEGFCDFLELILTRGGRLQLSFSIFCAEPATLLADTPVNDGAWHSVRIR |
| Q9ULB1-1) | RQFRNTTLFIDQVEAKWVEVKSKRRDMTVFSGLFVGGLPPELRAAALKLTLASVREREPF |
| (SEQ ID | KGWIRDVRVNSSQVLPVDSGEVKLDDEPPNSGGGSPCEAGEEGEGGVCLNGGVCSVVDDQ |
| NO: 104) | AVCDCSRTGFRGKDCSQEDNNVEGLAHLMMGDQGKSKGKEEYIATFKGSEYFCYDLSQNP |
| | IQSSSDEITLSFKTLQRNGLMLHTGKSADYVNLALKNGAVSLVINLGSGAFEALVEPVNG |
| | KFNDNAWHDVKVTRNLRQHSGIGHAMVTISVDGILTTTGYTQEDYTMLGSDDFFYVGGSP |
| | STADLPGSPVSNNFMGCLKEVVYKNNDVRLELSRLAKQGDPKMKIHGVVAFKCENVATLD |
| | PITFETPESFISLPKWNAKKTGSISFDFRTTEPNGLILFSHGKPRHQKDAKHPQMIKVDF |
| | FAIEMLDGHLYLLLDMGSGTIKIKALLKKVNDGEWYHVDFQRDGRSGTISVNTLRTPYTA |
| | PGESEILDLDDELYLGGLPENKAGLVFPTEVWTALLNYGYVGCIRDLFIDGQSKDIRQMA |
| | EVQSTAGVKPSCSKETAKPCLSNPCKNNGMCRDGWNRYVCDCSGTGYLGRSCEREATVLS |
| | YDGSMFMKIQLPVVMHTEAEDVSLRFRSQRAYGILMATTSRDSADTLRLELDAGRVKLTV |
| | NLDCIRINCNSSKGPETLFAGYNLNDNEWHTVRVVRRGKSLKLTVDDQQAMTGQMAGDHT |
| | RLEFHNIETGIITERRYLSSVPSNFIGHLQSLTFNGMAYIDLCKNGDIDYCELNARFGFR |
| | NIIADPVTFKTKSSYVALATLQAYTSMHLFFQFKTTSLDGLILYNSGDGNDFIVVELVKG |
| | YLHYVFDLGNGANLIKGSSNKPLNDNQWHNVMISRDTSNLHTVKIDTKITTQITAGARNL |
| | DLKSDLYIGGVAKETYKSLPKLVHAKEGFQGCLASVDLNGRLPDLISDALFCNGQIERGC |
| | EGPSTTCQEDSCSNQGVCLQQWDGFSCDCSMTSFSGPLCNDPGTTYIFSKGGGQITYKWP |
| | PNDRPSTRADRLAIGFSTVQKEAVLVRVDSSSGLGDYLELHIHQGKIGVKFNVGTDDIAI |
| | EESNAIINDGKYHVVRFTRSGGNATLQVDSWPVIERYPAGRQLTIFNSQATIIIGGKEQG |
| | QPFQGQLSGLYYNGLKVLNMAAENDANIAIVGNVRLVGEVPSSMTTESTATAMQSEMSTS |
| | IMETTTTLATSTARRGKPPTKEPISQTTDDILVASAECPSDDEDIDPCEPSSGGLANPTR |
| | AGGREPYPGSAEVIRESSSTTGMVVGIVAAAALCILILLYAMYKYRNRDEGSYHVDESRN |
| | YISNSAQSNGAVVKEKQPSSAKSSNKNKKNKDKEYYV |
| Isoform 2a | MGTALLQRGGCFLLCLSLLLLGCWAELGSGLEFPGAEGQWTRFPKWNACCESEMSFQLKT |
| (UniProt: | RSARGLVLYFDDEGFCDFLELILTRGGRLQLSFSIFCAEPATLLADTPVNDGAWHSVRIR |
| Q9ULB1-2) | RQFRNTTLFIDQVEAKWVEVKSKRRDMTVFSGLFVGGLPPELRAAALKLTLASVREREPF |
| (SEQ ID | KGWIRDVRVNSSQVLPVDSGEVKLDDEPPNSGGGSPCEAGEEGEGGVCLNGGVCSVVDDQ |
| NO: 105) | AVCDCSRTGFRGKDCSQEDNNVEGLAHLMMGDQGKSKGKEEYIATFKGSEYFCYDLSQNP |
| | IQSSSDEITLSFKTLQRNGLMLHTGKSADYVNLALKNGAVSLVINLGSGAFEALVEPVNG |
| | KFNDNAWHDVKVTRNLRQVTISVDGILTTTGYTQEDYTMLGSDDFFYVGGSPSTADLPGS |
| | PVSNNFMGCLKEVVYKNNDVRLELSRLAKQGDPKMKIHGVVAFKCENVATLDPITFETPE |
| | SFISLPKWNAKKTGSISFDFRTTEPNGLILFSHGKPRHQKDAKHPQMIKVDFFAIEMLDG |
| | HLYLLLDMGSGTIKIKALLKKVNDGEWYHVDFQRDGRSGTISVNTLRTPYTAPGESEILD |
| | LDDELYLGGLPENKAGLVFPTEVWTALLNYGYVGCIRDLFIDGQSKDIRQMAEVQSTAGV |
| | KPSCSKETAKPCLSNPCKNNGMCRDGWNRYVCDCSGTGYLGRSCEREATVLSYDGSMFMK |
| | IQLPVVMHTEAEDVSLRFRSQRAYGILMATTSRDSADTLRLELDAGRVKLTVNLDCIRIN |
| | CNSSKGPETLFAGYNLNDNEWHTVRVVRRGKSLKLTVDDQQAMTGQMAGDHTRLEFHNIE |
| | TGIITERRYLSSVPSNFIGHLQSLTFNGMAYIDLCKNGDIDYCELNARFGFRNIIADPVT |
| | FKTKSSYVALATLQAYTSMHLFFQFKTTSLDGLILYNSGDGNDFIVVELVKGYLHYVFDL |
| | GNGANLIKGSSNKPLNDNQWHNVMISRDTSNLHTVKIDTKITTQITAGARNLDLKSDLYI |
| | GGVAKETYKSLPKLVHAKEGFQGCLASVDLNGRLPDLISDALFCNGQIERGCEGPSTTCQ |
| | EDSCSNQGVCLQQWDGFSCDCSMTSFSGPLCNDPGTTYIFSKGGGQITYKWPPNDRPSTR |
| | ADRLAIGFSTVQKEAVLVRVDSSSGLGDYLELHIHQGKIGVKFNVGTDDIAIEESNAIIN |
| | DGKYHVVRFTRSGGNATLQVDSWPVIERYPAGNNDNERLAIARQRIPYRLGRVVDEWLLD |
| | KGRQLTIFNSQATIIIGGKEQGQPFQGQLSGLYYNGLKVLNMAAENDANIAIVGNVRLVG |
| | EVPSSMTTESTATAMQSEMSTSIMETTTTLATSTARRGKPPTKEPISQTTDDILVASAEC |
| | PSDDEDIDPCEPSSANPTRAGGREPYPGSAEVIRESSSTTGMWGIVAAAALCILIILLYA |
| | MYKYRNRDEGSYHVDESRNYISNSAQSNGAVVKEKQPSSAKSSNKNKKNKDKEYYV |
| Isoform 3a | MGTALLQRGGCFLLCLSLLLLGCWAELGSGLEFPGAEGQWTRFPKWNACCESEMSFQLKT |
| (UniProt: | RSARGLVLYFDDEGFCDFLELILTRGGRLQLSFSIFCAEPATLLADTPVNDGAWHSVRIR |
| Q9ULB1- | RQFRNTTLFIDQVEAKWVEVKSKRRDMTVFSGLFVGGLPPELRAAALKLTLASVREREPF |
| 3) (SEQ ID | KGWIRDVRVNSSQVLPVDSGEVKLDDEPPNSGGGSPCEAGEEGEGGVCLNGGVCSVVDDQ |
| NO: 106) | AVCDCSRTGFRGKDCSQEIKFGLQCVLPVLLHDNDQGKYCCINTAKPLTEKDNNVEGLAH |
| | LMMGDQGKSKGKEEYIATFKGSEYFCYDLSQNPIQSSSDEITLSFKTLQRNGLMLHTGKS |
| | ADYVNLALKNGAVSLVINLGSGAFEALVEPVNGKFNDNAWHDVKVTRNLRQHSGIGHAMV |
| | NKLHCSVTISVDGILTTTGYTQEDYTMLGSDDFFYVGGSPSTADLPGSPVSNNFMGCLKE |
| | VVYKNNDVRLELSRLAKQGDPKMKIHGVVAFKCENVATLDPITFETPESFISLPKWNAKK |
| | TGSISFDFRTTEPNGLILFSHGKPRHQKDAKHPQMIKVDFFAIEMLDGHLYLLLDMGSGT |
| | IKIKALLKKVNDGEWYHVDFQRDGRSGTISVNTLRTPYTAPGESEILDLDDELYLGGLPE |
| | NKAGLVFPTEVWTALLNYGYVGCIRDLFIDGQSKDIRQMAEVQSTAGVKPSCSKETAKPC |
| | LSNPCKNNGMCRDGWNRYVCDCSGTGYLGRSCEREATVLSYDGSMFMKIQLPVVMHTEAE |
| | DVSLRFRSQRAYGILMATTSRDSADTLRLELDAGRVKLTVNLDCIRINCNSSKGPETLFA |
| | GYNLNDNEWHTVRVVRRGKSLKLTVDDQQAMTGQMAGDHTRLEFHNIETGIITERRYLSS |
| | VPSNFIGHLQSLTFNGMAYIDLCKNGDIDYCELNARFGFRNIIADPVTFKTKSSYVALAT |
| | LQAYTSMHLFFQFKTTSLDGLILYNSGDGNDFIVVELVKGYLHYVFDLGNGANLIKGSSN |
| | KPLNDNQWHNVMISRDTSNLHTVKIDTKITTQITAGARNLDLKSDLYIGGVAKETYKSLP |
| | KLVHAKEGFQGCLASVDLNGRLPDLISDALFCNGQIERGCEGPSTTCQEDSCSNQGVCLQ |
| | QWDGFSCDCSMTSFSGPLCNDPGTTYIFSKGGGQITYKWPPNDRPSTRADRLAIGFSTVQ |
| | KEAVLVRVDSSSGLGDYLELHIHQGKIGVKFNVGTDDIAIEESNAIINDGKYHVVRFTRS |
| | GGNATLQVDSWPVIERYPAGNNDNERLAIARQRIPYRLGRVVDEWLLDKGRQLTIFNSQA |
| | TIIIGGKEQGQPFQGQLSGLYYNGLKVLNMAAENDANIAIVGNVRLVGEVPSSMTTESTA |
| | TAMQSEMSTSIMETTTTLATSTARRGKPPTKEPISQTTDDILVASAECPSDDEDIDPCEP |
| | SSGGLANPTRAGGREPYPGSAEVIRESSSTTGMVVGIVAAAALCILILLYAMYKYRNRDE |
| | GSYHVDESRNYISNSAQSNGAWKEKQPSSAKSSNKNKKNKDKEYYV |
| Isoform 4a | MDMRWHCENSQTTDDILVASAECPSDDEDIDPCEPSSANPTRAGGREPYPGSAEVIRESS |
| (UniProt: | STTGMVVGIVAAAALCILILLYAMYKYRNRDEGSYHVDESRNYISNSAQSNGAVVKEKQP |
| Q9ULB1-4) | SSAKSSNKNKKNKDKEYYV |
| (SEQ ID | |
| NO: 107) | |
| Isoform 1b | MYQRMLRCGAELGSPGGGGGGGGGGGAGGRLALLWIVPLTLSGLLGVAWGASSLGAHHIH |
| (UniProt: | HFHGSSKHHSVPIAIYRSPASLRGGHAGTTYIFSKGGGQITYKWPPNDRPSTRADRLAIG |
| P58400-2) | FSTVQKEAVLVRVDSSSGLGDYLELHIHQGKIGVKFNVGTDDIAIEESNAIINDGKYHVV |
| (SEQ ID | RFTRSGGNATLQVDSWPVIERYPAGNNDNERLAIARQRIPYRLGRVVDEWLLDKGRQLTI |
| NO: 108) | FNSQATIIIGGKEQGQPFQGQLSGLYYNGLKVLNMAAENDANIAIVGNVRLVGEVPSSMT |
| | TESTATAMQSEMSTSIMETTTTLATSTARRGKPPTKEPISQTTDDILVASAECPSDDEDI |
| | DPCEPSSGGLANPTRAGGREPYPGSAEVIRESSSTTGMWGIVIWkALCILILLYAMYKY |
| | RNRDEGSYHVDESRNYISNSAQSNGAVVKEKQPSSAKSSNKNKKNKDKEYYV |
| Isoform 3b | MYQRMLRCGAELGSPGGGGGGGGGGGAGGRLALLWIVPLTLSGLLGVAWGASSLGAHHIH |
| (UniProt: | HFHGSSKHHSVPIAIYRSPASLRGGHAGTTYIFSKGGGQITYKWPPNDRPSTRADRLAIG |
| P58400-1) | FSTVQKEAVLVRVDSSSGLGDYLELHIHQGKIGVKFNVGTDDIAIEESNAIINDGKYHVV |
| (SEQ ID | RFTRSGGNATLQVDSWPVIERYPAGRQLTIFNSQATIIIGGKEQGQPFQGQLSGLYYNGL |
| NO: 109) | KVLNMAAENDANIAIVGNVRLVGEVPSSMTTESTATAMQSEMSTSIMETTTTLATSTARR |
| | GKPPTKEPISQTTDDILVASAECPSDDEDIDPCEPSSGGLANPTRAGGREPYPGSAEVIR |
| | ESSSTTGMVVGIVAAAALCILILLYAMYKYRNRDEGSYHVDESRNYISNSAQSNGAVVKE |
| | KQPSSAKSSNKNKKNKDKEYYV |

As used herein, the term “NRXN1” (including its synonyms) includes any variants or isoforms of NRXN1 and NRXN1-beta which are naturally expressed by cells. Accordingly, in some aspects, a miR-485 inhibitor disclosed herein can increase the expression of NRXN1 isoform 1a. In some aspects, a miR-485 inhibitor disclosed herein can increase the expression of NRXN1 isoform 2a. In some aspects, a miR-485 inhibitor disclosed herein can increase the expression of NRXN1 isoform 3a. In some aspects, a miR-485 inhibitor disclosed herein can increase the expression of NRXN1 isoform 4. In some aspects, a miR-485 inhibitor disclosed herein can increase the expression of NRXN1-beta isoform 1b. In some aspects, a miR-485 inhibitor disclosed herein can increase the expression of NRXN1-beta isoform 3b. In further aspects, a miR-485 inhibitor disclosed herein can increase the expression of one or more of NRXN1 isoform 1a, NRXN1-beta isoform 1b, NRXN1 isoform, 2a, NRXN1 isoform 3a, NRXN1-beta isoform 3b, and NRXN1 isoform 4. Unless indicated otherwise, NRXN1 isoform 1a, NRXN1-beta isoform 1b, NRXN1 isoform, 2a, NRXN1 isoform 3a, NRXN1-beta isoform 3b, and NRXN1 isoform 4 are collectively referred to herein as “NRXN1.”

In some aspects, a miR-485 inhibitor of the present disclosure increases the expression of NRXN1 protein and/or NRXN1 gene by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% compared to a reference (e.g., expression of NRXN1 protein and/or NRXN1 gene in a corresponding subject that did not receive an administration of the miR-485 inhibitor).

Not to be bound by any one theory, in some aspects, a miR-485 inhibitor disclosed herein increases the expression of NRXN1 protein and/or NRXN1 gene by reducing the expression and/or activity of miR-485, e.g., miR-485-3p.

GRIA4 Regulation

In some aspects, the miR-485 inhibitors of the present disclosure can further regulate the expression of GRIA4, e.g., in a subject suffering from a disease or disorder disclosed herein (see, e.g., Alzheimer's disease). Therefore, in some aspects, the present disclosure provides a method of increasing an expression of a GRIA4 protein and/or a GRIA4 gene in a subject in need thereof, comprising administering to the subject a compound that inhibits miR-485 activity (i.e., miR-485 inhibitor). In certain aspects, inhibiting miR-485 activity increases the expression of a GRIA4 protein and/or GRIA4 gene in the subject.

Glutamate receptor 4 (GRIA4) is a member of a family of L-glutamate-gated ion channels that mediate fast synaptic excitatory neurotransmission. These channels are also responsive to the glutamate agonist, alpha-amino-3-hydroxy-5-methyl-4-isoxazolpropionate (AMPA). In humans, GRIA4 is encoded by the GRIA4 gene, which is located on chromosome 11 (nucleotides 105,609,540 to 105,982,092 of NC_000011.10). Synonyms of the GRIA4 gene, and the encoded protein thereof, are known and include: “GLUR4,” “GLURD,” “GluA4,” “GLUR4C,” “NEDSGA,” and “ glutamate ionotropic receptor AMPA type subunit 4.”

There are at least two known isoforms of human GRIA4, resulting from alternative splicing. GRIA4 isoform 1 (UniProt identifier: P48058-1) consists of 902 amino acids and has been chosen as the canonical sequence (SEQ ID NO: 113). GRIA4 isoform 2 (UniProt identifier: P48058-2) is 433 amino acids in length and differs from the canonical sequence as follows: (i) 424-433: ESPYVMYKKN→PLMKNPILRN; and (ii) 434-902: Missing (SEQ ID NO: 114).

Table 9 below provides the sequences for the different GRIA4 isoforms.

TABLE 9
GRIA4 Protein Isoforms

| | |
|---|---|
| Isoform 1 | MRIISRQIVLLFSGFWGLAMGAFPSSVQIGGLFIRNTDQEYTAFRLAIFLHNTSPNASEA |
| (UniProt: | PFNLVPHVDNIETANSFAVTNAFCSQYSRGVFAIFGLYDKRSVHTLTSFCSALHISLITP |
| P48058-1) | SFPTEGESQFVLQLRPSLRGALLSLLDHYEWNCFVFLYDTDRGYSILQAIMEKAGQNGWH |
| (SEQ ID | VSAICVENFNDVSYRQLLEELDRRQEKKFVIDCEIERLQNILEQIVSVGKHVKGYHYIIA |
| NO: 113) | NLGFKDISLERFIHGGANVTGFQLVDFNTPMVIKLMDRWKKLDQREYPGSETPPKYTSAL |
| | TYDGVLVMAETFRSLRRQKIDISRRGNAGDCLANPAAPWGQGIDMERTLKQVRIQGLTGN |
| | VQFDHYGRRVNYTMDVFELKSTGPRKVGYWNDMDKLVLIQDVPTLGNDTAAIENRTVVVT |
| | TIMESPYVMYKKNHEMFEGNDKYEGYCVDLASEIAKHIGIKYKIAIVPDGKYGARDADTK |
| | IWNGMVGELVYGKAEIAIAPLTITLVREEVIDFSKPFMSLGISIMIKKPQKSKPGVFSFL |
| | DPLAYEIWMCIVFAYIGVSVVLFLVSRFSPYEWHTEEPEDGKEGPSDQPPNEFGIFNSLW |
| | FSLGAFMQQGCDISPRSLSGRIVGGVWWFFTLIIISSYTANLAAFLTVERMVSPIESAED |
| | LAKQTEIAYGTLDSGSTKEFFRRSKIAVYEKMWTYMRSAEPSVFTRTTAEGVARVRKSKG |
| | KFAFLLESTMNEYIEQRKPCDTMKVGGNLDSKGYGVATPKGSSLRTPVNLAVLKLSEAGV |
| | LDKLKNKWWYDKGECGPKDSGSKDKTSALSLSNVAGVFYILVGGLGLAMLVALIEFCYKS |
| | RAEAKRMKLTFSEAIRNKARLSITGSVGENGRVLTPDCPKAVHTGTAIRQSSGLAVIASD |
| | LP |
| Isoform 2 | MRIISRQIVLLFSGFWGLAMGAFPSSVQIGGLFIRNTDQEYTAFRLAIFLHNTSPNASEA |
| (UniProt: | PFNLVPHVDNIETANSFAVTNAFCSQYSRGVFAIFGLYDKRSVHTLTSFCSALHISLITP |
| P48058-2) | SFPTEGESQFVLQLRPSLRGALLSLLDHYEWNCFVFLYDTDRGYSILQAIMEKAGQNGWH |
| (SEQ ID | VSAICVENFNDVSYRQLLEELDRRQEKKFVIDCEIERLQNILEQIVSVGKHVKGYHYIIA |
| NO: 114) | NLGFKDISLERFIHGGANVTGFQLVDFNTPMVIKLMDRWKKLDQREYPGSETPPKYTSAL |
| | TYDGVLVMAETFRSLRRQKIDISRRGNAGDCLANPAAPWGQGIDMERTLKQVRIQGLTGN |
| | VQFDHYGRRVNYTMDVFELKSTGPRKVGYWNDMDKLVLIQDVPTLGNDTAAIENRTVVVT |
| | TIMPLMKNPILRN |

As used herein, the term “GRIA4” (including its synonyms) includes any variants or isoforms of GRIA4 which are naturally expressed by cells. Accordingly, in some aspects, a miR-485 inhibitor disclosed herein can increase the expression of GRIA4 isoform 1 (i.e., canonical sequence). In some aspects, a miR-485 inhibitor disclosed herein can increase the expression of GRIA4 isoform 2. In some aspects, a miR-485 inhibitor disclosed herein can increase the expression of GRIA4 isoform 1 and GRIA4 isoform 2. Unless indicated otherwise, the above-described isoforms of GRIA4 are collectively referred to herein as “GRIA4.”

In some aspects, a miR-485 inhibitor of the present disclosure increases the expression of GRIA4 protein and/or GRIA4 gene by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% compared to a reference (e.g., expression of GRIA4 protein and/or GRIA4 gene in a corresponding subject that did not receive an administration of the miR-485 inhibitor).

Not to be bound by any one theory, in some aspects, a miR-485 inhibitor disclosed herein increases the expression of GRIA4 protein and/or GRIA4 gene by reducing the expression and/or activity of miR-485, e.g., miR-485-3p.

NXPH1 Regulation

In some aspects, the miR-485 inhibitors of the present disclosure can further regulate the expression of NXPH1, e.g., in a subject suffering from a disease or disorder disclosed herein (see, e.g., Alzheimer's disease). Therefore, in some aspects, the present disclosure provides a method of increasing an expression of a NXPH1 protein and/or a NXPH1 gene in a subject in need thereof, comprising administering to the subject a compound that inhibits miR-485 activity (i.e., miR-485 inhibitor). In certain aspects, inhibiting miR-485 activity increases the expression of a NXPH1 protein and/or NXPH1 gene in the subject.

Neurexophilin-1 (NXPH1) is a protein that in humans is encoded by the NXPH1 gene. The NXPH1 gene is a member of the neurexophilin family and encodes a secreted protein with a variable N-terminal domain, a highly conserved, N-glycosylated central domain, a short linker region, and a cysteine-rich C-terminal domain. This protein forms a very tight complex with alpha neurexins, a group of proteins that promote adhesion between dendrites and axons. In humans, the NXPH1 gene is located on chromosome 7 (nucleotides 8,433,609 to 8,752,961 of NC_000007.14). Synonyms of the NXPH1 gene, and the encoded protein thereof, are known and include: “NPH1” and “Nbla00697.”

Table 10 below provides the amino acid sequence for the NXPH1 protein.

TABLE 10
NXPH1 amino acid sequence

| NXPH1 | MQAACWYVLFLLQPTVYLVTCANLTNGGKSELLKSGSSKSTLKHIWTESSKDLSISRLLS |
|---|---|
| (UniProt: | QTFRGKENDTDLDLRYDTPEPYSEQDLWDWLRNSTDLQEPRPRAKRRPIVKTGKFKKMFG |
| P58417-1) | WGDFHSNIKTVKLNLLITGKIVDHGNGTFSVYFRHNSTGQGNVSVSLVPPTKIVEFDLAQ |
| (SEQ ID NO: | QTVIDAKDSKSFNCRIEYEKVDKATKNTLCNYDPSKTCYQEQTQSHVSWLCSKPFKVICI |
| 115) | YISFYSTDYKLVQKVCPDYNYHSDTPYFPSG |

As used herein, the term “NXPH1” (including its synonyms) includes any variants or isoforms of NXPH1 which are naturally expressed by cells.

In some aspects, a miR-485 inhibitor of the present disclosure increases the expression of NXPH1 protein and/or NXPH1 gene by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% compared to a reference (e.g., expression of NXPH1 protein and/or NXPH1 gene in a corresponding subject that did not receive an administration of the miR-485 inhibitor).

Not to be bound by any one theory, in some aspects, a miR-485 inhibitor disclosed herein increases the expression of NXPH1 protein and/or NXPH1 gene by reducing the expression and/or activity of miR-485, e.g., miR-485-3p.

PSD-95 Regulation

In some aspects, the miR-485 inhibitors of the present disclosure can regulate the expression of PSD-95, e.g., in a subject suffering from a disease or disorder disclosed herein (see, e.g., Alzheimer's disease). Therefore, in some aspects, the present disclosure provides a method of increasing an expression of a PSD-95 protein and/or a PSD-95 gene (i.e., DLG4) in a subject in need thereof, comprising administering to the subject a compound that inhibits miR-485 activity (i.e., miR-485 inhibitor). In certain aspects, inhibiting miR-485 activity increases the expression of a PSD-95 protein and/or PSD-95 gene in the subject.

Postsynaptic density protein 95 (PSD-95), also known as synapse-associated protein 90 (SAP-90) is a protein that in humans is encoded by the DLG4 (discs large homolog 4) gene (also referred to herein as “PSD-95 gene”). The DLG4 gene is located on chromosome 17 in humans (nucleotides 7,187,180-7,220,050 of GenBank Accession Number NC_000017.11, minus strand orientation). Synonyms of the DLG4 gene, and the encoded protein thereof, are known and include “discs large MAGUK scaffold protein 4,” “MRD62,” “PSD95,” and “SAP90.”

There are at least three known isoforms of human PSD-95 protein, resulting from alternative splicing. PSD-95 isoform 1 (also known as PSD95-alpha) (UniProt identifier: P78352-1) consists of 724 amino acids and has been chosen as the canonical sequence (SEQ ID NO: 116). PSD-95 isoform 2 (also known as PSD95-beta) (UniProt identifier: P78352-2) consists of 767 amino acids and differs from the canonical sequence as follows: 1-10: MDCLCIVTTK→MSQRPRAPRSALWLLAPPLLRWAPPLLTVLHSDLFQALLDILDYYEASLSESQ (SEQ ID NO: 1 17), PSD-95 isoform 3 (UniProt identifier: P78352-3) consists of 721 amino acids and differs from the canonical sequence as follows: 51-53: Missing (SEQ ID NO: 118).

Table 11 below provides the amino acid sequences for the PSD-95 protein, including known isoforms.

TABLE 11
PSD-95 Protein Sequence

| | |
|---|---|
| Isoform 1 | MDCLCIVTTKKYRYQDEDTPPLEHSPAHLPNQANSPPVIVNTDTLEAPGYELQVNGTEGE |
| (UniProt: | MEYEEITLERGNSGLGFSIAGGTDNPHIGDDPSIFITKIIPGGAAAQDGRLRVNDSILFV |
| P78352-1) (SEQ | NEVDVREVTHSAAVEALKEAGSIVRLYVMRRKPPAEKVMEIKLIKGPKGLGFSIAGGVGN |
| ID NO: 116) | QHIPGDNSIYVTKIIEGGAAHKDGRLQIGDKILAVNSVGLEDVMHEDAVAALKNTYDVVY |
| | LKVAKPSNAYLSDSYAPPDITTSYSQHLDNEISHSSYLGTDYPTAMTPTSPRRYSPVAKD |
| | LLGEEDIPREPRRIVIHRGSTGLGFNIVGGEDGEGIFISFILAGGPADLSGELRKGDQIL |
| | SVNGVDLRNASHEQAAIALKNAGQTVTIIAQYKPEEYSRFEAKIHDLREQLMNSSLGSGT |
| | ASLRSNPKRGFYIRALFDYDKTKDCGFLSQALSFRFGDVLHVIDASDEEWWQARRVHSDS |
| | ETDDIGFIPSKRRVERREWSRLKAKDWGSSSGSQGREDSVLSYETVTQMEVHYARPIIIL |
| | GPTKDRANDDLLSEFPDKFGSCVPHTTRPKREYEIDGRDYHFVSSREKMEKDIQAHKFIE |
| | AGQYNSHLYGTSVQSVREVAEQGKHCILDVSANAVRRLQAAHLHPIAIFIRPRSLENVLE |
| | INKRITEEQARKAFDRATKLEQEFTECFSAIVEGDSFEEIYHKVKRVIEDLSGPYIWVPA |
| | RERL |
| Isoform 2 | MSQRPRAPRSALWLLAPPLLRWAPPLLTVLHSDLFQALLDILDYYEASLSESQKYRYQDE |
| (UniProt: | DTPPLEHSPAHLPNQANSPPVIVNTDTLEAPGYELQVNGTEGEMEYEEITLERGNSGLGF |
| P78352-2) (SEQ | SIAGGTDNPHIGDDPSIFITKIIPGGAAAQDGRLRVNDSILFVNEVDVREVTHSAAVEAL |
| ID NO: 117) | KEAGSIVRLYVMRRKPPAEKVMEIKLIKGPKGLGFSIAGGVGNQHIPGDNSIYVTKIIEG |
| | GAAHKDGRLQIGDKILAVNSVGLEDVMHEDAVAALKNTYDVVYLKVAKPSNAYLSDSYAP |
| | PDITTSYSQHLDNEISHSSYLGTDYPTAMTPTSPRRYSPVAKDLLGEEDIPREPRRIVIH |
| | RGSTGLGFNIVGGEDGEGIFISFILAGGPADLSGELRKGDQILSVNGVDLRNASHEQAAI |
| | ALKNAGQTVTIIAQYKPEEYSRFEAKIHDLREQLMNSSLGSGTASLRSNPKRGFYIRALF |
| | DYDKTKDCGFLSQALSFRFGDVLHVIDASDEEWWQARRVHSDSETDDIGFIPSKRRVERR |
| | EWSRLKAKDWGSSSGSQGREDSVLSYETVTQMEVHYARPIIILGPTKDRANDDLLSEFPD |
| | KFGSCVPHTTRPKREYEIDGRDYHFVSSREKMEKDIQAHKFIEAGQYNSHLYGTSVQSVR |
| | EVAEQGKHCILDVSANAVRRLQAAHLHPIAIFIRPRSLENVLEINKRITEEQARKAFDRA |
| | TKLEQEFTECFSAIVEGDSFEEIYHKVKRVIEDLSGPYIWVPARERL |
| Isoform 3 | MDCLCIVTTKKYRYQDEDTPPLEHSPAHLPNQANSPPVIVNTDTLEAPGYVNGTEGEMEY |
| (UniProt: | EEITLERGNSGLGFSIAGGTDNPHIGDDPSIFITKIIPGGAAAQDGRLRVNDSILFVNEV |
| P78352-3) (SEQ | DVREVTHSAAVEALKEAGSIVRLYVMRRKPPAEKVMEIKLIKGPKGLGFSIAGGVGNQHI |
| ID NO: 118) | PGDNSIYVTKIIEGGAAHKDGRLQIGDKILAVNSVGLEDVMHEDAVAALKNTYDVVYLKV |
| | AKPSNAYLSDSYAPPDITTSYSQHLDNEISHSSYLGTDYPTAMTPTSPRRYSPVAKDLLG |
| | EEDIPREPRRIVIHRGSTGLGFNIVGGEDGEGIFISFILAGGPADLSGELRKGDQILSVN |
| | GVDLRNASHEQAAIALKNAGQTVTIIAQYKPEEYSRFEAKIHDLREQLMNSSLGSGTASL |
| | RSNPKRGFYIRALFDYDKTKDCGFLSQALSFRFGDVLHVIDASDEEWWQARRVHSDSETD |
| | DIGFIPSKRRVERREWSRLKAKDWGSSSGSQGREDSVLSYETVTQMEVHYARPIIILGPT |
| | KDRANDDLLSEFPDKFGSCVPHTTRPKREYEIDGRDYHFVSSREKMEKDIQAHKFIEAGQ |
| | YNSHLYGTSVQSVREVAEQGKHCILDVSANAVRRLQAAHLHPIAIFIRPRSLENVLEINK |
| | RITEEQARKAFDRATKLEQEFTECFSAIVEGDSFEEIYHKVKRVIEDLSGPYIWVPARER |

As used herein, the term “PSD-95” (including its synonyms) includes any variants or isoforms of PSD-95 which are naturally expressed by cells. Accordingly, in some aspects, a miR-485 inhibitor disclosed herein can increase the expression of PSD-95 isoform 1. In some aspects, a miR-485 inhibitor disclosed herein can increase the expression of PSD-95 isoform 2. In some aspects, a miR-485 inhibitor can increase the expression of PSD-95 isoform 3. In further aspects, a miR-485 inhibitor disclosed herein can increase the expression of PSD-95 isoform 1, PSD-95 isoform 2, and PSD-95 isoform 3. Unless indicated otherwise, the above-described isoforms of PSD-95 are collectively referred to herein as “PSD-95.”

In some aspects, a miR-485 inhibitor of the present disclosure increases the expression of PSD-95 protein and/or PSD-95 gene by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% compared to a reference (e.g., expression of PSD-95 protein and/or PSD-95 gene in a corresponding subject that did not receive an administration of the miR-485 inhibitor).

Not to be bound by any one theory, in some aspects, a miR-485 inhibitor disclosed herein increases the expression of PSD-95 protein and/or PSD-95 gene by reducing the expression and/or activity of miR-485, e.g., miR-485-3p.

Synaptophysin Regulation

The disclosures provided herein further demonstrates that the miR-485 inhibitors described herein can regulate the expression of synaptophysin, e.g., in a subject suffering from a disease or disorder disclosed herein (see, e.g., Parkinson's disease). Therefore, in some aspects, the present disclosure provides a method of increasing an expression of a synaptophysin protein and/or a synaptophysin gene (i.e., SYP) in a subject in need thereof, comprising administering to the subject a compound that inhibits miR-485 activity (i.e., miR-485 inhibitor). In certain aspects, inhibiting miR-485 activity increases the expression of a synaptophysin protein and/or synaptophysin gene in the subject.

Synaptophysin, also known as the major synaptic vesicle protein p38, is a protein that in human is encoded by the SYP gene (also referred to herein as “synaptophysin gene”). The SYP gene is located on the short arm of the X chromosome (nucleotides 49,187,804-49,200,259 of GenBank Accession Number NC_000023.11, minus strand orientation). Synonyms of the SYP gene, and the encoded protein thereof, are known and include “MRX96” and “MRXSYP.”

There are at least two known isoforms of the synaptophysin protein, resulting from alternative splicing. Synaptophysin isoform 1 (UniProt identifier: P08247-1) consists of 313 amino acids and has been chosen as the canonical sequence (SEQ ID NO: 119). Synaptophysin isoform 2 (UniProt identifier: P08247-2) is 195 amino acids in length and differs from the canonical sequence as follows: 1-118: missing (SEQ ID NO: 120).

Table 12 below provides the amino acid sequences for the synaptophysin protein, including any known isoforms.

TABLE 12
Synaptophysin Protein Sequence

| | |
|---|---|
| Isoform 1 | MLLLADMDVVNQLVAGGQFRVVKEPLGFVKVLQWVFAIFAFATCGSYSGELQLSVDCANK |
| (UniProt: | TESDLSIEVEFEYPFRLHQVYFDAPTCRGGTTKVFLVGDYSSSAEFFVTVAVFAFLYSMG |
| P08247-1) (SEQ | ALATYIFLQNKYRENNKGPMLDFLATAVFAFMWLVSSSAWAKGLSDVKMATDPENIIKEM |
| ID NO: 119) | PVCRQTGNTCKELRDPVTSGLNTSWFGFLNLVLWVGNLWFVFKETGWTVAPFLRAPPGAP |
| | EKQPAPGDAYGDAGYGQGPGGYGPQDSYGPQGGYQPDYGQPAGSGGSGYGPQGDYGQQGY |
| | GPQGAPTSFSNQM |
| Isoform 2 | MGALATYIFLQNKYRENNKGPMLDFLATAVFAFMWLVSSSAWAKGLSDVKMATDPENIIK |
| (UniProt: | EMPVCRQTGNTCKELRDPVTSGLNTSVVFGFLNLVLWVGNLWFVFKETGWAAPFLRAPPG |
| P08247-2) (SEQ | APEKQPAPGDAYGDAGYGQGPGGYGPQDSYGPQGGYQPDYGQPAGSGGSGYGPQGDYGQQ |
| ID NO: 120) | GYGPQGAPTSFSNQM |

As used herein, the term “synaptophysin” (including its synonyms) includes any variants or isoforms of synaptophysin which are naturally expressed by cells. Accordingly, in some aspects, a miR-485 inhibitor disclosed herein can increase the expression of synaptophysin isoform 1. In some aspects, a miR-485 inhibitor disclosed herein can increase the expression of synaptophysin isoform 2. In further aspects, a miR-485 inhibitor disclosed herein can increase the expression of both synaptophysin isoform 1 and synaptophysin isoform 2. Unless indicated otherwise, the above-described isoforms of synaptophysin are collectively referred to herein as “ synaptophysin.”

In some aspects, a miR-485 inhibitor of the present disclosure increases the expression of synaptophysin protein and/or synaptophysin gene by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% compared to a reference (e.g., expression of synaptophysin protein and/or synaptophysin gene in a corresponding subject that did not receive an administration of the miR-485 inhibitor).

Not to be bound by any one theory, in some aspects, a miR-485 inhibitor disclosed herein increases the expression of synaptophysin protein and/or synaptophysin gene by reducing the expression and/or activity of miR-485, e.g., miR-485-3p.

Caspase-3 Regulation

In some aspects, the disclosures provided herein demonstrates that the miR-485 inhibitors of the present disclosure can regulate the expression of caspase-3, e.g., in a subject suffering from a disease or disorder disclosed herein (e.g., Parkinson's disease). Therefore, in some aspects, the present disclosure provides a method of decreasing an expression of a caspase-3 protein and/or a caspase-3 gene (i.e., CASP3) in a subject in need thereof, comprising administering to the subject a compound that inhibits miR-485 activity (i.e., miR-485 inhibitor). In certain aspects, inhibiting miR-485 activity decreases the expression of a caspase-3 protein and/or caspase-3 gene in the subject.

Caspase-3 is a member of the cysteine-aspartic acid protease (caspase) family, and plays central role in cell apoptosis by interacting with caspase-8 and caspase-9. In humans, the caspase-3 protein is encoded by the CASP3 gene (also referred to herein as “caspase-3 gene”). The CASP3 gene is located on chromosome 4 in humans (nucleotides 184,627,696-184,649,509 of GenBank Accession Number NC_000004.12, minus strand orientation). Synonyms of the CASP3 gene, and the encoded protein thereof, are known and include “apopain,” “CPP32,” “SREBP cleavage activity 1,” protein yama,” “SCA-1,” “PARP cleavage protease,” “procaspase 3,” and “Yama.”

Table 13 below provides the amino acid sequence for the caspase-3 protein precursor, as well as the cleaved form of the caspase-3 protein.

TABLE 13
Caspase-3 Protein Sequence

| | |
|---|---|
| Isoform 1 | MENTENSVDSKSIKNLEPKIIHGSESMDSGISLDNSYKMDYPEMGLCIIINNKNFHKSTG |
| (UniProt: | MTSRSGTDVDAANLRETFRNLKYEVRNKNDLTREEIVELMRDVSKEDHSKRSSFVCVLLS |
| P42574-1) (SEQ | HGEEGIIFGTNGPVDLKKITNFFRGDRCRSLTGKPKLFIIQACRGTELDCGIETDSGVDD |
| ID NO: 121) | DMACHKIPVEADFLYAYSTAPGYYSWRNSKDGSWFIQSLCAMLKQYADKLEFMHILTRVN |
| | RKVATEFESFSFDATFHAKKQIPCIVSMLTKELYFYH |

As used herein, the term “caspase-3” (including its synonyms) includes any variants or isoforms of caspase-3 which are naturally expressed by cells (e.g., cleaved caspase-3).

In some aspects, a miR-485 inhibitor of the present disclosure decreases the expression of caspase-3 protein and/or caspase-3 gene by at least about 5%, at least about 10%, at least about 20%, at least about 30%, at least about 40%, at least about 50%, at least about 60%, at least about 70%, at least about 80%, at least about 90%, or at least about 100% compared to a reference (e.g., expression of caspase-3 protein and/or caspase-3 gene in a corresponding subject that did not receive an administration of the miR-485 inhibitor).

Not to be bound by any one theory, in some aspects, a miR-485 inhibitor disclosed herein decreases the expression of caspase-3 protein and/or caspase-3 gene by reducing the expression and/or activity of miR-485, e.g., miR-485-3p.

As will be apparent from the present disclosure, any disease or condition associated with abnormal (e.g., reduced) level of a SIRT1 protein and/or SIRT1 gene can be treated with the present disclosure. In some aspects, the present disclosure can be useful in treating any disease or condition associated with abnormal (e.g., reduced) level of a CD36 protein and/or CD36 gene. In some aspects, the present disclosure can also be used to treat a disease or disorder associated with abnormal (e.g., reduced) level of a PGC1-α protein and/or PGC1-α gene. In some aspects, the present disclosure can also be used to treat a disease or disorder associated with abnormal (e.g., reduced) level of a LRRK2 protein and/or LRRK2 gene. In some aspects, the present disclosure can also be used to treat a disease or disorder associated with abnormal (e.g., reduced) level of a NRG1 protein and/or NRG1 gene. In some aspects, the present disclosure can also be used to treat a disease or disorder associated with abnormal (e.g., reduced) level of a STMN2 protein and/or STMN2 gene. In some aspects, the present disclosure can also be used to treat a disease or disorder associated with abnormal (e.g., reduced) level of a VLDLR protein and/or VLDLR gene. In some aspects, the present disclosure can also be used to treat a disease or disorder associated with abnormal (e.g., reduced) level of a NRXN1 protein and/or NRXN1 gene. In some aspects, the present disclosure can also be used to treat a disease or disorder associated with abnormal (e.g., reduced) level of a GRIA4 protein and/or GRIA4 gene. In some aspects, the present disclosure can also be used to treat a disease or disorder associated with abnormal (e.g., reduced) level of a NXPH1 protein and/or NXPH1 gene. In some aspects, the present disclosure can be used to treat a disease or disorder associated with abnormal (e.g., reduced) level of a PSD-95 protein and/or PSD-95 gene. In some aspects, the present disclosure can be used to treat a disease or disorder associated with abnormal (e.g., reduced) level of a synaptophysin protein and/or synaptophysin gene. In some aspects, the present disclosure can be used to treat a disease or disorder associated with abnormal (e.g., increased) level of a caspase-3 protein and/or caspase-3 gene.

In some aspects, a disease or condition associated with abnormal (e.g., reduced or increased) level of such proteins and/or genes comprises a neurodegenerative disease or disorder. As used herein, the term “neurodegenerative disease or disorder” refers to a disease or disorder caused by the progressive pathologic changes within the nervous system, particularly within the neurons of the brain. In some aspects, such progressive destruction of the nervous system can result in physical (e.g., ataxias) and/or mental (e.g., dementia) impairments. Non-limiting examples of neurodegenerative diseases or disorders that can be treated with the present disclosure include Alzheimer's disease, Parkinson's disease, or any combination thereof. Other diseases or conditions that can be treated with the present disclosure include, but are not limited to, autism spectrum disorder, mental retardation, seizure, stroke, spinal cord injury, or any combination thereof.

In some aspects, a disease or disorder that can be treated with the present disclosure comprises Alzheimer's disease. In certain aspects, Alzheimer's disease comprises pre-dementia Alzheimer's disease, early Alzheimer's disease, moderate Alzheimer's disease, advanced Alzheimer's disease, early onset familial Alzheimer's disease, inflammatory Alzheimer's disease, non-inflammatory Alzheimer's disease, cortical Alzheimer's disease, early-onset Alzheimer's disease, late-onset Alzheimer's disease, or any combination thereof.

In some aspects, a disease or disorder that can be treated comprises a parkinsonism. As used herein, the term “parkinsonism” refers to a group of neurological disorders that causes a combination of the movement abnormalities seen in Parkinson's disease. Non-limiting examples of such movement abnormalities include tremor, slow movement (bradykinesia), postural instability, loss of postural reflexes, flexed posture, freezing phenomenon (when the feet are transiently “glued” to the ground), impaired speech, muscle stiffness (rigidity), or combinations thereof. In some aspects, parkinsonism comprises a Parkinson's disease, progressive supranuclear palsy (PSP), multiple system atrophy (MSA), corticalbasal degeneration (CBD), normal pressure hydrocephalus (NSA), vascular parkinsonism (also known as cerebrovascular disease), diffuse Lewy body disease, Parkinson-dementia, X-linked dystonia-parkinsonism, secondary Parkinsonism (resulting from environmental etiology, e.g., toxins, drugs, post encephalitic, brain tumors, head trauma, normal pressure hydrocephalus), or combinations thereof.

In some aspects, a parkinsonism that can be treated with the present disclosure is a Parkinson's disease. As used herein, the term “Parkinson's disease” (PD) refers to neurodegenerative disorder leading to motor and non-motor manifestations (i.e., symptoms) and characterized by extensive degeneration of dopaminergic neurons in the nigrostriatal system. Non-limiting examples of motor and non-motor manifestations of PD are provided elsewhere in the present disclosure. Proteinopathy (α-synuclein abnormal aggregation) is a hallmark of PD. Other exemplary features of PD include dopaminergic neuron damage, mitochondrial dysfunction, neuroinflammation, protein homeostasis (e.g., autophagic clearance of damaged proteins and organelles glial cell dysfunction), and combinations thereof. Not to be bound by any one theory, in some aspects, miR-485 inhibitors of the present disclosure can treat PD by improving one or more of these features of PD.

In some aspects, administering a miR-485 inhibitor disclosed herein can improve one or more symptoms of a disease or condition associated with abnormal (e.g., reduced) levels of SIRT1 protein and/or SIRT1 gene. In some aspects, administering a miR-485 inhibitor disclosed herein can improve one or more symptoms of a disease or condition associated with abnormal (e.g., reduced) levels of CD36 protein and/or CD36 gene. In some aspects, administering a miR-485 inhibitor disclosed herein can improve one or more symptoms of a disease or condition associated with abnormal (e.g., reduced) levels of PGC1-α protein and/or PGC1-α gene. In some aspects, administering a miR-485 inhibitor disclosed herein can improve one or more symptoms of a disease or condition associated with abnormal (e.g., reduced) levels of LRRK2 protein and/or LRRK2 gene. In some aspects, administering a miR-485 inhibitor disclosed herein can improve one or more symptoms of a disease or condition associated with abnormal (e.g., reduced) levels of NRG1 protein and/or NRG1 gene. In some aspects, administering a miR-485 inhibitor disclosed herein can improve one or more symptoms of a disease or condition associated with abnormal (e.g., reduced) levels of STMN2 protein and/or STMN2 gene. In some aspects, administering a miR-485 inhibitor disclosed herein can improve one or more symptoms of a disease or condition associated with abnormal (e.g., reduced) levels of VLDLR protein and/or VLDLR gene. In some aspects, administering a miR-485 inhibitor disclosed herein can improve one or more symptoms of a disease or condition associated with abnormal (e.g., reduced) levels of NRXN1 protein and/or NRXN1 gene. In some aspects, administering a miR-485 inhibitor disclosed herein can improve one or more symptoms of a disease or condition associated with abnormal (e.g., reduced) levels of GRIA4 protein and/or GRIA4 gene. In some aspects, administering a miR-485 inhibitor disclosed herein can improve one or more symptoms of a disease or condition associated with abnormal (e.g., reduced) levels of NXPH1 protein and/or NXPH1 gene. In some aspects, administering a miR-485 inhibitor disclosed herein can improve one or more symptoms of a disease or condition associated with abnormal (e.g., reduced) levels of PSD-95 protein and/or PSD-95 gene. In some aspects, administering a miR-485 inhibitor disclosed herein can improve one or more symptoms of a disease or condition associated with abnormal (e.g., reduced) levels of synaptophysin protein and/or synaptophysin gene. In some aspects, administering a miR-485 inhibitor disclosed herein can improve one or more symptoms of a disease or condition associated with abnormal (e.g., increased) levels of caspase-3 protein and/or caspase-3 gene. Non-limiting examples of such symptoms are described below.

In some aspects, administering a miR-485 inhibitor of the present disclosure reduces the occurrence or risk of occurrence of one or more symptoms of cognitive impairments in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., subjects that did not receive an administration of the miR-485 inhibitor).

In some aspects, administering a miR-485 inhibitor of the present disclosure reduces memory loss in a subject (e.g., suffering from a neurodegenerative disease) compared to a reference (e.g., memory loss in the subject prior to the administering). In some aspects, administering a miR-485 inhibitor of the present disclosure reduces memory loss or the risk of occurrence of memory loss in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., subjects that did not receive an administration of the miR-485 inhibitor).

In some aspects, administering a miR-485 inhibitor of the present disclosure improves memory retention in a subject (e.g., suffering from a neurodegenerative disease) compared to a reference (e.g., memory retention in the subject prior to the administering). In some aspects, administering a miR-485 inhibitor of the present disclosure improves and/or increases memory retention in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 250%, or at least about 300% or more compared to a reference (e.g., subjects that did not receive an administration of the miR-485 inhibitor).

In some aspects, administering a miR-485 inhibitor of the present disclosure improves spatial working memory in a subject (e.g., suffering from a neurodegenerative disease) compared to a reference (e.g., spatial working memory in the subject prior to the administering). As used herein, the term “spatial working memory” refers to the ability to keep spatial information activity in working memory over a short period of time. In some aspects, spatial working memory is improved and/or increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., subjects that did not receive an administration of the miR-485 inhibitor).

In some aspects, administering a miR-485 inhibitor of the present disclosure increases the phagocytic activity of scavenger cells (e.g., glial cells) (e.g., by increasing the expression of CD36 protein and/or CD36 gene) in a subject (e.g., suffering from a neurodegenerative disease) compared to a reference (e.g., phagocytic activity in the subject prior to the administering). In some aspects, administering a miR-485 inhibitor of the present disclosure increases dendritic spine density of a neuron in a subject (e.g., suffering from a neurodegenerative disease) by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., subjects that did not receive an administration of the miR-485 inhibitor).

In some aspects, administering a miR-485 inhibitor of the present disclosure reduces an amyloid beta (Aβ) plaque load in a subject (e.g., suffering from a neurodegenerative disease) (e.g., by increasing the expression of CD36 protein and/or CD36 gene) compared to a reference (e.g., amyloid beta (Aβ) plaque load in the subject prior to the administering). As used herein, “amyloid beta plaque” refers to all forms of aberrant deposition of amyloid beta including large aggregates and small associations of a few amyloid beta peptides and can contain any variation of the amyloid beta peptides. Amyloid beta (Aβ) plaque is known to cause neuronal changes, e.g., aberrations in synapse composition, synapse shape, synapse density, loss of synaptic conductivity, changes in dendrite diameter, changes in dendrite length, changes in spine density, changes in spine area, changes in spine length, or changes in spine head diameter. In some aspects, administering a miR-485 inhibitor of the present disclosure reduces an amyloid beta plaque load in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 85%, at least about 90%, at least about 95%, or about 100% compared to a reference (e.g., subjects that did not receive an administration of the miR-485 inhibitor).

In some aspects, administering a miR-485 inhibitor disclosed herein increases neurogenesis in a subject (e.g., suffering from a neurodegenerative disease) (e.g., by increasing the expression of CD36 protein and/or CD36 gene) compared to a reference (e.g., neurogenesis in the subject prior to the administering). As used herein, the term “neurogenesis” refers to the process by which neurons are created. Neurogenesis encompasses proliferation of neural stem and progenitor cells, differentiation of these cells into new neural cell types, as well as migration and survival of the new cells. The term is intended to cover neurogenesis as it occurs during normal development, predominantly during pre-natal and peri-natal development, as well as neural cells regeneration that occurs following disease, damage or therapeutic intervention. Adult neurogenesis is also termed “nerve” or “neural” regeneration. In some aspects, administering a miR-485 inhibitor of the present disclosure increases neurogenesis in a subject (e.g., suffering from a neurodegenerative disease) by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., subjects that did not receive an administration of the miR-485 inhibitor).

In some aspects, increasing and/or inducing neurogenesis is associated with increased proliferation, differentiation, migration, and/or survival of neural stem cells and/or progenitor cells. Accordingly, in some aspects, administering a miR-485 inhibitor of the present disclosure can increase the proliferation of neural stem cells and/or progenitor cells in the subject. In certain aspects, the proliferation of neural stem cells and/or progenitor cells is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., subjects that did not receive an administration of the miR-485 inhibitor). In some aspects, the survival of neural stem cells and/or progenitor cells is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., subjects that did not receive an administration of the miR-485 inhibitor).

In some aspects, increasing and/or inducing neurogenesis is associated with an increased number of neural stem cells and/or progenitor cells. In certain aspects, the number of neural stem cells and/or progenitor cells is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., subjects that did not receive an administration of the miR-485 inhibitor).

In some aspects, increasing and/or inducing neurogenesis is associated with increased axon, dendrite, and/or synapse development. In certain aspects, axon, dendrite, and/or synapse development is increased by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, or at least about 300% or more compared to a reference (e.g., subjects that did not receive an administration of the miR-485 inhibitor).

In some aspects, administering a miR-485 inhibitor disclosed herein prevents and/or inhibits the development of an amyloid beta plaque load in a subject (e.g., suffering from a neurodegenerative disease). In some aspects, administering a miR-485 inhibitor disclosed herein delays the onset of the development of an amyloid beta plaque load in a subject (e.g., suffering from a neurodegenerative disease). In some aspects, administering a miR-485 inhibitor of the present disclosure lowers the risk of development an amyloid beta plaque load in a subject (e.g., suffering from a neurodegenerative disease).

In some aspects, administering a miR-485 inhibitor of the present disclosure increases dendritic spine density of a neuron in a subject (e.g., suffering from a neurodegenerative disease) compared to a reference (e.g., dendritic spine density of a neuron in the subject prior to the administering). In some aspects, administering a miR-485 inhibitor of the present disclosure increases dendritic spine density of a neuron in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 250%, or at least about 300% or more compared to a reference (e.g., subjects that did not receive an administration of the miR-485 inhibitor).

In some aspects, administering a miR-485 inhibitor disclosed herein decreases the loss of dendritic spines of a neuron in a subject (e.g., suffering from a neurodegenerative disease) compared to a reference (e.g., loss of dendritic spines of a neuron in the subject prior to the administering). In certain aspects, administering a miR-485 inhibitor decreases the loss of dendritic spines of a neuron in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., subjects that did not receive an administration of the miR-485 inhibitor).

In some aspects, administering a miR-485 inhibitor of the present disclosure decreases neuroinflammation (e.g., by increasing the expression of SIRT1 protein and/or SIRT1 gene) in a subject (e.g., suffering from a neurodegenerative disease) compared to a reference (e.g., neuroinflammation in the subject prior to the administering). In certain aspects, administering a miR-485 inhibitor decreases neuroinflammation in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., subjects that did not receive an administration of the miR-485 inhibitor). In some aspects, decreased neuroinflammation comprises glial cells producing decreased amounts of inflammatory mediators. Accordingly, in certain aspects, administering a miR-485 inhibitor disclosed herein to a subject (e.g., suffering from a neurodegenerative disease) decreases the amount of inflammatory mediators produced by glial cells by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., subjects that did not receive an administration of the miR-485 inhibitor). In some aspects, an inflammatory mediator produced by glial cells comprises TNF-α. In some aspects, the inflammatory mediator comprises IL-1β. In some aspects, an inflammatory mediator produced by glial cells comprises both TNF-α and IL-1β.

In some aspects, administering a miR-485 inhibitor disclosed herein increases autophagy (e.g., by increasing the expression of a SIRT1 protein and/or SIRT1 gene) in a subject (e.g., suffering from a neurodegenerative disease). As used herein, the term “autophagy” refers to cellular stress response and a survival pathway that is responsible for the degradation of long-lived proteins, protein aggregates, as well as damaged organelles in order to maintain cellular homeostasis. Not surprisingly, abnormalities of autophagy have been associated with number of diseases, including many neurodegenerative diseases (e.g., Alzheimer's disease and Parkinson's disease). In some aspects, administering a miR-485 inhibitor disclosed herein to a subject (e.g., suffering from a degenerative disease) increases autophagy by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, or at least about 300% or more, compared to a reference (e.g., subjects that did not receive an administration of the miR-485 inhibitor). Increase in autophagy can be measured by any suitable methods known in the art. For instance, in some aspects, increase in autophagy can be observed by measuring the expression of a gene associated with autophagosome biogenesis (e.g., LC3B).

In some aspects, administering a miR-485 inhibitor disclosed herein increases alpha-secretase activity (e.g., by increasing the expression of a SIRT1 protein and/or SIRT1 gene) in a subject (e.g., suffering from a neurodegenerative disease). As used herein, the term “alpha-secretase” refers to a family of proteolytic enzymes that cleave amyloid precursor protein (APP) in its transmembrane region. Alpha secretases are members of the ADAM (“a disintegrin and metalloprotease domain”) family (e.g., ADAM10), which are expressed on the surfaces of cells and anchored in the cell membrane. Specifically, alpha secretases cleave within the fragment that gives rise to the Alzheimer's disease-associated peptide amyloid beta when APP is instead processed by beta secretase and gamma secretase. Thus, in some aspects, alpha-secretase cleavage precludes amyloid beta formation and is considered to be part of the non-amyloidogenic pathway in APP processing. In some aspects, administering a miR-485 inhibitor disclosed herein to a subject (e.g., suffering from a neurodegenerative disease) increases alpha-secretase activity by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, or at least about 300% or more, compared to a reference (e.g., subjects that did not receive an administration of the miR-485 inhibitor).

In some aspects, administering a miR-485 inhibitor disclosed herein decreases beta-secretase 1 (BACE1) activity (e.g., by increasing the expression of a SIRT1 protein and/or SIRT1 gene) in a subject (e.g., suffering from a neurodegenerative disease). As used herein, the term “beta-secretase 1” or “BACE1” refers to an enzyme that is expressed mainly in neurons. BACE1 is an aspartic acid protease important in the formation of myelin sheaths in peripheral nerve cells. In some aspects, administering a miR-485 inhibitor disclosed herein to a subject (e.g., suffering from a neurodegenerative disease) decreases BACE1 activity by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., subjects that did not receive an administration of the miR-485 inhibitor).

As is known in the art, many neurodegenerative diseases exhibit certain motor and/or non-motor symptoms. For instance, non-limiting examples of motor symptoms associated with Parkinson's disease include resting tremor, reduction of spontaneous movement (bradykinesia), rigidity, postural instability, freezing of gait, impaired handwriting (micrographia), decreased facial expression, and uncontrolled rapid movements. Non-limiting examples of non-motor symptoms associated with Parkinson's disease include autonomic dysfunction, neuropsychiatric problems (mood, cognition, behavior, or thought alterations), sensory alterations (especially altered sense of smell), and sleep difficulties.

In some aspects, administering a miR-485 inhibitor of the present disclosure improves one or more motor symptoms in a subject (e.g., suffering from a neurodegenerative disease) compared to a reference (e.g., corresponding motor symptoms in the subject prior to the administering). In certain aspects, administering a miR-485 inhibitor of the present disclosure improves one or more motor symptoms in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 250%, or at least about 300% or more compared to a reference (e.g., subjects that did not receive an administration of the miR-485 inhibitor).

In some aspects, administering a miR-485 inhibitor of the present disclosure improves one or more non-motor symptoms in a subject (e.g., suffering from a neurodegenerative disease) compared to a reference (e.g., corresponding non-motor symptom in the subject prior to the administering). In certain aspects, administering a miR-485 inhibitor disclosed herein improves one or more non-motor symptoms in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 250%, or at least about 300% or more compared to a reference (e.g., subjects that did not receive an administration of the miR-485 inhibitor).

In some aspects, administering a miR-485 inhibitor disclosed herein improves synaptic function in a subject (e.g., suffering from a neurodegenerative disease) compared to a reference (e.g., synaptic function in the subject prior to the administering). As used herein, the term “synaptic function,” refers to the ability of the synapse of a cell (e.g., a neuron) to pass an electrical or chemical signal to another cell (e.g., a neuron). In some aspects, administering a miR-485 inhibitor of the present disclosure improves synaptic function in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 150%, at least about 200%, at least about 250%, or at least about 300% or more compared to a reference (e.g., subjects that did not receive an administration of the miR-485 inhibitor).

In some aspects, administering a miR-485 inhibitor of the present disclosure can prevent, delay, and/or ameliorate the loss of synaptic function in a subject (e.g., suffering from a neurodegenerative disease) compared to a reference (e.g., loss of synaptic function in the subject prior to the administering). In some aspects, administering a miR-485 inhibitor prevents, delays, and/or ameliorates the loss of synaptic function in a subject (e.g., suffering from a neurodegenerative disease) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to a reference (e.g., subjects that did not receive an administration of the miR-485 inhibitor).

In some aspects, a miR-485 inhibitor disclosed herein can be administered by any suitable route known in the art. In certain aspects, a miR-485 inhibitor is administered parenthetically, intramuscularly, subcutaneously, ophthalmic, intravenously, intraperitoneally, intradermally, intraorbitally, intracerebrally, intracranially, intracerebroventricularly, intraspinally, intraventricular, intrathecally, intracistemally, intracapsularly, intratumorally, or any combination thereof. In certain aspects, a miR-485 inhibitor is administered intracerebroventricularly (ICV). In certain aspects, a miR-485 inhibitor is administered intravenously.

In some aspects, a miR-485 inhibitor of the present disclosure can be used in combination with one or more additional therapeutic agents. In some aspects, the additional therapeutic agent and the miR-485 inhibitor are administered concurrently. In certain aspects, the additional therapeutic agent and the miR-485 inhibitor are administered sequentially.

In some aspects, the administration of a miR-485 inhibitor disclosed herein does not result in any adverse effects. In certain aspects, miR-485 inhibitors of the present disclosure do not adversely affect body weight when administered to a subject. In some aspects, miR-485 inhibitors disclosed herein do not result in increased mortality or cause pathological abnormalities when administered to a subject.

III. miRNA-485 Inhibitors Useful for the Present Disclosure

Disclosed herein are compounds that can inhibit miR-485 activity (miR-485 inhibitor). In some aspects, a miR-485 inhibitor of the present disclosure comprises a nucleotide sequence encoding a nucleotide molecule that comprises at least one miR-485 binding site, wherein the nucleotide molecule does not encode a protein. As described herein, in some aspects, the miR-485 binding site is at least partially complementary to the target miRNA nucleic acid sequence (i.e., miR-485), such that the miR-485 inhibitor hybridizes to the miR-485 nucleic acid sequence.

In some aspects, the miR-485 binding site of a miR inhibitor disclosed herein has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence complementarity to the nucleic acid sequence of a miR-485. In certain aspects, the miR-485 binding site is fully complementary to the nucleic acid sequence of a miR-485.

The miR-485 hairpin precursor can generate both miR-485-5p and miR-485-3p. In the context of the present disclosure “miR-485” encompasses both miR-485-5p and miR-485-3p unless specified otherwise. The human mature miR-485-3p has the sequence 5′-GUCAUACACGGCUCUCCUCUCU-3′ (SEQ ID NO: 1; miRBase Acc. No. MIMAT0002176). A 5′ terminal subsequence of miR-485-3p 5′-UCAUACA-3′ (SEQ ID NO: 49) is the seed sequence. The human mature miR-485-5p has the sequence 5′-AGAGGCUGGCCGUGAUGAAUUC-3′ (SEQ ID NO: 33; miRBase Acc. No. MIMAT0002175). A 5′ terminal subsequence of miR-485-5p 5′-GAGGCUG-3′ (SEQ ID NO: 50) is the seed sequence.

As will be apparent to those in the art, the human mature miR-485-3p has significant sequence similarity to that of other species. For instance, the mouse mature miR-485-3p differs from the human mature miR-485-3p by a single amino acid at each of the 5′- and 3′-ends (i.e., has an extra “A” at the 5′-end and missing “C” at the 3′-end). The mouse mature miR-485-3p has the following sequence:

5′-AGUCAUACACGGCUCUCCUCUC-3′ (SEQ ID NO: 34;
miRBase Acc. No. mature miR-485-3p).

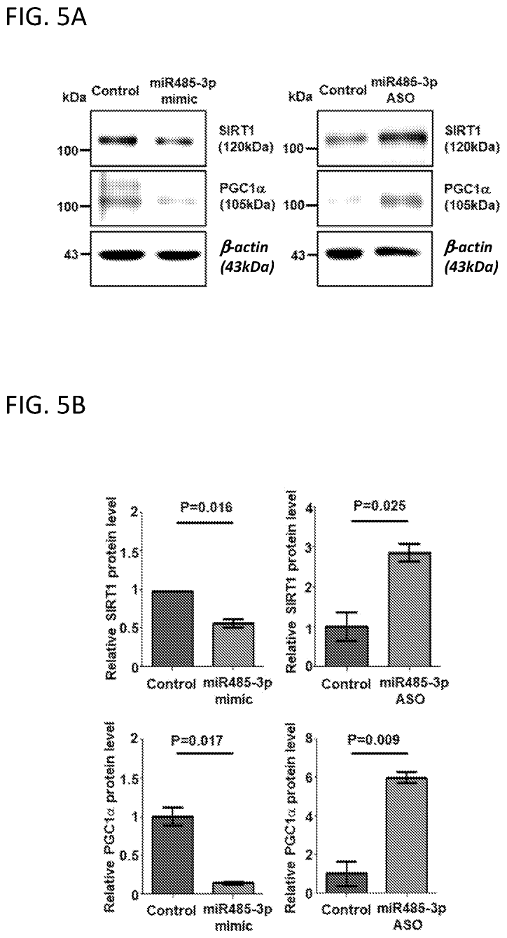

The sequence for the mouse mature miR-485-5p is identical to that of the human: 5′-agaggcuggccgugaugaauuc-3′ (SEQ ID NO: 33; miRBase Acc. No. MIMAT0003128). Sequence alignments for human mature miR-485-3p and miR-485-5p to the corresponding sequences from other exemplary mammalian species are provided in FIGS. 5A and 5B. Because of the similarity in sequences, in some aspects, a miR-485 inhibitor of the present disclosure is capable of binding miR-485-3p and/or miR-485-5p from one or more species shown in FIGS. 5A and 5B. In certain aspects, a miR-485 inhibitor disclosed herein is capable of binding to miR-485-3p and/or miR-485-5p from both human and mouse.

In some aspects, the miR-485 binding site is a single-stranded polynucleotide sequence that is complementary (e.g., fully complementary) to a sequence of a miR-485-3p (or a subsequence thereof). In some aspects, the miR-485-3p subsequence comprises the seed sequence. Accordingly, in certain aspects, the miR-485 binding site has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence complementarity to the nucleic acid sequence set forth in SEQ ID NO: 49. In certain aspects, the miR-485 binding site is complementary to miR-485-3p except for 1, 2, 3, 4, 5, 6, 7, 8, 9, or 10 mismatches. In further aspects, the miR-485 binding site is fully complementary to the nucleic acid sequence set forth in SEQ ID NO: 1.

In some aspects, the miR-485 binding site is a single-stranded polynucleotide sequence that is complementary (e.g., fully complementary) to a sequence of a miR-485-5p (or a subsequence thereof). In some aspects, the miR-485-5p subsequence comprises the seed sequence. In certain aspects, the miR-485 binding site has at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence complementarity to the nucleic acid sequence set forth in SEQ ID NO: 50. In certain aspects, the miR-485 binding site is complementary to miR-485-5p except for 1, 2, 3, 4, 5, 6, 7, 8, 9, or 10 mismatches. In further aspects, the miR-485 binding site is fully complementary to the nucleic acid sequence set forth in SEQ ID NO: 35.

The seed region of a miRNA forms a tight duplex with the target mRNA. Most miRNAs imperfectly base-pair with the 3′ untranslated region (UTR) of target mRNAs, and the 5′ proximal “seed” region of miRNAs provides most of the pairing specificity. Without being bound to any theory, it is believed that the first nine miRNA nucleotides (encompassing the seed sequence) provide greater specificity whereas the miRNA ribonucleotides 3′ of this region allow for lower sequence specificity and thus tolerate a higher degree of mismatched base pairing, with positions 2-7 being the most important. Accordingly, in specific aspects of the present disclosure, the miR-485 binding site comprises a subsequence that is fully complementary (i.e., 100% complementary) over the entire length of the seed sequence of miR-485.

miRNA sequences and miRNA binding sequences that can be used in the context of the disclosure include, but are not limited to, all or a portion of those sequences in the sequence listing provided herein, as well as the miRNA precursor sequence, or complement of one or more of these miRNAs. Any aspects of the disclosure involving specific miRNAs or miRNA binding sites by name is contemplated also to cover miRNAs or complementary sequences thereof whose sequences are at least about at least about 50%, at least about 55%, at least about 60%, at least about 65%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to the mature sequence of the specified miRNA sequence or complementary sequence thereof.

In some aspects, miRNA binding sequences of the present disclosure can include additional nucleotides at the 5′, 3′, or both 5′ and 3′ ends of those sequences in the sequence listing provided herein, as long as the modified sequence is still capable of specifically binding to miR-485. In some aspects, miRNA binding sequences of the present disclosure can differ in at least 1, 2, 3, 4, 5, 6, 7, 8, 9, 10 or more nucleotides with respect to those sequence in the sequence listing provided, as long as the modified sequence is still capable of specifically binding to miR-485.

It is also specifically contemplated that any methods and compositions discussed herein with respect to miRNA binding molecules or miRNA can be implemented with respect to synthetic miRNAs binding molecules. It is also understood that the disclosures related to RNA sequences in the present disclosure are equally applicable to corresponding DNA sequences.

In some aspects, a miRNA-485 inhibitor of the present disclosure comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 5′ of the nucleotide sequence. In some aspects, a miRNA-485 inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 3′ of the nucleotide sequence.

In some aspects, a miR-485 inhibitor disclosed herein is about 6 to about 30 nucleotides in length. In certain aspects, a miR-485 inhibitor disclosed herein is 7 nucleotides in length. In further aspects, a miR-485 inhibitor disclosed herein is 8 nucleotides in length. In some aspects, a miR-485 inhibitor is 9 nucleotides in length. In some aspects, a miR-485 inhibitor of the present disclosure is 10 nucleotides in length. In certain aspects, a miR-485 inhibitor is 11 nucleotides in length. In further aspects, a miR-485 inhibitor is 12 nucleotides in length. In some aspects, a miR-485 inhibitor disclosed herein is 13 nucleotides in length. In certain aspects, a miR-485 inhibitor disclosed herein is 14 nucleotides in length. In some aspects, a miR-485 inhibitor disclosed herein is 15 nucleotides in length. In further aspects, a miR-485 inhibitor is 16 nucleotides in length. In certain aspects, a miR-485 inhibitor of the present disclosure is 17 nucleotides in length. In some aspects, a miR-485 inhibitor is 18 nucleotides in length. In some aspects, a miR-485 inhibitor is 19 nucleotides in length. In certain aspects, a miR-485 inhibitor is 20 nucleotides in length. In further aspects, a miR-485 inhibitor of the present disclosure is 21 nucleotides in length. In some aspects, a miR-485 inhibitor is 22 nucleotides in length.

In some aspects, a miR-485 inhibitor disclosed herein comprises a nucleotide sequence that is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to a sequence selected from SEQ ID NOs: 2 to 30. In certain aspects, a miR-485 inhibitor comprises a nucleotide sequence selected from the group consisting of SEQ ID NOs: 2 to 30, wherein the nucleotide sequence can optionally comprise 1, 2, 3, 4, 5, 6, 7, 8, 9, or 10 mismatches.

In some aspects, a miRNA inhibitor comprises 5′-UGUAUGA-3′ (SEQ ID NO: 2), 5′-GUGUAUGA-3′ (SEQ ID NO: 3), 5′-CGUGUAUGA-3′ (SEQ ID NO: 4), 5′-CCGUGUAUGA-3′ (SEQ ID NO: 5), 5′-GCCGUGUAUGA-3′ (SEQ ID NO: 6), 5′-AGCCGUGUAUGA-3′ (SEQ ID NO: 7), 5′-GAGCCGUGUAUGA-3′ (SEQ ID NO: 8), 5′-AGAGCCGUGUAUGA-3′ (SEQ ID NO: 9), 5′-GAGAGCCGUGUAUGA-3′ (SEQ ID NO: 10), 5′-GGAGAGCCGUGUAUGA-3′ (SEQ ID NO: 11), 5′-AGGAGAGCCGUGUAUGA-3′ (SEQ ID NO: 12), 5′-GAGGAGAGCCGUGUAUGA-3′ (SEQ ID NO: 13), 5′-AGAGGAGAGCCGUGUAUGA-3′ (SEQ ID NO: 14), or 5′-GAGAGGAGAGCCGUGUAUGA-3′ (SEQ ID NO: 15).

In some aspects, the miRNA inhibitor has 5′-UGUAUGAC-3′ (SEQ ID NO: 16), 5′-GUGUAUGAC-3′ (SEQ ID NO: 17), 5′-CGUGUAUGAC-3′ (SEQ ID NO: 18), 5′-CCGUGUAUGAC-3′ (SEQ ID NO: 19), 5′-GCCGUGUAUGAC-3′ (SEQ ID NO: 20), 5′-AGCCGUGUAUGAC-3′ (SEQ ID NO: 21), 5′-GAGCCGUGUAUGAC-3′ (SEQ ID NO: 22), 5′-AGAGCCGUGUAUGAC-3′ (SEQ ID NO: 23), 5′-GAGAGCCGUGUAUGAC-3′ (SEQ ID NO: 24), 5′-GGAGAGCCGUGUAUGAC-3′ (SEQ ID NO: 25), 5′-AGGAGAGCCGUGUAUGAC-3′ (SEQ ID NO: 26), 5′-GAGGAGAGCCGUGUAUGAC-3′ (SEQ ID NO: 27), 5′-AGAGGAGAGCCGUGUAUGAC-3′ (SEQ ID NO: 28), 5′-GAGAGGAGAGCCGUGUAUGAC-3′ (SEQ ID NO: 29), or AGAGAGGAGAGCCGUGUAUGAC (SEQ ID NO: 30).

In some aspects, the miRNA inhibitor has a sequence selected from the group consisting of: 5′-TGTATGA-3′ (SEQ ID NO: 62), 5′-GTGTATGA-3′ (SEQ ID NO: 63), 5′-CGTGTATGA-3′ (SEQ ID NO: 64), 5′-CCGTGTATGA-3′ (SEQ ID NO: 65), 5′-GCCGTGTATGA-3′ (SEQ ID NO: 66), 5′-AGCCGTGTATGA-3′ (SEQ ID NO: 67), 5′-GAGCCGTGTATGA-3′ (SEQ ID NO: 68), 5′-AGAGCCGTGTATGA-3′ (SEQ ID NO: 69), 5′-GAGAGCCGTGTATGA-3′ (SEQ ID NO: 70), 5′-GGAGAGCCGTGTATGA-3′ (SEQ ID NO: 71), 5′-AGGAGAGCCGTGTATGA-3′ (SEQ ID NO: 72), 5′-GAGGAGAGCCGTGTATGA-3′ (SEQ ID NO: 73), 5′-AGAGGAGAGCCGTGTATGA-3′ (SEQ ID NO: 74), 5′-GAGAGGAGAGCCGTGTATGA-3′ (SEQ ID NO: 75); 5′-TGTATGAC-3′ (SEQ ID NO: 76), 5′-GTGTATGAC-3′ (SEQ ID NO: 77), 5′-CGTGTATGAC-3′ (SEQ ID NO: 78), 5′-CCGTGTATGAC-3′ (SEQ ID NO: 79), 5′-GCCGTGTATGAC-3′ (SEQ ID NO: 80), 5′-AGCCGTGTATGAC-3′ (SEQ ID NO: 81), 5′-GAGCCGTGTATGAC-3′ (SEQ ID NO: 82), 5′-AGAGCCGTGTATGAC-3′ (SEQ ID NO: 83), 5′-GAGAGCCGTGTATGAC-3′ (SEQ ID NO: 84), 5′-GGAGAGCCGTGTATGAC-3′ (SEQ ID NO: 85), 5′-AGGAGAGCCGTGTATGAC-3′ (SEQ ID NO: 86), 5′-GAGGAGAGCCGTGTATGAC-3′ (SEQ ID NO: 87), 5′-AGAGGAGAGCCGTGTATGAC-3′ (SEQ ID NO: 88), 5′-GAGAGGAGAGCCGTGTATGAC-3′ (SEQ ID NO: 89); and AGAGAGGAGAGCCGTGTATGAC (SEQ ID NO: 90).

In some aspects, a miRNA inhibitor disclosed herein (i.e., miR-485 inhibitor) comprises a nucleotide sequence that is at least about 50%, at least about 55%, at least about 60%, at least about 65%, at least about 70%, at least about 75%, at least about 80%, at least about 85%, at least about 90%, or at least about 95% identical to 5′-AGAGGAGAGCCGUGUAUGAC-3′ (SEQ ID NO: 28) or 5′-AGAGGAGAGCCGTGTATGAC-3′ (SEQ ID NO: 88). In some aspects, the miRNA inhibitor comprises a nucleotide sequence that has at least 90% similarity to 5′-AGAGGAGAGCCGUGUAUGAC-3′ (SEQ ID NO: 28) or 5′-AGAGGAGAGCCGTGTATGAC-3′ (SEQ ID NO: 88). In some aspects, the miRNA inhibitor comprises the nucleotide sequence 5′-AGAGGAGAGCCGUGUAUGAC-3′ (SEQ ID NO: 28) or 5′-AGAGGAGAGCCGTGTATGAC-3′ (SEQ ID NO: 88) with one substitution or two substitutions. In some aspects, the miRNA inhibitor comprises the nucleotide sequence 5′-AGAGGAGAGCCGUGUAUGAC-3′ (SEQ ID NO: 28) or 5′-AGAGGAGAGCCGTGTATGAC-3′ (SEQ ID NO: 88). In certain aspects, the miRNA inhibitor comprises the nucleotide sequence 5′AGAGGAGAGCCGUGUAUGAC-3′ (SEQ ID NO: 28).

In some aspects, a miR-485 inhibitor of the present disclosure comprises the sequence disclosed herein, e.g., any one of SEQ ID NOs: 2 to 30, and at least one, at least two, at least three, at least four or at least five additional nucleic acid at the N terminus, at least one, at least two, at least three, at least four, or at least five additional nucleic acid at the C terminus, or both. In some aspects, a miR-485 inhibitor of the present disclosure comprises the sequence disclosed herein, e.g., any one of SEQ ID NOs: 2 to 30, and one additional nucleic acid at the N terminus and/or one additional nucleic acid at the C terminus. In some aspects, a miR-485 inhibitor of the present disclosure comprises the sequence disclosed herein, e.g., any one of SEQ ID NOs: 2 to 30, and one or two additional nucleic acids at the N terminus and/or one or two additional nucleic acids at the C terminus. In some aspects, a miR-485 inhibitor of the present disclosure comprises the sequence disclosed herein, e.g., any one of SEQ ID NOs: 2 to 30, and one to three additional nucleic acids at the N terminus and/or one to three additional nucleic acids at the C terminus. In some aspects, a miR-485 inhibitor comprises 5′-GAGAGGAGAGCCGUGUAUGAC-3′ (SEQ ID NO: 29).

In some aspects, a miR-485 inhibitor of the present disclosure comprises one miR-485 binding site. In further aspects, a miR-485 inhibitor disclosed herein comprises at least two miR-485 binding sites. In certain aspects, a miR-485 inhibitor comprises three miR-485 binding sites. In some aspects, a miR-485 inhibitor comprises four miR-485 binding sites. In some aspects, a miR-485 inhibitor comprises five miR-485 binding sites. In certain aspects, a miR-485 inhibitor comprises six or more miR-485 binding sites. In some aspects, all the miR-485 binding sites are identical. In some aspects, all the miR-485 binding sites are different. In some aspects, at least one of the miR-485 binding sites is different. In some aspects, all the miR-485 binding sites are miR-485-3p binding sites. In other aspects, all the miR-485 binding sites are miR-485-5p binding sites. In further aspects, a miR-485 inhibitor comprises at least one miR-485-3p binding site and at least one miR-485-5p binding site.

III.a. Chemically Modified Polynucleotides

In some aspects, a miR-485 inhibitor disclosed herein comprises a polynucleotide which includes at least one chemically modified nucleoside and/or nucleotide. When the polynucleotides of the present disclosure are chemically modified the polynucleotides can be referred to as “modified polynucleotides.”

A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside including a phosphate group. Modified nucleotides can be synthesized by any useful method, such as, for example, chemically, enzymatically, or recombinantly, to include one or more modified or non-natural nucleosides.

Polynucleotides can comprise a region or regions of linked nucleosides. Such regions can have variable backbone linkages. The linkages can be standard phosphodiester linkages, in which case the polynucleotides would comprise regions of nucleotides.

The modified polynucleotides disclosed herein can comprise various distinct modifications. In some aspects, the modified polynucleotides contain one, two, or more (optionally different) nucleoside or nucleotide modifications. In some aspects, a modified polynucleotide can exhibit one or more desirable properties, e.g., improved thermal or chemical stability, reduced immunogenicity, reduced degradation, increased binding to the target microRNA, reduced non-specific binding to other microRNA or other molecules, as compared to an unmodified polynucleotide.

In some aspects, a polynucleotide of the present disclosure (e.g., a miR-485 inhibitor) is chemically modified. As used herein, in reference to a polynucleotide, the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population, including, but not limited to, its nucleobase, sugar, backbone, or any combination thereof.

In some aspects, a polynucleotide of the present disclosure (e.g., a miR-485 inhibitor) can have a uniform chemical modification of all or any of the same nucleoside type or a population of modifications produced by downward titration of the same starting modification in all or any of the same nucleoside type, or a measured percent of a chemical modification of all any of the same nucleoside type but with random incorporation In further aspects, the polynucleotide of the present disclosure (e.g., a miR-485 inhibitor) can have a uniform chemical modification of two, three, or four of the same nucleoside type throughout the entire polynucleotide (such as all uridines and/or all cytidines, etc. are modified in the same way).

Modified nucleotide base pairing encompasses not only the standard adenine-thymine, adenine-uracil, or guanine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleobase inosine and adenine, cytosine or uracil. Any combination of base/sugar or linker can be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U”s. For example, TD's of the present disclosure can be administered as RNAs, as DNAs, or as hybrid molecules comprising both RNA and DNA units.

In some aspects, the polynucleotide (e.g., a miR-485 inhibitor) includes a combination of at least two (e.g., 2, 3, 4, 5, 6, 7, 8, 8, 10, 11, 12, 13, 14, 15, 16, 17, 18, 18, 20 or more) modified nucleobases.

In some aspects, the nucleobases, sugar, backbone linkages, or any combination thereof in a polynucleotide are modified by at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100%.

(i) Base Modification

In certain aspects, the chemical modification is at nucleobases in a polynucleotide of the present disclosure (e.g., a miR-485 inhibitor). In some aspects, the at least one chemically modified nucleoside is a modified uridine (e.g., pseudouridine (ψ), 2-thiouridine (s2U), 1-methyl-pseudouridine (m1ψ), 1-ethyl-pseudouridine (e1ψ), or 5-methoxy-uridine (mo5U)), a modified cytosine (e.g., 5-methyl-cytidine (m5C)) a modified adenosine (e.g, 1-methyl-adenosine (m1A), N6-methyl-adenosine (m6A), or 2-methyl-adenine (m2A)), a modified guanosine (e.g., 7-methyl-guanosine (m7G) or 1-methyl-guanosine (m1G)), or a combination thereof.

In some aspects, the polynucleotide of the present disclosure (e.g., a miR-485 inhibitor) is uniformly modified (e.g., fully modified, modified throughout the entire sequence) for a particular modification. For example, a polynucleotide can be uniformly modified with the same type of base modification, e.g., 5-methyl-cytidine (m5C), meaning that all cytosine residues in the polynucleotide sequence are replaced with 5-methyl-cytidine (m5C). Similarly, a polynucleotide can be uniformly modified for any type of nucleoside residue present in the sequence by replacement with a modified nucleoside such as any of those set forth above.

In some aspects, the polynucleotide of the present disclosure (e.g., a miR-485 inhibitor) includes a combination of at least two (e.g., 2, 3, 4 or more) of modified nucleobases. In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of a type of nucleobases in a polynucleotide of the present disclosure (e.g., a miR-485 inhibitor) are modified nucleobases.

(ii) Backbone Modifications

In some aspects, the polynucleotide of the present disclosure (i.e., miR-485 inhibitor) can include any useful linkage between the nucleosides. Such linkages, including backbone modifications, that are useful in the composition of the present disclosure include, but are not limited to the following: 3′-alkylene phosphonates, 3′-amino phosphoramidate, alkene containing backbones, aminoalkylphosphoramidates, aminoalkylphosphotriesters, boranophosphates, —CH₂—O—N(CH₃)—CH₂—, —CH₂—N(CH₃)—N(CH₃)—CH₂—, —CH₂—NH—CH₂—, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

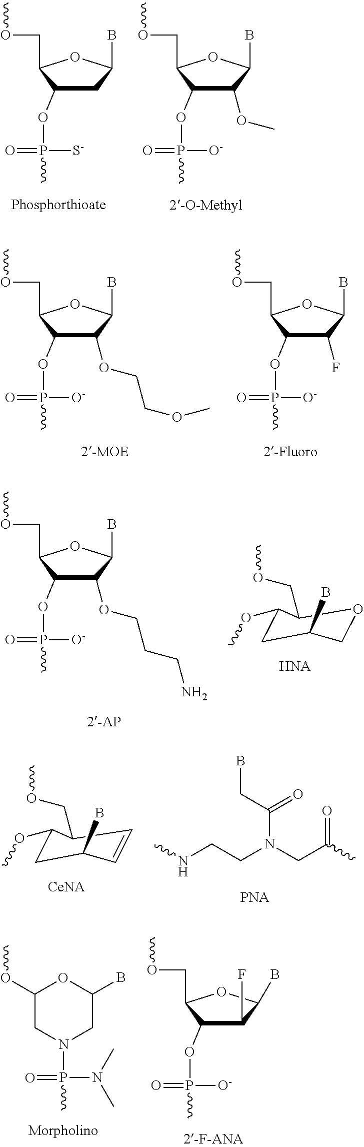

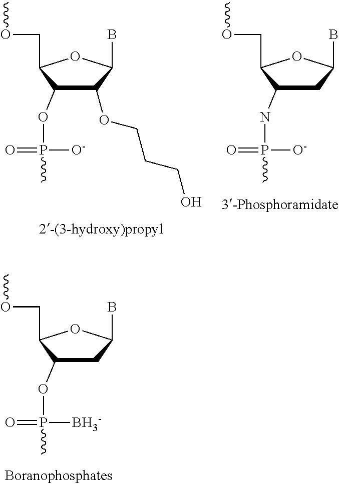

In some aspects, the presence of a backbone linkage disclosed above increase the stability and resistance to degradation of a polynucleotide of the present disclosure (i.e., miR-485 inhibitor).

In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the backbone linkages in a polynucleotide of the present disclosure (i.e., miR-485 inhibitor) are modified (e.g., all of them are phosphorothioate).

In some aspects, a backbone modification that can be included in a polynucleotide of the present disclosure (i.e., miR-485 inhibitor) comprises phosphorodiamidate morpholino oligomer (PMO) and/or phosphorothioate (PS) modification.

(iii) Sugar Modifications

The modified nucleosides and nucleotides which can be incorporated into a polynucleotide of the present disclosure (i.e., miR-485 inhibitor) can be modified on the sugar of the nucleic acid. In some aspects, the sugar modification increases the affinity of the binding of a miR-485 inhibitor to miR-485 nucleic acid sequence. Incorporating affinity-enhancing nucleotide analogues in the miR-485 inhibitor, such as LNA or 2′-substituted sugars, can allow the length and/or the size of the miR-485 inhibitor to be reduced.

In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the nucleotides in a polynucleotide of the present disclosure (i.e., miR-485 inhibitor) contain sugar modifications (e.g., LNA).

In some aspects, 1, 2, 3, 4, 5, 6, 7, 8, 9, 10, 11, 12, 13, 14, 15, 16, 17, 18, 19, 20, 21, or 22 nucleotide units in a polynucleotide of the present disclosure are sugar modified (e.g., LNA).

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2)) , and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

The 2′ hydroxyl group (OH) of ribose can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges include methylene, propylene, ether, amino bridges, aminoalkyl, aminoalkoxy, amino, and amino acid.

In some aspects, nucleotide analogues present in a polynucleotide of the present disclosure (i.e., mir-485 inhibitor) comprise, e.g., 2′-O-alkyl-RNA units, 2′-OMe-RNA units, 2′-O-alkyl-SNA, 2′-amino-DNA units, 2′-fluoro-DNA units, LNA units, arabino nucleic acid (ANA) units, 2′-fluoro-ANA units, HNA units, INA (intercalating nucleic acid) units, 2′MOE units, or any combination thereof. In some aspects, the LNA is, e.g., oxy-LNA (such as beta-D-oxy-LNA, or alpha-L-oxy-LNA), amino-LNA (such as beta-D-amino-LNA or alpha-L-amino-LNA), thio-LNA (such as beta-D-thio0-LNA or alpha-L-thio-LNA), ENA (such a beta-D-ENA or alpha-L-ENA), or any combination thereof. In further aspects, nucleotide analogues that can be included in a polynucleotide of the present disclosure (i.e., miR-485 inhibitor) comprises a locked nucleic acid (LNA), an unlocked nucleic acid (UNA), an arabino nucleic acid (ABA), a bridged nucleic acid (BNA), and/or a peptide nucleic acid (PNA).

In some aspects, a polynucleotide of the present disclosure (i.e., miR-485 inhibitor) can comprise both modified RNA nucleotide analogues (e.g., LNA) and DNA units. In some aspects, a miR-485 inhibitor is a gapmer. See, e.g., U.S. Pat. Nos. 8,404,649; 8,580,756; 8,163,708; 9,034,837; all of which are herein incorporated by reference in their entireties. In some aspects, a miR-485 inhibitor is a micromir. See U.S. Pat. Appl. Publ. No. US20180201928, which is herein incorporated by reference in its entirety.

In some aspects, a polynucleotide of the present disclosure (i.e., miR-485 inhibitor) can include modifications to prevent rapid degradation by endo- and exo-nucleases. Modifications include, but are not limited to, for example, (a) end modifications, e.g., 5′ end modifications (phosphorylation, dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages.

### IV. Vectors and Delivery Systems

In some aspects, the miR-485 inhibitors of the present disclosure can be administered, e.g., to a subject suffering from a disease or condition associated with abnormal (e.g., reduced) level of a SIRT1 protein and/or SIRT1 gene, using any relevant delivery system known in the art. In certain aspects, the delivery system is a vector. Accordingly, in some aspects, the present disclosure provides a vector comprising a miR-485 inhibitor of the present disclosure.

In some aspects, the vector is viral vector. In some aspects, the viral vector is an adenoviral vector or an adeno-associated viral vector. In certain aspects, the viral vector is an AAV that has a serotype of AAV2, AAV3, AAV4, AAVS, AAV6, AAV7, AAV8, AAV9, AAV10, or any combination thereof. In some aspects, the adenoviral vector is a third generation adenoviral vector. ADEASY™ is by far the most popular method for creating adenoviral vector constructs. The system consists of two types of plasmids: shuttle (or transfer) vectors and adenoviral vectors. The transgene of interest is cloned into the shuttle vector, verified, and linearized with the restriction enzyme PmeI. This construct is then transformed into ADEASIER-1 cells, which are BJ5183 E. coli cells containing PADEASY™. PADEASY™ is a ˜33 Kb adenoviral plasmid containing the adenoviral genes necessary for virus production. The shuttle vector and the adenoviral plasmid have matching left and right homology arms which facilitate homologous recombination of the transgene into the adenoviral plasmid. One can also co-transform standard BJ5183 with supercoiled PADEASY™ and the shuttle vector, but this method results in a higher background of non-recombinant adenoviral plasmids. Recombinant adenoviral plasmids are then verified for size and proper restriction digest patterns to determine that the transgene has been inserted into the adenoviral plasmid, and that other patterns of recombination have not occurred. Once verified, the recombinant plasmid is linearized with PacI to create a linear dsDNA construct flanked by ITRs. 293 or 911 cells are transfected with the linearized construct, and virus can be harvested about 7-10 days later. In addition to this method, other methods for creating adenoviral vector constructs known in the art at the time the present application was filed can be used to practice the methods disclosed herein.

In some aspects, the viral vector is a retroviral vector, e.g., a lentiviral vector (e.g., a third or fourth generation lentiviral vector). Lentiviral vectors are usually created in a transient transfection system in which a cell line is transfected with three separate plasmid expression systems. These include the transfer vector plasmid (portions of the HIV provirus), the packaging plasmid or construct, and a plasmid with the heterologous envelop gene (env) of a different virus. The three plasmid components of the vector are put into a packaging cell which is then inserted into the HIV shell. The virus portions of the vector contain insert sequences so that the virus cannot replicate inside the cell system. Current third generation lentiviral vectors encode only three of the nine HIV-1 proteins (Gag, Pol, Rev), which are expressed from separate plasmids to avoid recombination-mediated generation of a replication-competent virus. In fourth generation lentiviral vectors, the retroviral genome has been further reduced (see, e.g., TAKARA® LENTI-X™ fourth-generation packaging systems).

Any AAV vector known in the art can be used in the methods disclosed herein. The AAV vector can comprise a known vector or can comprise a variant, fragment, or fusion thereof. In some aspects, the AAV vector is selected from the group consisting of AAV type 1 (AAV1), AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, bovine AAV, shrimp AVV, snake AVV, and any combination thereof.

In some aspects, the AAV vector is derived from an AAV vector selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof.

In some aspects, the AAV vector is a chimeric vector derived from at least two AAV vectors selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof.

In certain aspects, the AAV vector comprises regions of at least two different AAV vectors known in the art.

In some aspects, the AAV vector comprises an inverted terminal repeat from a first AAV (e.g., AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, or any derivative thereof) and a second inverted terminal repeat from a second AAV (e.g., AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, or any derivative thereof).

In some aspects, the AVV vector comprises a portion of an AAV vector selected from the group consisting of AAV1, AAV2, AAV3A, AVV3B, AAV4, AAV5, AAV6, AAV7, AAV8, AVV9, AVV10, AVV11, AVV12, AVV13, AAVrh.74, avian AAV, bovine AAV, canine AAV, equine AAV, goat AVV, primate AAV, non-primate AAV, ovine AAV, shrimp AVV, snake AVV, and any combination thereof. In some aspects, the AAV vector comprises AAV2.

In some aspects, the AVV vector comprises a splice acceptor site. In some aspects, the AVV vector comprises a promoter. Any promoter known in the art can be used in the AAV vector of the present disclosure. In some aspects, the promoter is an RNA Pol III promoter. In some aspects, the RNA Pol III promoter is selected from the group consisting of the U6 promoter, the H1 promoter, the 7SK promoter, the 5S promoter, the adenovirus 2 (Ad2) VAI promoter, and any combination thereof. In some aspects, the promoter is a cytomegalovirus immediate-early gene (CMV) promoter, an EF1a promoter, an SV40 promoter, a PGK1 promoter, a Ubc promoter, a human beta actin promoter, a CAG promoter, a TRE promoter, a UAS promoter, a Ac5 promoter, a polyhedrin promoter, a CaMKIIa promoter, a GAL1 promoter, a GAL10 promoter, a TEF promoter, a GDS promoter, a ADH1 promoter, a CaMV35S promoter, or a Ubi promoter. In a specific aspect, the promoter comprises the U6 promoter.

In some aspects, the AAV vector comprises a constitutively active promoter (constitutive promoter). In some aspects, the constitutive promoter is selected from the group consisting of hypoxanthine phosphoribosyl transferase (HPRT), adenosine deaminase, pyruvate kinase, beta-actin promoter, cytomegalovirus (CMV), simian virus (e.g., SV40), papilloma virus, adenovirus, human immunodeficiency virus (HIV), Rous sarcoma virus, a retrovirus long terminal repeat (LTR), Murine stem cell virus (MSCV) and the thymidine kinase promoter of herpes simplex virus.

In some aspects, the promoter is an inducible promoter. In some aspects, the inducible promoter is a tissue specific promoter. In certain aspects, the tissue specific promoter drives transcription of the coding region of the AVV vector in a neuron, a glial cell, or in both a neuron and a glial cell.

In some aspects, the AVV vector comprises one or more enhancers. In some aspects, the one or more enhancer are present in the AAV alone or together with a promoter disclosed herein. In some aspects, the AAV vector comprises a 3′UTR poly(A) tail sequence. In some aspects, the 3′UTR poly(A) tail sequence is selected from the group consisting of bGH poly(A), actin poly(A), hemoglobin poly(A), and any combination thereof. In some aspects, the 3′UTR poly(A) tail sequence comprises bGH poly(A).

In some aspects, a miR-485 inhibitor disclosed herein is administered with a delivery agent. Non-limiting examples of delivery agents that can be used include a lipidoid, a liposome, a lipoplex, a lipid nanoparticle, a polymeric compound, a peptide, a protein, a cell, a nanoparticle mimic, a nanotube, a micelle, or a conjugate.

Thus, in some aspects, the present disclosure also provides a composition comprising a miRNA inhibitor of the present disclosure (i.e., miR-485 inhibitor) and a delivery agent. In some aspects, the delivery agent comprises a cationic carrier unit comprising

[WP]-L1-[CC]-L2-[AM] (formula I)

or

[WP]-L1-[AM]-L2-[CC] (formula II)
wherein
WP is a water-soluble biopolymer moiety;
CC is a positively charged carrier moiety;
AM is an adjuvant moiety; and,
L1 and L2 are independently optional linkers, and
wherein when mixed with a nucleic acid at an ionic ratio of about 1:1, the cationic carrier unit forms a micelle.

In some aspects, composition comprising a miRNA inhibitor of the present disclosure (i.e., miR-485 inhibitor) interacts with the cationic carrier unit via an ionic bond.

In some aspects, the water-soluble polymer comprises poly(alkvlene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (“POZ”) poly(N-acryloylmorpholine), or any combinations thereof. In some aspects, the water-soluble polymer comprises polyethylene glycol (“PEG”), polyglycerol, or poly(propylene glycol) (“PPG”). In some aspects, the water-soluble polymer comprises:

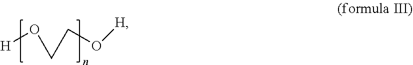

wherein n is 1-1000.

in some aspects, the n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141. In some aspects, the n is about 80 to about 90, about 90 to about 100, about 100 to about 110, about 110 to about 120, about 120 to about 130, about 140 to about 150, about 150 to about 160.

In some aspects, the water-soluble polymer is linear, branched, or dendritic. In some aspects, the cationic carrier moiety comprises one or more basic amino acids. In some aspects, the cationic carrier moiety comprises at least three, at least four, at least five, at least six, at least seven, at least eight, at least nine, at least ten, at least 11, at least 12, at least 13, at least 14, at last 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 basic amino acids. In some aspects, the cationic carrier moiety comprises about 30 to about 50 basic amino acids. In some aspects, the basic amino acid comprises arginine, lysine, histidine, or any combination thereof. In some aspects, the cationic carrier moiety comprises about 40 lysine monomers.

In some aspects, the adjuvant moiety is capable of modulating an immune response, an inflammatory response, and/or a tissue microenvironment. In some aspects, the adjuvant moiety comprises an imidazole derivative, an amino acid, a vitamin, or any combination thereof. In some aspects, the adjuvant moiety comprises:

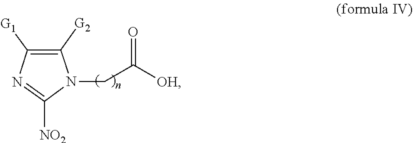

wherein each of G1 and G2 is H, an aromatic ring, or 1-10 alkyl, or G1 and G2 together form an aromatic ring, and wherein n is 1-10.

In some aspects, the adjuvant moiety comprises nitroimidazole. In some aspects, the adjuvant moiety comprises metronidazole, tinidazole, nimorazole, dimetridazole, pretomanid, ornidazole, megazol, azanidazole, benznidazole, or any combination thereof. In some aspects, the adjuvant moiety comprises an amino acid.

In some aspects, the adjuvant moiety comprises

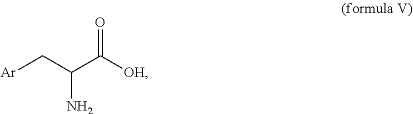

wherein Ar is

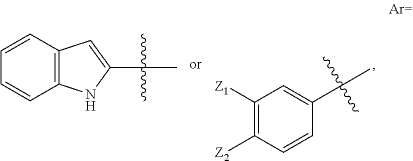

and
wherein each of Z1 and Z2 is H or OH.

In some aspects, the adjuvant moiety comprises a vitamin. In some aspects, the vitamin comprises a cyclic ring or cyclic hetero atom ring and a carboxyl group or hydroxyl group. In some aspects, the vitamin comprises:

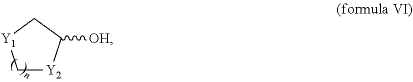

wherein each of Y1 and Y2 is C, N, O, or S, and wherein n is 1 or 2.

In some aspects, the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof. In some aspects, the vitamin is vitamin B3.

In some aspects, the adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, or at least about 20 vitamin B3. In some aspects, the adjuvant moiety comprises about 10 vitamin B3.

In some aspects, the composition comprises a water-soluble biopolymer moiety with about 120 to about 130 PEG units, a cationic carrier moiety comprising a poly-lysine with about 30 to about 40 lysines, and an adjuvant moiety with about 5 to about 10 vitamin B3.

In some aspects, the composition comprises (i) a water-soluble biopolymer moiety with about 100 to about 200 PEG units, (ii) about 30 to about 40 lysines with an amine group (e.g., about 32 lysines), (iii) about 15 to 20 lysines, each having a thiol group (e.g., about 16 lysines, each with a thiol group), and (iv) about 30 to 40 lysines fused to vitamin B3 (e.g., about 32 lysines, each fused to vitamin B3). In some aspects, the composition further comprises a targeting moiety, e.g., a LAT1 targeting ligand, e.g., phenyl alanine, linked to the water soluble polymer. In some aspects, the thiol groups in the composition form disulfide bonds.

In some aspects, the composition comprises (1) a micelle comprising (i) about 100 to about 200 PEG units, (ii) about 30 to about 40 lysines with an amine group (e.g., about 32 lysines), (iii) about 15 to 20 lysines, each having a thiol group (e.g., about 16 lysines, each with a thiol group), and (iv) about 30 to 40 lysines fused to vitamin B3 (e.g., about 32 lysines, each fused to vitamin B3), and (2) a miR485 inhibitor (e.g., SEQ ID NO: 30), wherein the miR485 inhibitor is encapsulated within the micelle. In some aspects, the composition further comprises a targeting moiety, e.g., a LAT1 targeting ligand, e.g., phenyl alanine, linked to the PEG units. In some aspects, the thiol groups in the micelle form disulfide bonds.

The present disclosure also provides a micelle comprising a miRNA inhibitor of the present disclosure (i.e., miR-485 inhibitor, e.g., SEQ ID NO: 30) wherein the miRNA inhibitor and the delivery agent are associated with each other.

In some aspects, the association is a covalent bond, a non-covalent bond, or an ionic bond. In some aspects, the positive charge of the cationic carrier moiety of the cationic carrier unit is sufficient to form a micelle when mixed with the miR-485 inhibitor disclosed herein in a solution, wherein the overall ionic ratio of the positive charges of the cationic carrier moiety of the cationic carrier unit and the negative charges of the miR-485 inhibitor (or vector comprising the inhibitor) in the solution is about 1: 1.

In some aspects, the cationic carrier unit is capable of protecting the miRNA inhibitor of the present disclosure (i.e., miR-485 inhibitor) from enzymatic degradation. See PCT Publication No. WO2020/261227, published Dec. 30, 2020, which is herein incorporated by reference in its entirety.

### V. Pharmaceutical Compositions

In some aspects, the present disclosure also provides pharmaceutical compositions comprising a miR-485 inhibitor disclosed herein (e.g., a polynucleotide or a vector comprising the miR-485 inhibitor) that are suitable for administration to a subject. The pharmaceutical compositions generally comprise a miR-485 inhibitor described herein (e.g., a polynucleotide or a vector) and a pharmaceutically-acceptable excipient or carrier in a form suitable for administration to a subject. Pharmaceutically acceptable excipients or carriers are determined in part by the particular composition being administered, as well as by the particular method used to administer the composition.

Accordingly, there is a wide variety of suitable formulations of pharmaceutical compositions comprising a miR-485 inhibitor of the present disclosure. (See, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration.

### VI. Kits

The present disclosure also provides kits or products of manufacture, comprising a miRNA inhibitor of the present disclosure (e.g., a polynucleotide, vector, or pharmaceutical composition disclosed herein) and optionally instructions for use, e.g., instructions for use according to the methods disclosed herein. In some aspects, the kit or product of manufacture comprises a miR-485 inhibitor (e.g., vector, e.g., an AAV vector, a polynucleotide, or a pharmaceutical composition of the present disclosure) in one or more containers. In some aspects, the kit or product of manufacture comprises miR-485 inhibitor (e.g., a vector, e.g., an AAV vector, a polynucleotide, or a pharmaceutical composition of the present disclosure) and a brochure. One skilled in the art will readily recognize that miR-485 inhibitors disclosed herein (e.g., vectors, polynucleotides, and pharmaceutical compositions of the present disclosure, or combinations thereof) can be readily incorporated into one of the established kit formats which are well known in the art.

The following examples are offered by way of illustration and not by way of limitation.

### EXAMPLES

### Example 1

### Materials and Methods

Unless provided otherwise, the Examples described below use one or more of the following materials and methods:

### Human Tissue

Brain precentral gyrus samples from patients with Alzheimer's disease (AD) and from controls were purchased from Netherlands brain bank. Information related to these patients and controls are shown in Table 1.

### Mice

B6SJLF1/J (JAX#100012), and five familial AD mutation (5×FAD) transgenic mice (#MMRRC#034848) were purchased from The Jackson Laboratory (Bar Harbor, Me., USA). 5×FAD mice overexpress mutant human amyloid precursor protein (APP) with the Swedish (K670N, M671L), Florida (I716V), and London (V7171) mutations, along with mutant human presenilin 1 (PS1) that carries two FAD mutations (M146L and L286V). These transgenes are regulated by the Thy1 promoter in neurons. The genotype of 5×FAD mice was confirmed by PCR analysis of tail DNA following standard PCR conditions provided by The Jackson Laboratory. Mice of mixed genotypes were housed four to five per cage with a 12-hour light/12-hour dark cycle and food and water ad libitum. All animal procedures were performed according to the Konyang University guidelines for care and use of laboratory animals. The animal studies were approved by the Konyang University Committee (Permit number: P-18-18-A-01).

6-hydroxydopamine (6-OHDA) mice (C57BL/6; 8 weeks old; 20-23 g) were obtained from KOATECH (Pyeongtaek, Korea). The mice were housed in a controlled environment and provided with food and water ad libitum.

### Next Generation Sequencing Using Mouse Frontal Cortex Tissue

NGS was performed in a NovaSeq 6000 system (Illumina) by the Theragen Etex Bio Institute (Seoul, Republic of Korea, woldwideweb.theragenetex.com/kr/bio). TruSeq Stranded mRNA Library Kit (Illumina) was used to build the library. Afterwards, data was processed using ‘Raw read’ for mRNA sequencing. Raw reads were aligned to GRCm38.96 (NCBI) using STAR aligner v2.7.1 for calculation of ‘RSEM’ expression values. Dobin et al., Bioinformatics 29(1): 15-21 (2013). We performed the STAR aligner as the default option. Since the total number of reads for each sample was different, normalization was performed by TMM method. Thirteen mouse samples were processed in the same way. All data is available in the GEO (Gene Expression Omnibus, worldwideweb.ncbi.nlm.nih.gov/geo/) as GSE142633.

### Public Database Usage, Reanalysis, and Network Analysis

We used results from Weinberg et al. to confirm miRNAs that are highly related to cognitive impairment. Weinberg et al., Front Neurosci 9:430 (2015). The 100 genes shown in Figure EV1 were extracted from Table 1 of Weinberg et al. We took log2 in Weinberg et al.'s results, ordered them, and marked the target miRNAs. The “miRDB” was used to search for miRNA targeting specific genes. Wong et al., Nucleic Acids Res 43:D146-52 (2015). The “Genecard” database was used to search for genes related to disease or biological symptoms. Rebhan et al., Bioinformatics 14(8):656-64 (1998). The results in Figure EV2A show search results from using keywords, “Inflammation”, “Amyloid beta degradation” and “Alzheimer” in August 2019. We used “VennDiagram” package of R for analysis for Venn diagram. The “GeneMAINA” (version 3.5.1) package of Cytoscape (version 3.7.1) was used for protein to protein interaction analysis. Franz et al., Nucleic Acids Res 46(W1):W60-W64 (2018). We used 265 common genes that included hsa-miR-485-3p target genes and Alzheimer-related genes as inputs for protein interaction analysis. Among them, 139 genes interacted without neighbor gene. In addition, 9 genes were highly associated with cerebral nervous system diseases (including AD) and at the same time, low expression was reported in the patient group or in a dementia mouse model.

Intraventricular Injection of the miR-485 Inhibitor

The miR485-3p antisense oligonucleotide (ASO) (i.e., miR-485 inhibitor) (AGAGAGGAGAGCCGUGUAUGAC) (SEQ ID NO: 30) and a control oligonucleotide (“miR-control”) (CCTTCCCTGAAGGTTCCTCCTT) (SEQ ID NO: 61) were synthesized by Integrated DNA Technologies (USA). All animals were initially anesthetized with 3-5% isoflurane in oxygen and fixed on a stereotaxic frame (JeongDo). For intracerebroventricular (ICV) injection, miR-485 inhibitor or non-targeting control oligonucleotides were formulated with in vivo jetPEI reagent (Polyplus). miR-485 inhibitor (1.5 μg) or control oligonucleotide, formulated with in vivo jetPEI reagent, was injected with a 10 μL Hamilton syringe (26-gauge blunt needle) at 1.5 μL/min. The miR-485 inhibitor and the control oligonucleotides were infused in a volume of 5μL into 10-month old 5×FAD mice by intracerebroventricular (ICV). miR-485 inhibitor or non-targeting control oligonucleotides were given once a week for 2 weeks. Intracerebroventricular (ICV) position was identified using the coordinates from the bregma: AP=−0.2 mm, L=±1.0 mm, ventral (V)=−2.5 mm.

Intravenous Injection of the miR-485 Inhibitor

The miR485-3p antisense oligonucleotide (ASO) (i.e., miR-485 inhibitor) (AGAGAGGAGAGCCGUGUAUGAC) (SEQ ID NO: 30) or a control oligonucleotide (“miR-control”) (CCTTCCCTGAAGGTTCCTCCTT) (SEQ ID NO: 61) were loaded into a nanoparticle, which comprise a pegylated (PEG) shell, a cross-linked core, and one or more brain targets. In some aspects, the ASOs were fluorescently tagged (e.g., Cy5.5) to allow for tracking using in vivo imaging. Before the injection of micelle or ASO, fluorescence images were taken as pre-injection images. The ASO loaded nanoparticles (25 μg of ASO) were intravenously administered (tail-vein injection) to the mice and fluorescence images of mice were taken at desired time using IVIS in vivo imaging system. Unless indicated otherwise, the mice received a single dose of the ASO loaded nanoparticle. The fluorescence images were observed up to 16 hours, and time dependent fluorescence intensities of ASO loaded micelles were compared to naked ASO injected mice. The fluorescence images of ASO loaded micelles and ASOs were regarded as ASO's distribution, and bio-distribution behavior of two groups were compared.

All animals were initially anesthetized with 3-5% isoflurane in oxygen and fixed on a stereotaxic frame (JeongDo). For intracerebroventricular (ICV) injection, miR-485 inhibitor or non-targeting control oligonucleotides were formulated with in vivo jetPEI reagent (Polyplus). miR-485 inhibitor (1.5 μg) or control oligonucleotide, formulated with in vivo jetPEI reagent, was injected with a 10 μL Hamilton syringe (26-gauge blunt needle) at 1.5 μL/min. The miR-485 inhibitor and the control oligonucleotides were infused in a volume of 5 μL into 10-month old 5×FAD mice by intracerebroventricular (ICV). miR-485 inhibitor or non-targeting control oligonucleotides were given once a week for 2 weeks. Intracerebroventricular (ICV) position was identified using the coordinates from the bregma: AP=−0.2 mm, L=±1.0 mm, ventral (V)=−2.5 mm.

### Glial Cell and Cortical Neuron Culture and Transfection

Mouse primary mixed glial cells were cultured from the cerebral cortices of 1- to 3-day-old C57BL/6 mice. The cerebral cortex was dissected and triturated into single-cell suspensions by pipetting. Then, single-cell suspensions were plated into 6-well plates pre-coated with 0.05 mg/ml poly-D-lysine (PDL) and cultured in DMEM medium supplemented with 25 mM glucose, 10% (vol/vol) heat-inactivated foetal bovine serum, 2 mM glutamine and 1,000 units/mL penicillin-streptomycin (P/S) for 2 weeks. Primary cortical neurons were cultured from embryonic day 17 mice. In brief, cortices were dissected and incubated in ice-cold HBSS (Welgene, LB003-02) solution and dissociated in accumax (Sigma, Cat#A7089) for 15 min at 37° C. The cultures were rinsed twice in HBSS. Mouse neurons were resuspended in neurobasal media (Gibco, Cat#21103049) containing 2% B27 (Gibco, Cat#17504), 1% sodium pyruvate, and 1% P/S. Cells were filtered through a 70 μM cell strainer (SPL, 93070), plated on culture plates and maintained at 37° C. in a humidified 5% CO2 incubator. The medium was changed every 3 days and then after 12-13 days in vitro, cells were used for experiments. Primary glial cell or cortical neurons were transfected with 100 nM miR-control, 100 nM has-miR485-3p mimic or 100 nM miR-485 inhibitor using TRANSIT-X2® Transfection Reagent (Minis Bio).

### Luciferase Assays

Human SIRT1 3′-UTR containing the target site for miR-485-3p was amplified from cDNA by PCR amplification and inserted into the psiCHECK2 vector (Promega, Cat#C8021). HEK293T cells in a 96-well plate were co-transfected with psiCHECK2-Sirt1-3′UTR wild-type (WT) or psiCHECK2-Sirt1-3′UTR mutant (MT) and miR-485-3p using Lipofectamine 2000 (Invitrogen, Cat#11668-027). Cells were harvested 48 hours later, and the Dual Luciferase Assay System (Promega, Cat#E1910) was used to measure the luciferase reporter activities. Three independent experiment were performed in triplicate.

Human CD36 3′-UTR containing the target site for miR-485-3p was amplified from cDNA by PCR amplification and inserted into the pMir-Target vector (Addgene). HEK293T cells in 96-well plates were co-transfected with pMir-CD36-3′UTR WT or pMir-CD36-3′UTR MT and pRL-SV40 vector (Addgene) and miR-485-3p using Lipofectamine 2000 (Invitrogen, Cat#11668-027). Cells were harvested 24˜48 hours later, and the Dual Luciferase Assay System (Promega, Cat#E1910) was used to measure the luciferase reporter activities. Three independent experiment were performed in triplicate.

### In Vitro Binding Assay

Streptavidin magnetic beads (Invitrogen, Cat#11205D) were prepared for in vitro binding assay as follows. Beads (50 μL) were washed five times with 500 μL of 1× B&W buffer (5 mM Tris-HCl , pH 7.4; 0.5 mM EDTA; 1 M NaCl). After removing the supernatant, beads were incubated with 500 μL of 1× B&W buffer containing 100 μg of yeast tRNA (Invitrogen, Cat#AM7119) for 2 hours at 4° C. Beads were washed twice with 500 μL of 1× B&W buffer and incubated with 200 μL of 1× B&W buffer containing 400 pmol of biotin-miR485-3p for 10 minutes at room temperature. The supernatant was removed and beads were washed twice with 500 μL of 1× B&W buffer and collected with a magnetic stand. miRNA-coated beads were incubated with 500 μL of 1× B&W buffer containing 1 μg of in vitro transcribed target mRNA overnight at 4° C. The following day, beads were washed with 1 ml of 1× B&W buffer five times and then resuspended in 200 μL of RNase-free water. Bound RNA was extracted with QiaZol Lysis reagent (Qiagen, Cat#79306) under manufacturer's instructions. Extracted RNA was quantified by StepOnePlus Real-time PCR system (Applied Biosystems, REF: 4376592).

### Western Blot

Brain tissue, primary glial cells or cortical neuron cells were homogenized in ice-cold RIPA buffer (iNtRON Biotechnology) containing protease/phosphatase inhibitor cocktail (Cell Signaling Technology, Cat#5872) on ice for 30 min. The lysates were centrifuged at 13,000 rpm for 15 min at 4° C., and supernatants were collected. The samples were separated by SDS—polyacrylamide gel electrophoresis, transferred to PVDF membranes and incubated with the following primary antibodies: rabbit anti-PGC-1α (Abeam, Cat#ab54481, 1:1000), rabbit anti-APP (Cell Signaling Technology, Cat#2452, 1:1000), mouse anti-sAPPα (IBL, Cat#11088, 1:1000), mouse anti-sAPPα (IBL, Cat#10321, 1:1000), rabbit anti-Adam10 (Abeam, Cat#ab1997, 1:100), mouse anti-CTFs (Biolegend, Cat#SIG-39152, 1:1000), rabbit anti-β-amyloid (1-42) (Cell Signaling Technology, Cat#14974, 1:1000), rabbit anti-BACE1 (Abeam, Cat#ab2077, 1:1000), mouse anti-NeuN (Millipore, #MAB377, 1:1000), rabbit anti-cleaved caspase 3 (Cell Signaling Technology, Cat#9664, 1:1000), mouse anti-GFAP (Merck, Cat#MAB360, 1:1000), rabbit anti-IL-1β (abeam, Cat#9722, 1:1000), rabbit anti-NF-kB(p65) (Cell Signaling Technology, Cat#8242, 1:1000), goat anti-Iba1 (Abeam, Cat#ab5076, 1:1000), rabbit anti-SIRT1 (Abeam, Cat#04-1557), mouse anti-TNF-α (Santa Cruz, Cat#sc-52746), anti-actin (Santa Cruz, Cat#sc-47778). The results were visualized using an enhanced chemiluminescence system, and quantified by densitometric analysis (Image J software, NIH). All experiments were performed independently at least three times.

To measure tyrosine hydroxylase expression in brain tissue (see Example 15), brain tissues were homogenized in ice-cold RIPA buffer (iNtRON Biotechnology) containing protease/phosphatase inhibitor cocktail (Cell Signaling Technology, Cat#5872) on ice for 30 min. The lysates were centrifuged at 13,000 rpm for 15 min at 4° C., and supernatants were collected. The samples were separated by SDS-polyacrylamide gel electrophoresis, transferred to PVDF membranes and incubated with the following primary antibodies: Rabbit anti-tyrosine hydroxylase (TH; 1:2000; Pel-Freez, Brown Beer, Wisconsin, USA), and mouse anti-β-actin (Santa Cruz Biotechnology, Santa Cruz, Calif., USA). Subsequently, the membranes were incubated with secondary antibodies for 1 h at room temperature, and the bands were finally detected using Western-blot detection reagents (Thermo Fisher Scientific, Rockford, Ill., USA). For quantitative analyses, the density of each band was measured using a Computer Imaging Device and accompanying software (Fuji Film, Tokyo, Japan), and the levels were quantitatively expressed as the density normalized to the housekeeping protein band for each sample. All experiments were performed independently at least three times.

### Soluble and Insoluble Aβ Extraction

Brain tissue samples were homogenized with RIPA buffer containing protease/phosphatase inhibitors on ice, followed by centrifugation at 12,000 rpm for 15 min. The supernatants were collected. To obtain the insoluble fraction from brain tissues, the pellet of brain lysates was lysed in insoluble extraction buffer [50 mM Tris-HCl (pH7.5)+2% SDS] containing protease/phosphatase inhibitor cocktail on ice for 30 min. The lysates were centrifuged at 4° C. for 15 min at 13,000 rpm. Protein was quantified using bicinchoninic acid (BCA) assay kit (Bio-Rad Laboratories, Cat#5000116) and adjusted to the same final concentration. After denaturation, the lysates were processed for western blotting to measure insoluble Aβ.

### Immunohistochemistry

For immunohistochemistry, miR-485 inhibitor or control oligonucleotide injected 5×FAD brains were removed, post-fixed and embedded in paraffin. Coronal sections (10-μM thick) through the infarct were cut using a microtome and mounted on slides. The paraffin was removed, and the sections were washed with PBS-T and blocked in 10% bovine serum albumin for 2 hours. Thereafter, the following primary antibodies were applied: purified mouse anti-β-Amyloid, 1-16 (Biolegend, #803001, 1 μg/ml ), rabbit anti-β-amyloid (1-42) (Cell Signaling Technology, #14974s, 1:100), rabbit anti-Iba-1 (Wako, #019-19741, 2 μg/ml), goat anti-Iba-1 (Abcam, #ab5076, 2 μg/ml), rabbit anti-CD68 (Abcam, #ab125212, 1 μg/ml), rabbit anti-GFAP (Abcam, #ab16997, 1:100), mouse anti-GFAP (Millipore, #MAB360, 1:500) rat anti-CD36 (Abcam, #ab80080, 1:100), mouse anti-TNF-α (Santa Cruz, #sc-52746, 1:100), rabbit anti-IL-1β (Abcam, #ab9722, 1 μg/ml), rabbit anti-cleaved caspase-3 (Cell Signaling Technology, #9662S, 1:300), mouse anti-NeuN (Millipore, #MAB377, 10 μg/ml). Images were obtained using a confocal microscope (Leica DMi8). Relative band intensity was normalized relative to actin using ImageJ software (NIH).

### Thioflavin-S Staining

For thioflavin-S(ThS) staining, the sliced brains were stained with filtered 1% aqueous Thioflavin-S solution for 8 minutes. The sections were then rinsed with 80%, 95% ethanol and three washes with distilled water. Afterward, brain slices were mounted and slides allowed to dry in the dark overnight. Images were taken on a Leica fluorescence microscope.

### Preparation of Aβ¹⁻⁴² Fibrils

Aβ¹⁻⁴² Hexafluoroisoproponal (HFIP) peptide (#AS-64129) was obtained from AnaSpec (Fremont, Calif., USA). Aβ0 1-42 fibrils was prepared as described previously. Coraci et al., American J of Pathology 160(1): 101-12 (2002). To form fAβ synthetic human Aβ₁₋₄₂, Aβ₁₋₄₂. HFIP peptide was dissolved in DMSO to a stock concentration of 5 mM. Stocks were then diluted to 100 μM in serum free DMEM and incubated at 37° C. for 72 hours. Fibrillar Aβ (fAβ) were confirmed by SDS-PAGE.

In vitro Phagocytosis Assays (ELISA and Immunocytochemistry)

BV2 microglial cells (2×10⁵) were plated in 6-well plates overnight. Cells were transfected using a TRANSIT-X2® Transfection Reagent (Minis Bio, Cat#MIR6000) according to the manufacturer s instructions and treated with fAβ for 4 hours at a final concentration of 1 μM. When applicable, anti-CD36 antibody was applied to the media with fAβ. After 4 hours, media was collected from BV2 microglia. Levels of human Aβ (1-42) in supernatant were measured by the human Aβ42 ELISA kit (Invitrogen, Cat#KHB3441), according to the manufacturer's instructions.

In addition, glial phagocytosis was verified by fluorescence microscope. Coverslips were coated with poly-1-lysine before plating 8×10⁴ primary glial cells per coverslip resting in wells of a 24-well plate overnight. Primary glial cells were transfected using TRANSIT-X2® Transfection Reagent (Minis Bio) according to the manufacturer's instructions and incubated in unlabeled fAβ for 4 hours at a final concentration of 1 μM. After the four-hour incubation, the cells were washed with cold PBS. For Aβ uptake measurement, primary glial cells were then fixed with 100% methanol for 1 hour at −20° C., washed with PBS-T and incubated at 4° C. with mouse anti-β-Amyloid 1-16, rabbit anti-GFAP (abcam, #ab16997, 1:100) and rabbit anti-Iba-1 (Wako, #019-19741, 2 μg/ml)

### FACS Analysis

All staining steps were performed in the dark and blocked with BD Fc Block. Primary glial cells were stained using the following antibodies: Alexa 488-conjugated anti-mouse CD36 (Biolegend, Cat#102607, 5 μg/ml) or isotype control Ab (Biolegend, Cat#400923, 5 μg/ml) for 30 min at 4° C. After 30 min, cells were washed with FACS buffer (PBS+1%). Data were analyzed with CellQuest (BD Bioscience) and FlowJo software (Treestar) packages.

### Real Time PCR

Total RNA was isolated using the Isolation of small and large RNA kit (Macherey Nagel, Dfiren). cDNA was synthesized using miScript II RT Kit (Qiagen, Hilden, Germany). For analysis the expression of miR-485-3p was performed by TaqMan miRNA analysis using TOPREAL™ qPCR 2× PreMIX (Enzynomics, Korea) on CFX connect system (Bio-Rad). The real-time PCR measurement of individual cDNAs was performed using SYBR green and Taq man probe to measure duplex DNA formation with the Bio-Rad real-time PCR system. Primers were as follows: Probe: FAM-CGAGGTCGACTTCCTAGA-NFQ. (SEQ ID NO: 51) miR-485-3p forward: 5′-CATACACGGCTCTCCTCTCTAAA-3′ (SEQ ID NO: 52); Mouse primer: Actin forward: 5′ -TCCTGTGGCATCCATGAAAC-3′ (SEQ ID NO: 53), reverse: 5′-CAATGCCTGGGTACATGGTG-3′ (SEQ ID NO: 54); TNF forward: 5′-CCAAGTGGAGGAGCAGCT-3′ (SEQ ID NO: 55), reverse: 5′-GACAAGGTACAACCCATCGG-3′ (SEQ ID NO: 56); IL-1β forward: 5′-TTCGACACATGGGATAACGAGG-3′ (SEQ ID NO: 57), reverse: 5′-TTTTTGCTGTGAGTCCCGGAG-3′ (SEQ ID NO: 58); miR-16 forward: 5′-CAGCCTAGCAGCACGTAAAT-3′ (SEQ ID NO: 59); reverse: 5′-GAATCGAGCACCAGTTACG-3′ (SEQ ID NO: 60); miR-16 level was used for normalization. The relative gene expression was analyzed by the 2-ΔΔct method.

### ELISA (TNF-α and IL-1β)

Primary mixed glial (2×10⁵) cells were plated in 6-well plates overnight. Cells were treated a miR-485 inhibitor with mouse a-synuclein PFF (aggrergated form) for 18 h at a final concentration of 1 μg/ml. After 18 h, media was collected from primary mixed glial cells. Levels of TNF-α and IL-1b in supernatant were measured by the mouse TNF-α ELISA kit (R&D system, Cat#MTA00B) and the mouse IL-1b ELISA kit (R&D system, Cat#MLB00C). The ELISA was performed according to the manufacturer's instructions.

### Behavioral Tests (1⁷-Maze and Passive Avoidance)

The Y-maze consisted of three black, opaque, plastic arms (30 cm×8 cm×15 cm) 120° from each other. The 5×FAD mice were placed in the center and were allowed to explore all three arms. The number of arm entries and number of trials (a shift is 10 cm from the center, entries into three separate arms) were recorded to calculate the percentage of alternation. An entry was defined as all three appendages entering a Y-maze arm. Alternation behavior was defined as the number of triads divided by the number of arm entries minus 2 and multiplied by 100.

The passive avoidance chamber was divided into a white (light) and a black (dark) compartment (41 cm×21 cm×30 cm). The light compartment contained a 60 W electric lamp. The floor (of the dark) department contained a number of (2-mm) stainless steel rods spaced 5 mm apart. The test was done for 3 days. The first day adapts the mouse for 5 minutes in a bright zone. The second day is the training phase. The study consists of two steps. The first step places each mouse in the light zone which is then moved to the dark zone twice. One hour after the first step, each mouse is placed in the light compartment. The door separating the two compartments was opened 30 seconds later and after mice enter the dark compartment, the door was closed and an electrical foot shock (0.3 mA/10 g) was delivered through the grid floor for 3 seconds. If the mouse does not go into the dark zone for more than 5 minutes, it is considered to have learned avoidance, and the training was done up to 5 times. Twenty-four hours after the training trial, mice were placed in the light chamber for testing. Latency was defined as the time it took for a mouse to enter the dark chamber after the door separating the two compartments opened. The time taken for the mouse to enter the dark zone and exit to the bright zone was defined as TDC (time spent in the dark compartment).

### Behavioral Tests (Rotarod)

Mice were trained on the rotarod apparatus (3 cm rod diameter) at a fixed speed of 10 rpm for 600 s once daily for 3 consecutive days. Performance on the rod was evaluated at a constant acceleration rate of 4-40 rpm in 300 s. Two consecutive trials were performed at 60 min intervals.

### Behavior Tests (Hang Wire Test)

For the wire hang test of motor coordination, mice were tested on 2 mm thick and 55 cm long taut metal wires. The custom-built were hang apparatus consisted of a black polystyrene box that was 60 cm long into which mice could fall. The latency of the mice to fall from the wire after being suspended was recorded measuring the longest suspension time in 3 trials per mouse.

### Behavior Tests (Pole Test)

The pole test assesses the agility of animals and may be a measure of bradykinesia. Mice were placed head-upward at the top of a rough-surfaced pole (8 mm in diameter and 55 cm in height). Performance was measured as the total time it took each mouse to arrive at the floor form the top. Before actual test, mice were trained in 5 trials/d for 3 d. the locomotor activity of each mouse was evaluated as the average of 5 trials performed at 6 d after 6-OHDA and miR-485 inhibitor i.v. administration.

### Behavior Tests (Balance Beam Test)

Mice were on a 0.5 cm wide, 1 m long balance beam apparatus. The balance beam consisted of a transparent Plexiglas structure that was 50 cm high with a dark resting box at the end of the runway. Mice were trained on the beam for three times in the morning, allowing for a resting inter-trial period of a least 15 min. Mice were left in the dark resting box for at least 10 s before being placed back in their home cage. Mice were then re-tested in the afternoon, at least 2 h after the training session. During test session, mice performance was recorded. The test consisted of three trials with a resting inter-trial period of at least 10 min. The number of total paw slips was calculated manually for the last of the three tests. For SOD1G93A mutant mice were tested at 44 or 48 days after PBS or miR-485 inhibitor injection.

### Data Analysis

All data are presented as the mean±SD. NGS data were analyzed using R (version 3.5.2). Statistical significance in the values obtained for two different groups were determined using unpaired t-test. Statistical tests were performed using GraphPad Prism 5 or 8 (GraphPad Software, La Jolla, Calif.). Statistical significance between the two groups was analyzed by two-way Anova and unpaired t-test. Behavior tests were assessed by nonparametric statistical procedures.

### Example 2

### Preparation of miR-485 Inhibitor

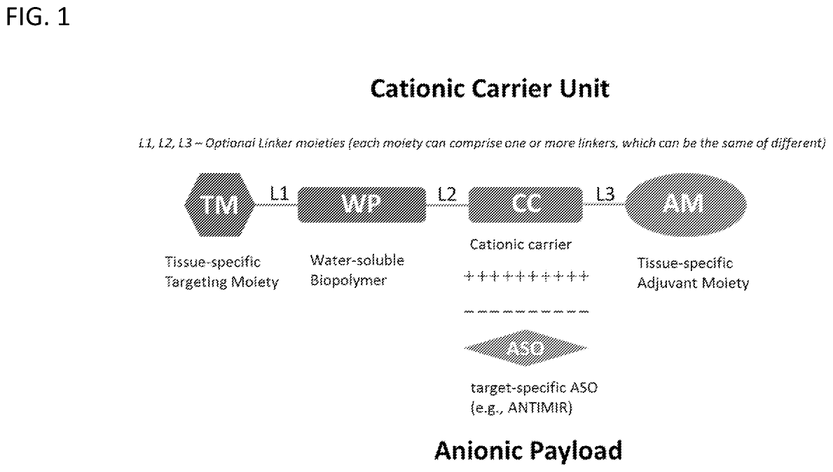

(a) Synthesis of alkyne modified tyrosine: An alkyne modified tyrosine was generated as an intermediate for the synthesis of a tissue specific targeting moiety (TM, see FIG. 1) of a cationic carrier unit to direct micelles of the present disclosure to the LAT1 transporter in the BBB.

A mixture of N-(tert-butoxycarbonyl)-L-tyrosine methyl ester (Boc-Tyr-OMe) (0.5 g, 1.69 mmol) and K₂CO₃ (1.5 equiv., 2.54 mmol) in acetonitrile (4.0 ml) was added drop by drop to propargyl bromide (1.2 equiv., 2.03 mmol). The reaction mixture was heated at 60° C. overnight. After the reaction, the reaction mixture was extracted using water:ethyl acetate (EA). Then, the organic layer was washed using a brine solution. The crude material was purified by flash column (EA in hexane 10%). Next, the resulting product was dissolved in 1,4-dioxane (1.0 ml) and 6.0 M HCl (1.0 ml). The reaction mixture was heated at 100° C. overnight. Next, the dioxane was removed and extracted by EA. Aqueous NaOH (0.5 M) solution was added to the mixture until the pH value become 7. The reactant was concentrated by evaporator and centrifuged at 12,000 rpm at 0° C. The precipitate was washed with deionized water and lyophilized.

(b) Synthesis of poly(ethylene glycol)-b-poly(L-lysine) (PEG-PLL): This synthesis step generated the water-soluble biopolymer (WP) and cationic carrier (CC) of a cationic carrier unit of the present disclosure (see FIG. 1).

Poly(ethylene glycol)-b-poly(L-lysine) was synthesized by ring opening polymerization of Lys(TFA)-NCA with monomethoxy PEG (MeO-PEG) as a macroinitiator. In brief, MeO-PEG (600 mg, 0.12 mmol) and Lys(TFA)-NCA (2574 mg, 9.6 mmol) were separately dissolved in D1VIF containing 1M thiourea and DMF(or NMP). Lys(TFA)-NCA solution was dropped into the MeO-PEG solution by micro syringe and the reaction mixture was stirred at 37° C. for 4 days. The reaction bottles were purged with argon and vacuum. All reactions were conducted in argon atmosphere. After the reaction, the mixture was precipitated into an excess amount of diethyl ether. The precipitate was re-dissolved in methanol and precipitated again into cold diethyl ether. Then it was filtered and white powder was obtained after drying in vacuo. For the deprotection of TFA group in PEG-PLL(TFA), the next step was followed.

MeO-PEG-PLL(TFA) (500 mg) was dissolved in methanol (60 mL) and 1N NaOH (6 mL) was dropped into the polymer solution with stirring. The mixture was maintained for 1 day with stirring at 37° C. The reaction mixture was dialyzed against 10 mM HEPES for 4 times and distilled water. White powder of PEG-PLL was obtained after lyophilization.

(b) Synthesis of azido-poly(ethylene glycol)-b-poly(L-lysine) (N₃-PEG-PLL): This synthesis step generated the water-soluble biopolymer (WP) and cationic carrier (CC) of a cationic carrier unit of the present disclosure (see FIG. 1).

Azido-poly(ethylene glycol)-b-poly(L-lysine) was synthesized by ring opening polymerization of Lys(TFA)-NCA with azido-PEG (N₃-PEG). In brief, N₃-PEG (300 mg, 0.06 mmol) and Lys(TFA)-NCA (1287 mg, 4.8 mmol) were separately dissolved in DMF containing 1M thiourea and DMF(or NMP). Lys(TFA)-NCA solution was dropped into the N₃-PEG solution by micro syringe and the reaction mixture was stirred at 37° C. for 4 days. The reaction bottles were purged with argon and vacuum. All reactions were conducted in argon atmosphere. After the reaction, the mixture was precipitated into an excess amount of diethyl ether. The precipitate was re-dissolved in methanol and precipitated again into cold diethyl ether. Then it was filtered and white powder was obtained after drying in vacuo. For the deprotection of TFA group in PEG-PLL(TFA), the next step was followed.

N₃-PEG-PLL (500 mg) was dissolved in methanol (60 mL) and 1N NaOH (6 mL) was dropped into the polymer solution with stirring. The mixture was maintained for 1 day with stirring at 37° C. The reaction mixture was dialyzed against 10 mM HEPES for 4 times and distilled water. White powder of N₃-PEG-PLL was obtained after lyophilization.

(c) Synthesis of (methoxy or) azido-poly(ethylene glycol)-b-poly(L-lysine/nicotinamide/mercaptopropanamide) (N₃-PEG-PLL(Nic/SH)): In this step, the tissue-specific adjuvant moieties (AM, see FIG. 1) were attached to the WP-CC component of a cationic carrier unit of the present disclosure. The tissue-specific adjuvant moiety (AM) used in the cationic carrier unit was nicotinamide (vitamin B3). This step would yield the WP-CC-AM components of the cationic carrier unit depicted in FIG. 1.

Azido-poly(ethylene glycol)-b-poly(L-lysine/nicotinamide/mercaptopropanamide) (N₃-PEG-PLL(Nic/SH)) was synthesized by chemical modification of N₃-PEG-PLL and nicotinic acid in the presence of EDC/NHS. N₃-PEG-PLL (372 mg, 25.8 μmol) and nicotinic acid (556.7 mg, 1.02 equiv. to NH2 of PEG-PLL) were separately dissolved in mixture of deionized water and methanol (1:1). EDC.HCl (556.7 mg, 1.5 equiv. to NH₂ of N₃-PEG-PLL) was added into nicotinic acid solution and NHS (334.2 mg, 1.5 equiv. to NH2 of PEG-PLL) stepwise added into the mixture.

The reaction mixture was added into the N₃-PEG-PLL solution. The reaction mixture was maintained at 37° C. for 16 hours with stirring. After 16 hours, 3,3′ -dithiodiproponic acid (36.8 mg, 0.1 equiv.) was dissolved in methanol, EDC.HCl (40.3 mg, 0.15 equiv.), and NHS (24.2 mg, 0.15 equiv.) were dissolved each in deionized water. Then, NHS and EDC.HCl were added sequentially into 3,3′-dithiodiproponic acid solution. The mixture solution was stirred for 4 hours at 37° C. after adding crude N₃-PEG-PLL(Nic) solution.

For purification, the mixture was dialyzed against methanol for 2 hours, added DL-dithiothreitol (DTT, 40.6 mg, 0.15 equiv.), then activated for 30 min.

For removing the DTT, the mixture was dialyzed sequentially methanol, 50% methanol in deionized water, deionized water.

d) Synthesis of Phenyl alanine-poly(ethylene glycol)-b-poly(L-lysine/nicotinamide/mercaptopropanamide) (Phe-PEG-PLL(Nic/SH)): In this step, the tissue-specific targeting moiety (TM) was attached to the WP-CC-AM component synthesized in the previous step. The TM component (phenyl alanine) was generated by reaction of the intermediate generated in step (a) with the product of step (c).

To target brain endothelial tissue in blood vessels, as a LAT1 targeting amino acid, phenyl alanine was introduced by click reaction between N₃-PEG-PLL(Nic/SH) and alkyne modified tyrosine in the presence of copper catalyst In brief, N₃-PEG-PLL(Nic/SH) (130 mg, 6.5 μmol) and alkyne modified phenyl alanine (5.7 mg, 4.0 equiv.) were dissolved in deionized water (or 50 mM sodium phosphate buffer). Then, CuSO4.H₂O (0.4 mg, 25 mol%) and Tris(3-hydroxypropyltriazolylmethyl)amine (THPTA, 3.4 mg, 1.2 equiv.) were dissolved deionized water and added N₃-PEG-PLL(Nic/SH) solution. Then, sodium ascorbate (3.2 mg, 2.5 equiv.) were added into the mixture solution. The reaction mixture was maintained with stirring for 16 hours at room temperature. After the reaction, the mixture was transferred into dialysis membranes (MWCO=7,000) and dialyzed against deionized water for 1 day. The final product was obtained after lyophilization.

(e) Polyion Complex (PIC) micelle preparation—Once the cationic carrier units of the present disclosure were generated as described above, micelles were produced. The micelles described in the present example comprised cationic carrier units combined with an antisense oligonucleotide payload.

Nano sized PIC micelles were prepared by mixing MeO- or Phe-PEG-PLL(Nic) and miRNA. PEG-PLL(Nic) was dissolved in HEPES buffer (10 mM) at 0.5 mg/mL concentration. Then a miRNA solution (22.5 μM) in RNAse free water was mixed with the polymer solution at 2:1 (v/v) ratio of miRNA inhibitor (SEQ ID NOs: 2-30) (e.g., AGAGAGGAGAGCCGUGUAUGAC; SEQ ID NO: 30) to polymer.

The mixing ratio of polymer to anti-miRNA was determined by optimizing micelle forming conditions, i.e., ratio between amine in polymer (carrier of the present disclosure) to phosphate in anti-miRNA (payload). The mixture of polymer (carrier) and anti-miRNA (payload) was vigorously mixed for 90 seconds by multi-vortex at 3000 rpm, and kept at room temperature for 30 min to stabilize the micelles.

Micelles (10 μM of Anti-miRNA concentration) were stored at 4° C. prior to use. MeO- or Phe-micelles were prepared using the same method, and different amounts of Phe-containing micelles (25%˜75%) were also prepared by mixing both polymers during micelle preparation.

### Example 3

### Analysis of SIRT1 Expression in Alzheimer's Disease

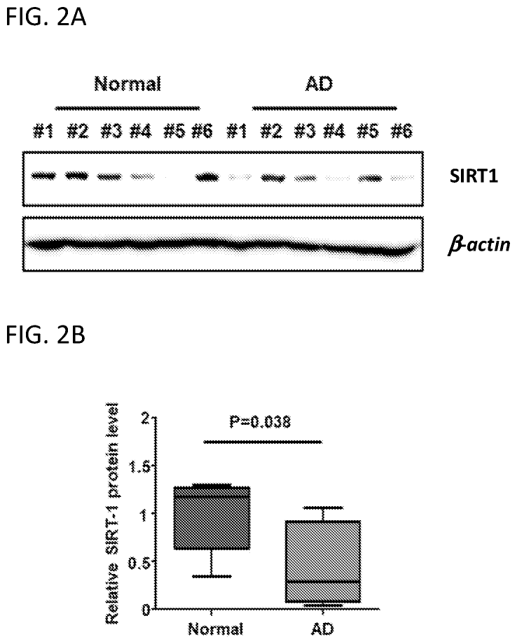

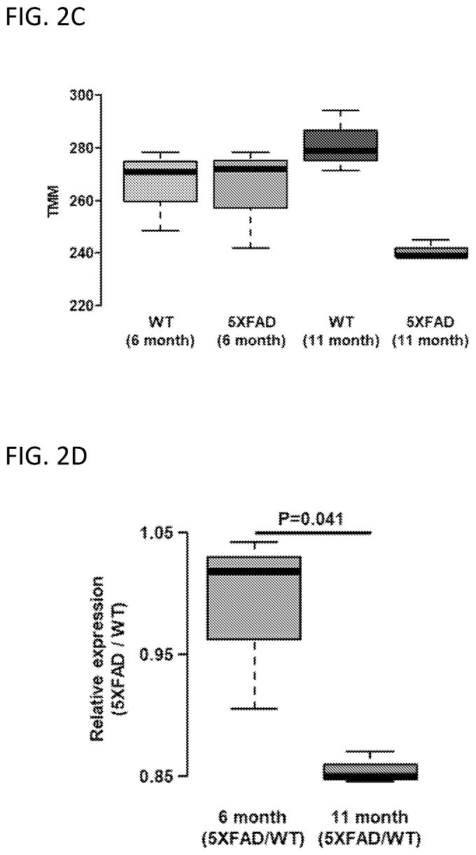

Previous studies reported that SIRT1 levels were reduced in brains of human AD patients and this reduction affected AD progression from early to late stages (Julien et al, 2009, Lutz et al, 2014). To begin assessing the potential therapeutic effects of miR-485 inhibitors disclosed herein, SIRT1 expression was assessed in postmortem brain (precentral gyrus) samples from Alzheimer's disease (AD) patients. As shown in FIGS. 2A and 2B, SIRT1 protein levels were notably reduced in AD patient brains compared to normal human brains.

To confirm the above results, SIRT1 expression was assessed in an established AD animal model (i.e., five familial AD mutation (5×FAD) transgenic mice). As shown in 1C, there was no significant difference in SIRT1 expression between the 6-month old AD mice compared to the wild-type control animals. However, in the 11-month old AD mice, there was a significant reduction in SIRT1 expression (see FIG. 2C). SIRT1 expression was gradually reduced as the 5×FAD aged mice (FIG. 2D).

The above results confirm the earlier studies and demonstrate that SIRT1 expression is down-regulated in AD, suggesting that SIRT1 can play a role in AD pathogenesis.

### Example 4

### Analysis of the Potency of miR-485 Inhibitors in Regulating miR485-3p Expression

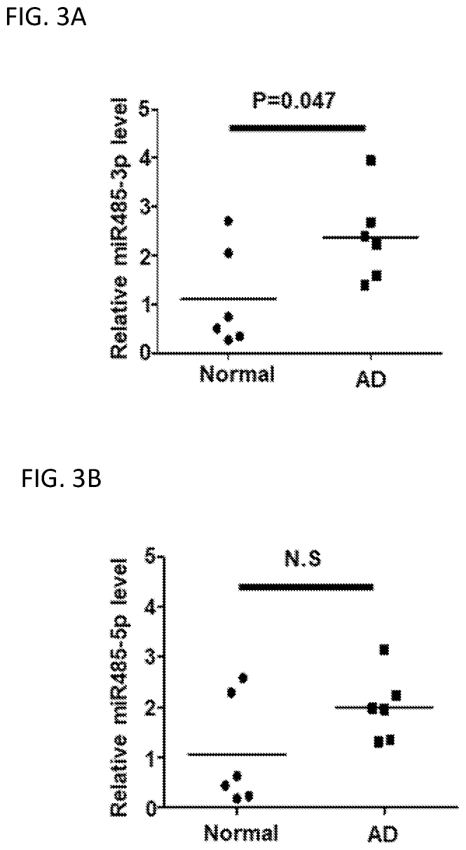

To identify potential miRNA candidates that could regulate SIRT1 expression, brain samples of AD patients were further analyzed for miRNAs that were overexpressed in the samples. As shown in FIG. 3A, miR485-3p expression was significantly higher in precentral gyrus tissue of AD patients compared to normal healthy tissue. No significant differences were observed for other SIRT1 related miRNAs, including miR485-5p (see FIG. 3B).

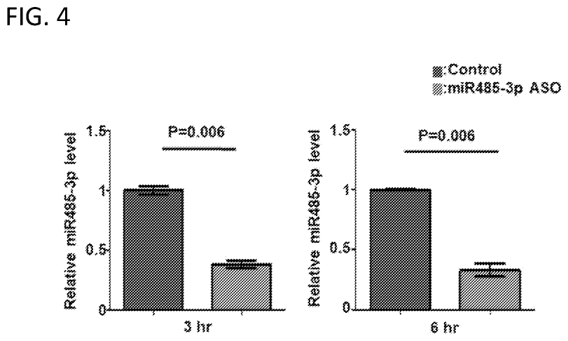

Next, using publicly available algorithms (e.g., Targetscan and miRbase programs), it was predicted that miR-485-3p has a binding site in the 3′UTR of SIRT1. To confirm, the ability of human miR485-3p mimic and inhibitor to regulate miR485-3p expression in mice was assessed. As shown in FIG. 4, using real time PCR analysis, a significant reduction in miR485-3p expression was observed in mouse primary cortical neurons when transfected with a human miR485-3p inhibitor.

This result confirms that the miR485 inhibitors disclosed herein can reduce and/or inhibit miR485-3p expression.

### Example 5

### Analysis of miR-485 Inhibitor Regulation of SIRT1

To understand the relationship between miR485-3p and SIRT1 expression, mouse primary cortical neurons were transfected with one of the following: (i) human miR-control, (ii) human miR485-3p, or (iii) miR485-3p inhibitor. Then, the expression of SIRT1 was assessed in the transfected cells. As shown in FIGS. 5A and 5B, SIRT1 protein expression was reduced in miR485-3p transfected primary cortical neurons compared to miR-control transfected neurons. In contrast, primary cortical neurons transfected with the miRNA inhibitor disclosed herein expressed significantly higher level of SIRT1 protein. And, as shown in FIGS. 5A and 5B, SIRT1 expression appeared to be correlated with PGC-1α expression.

These results demonstrate that the miR485 inhibitors of the present disclosure can increase SIRT1 expression by regulating miR485-3p expression. The results further demonstrate that the miRNA inhibitors disclosed herein can also be useful in increasing PGC-1α protein expression in cells.

### Example 6

### Analysis of the Binding of miR485-3p to SIRT1

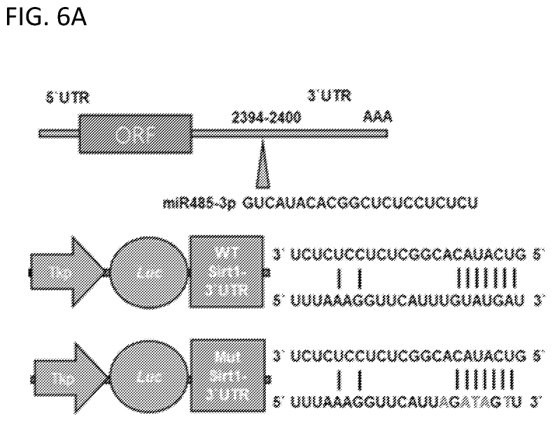

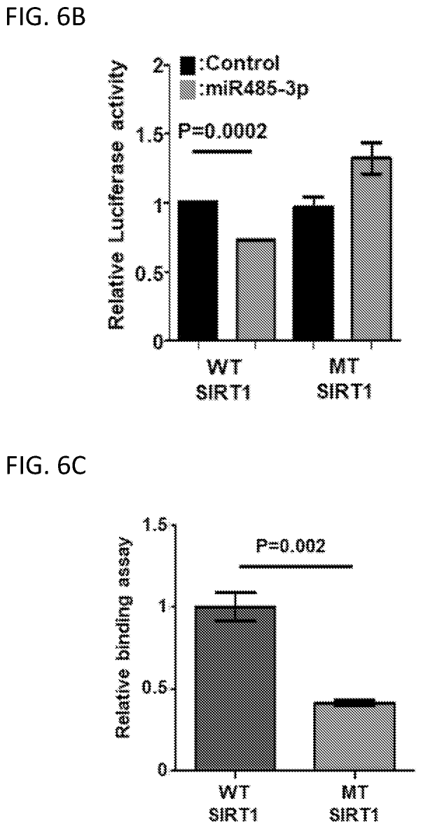

To confirm the target site for miR485-3p on SIRT1, luciferase reporter plasmids of the SIRT1 3′-UTR containing either wild-type or mutated sequence of the potential miR485-3p site were constructed (see FIG. 6A). Then, HEK293T cells were transfected with the plasmids, and promoter activity was measured in the transfected cells. As shown in FIG. 6B, wild type promoter activity was significantly reduced but the mutant form was not different in miR485-3p transfected cells.

Next, the physical binding of miR485-3p to the 3′ UTR of SIRT1 was assessed using an in vitro binding assay. Briefly, streptavidin-miR483-3p-coated magnetic beads were incubated with in vitro transcribed wild type 3′ UTR and mutant 3′ UTR of SIRT1 respectively. Binding RNA was eluted and quantified by realtime PCR. Relative binding was calculated using the formula: Relative Binding=100×2^{((Adjusted input)-(Ct IP))}/100×2^{((Adjusted input)-(Ct WT))}. Compared to wild-type seed (i.e., site where miRNA binds) sequences, the relative binding efficiency was significantly reduced in 3′ UTR containing mutant seed sequences (see FIG. 6C).

The above results collectively demonstrate that miR485-3p can directly target the 3′-UTR of SIRT1 and that this interaction can negatively regulate SIRT1 expression.

### Example 7

### Analysis of miR-485 Inhibitor on Beta Amyloid (AD) Plaque Formation

To explore the potential therapeutic benefits of miR-485 inhibitors disclosed herein on Alzheimer's disease, the effect of miR-485 inhibitors on amyloid plaque formation and insoluble Aβ levels was assessed in 10-month old 5×FAD mice. It has been shown that 5×FAD transgenic mice exhibit show amyloid plaque deposition starting at 2 months and that the aggregation of Aβ into such plaques worsens as AD progresses. Eimer et al., Mol Neurodegener 8:2 (2013); and Näslund et al., Proc Natl Acad Sci 91:8378-08382 (1994).

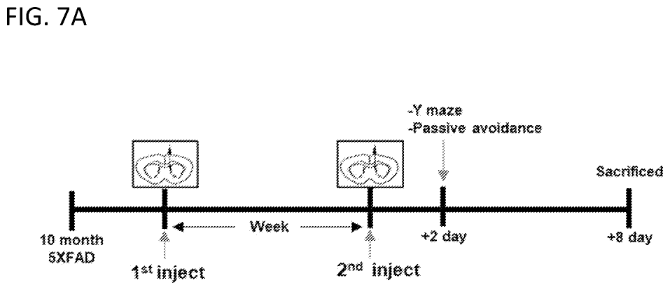

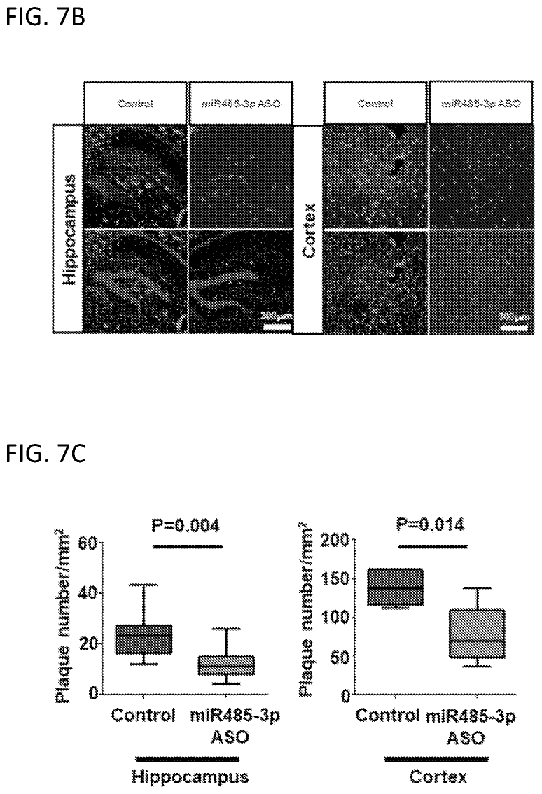

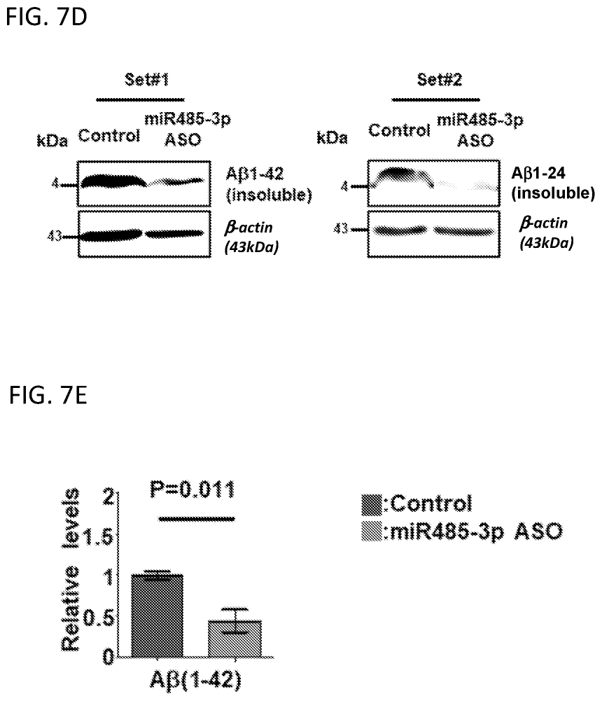

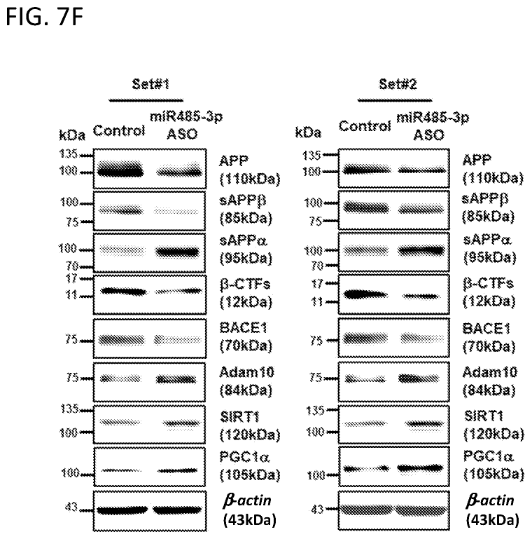

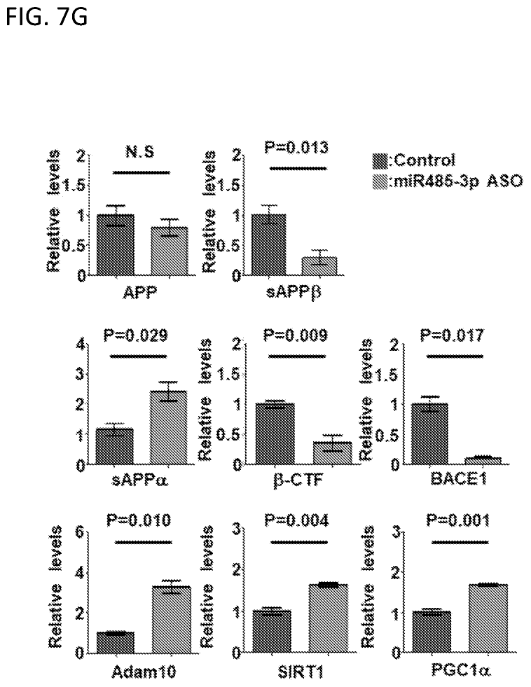

Briefly, miR-485 inhibitor formulated with in vivo jetPEI reagent was injected in the right lateral ventricle of the animals by stereotaxic injection. The animals received a second administration a week later (see FIG. 7A). Then, the number of amyloid plaque formation was quantified using immunofluorescence microscopy using 6E10 staining and thioflavin S. As shown in FIGS. 7B and 7C, the number of amyloid plaques was markedly decreased in 5×FAD animals treated with the miR-485 inhibitor compared to the animals treated with the miR-control, suggesting that the miR-485 inhibitor can ameliorate amyloid burden in AD mice.

To further investigate the effect of miR-485 inhibitors on Aβ production, the levels of insoluble Aβ 1-42, amyloid precursor protein (APP) and APP processing enzymes, including α-secretase, ADAM (A disintegrin and metalloprotease) 10, and β-secretase BACE1 were assessed in the frontal cortex of the AD mice from the different treatment groups.

As shown in FIGS. 7D and 7E, there was a significant reduction in insoluble Aβ 1-42 production in AD mice treated with the miR-485 inhibitor compared to the control animals (i.e., treated with miR-control). miR-485 treated animals also exhibited decreased levels of β-CTFs and sAPPβ (i.e., the main products of BACE) in the frontal cortex, compared to the control animals (see FIGS. 7F and 7G). Accordingly, there was also reduced expression of BACE1 in the inhibitor treated AD animals. And, confirming the results shown earlier (see Example 4), AD mice treated with the miR-485 inhibitor had significantly reduced levels of SIRT1 and PGC-1α protein. However, some of the proteins tested were not negatively regulated by miR-485 administration. For instance, there was no significant difference in total APP levels among the animals from the different treatment groups (see FIGS. 7F and 7G). And, for Adam10 and sAPPα proteins, administration of the miR-485 inhibitor significantly increased the expression of these proteins compared to the control animals.

The above results demonstrate that the miR-485 inhibitors disclosed herein can regulate different genes and thereby, reduce both Aβ production and plaque formation in vivo.

### Example 8

### Analysis of miR-485 Inhibitor on Aβ Plaque Phagocytosis

Alzheimer's disease is caused by imbalances between Aβ production and clearance. Previous studies have shown that glial cells mediate clearance and phagocytosis of aggregated Aβ in AD brain, where they contribute to the alleviation of AD. Ries et al., Front Aging Neurosci 8:160 (2016). Therefore, to further explore the role of glial cells in AD, the co-localization of glial cells and Aβ plaque was assessed in AD mice using immunohistochemistry analysis using Iba1 and 6E10 antibodies.

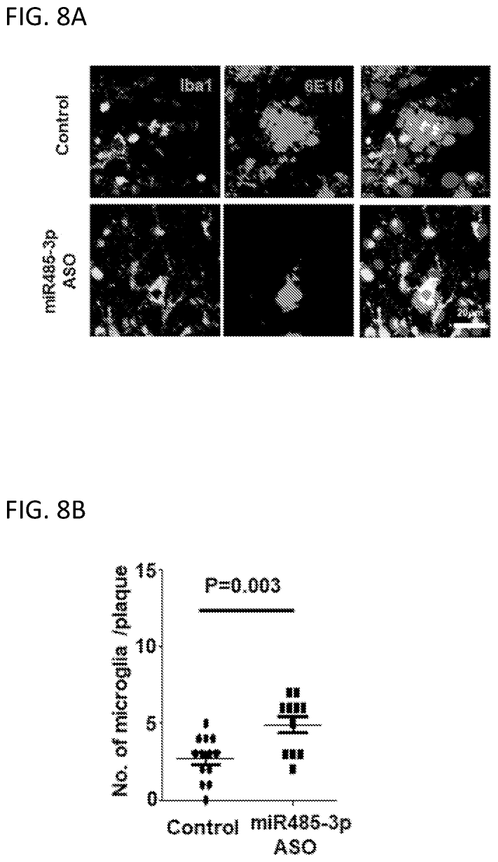

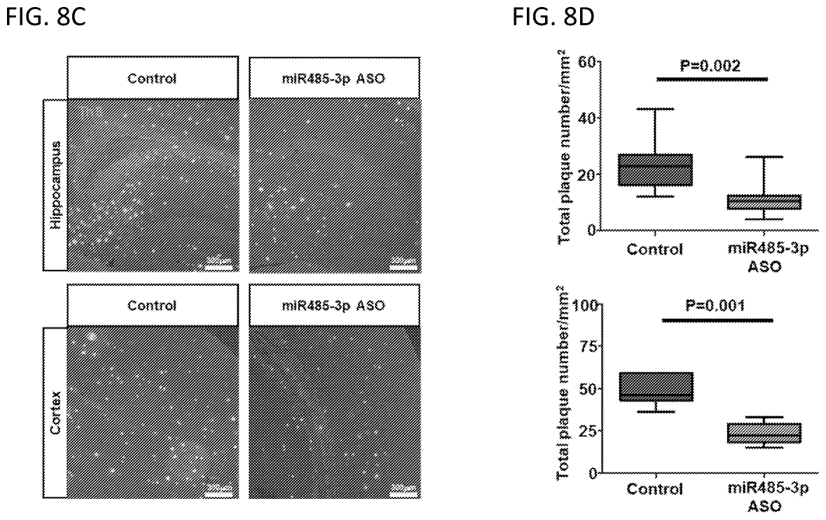

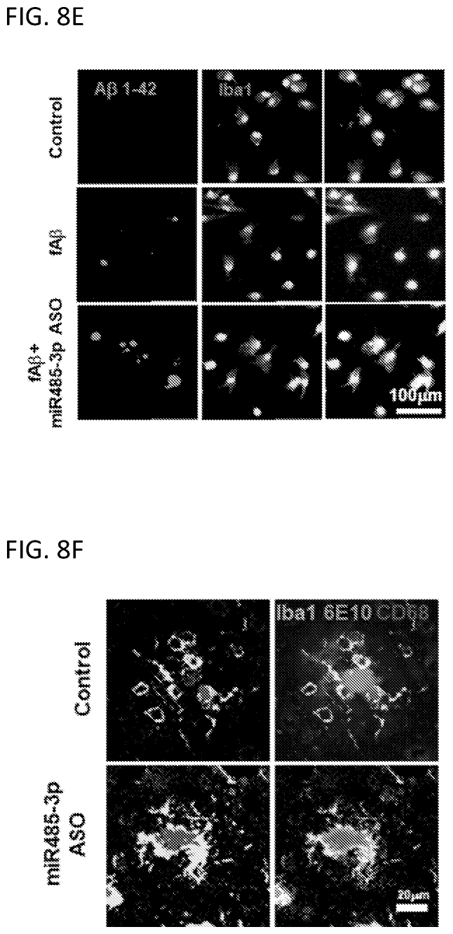

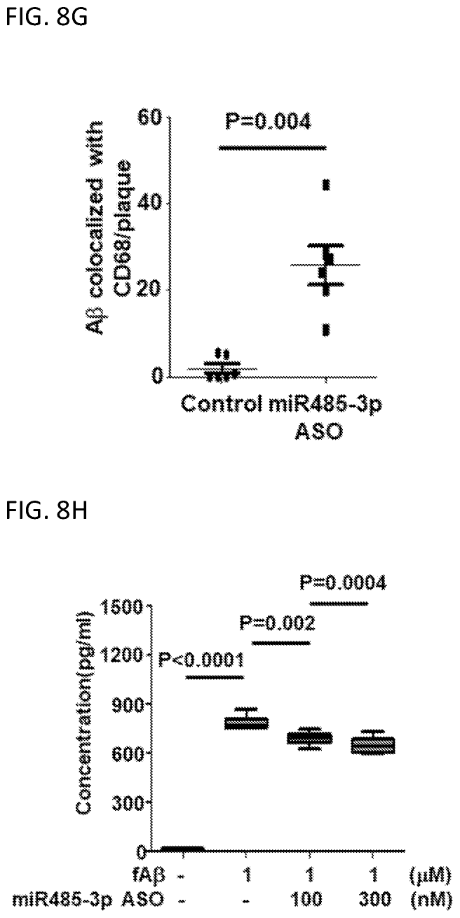

As shown in FIGS. 8A-8D, there was significantly higher colocalization of Aβ plaque and glial cells in AD mice treated with miR-485 inhibitor. In addition, administration of the miR-485 inhibitor to the AD mice consistently increased the uptake of Aβ plaques by the primary glial cells (see FIG. 8E).

Next, to further assess Aβ engulfment and clearance by glial cell, the number of CD68+ microglial phagosomes that had internalized Aβ plaques was quantified using CD68, 6E10, and Iba1 co-immunostaining. CD68, a transmembrane glycoprotein of the lysosome/endosome-associated membrane glycoprotein family, acts as a scavenger receptor for debris clearance. Yamada et al., Cell Mol Life Sci 54(7):628-40 (1998).

As shown in FIGS. 8F and 8G, the clustering of Iba1+ microglia surrounding amyloid plaques exhibited a diffuse CD68 distribution in AD mice treated with the miR-485 inhibitor, compared to the control animals (i.e., treated with miR-control).

To confirm the above results, Aβ aggregates were prepared by incubating Aβ monomers (100 μM) at 37° C. overnight then diluting the peptide stock with cell culture medium. Then, primary glial cells were transfected with the miR-485 inhibitor and further treated with 1 μM fibrillar amyloid beta (fAβ) for 4 hours. Consistent with the above results, Aβ levels in conditioned media were considerably reduced in miR485-3p ASO transfected cells compare to control transfected cells (FIG. 8H).

The above results demonstrate that the miR-485 inhibitors disclosed herein can enhance microglial Aβ phagocytosis.

### Example 9

### Analysis of miR-485 Inhibitor Regulation of CD36

As described herein, CD36/SR-BII can contribute to the phagocytosis of Aβ by glial cells. Using publicly available algorithms (see Example 3), it was predicted that miR-485-3p also has a binding site in the 3′UTR of CD36. Accordingly, to assess whether the miR-485 inhibitors disclosed herein can also regulate CD36 expression, AD mice were treated with either a miR-485 inhibitor or miR-control (as described in the earlier examples), and then the expression of CD36 was assessed in the animals.

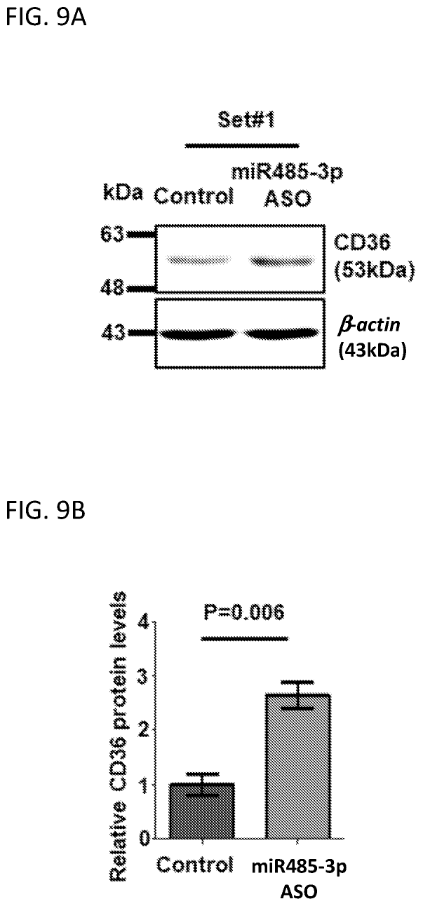

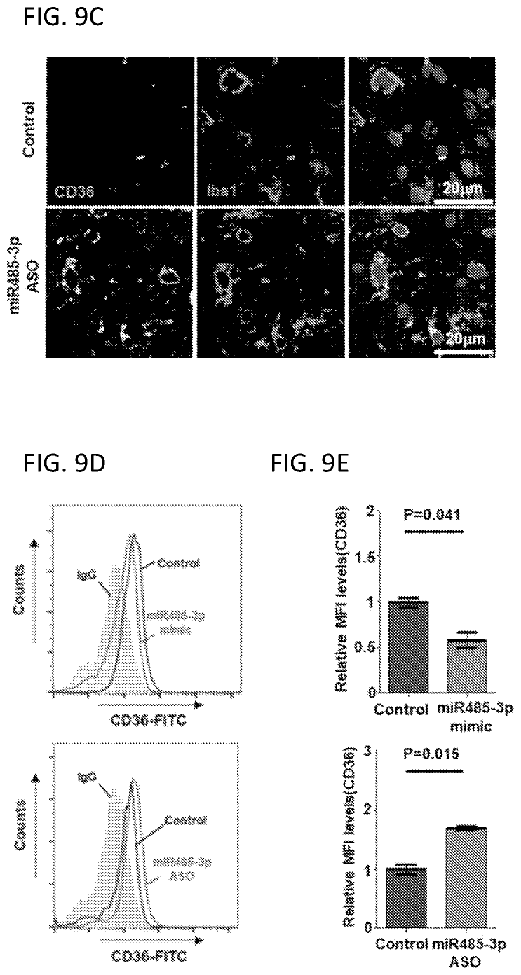

As shown in FIGS. 9A and 9B, AD mice treated with the miR-485 inhibitor exhibited significantly higher CD36 expression compared to the control animals. In addition, CD36 expression was noticeably higher in Iba-1-positive microglial cells using immunohistochemistry (FIG. 9C).

Based on the above observations, it was next examined whether transfection with miR485-3p or miR-485 inhibitor could alter CD36 expression in mouse primary glial cells. As shown in FIGS. 9D and 9E, CD36 expression was markedly decreased in miR485-3p transfected primary glial cell compared to miR-controls. In contrast, cells transfected with the miR-485 inhibitor exhibited significantly higher CD36 expression.

The above results demonstrate that the miR-485 inhibitors of the present disclosure can also increase CD36 expression by regulating the expression of miR-485-3p.

### Example 10

### Analysis of the Binding of miR485-3p to CD36

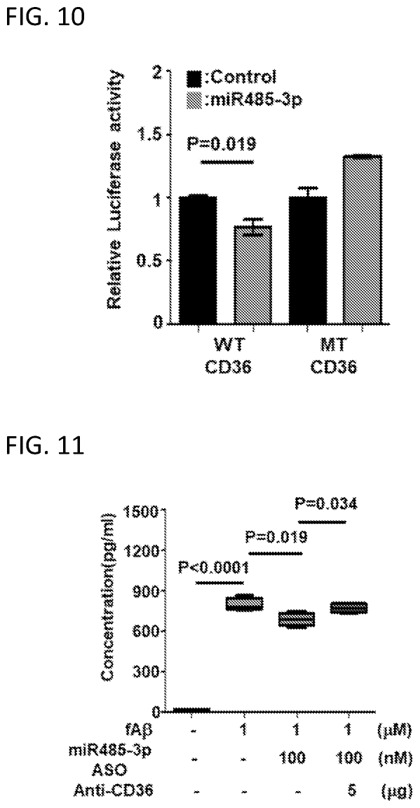

To confirm the target site for miR485-3p within the 3′-UTR of CD36, luciferase reporter plasmids containing either wild-type or mutated sequence of the potential miR485-3p site were constructed. Then, HEK293T cells were transfected with the plasmids, and promoter activity was measured in the transfected cells. As shown in FIG. 10, wild type promoter activity was significantly reduced but the mutant form was not different in miR485-3p transfected cells.

Next, the physical binding of miR485-3p to the 3′ UTR of SIRT1 was assessed using an in vitro binding assay as described in Example 5. The relative binding efficiency was significantly reduced in 3′ UTR-containing mutant seed sequences.

The above results collectively demonstrate that miR485-3p can directly target the 3′-UTR of CD36 and that this interaction can negatively regulate CD36 expression.

### Example 11

### Analysis of CD36 Regulation on Aβ Phagocytosis

To further assess the role of CD36+ glial cells on Aβ phagocytosis, it was examined whether a CD36 inhibitory antibody can influence glial phagocytosis. Briefly, primary glial cells were transfected with either the miR-485 inhibitor or miR-control. The transfected cells were treated with either CD36 blocking antibody or control IgG, and then treated with 1 μM fibrillar amyloid beta (fAβ) for 4 hours. An ELISA assay was used to determine Aβ phagocytosis in the conditioned media collected from the different transfected cells.

As shown in FIG. 11, Aβ levels were considerably decreased in cells transfected with the miR-485 inhibitor compared to the control transfected cells. However, this effect was significantly abrogated in cells treated with the CD36 blocking antibody.

These results confirm that the miR-485 inhibitors disclosed herein can regulate CD36 expression in a miR485-3p dependent manner, and can thereby, affect Aβ phagocytosis.

### Example 12

### Analysis of miR-485 Inhibitor on Neuroinflammation

AD is known to be associated with inflammation within the brain, and the secretion of inflammatory mediators by fAβ-stimulated-glia can contribute to neuronal loss and cognitive decline. Cunningham et al., J Neurosci 25(40):9275-84 (2005). Therefore, to assess whether the miR-485 inhibitors disclosed herein has any effect on neuroinflammation, primary glial cells were transfected with the miR-485 inhibitor or miR-control, and subsequently treated with 1 μM fibrillar amyloid beta (fAβ). Then, the levels of SIRT1 and different inflammatory mediators (i.e., NF-κB, TNF-α, and IL-1β) were examined in the cells.

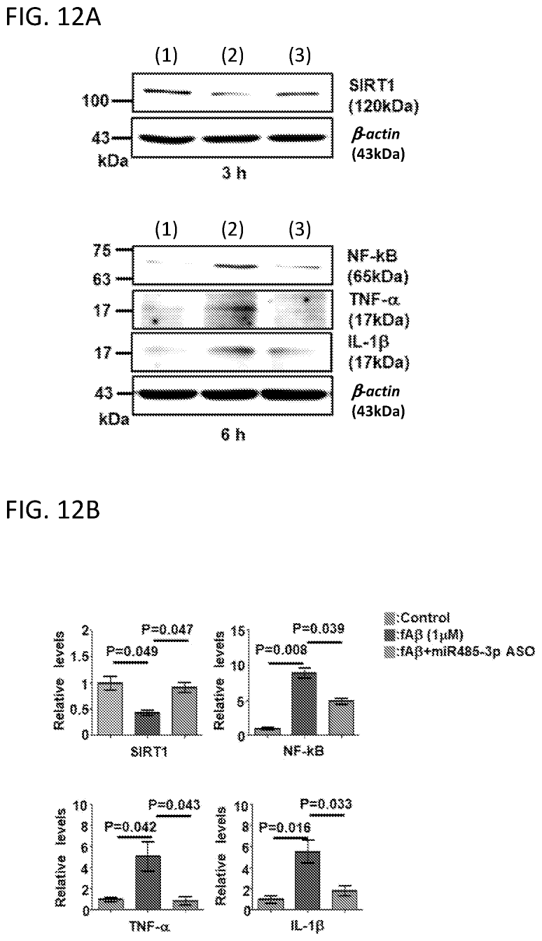

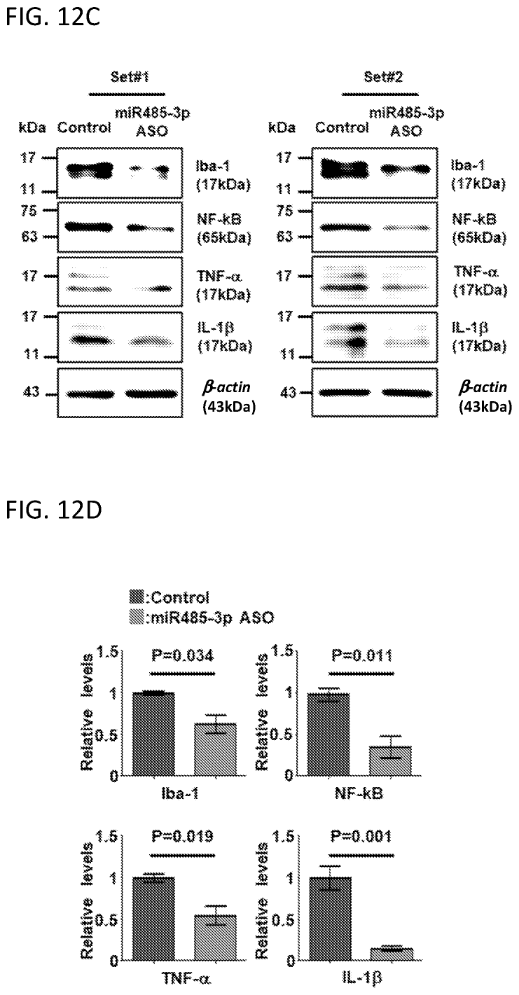

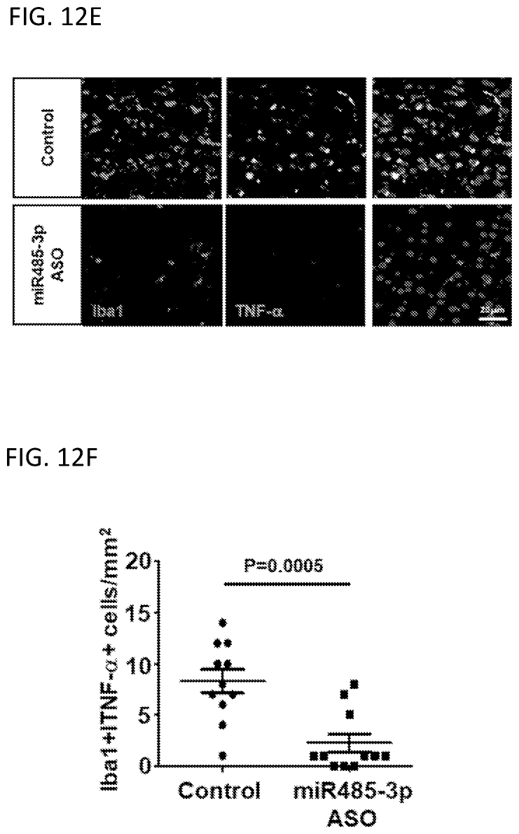

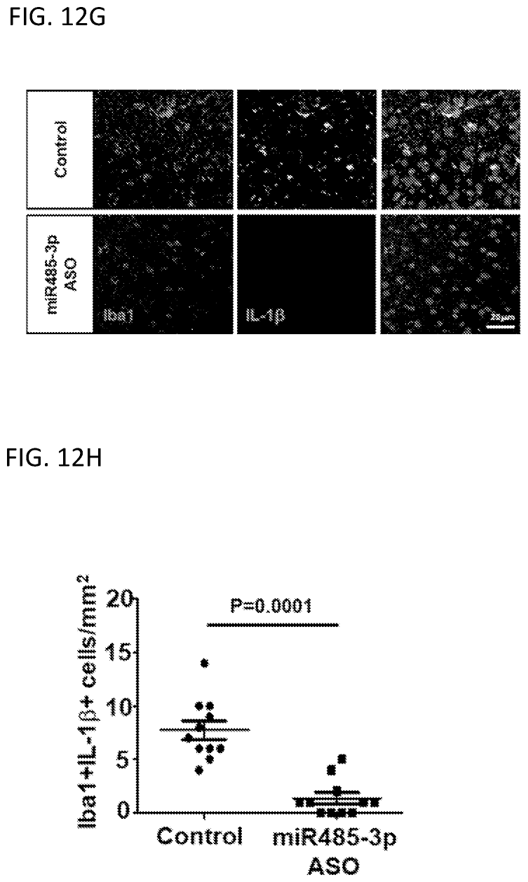

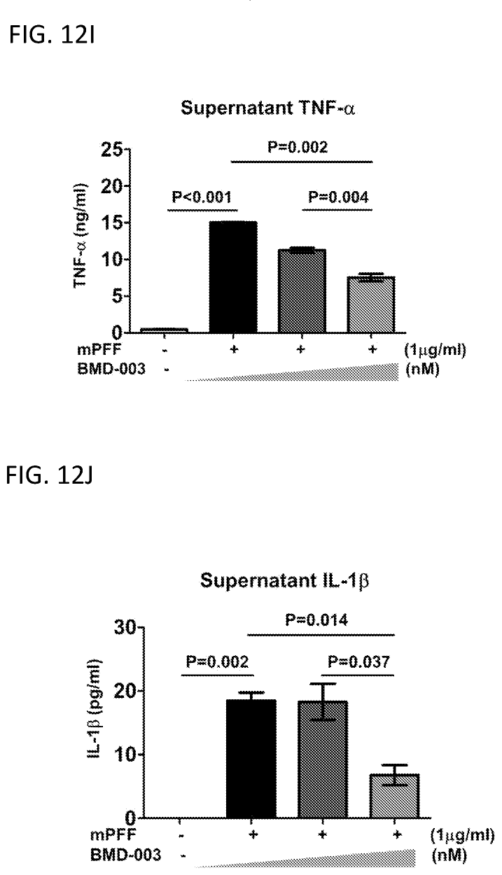

As shown in FIGS. 12A and 12B (and in agreement with the earlier data—see Example 4), SIRT1 expression was markedly decreased in fAβ treated primary glial cells, but this reduction was significantly recovered in cells transfected with the miR-485 inhibitor. The observed SIRT1 expression correlated with NF-κB expression, as well as expression levels of TNF-α and IL-1β (see FIGS. 12A and 12B). In cells transfected with the miR-485 inhibitor, there was significantly reduced levels of these inflammatory mediators, which appeared to be dose dependent (see FIGS. 12I and 12J).

To further characterize the effect of miR-485 inhibitor on neuroinflammation, AD mice were treated with the miR-485 inhibitor as described earlier (see Example 1). Then, the expression pattern of Iba-1 (i.e., activated microglial marker) and GFAP (i.e., activated astrocyte marker) was assessed.

As shown in FIGS. 12C and 12D, microglia expressing high levels of Iba-1 and astrocytes expressing high levels of GFAP were significantly decreased in AD mice treated with the miR-485 inhibitor. And, as observed above with the transfected cells, expression levels of NF-κB, TNF-α, and IL-1β were also significantly lower in the miR-485 inhibitor treated animals, as measured using real time PCR, Western blot, and immunohistochemistry (see FIGS. 12E-12H).

The above results demonstrate that by reducing miR485-3p expression, the miR-485 inhibitors disclosed herein can affect glial cell activation and reduce proinflammatory cytokine production via regulation SIRT1/NF-κB signaling.

### Example 13

### Analysis of miR-485 Inhibitor on Neuronal Loss and Post-Synapse

As described earlier, 5×FAD transgenic mice exhibit amyloid plaque deposition starting at 2 months and neuronal loss in cortical layer V at 9 months (see Example 7). Synaptic and neuronal loss in 5×FAD mice have been correlated with Aβ accumulation and neuroinflammation. Eimer et al., Mol Neurodegener 8:2 (2013). In light of the results from the earlier examples (e.g., that the regulation of SIRT1 and CD36 expression with an miR-485 inhibitor can control Aβ processing, phagocytosis, and inflammation in AD mice), whether the miR-485 inhibitors disclosed herein have any effect on neuronal cell death was examined by assessing NeuN (a neuronal cell marker) and cleaved caspase-3.

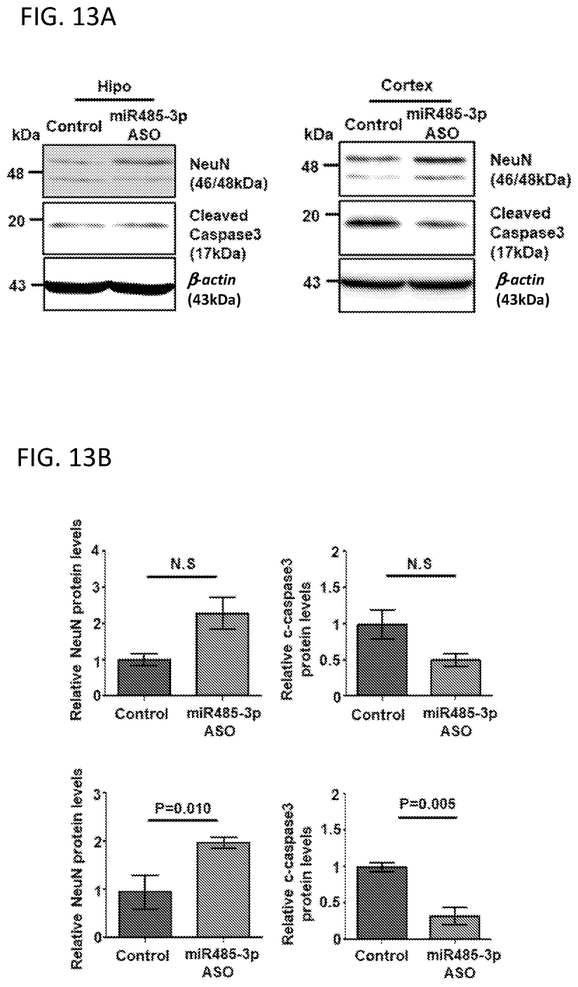

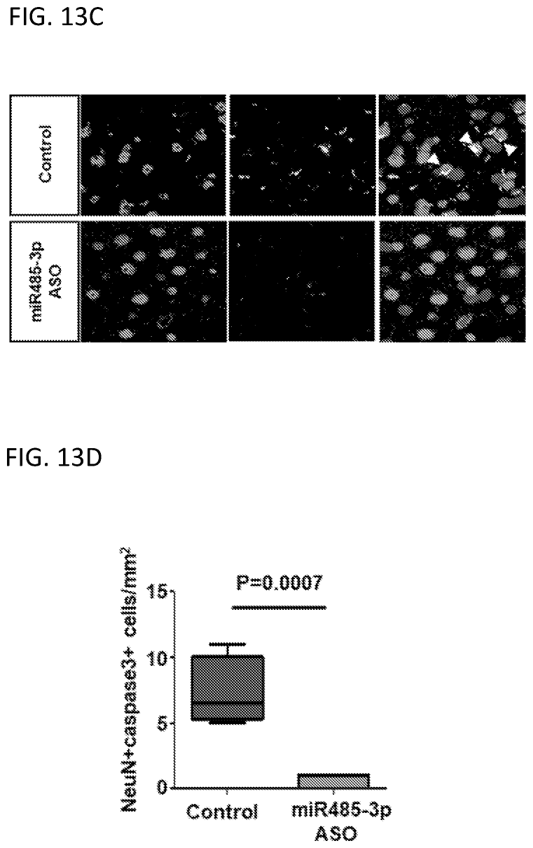

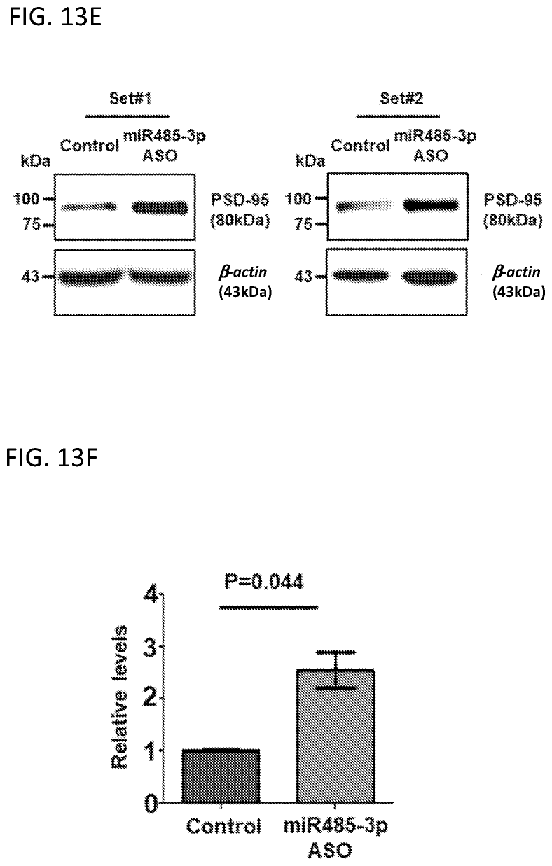

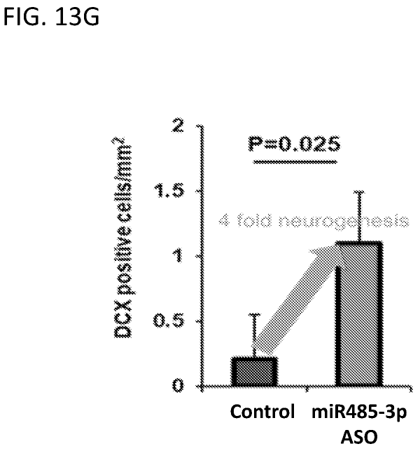

Based on western blot analysis, the expression of NeuN was increased, while the protein expression of caspase-3 was reduced, in the cortical region of miR-485 inhibitor treated animals (see FIGS. 13A and 13B). This effect, however, was not seen in the hippocampus under the same conditions. Similar results were observed using immunohistochemistry (see FIGS. 13C and 13D).

Next, the effect of miR-485 inhibitors on post-synapse was examined by assessing PSD-95 expression. As shown in FIGS. 13E and 13F, PSD-95 protein expression was significantly higher in the frontal cortex of AD mice treated with the miR-485 inhibitor, compared to the control animals.

The above results further demonstrate the therapeutic effects of the miR-485 inhibitors disclosed herein on AD by showing that the inhibitors can not only minimize neuronal loss but can also increase post-synapse.

### Example 14

### Analysis of miR-485 Inhibitor on Cognitive Function

To determine whether the results observed above in Example 13 (i.e., increased post-synapse and reduced neuronal loss) are correlated with improvement in cognitive functions, AD mice were again treated with the miR-485 inhibitor or miR-control as described in the earlier examples. Then, cognitive functions were assessed in the animals using Y-maze and passive avoidance task (PAT), which are widely accepted as behavior paradigms for evaluating spatial working memory.

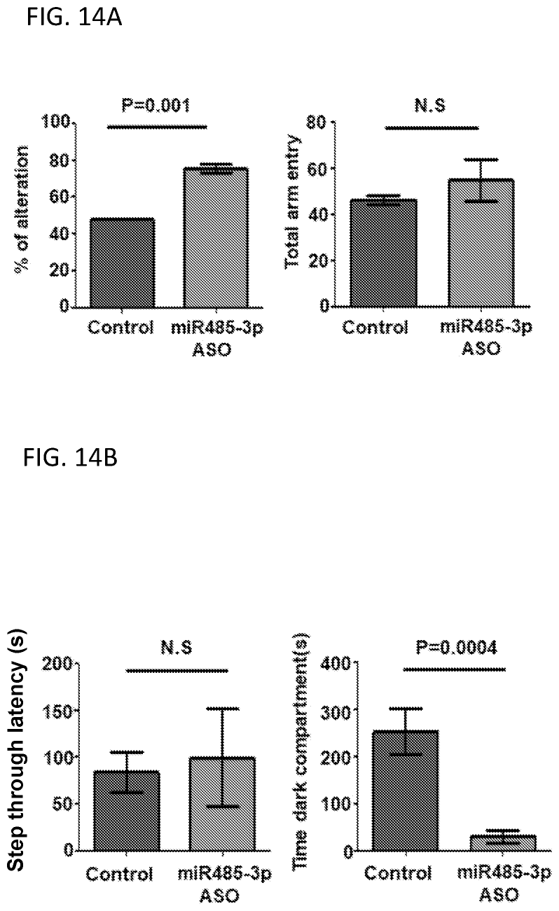

Two days after the last injection, we found that the spontaneous alternation percentage was significantly increased in miR-485 inhibitor treated mice. The total number of arm entries did not differ significantly between control and miR-485 inhibitor treated 5×FAD, indicating that levels of general motor and exploratory activity in the Y-maze were not changed (FIG. 14A). In addition, we examined associative memory in the passive avoidance task, based on the association formed between an electrical foot shock and a spontaneously preferred specific environmental context (darkness vs light). Step-through latency was similar between control and miR-485 inhibitor treated 5×FAD. However, miR-485 inhibitor treated mice showed a significant reduction in the latency to spend time the dark compartment 24hr after receiving an electrical shock (FIG. 14B).

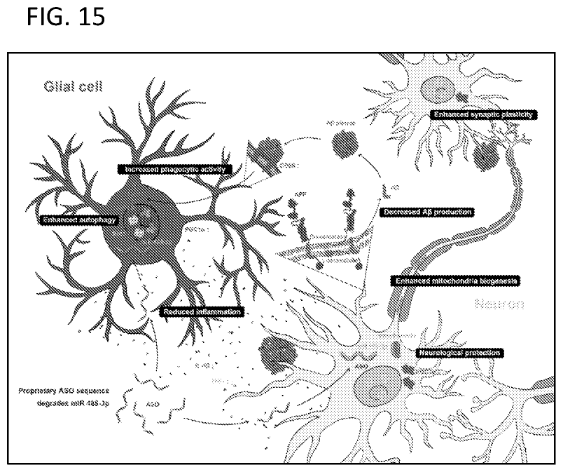

The above results collectively demonstrate that the miR-485 inhibitors disclosed herein can regulate (i.e., increase) the expression of different genes involved in neurodegenerative diseases, such as AD. As shown in the above Examples, such genes include SIRT1, CD36, and PGC-1α. Not to be bound by any one theory, the above results show that by regulating the expression of these genes, miR-485 inhibitors disclosed herein can treat many aspects of AD (e.g., reduce both Aβ production and plaque formation, promote Aβ plaque phagocytosis, reduce neuroinflammation, reduce neuronal loss, increase post-synapse, and improve cognitive functions) (see FIG. 15).

### Example 15

### Analysis of the Potency of miR-485 Inhibitors in Regulating SIRT1, PGC-1α, and CD36 Expression In Vivo

To further assess the potency of miR-485 inhibitors disclosed herein in regulating the expression of SIRT1, PGC-1α, and CD36, a single dose (100 μg/mouse; 5 mg/kg) of the miR-485 inhibitor (see Example 1) was administered (via intravenous administration) to wild-type male Crl:CD1 (ICR) mice, which were purchased from KOATECH (Korea). Control animals received the miR-control (see Example 1). Then, the animals were sacrificed at various time points post-administration, and the expression level of SIRT1, PGC-1α, and CD36 was assessed in both the cortex and hippocampus of the brain using Western blot.

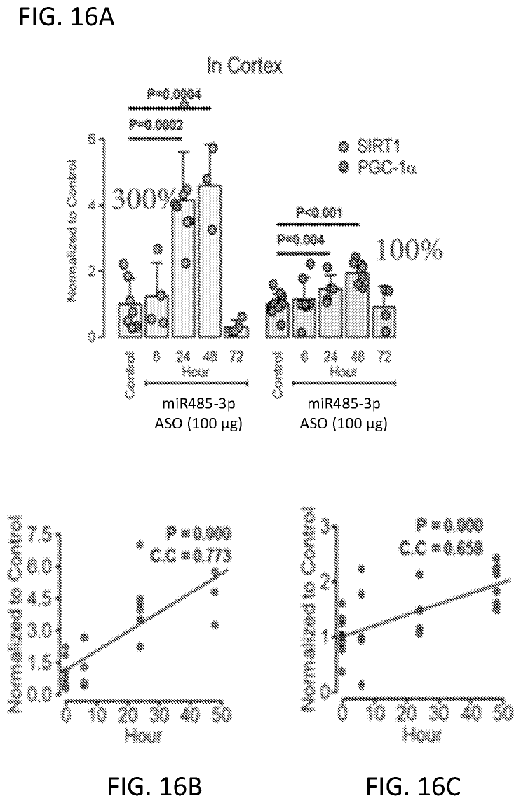

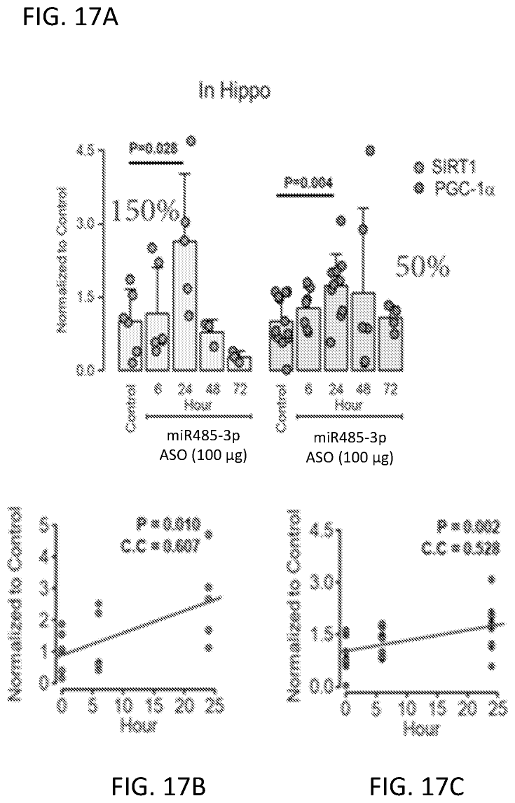

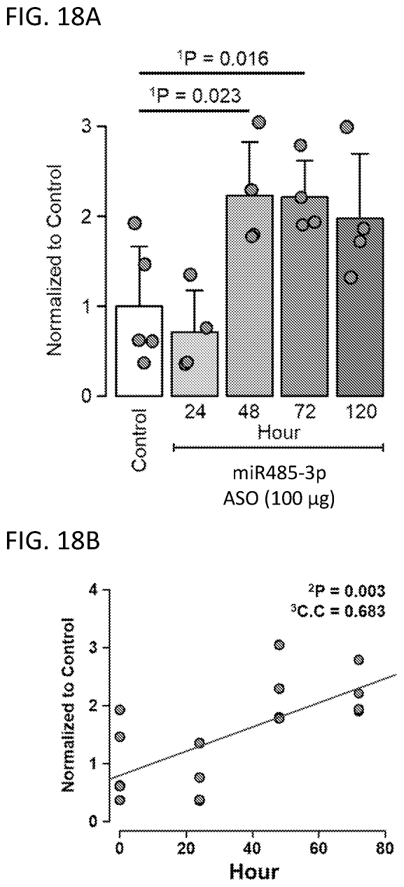

As shown in FIGS. 16A-16C, 17A-17 C, and 18A-18B, a single administration of the miR-485 inhibitor resulted in rapid increase in SIRT1, PGC-1α, and CD36 expression in both the cortex and the hippocampus. For SIRT1, peak expression was observed in the cortex at about 48 hours post-administration (approximately 300% increase over the expression in control animals) and in the hippocampus at about 24 hours post-administration (approximately 150% increase over the control) (see FIGS. 16A and 17A, respectively). The peak expression for PGC-1α was also observed at about 48 hours post-administration in the cortex (approximately 100% increase over the control) and at about 24 hours post-administration in the hippocampus (approximately 50% increase over the control) (see FIGS. 16B and 17B, respectively). Similar results were observed for CD36 (see FIG. 18A).

The results confirm the potency of the miR-485 inhibitors disclosed herein in regulating SIRT1, PGC-1α, and CD36 expression. For comparison, a small molecule ApoE4 has previously been shown to have positive effects on normalizing SIRT1 expression in vivo. See Campagna et al., Rep 8(1):17574 (Dec. 2018). After 56 days of daily administration (at a dose of 40 mg/kg per day), there was approximately a 20% increase in SIRT1 expression in the hippocampus but no increase in the cortex. With the miR-485 inhibitor (e.g., SEQ ID NO: 28) disclosed herein, a single administration at a much lower dose (i.e., 5 mg/kg) resulted in significantly greater SIRT1 expression both in the hippocampus and the cortex.

### Example 16

### Analysis of the Therapeutic Effects of miR-485 Inhibitor on Parkinson's Disease

Further to the examples provided above, the therapeutic effects of the miR-485 inhibitors disclosed herein on Parkinson's disease was examined using the 6-OHDA mouse model described in, e.g., Thiele et al., J Vis Exp. 60:3234 (2012), which is incorporated herein by reference in its entirety. Specifically, the effect of miR-485 inhibitors on dopaminergic degeneration was assessed. The unilateral 6-OHDA model induces a partial striatal lesion with progressive retrograde nigrostriatal pathology and allows assessment based on behavioral and neurochemical parameters relevant to PD.

Briefly, the mice were intraperitoneally injected with desipramine (25 mg/kg in 0.9% NaCl) approximately 30 minutes prior to the administration of 6-hydroxydopamine (6-OHDA) and then anesthetized by inhalation of vapor Isotroy (Toikaa Pharmaceuticals Limited, Gujarat, India). Anesthetized mice were placed in a stereotaxic frame (JEUNG DO BIO & PLANT CO., LTD, Seoul, Korea) and received a unilateral injection of 6-OHDA (5 μg/μl in 0.02% ascorbic acid dissolved in 0.9% NaCl; Sigma Aldrich) into the right striatum (anteroposterior: +0.9 mm; mediolateral: −2.2 mm; dorsoventral: −2.5 mm, relative to the bregma) at a rate of 0.5 μl/min for a total dose of 15 μg/3 μl. All injections were performed using a Hamilton syringe (30 S needle) attached to a syringe pump (Harvard Apparatus, Holliston, Mass., USA). After the injection, the needle was withdrawn slowly after 5 min.

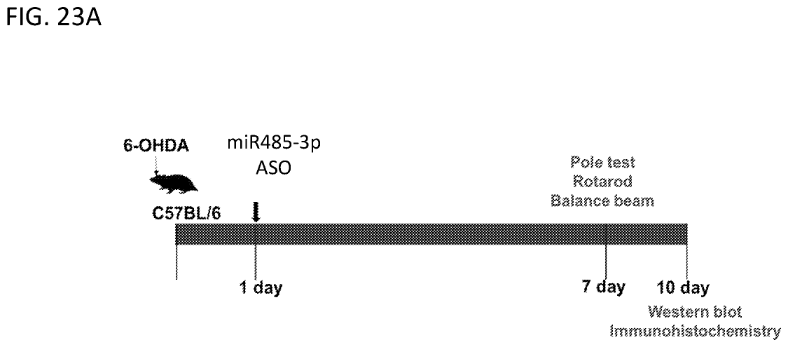

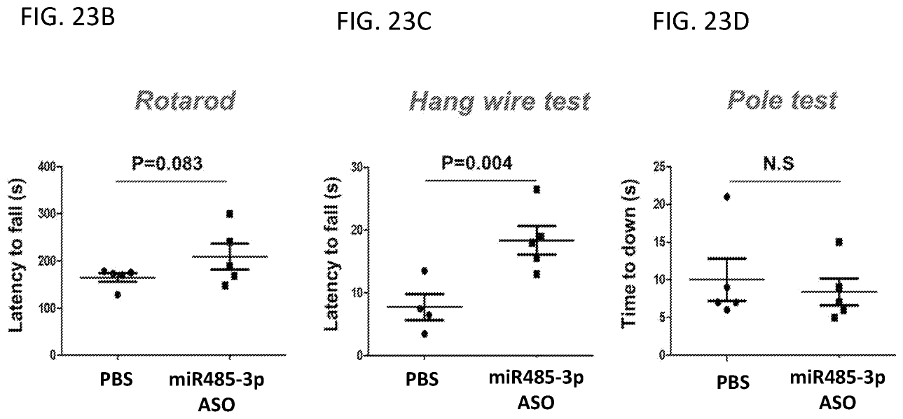

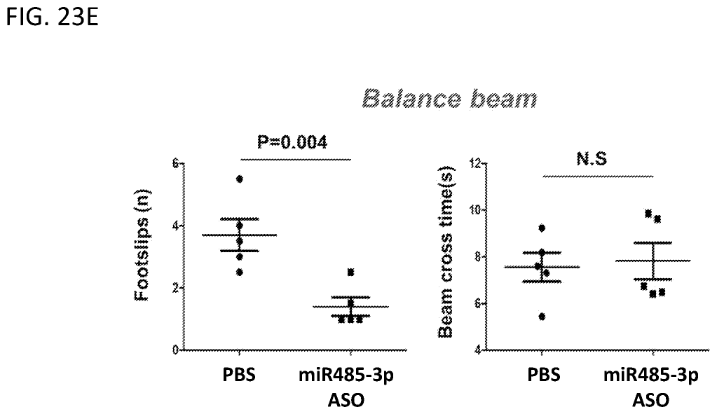

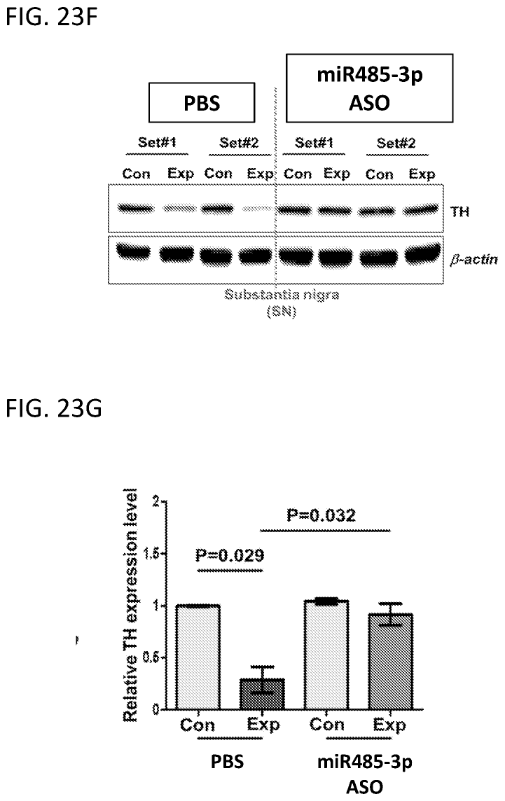

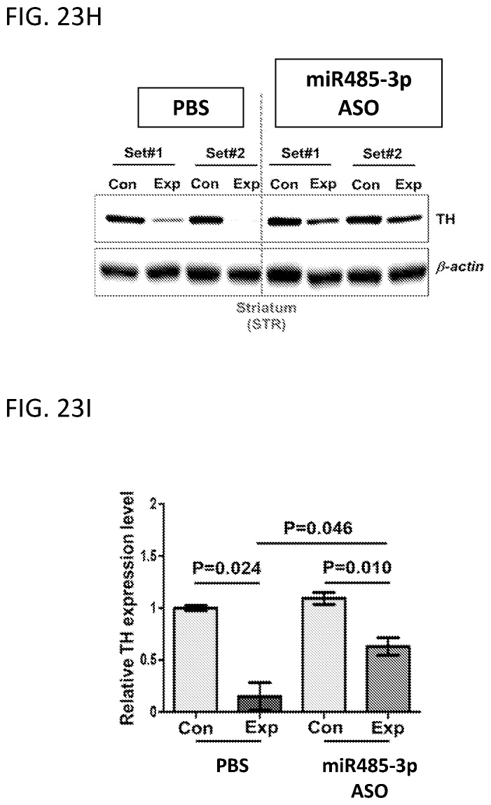

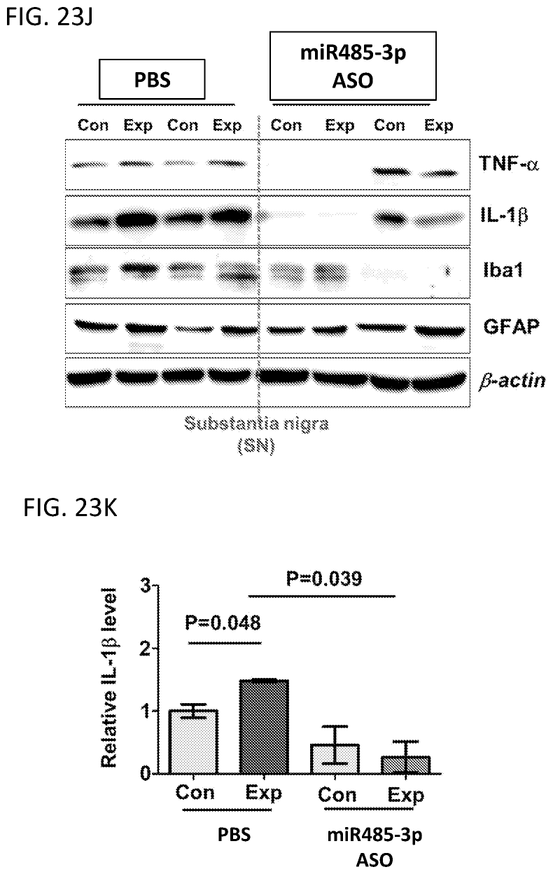

After the administration of the 6-OHDA to induce brain lesions in the animals, a day later, the animals received a single dose of either miR-485 inhibitor (50 μg/head) (SEQ ID NO: 30) or control oligonucleotide (SEQ ID NO: 61) via intravenous administration (tail-vein injection). See FIG. 23A. At day 6 post miR-485 inhibitor administration, motor function in the animals was assessed using one or more of the following tests: pole test, rotarod, hang wire test, and balance beam (see Example 1). At day 9 post miR-485 inhibitor administration, the animals were sacrificed and the effect of miR-485 inhibitor administration on brain tissue was assessed by measuring tyrosine hydroxylase expression using Western blot. Effect of miR-485 inhibitor on neuroinflammation was also assessed using western blot analysis.

As shown in FIGS. 23B-23E, 6-OHDA mice treated with the miR-485 inhibitor exhibited improved motor function as measured using at least the rotarod (exhibited increased latency to fall time), hang wire test (exhibited increased latency to fall time), and balance beam (decreased number of foot slips). The miR-485 inhibitor treated animals also exhibited higher tyrosine hydroxylase expression (i.e., marker for dopaminergic neurons) in both the substantia nigra (SN) and the striatum (STR), indicating reduced dopamine neuron damage (see FIGS. 23F-23I). Significantly decreased expression of IL-1β was observed in the substantia nigra of the miR-485 inhibitor treated animals compared to the control animals (see FIGS. 23J and 23K). However, miR-485 inhibitor administration did not appear to significantly affect Iba-1 (an activated microglial marker), GFAP (an activated astrocyte marker), and TNF-α expression.

Next, to further assess the therapeutic effects of the miR-485 inhibitors on Parkinson's Disease, additional 6-OHDA mice were treated with a single intravenous administration of the miR-485 inhibitor at one of the following doses: 2.5 mg/kg or 5 mg/kg. Healthy and 6-OHDA mice treated with PBS or control oligonucleotide were used as controls. Then, at day 6 post miR-485 inhibitor administration, motor function in the animals was again assessed using one or more of the following tests: pole test, rotarod, hang wire test, and balance beam (see Example 1).

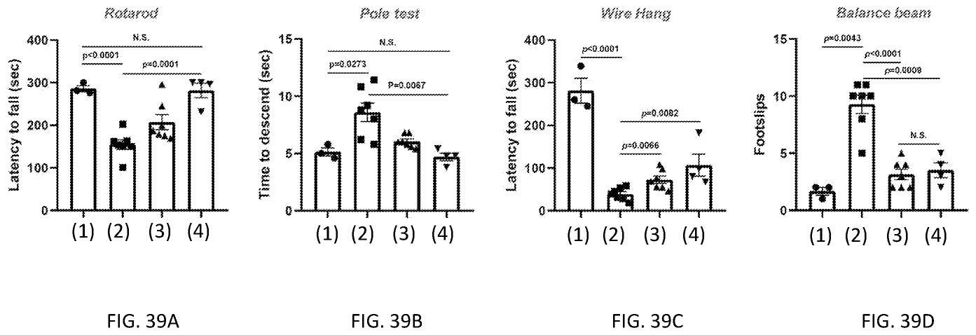

As shown in FIGS. 39A-39D, and in agreement with the earlier data, animals treated with the miR-485 inhibitor, at either of the doses, exhibited significantly improved motor function. Collectively, the results provided in this Example suggest that the miR-485 inhibitors disclosed herein can improve neuron damage associated with diseases such as Parkinson's (e.g., reduced dopamine neuron damage and/or reduced neuroinflammation), which can, in turn, improve motor function.

### Example 17

### Analysis of the Effect of miR-485 Inhibitor on Autophagy

As described herein, autophagy plays an important role in the proper degradation of long-lived proteins, protein aggregates, as well as damaged organelles in order to maintain cellular homeostasis. Therefore, to assess whether the miR-485 inhibitors disclosed herein has any effect on autophagy in cells of the CNS, both primary cortical neurons and primary mixed glial cells were treated with varying concentrations of the miR-485 inhibitor in combination with mouse a-synuclein PFF (aggrergated form) (mPFF) (1 μg/mL) for 24 or 48 hours. Then, the expression levels of p62 (an adaptor molecule that recruits substrates to autophagosomes) and LC3B (marker of autophagosome biogenesis) were assessed in the cells using western blot analysis.

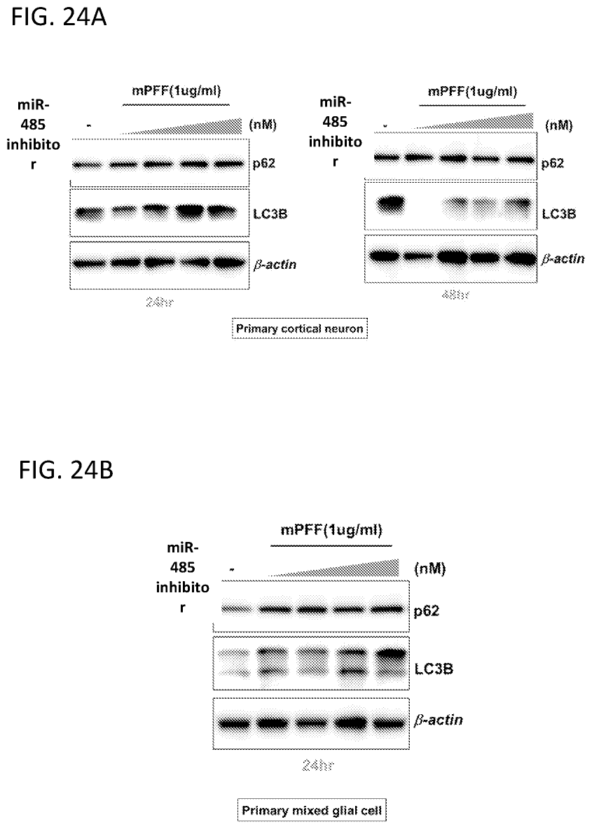

As shown in FIGS. 24A and 24B, for all concentrations tested, miR-485 inhibitor did not have any significant effect on p62 expression in the primary cortical neurons. However, miR-485 inhibitor treatment resulted in significant recovery of LC3B expression in the mPFF treated primary cortical neurons. For instance, at 48 hours, there was minimal LC3B expression detected in the primary cortical neurons treated with only mPFF (see FIG. 24A, right gel, 2^{nd} vertical lane). However, with increase in miR-485 inhibitor concentration, there was a gradual increase expression of LC3B (see FIG. 24A, right gel, 3^{rd}, 4^{th}, and 5^{th} vertical lanes). Similar results were observed in the primary mixed glial cells (see FIG. 24B).

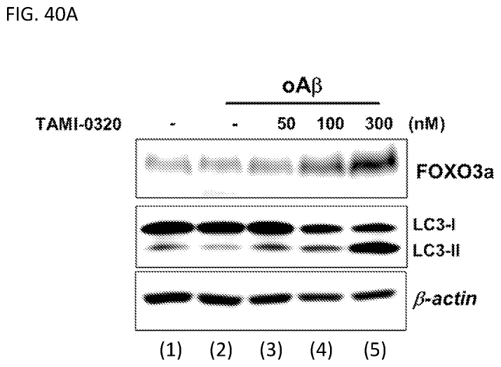

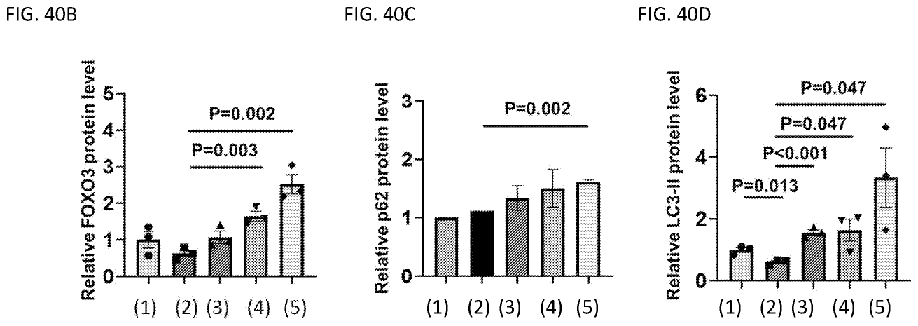

To confirm the above results, BV2 microglial cells were transfected with varying doses of the miR-485 inhibitor (0 nM, 50 nM, 100 nM, and 300 nM) and subsequently treated with fibrillar amyloid beta (oAβ) for 24 h at a final concentration of 1 μM. Then, the expression levels of different proteins associated with autophagy, i.e., FOXO3a, LC3, and p62, were assessed using western blot analysis. As shown in FIGS. 40A-40D, there was a dose-dependent increase in the expression of these proteins in cells transfected with the miR-485 inhibitor.

The above results collectively demonstrate that the miR-485 inhibitors disclosed herein can enhance autophagy flux in both primary cortical neurons and glial cells, which could be useful in treating the neurodegenerative diseases disclosed herein (e.g., Parkinson's disease and/or Alzheimer's disease).

### Example 18

### Analysis of the Safety Profile of miR-485 Inhibitors

To assess whether the in vivo administration of miR-485 inhibitors could result in any adverse effects, a single dose toxicity test was performed. Briefly, the miR-485 inhibitor was administered to male and female rats at one of the following doses: (i) 0 mg/kg (G1), (ii) 3.75 mg/kg (G2), (iii) 7.5 mg/kg (G3), and (iv) 15 mg/kg (G4). Then, any abnormalities in body weight, mortality, clinical signs, and pathology were observed in the animals at various time points post-transfer.

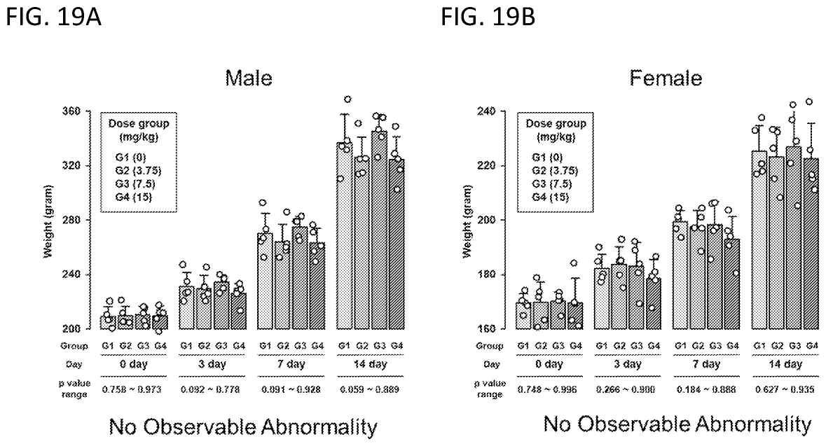

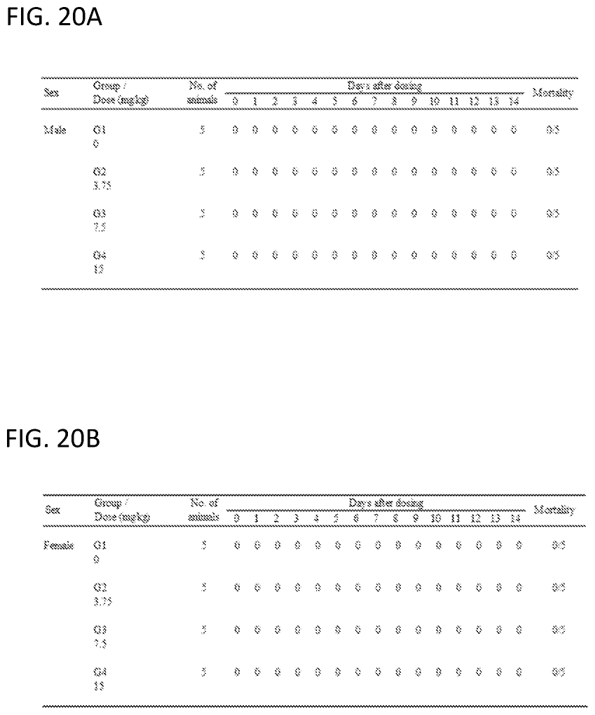

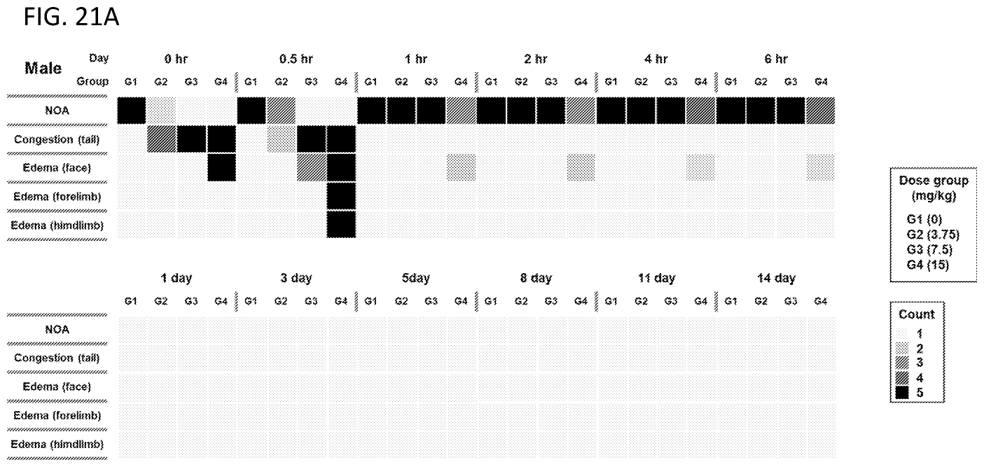

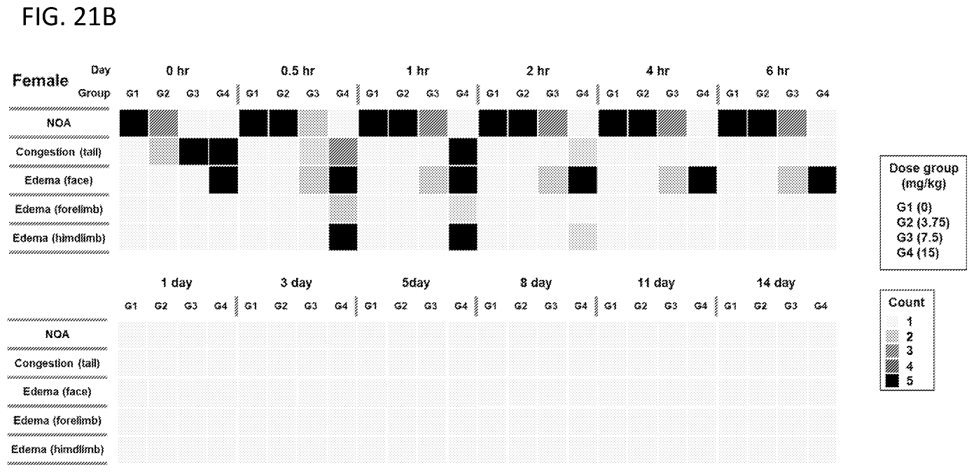

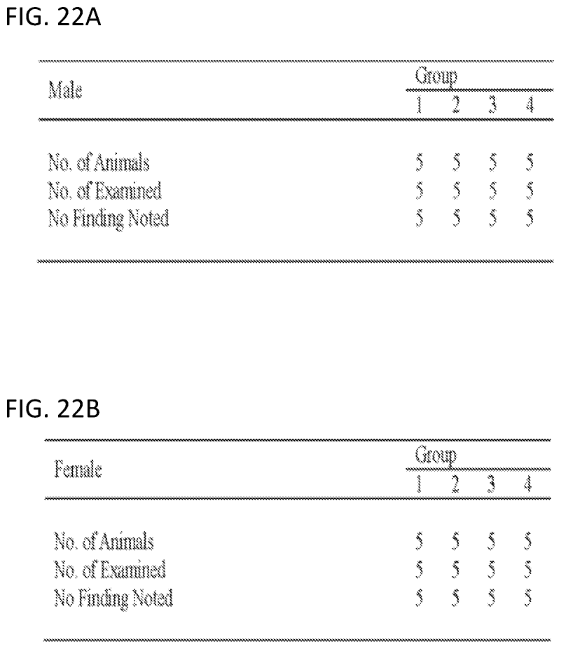

As shown in FIGS. 19A and 19B, the administration of the miR-485 inhibitor (at all doses tested) did not appear to have any abnormal effects on body weight in both the male and female rats. Similarly, no mortality and pathological abnormalities were observed in any of the treated animals (see FIGS. 20A, 20B, 22A, and 22B). As for possible clinically relevant side effects (e.g., NOA, congestion (tail), and edema (face, forelimb, or hind limb)), any such effects were gone by 1 day post-administration in all the treated animals (see FIGS. 21A and 21B).

Collectively, the above results demonstrate that the miR-485 inhibitors disclosed herein are not only potent in regulating the expression of SIRT1, PGC-1α, and CD36, but are also safe when administered in vivo.

### Example 19

### Development of New Alzheimer's Disease Animal Model by miRNA Viral-Delivery System

To further assess the therapeutic effects of miR-485 inhibitors disclosed herein and the role that miR485-3p expression has on Alzheimer's disease (AD), a new animal model was developed. Briefly, one or more of the following materials and methods were used in constructing the new AD animal model:

### Lentiviral Transfer Plasmid

The sequence of pLenti-III-mir-GFP vector containing mature mouse miR-485-3p (e.g., 485-3p-lenti-mini-7-GFP-F) was as follows (the miR-485-3p sequence is noted in capital letters):

Lenti-mir-GFP-Cloning vector sequence:
(SEQ ID NO: 122)
ttttggattgaagccaatatgataatgagggggtggagtttgtgacgtggcgcggggcgtgggaacggggcgggtgacgtagtagtg
tggcggaagtgtgatgttgcaagtgtggcggaacacatgtaagcgacggatgtggcaaaagtgacgtttttggtgtgcgccggtgtac
acaggaagtgacaattttcgcgcggttttaggcggatgttgtagtaaatttgggcgtaaccgagtaagatttggccattttcgcgggaaa
actgaataagaggaagtgaaatctgaataattttgtgttactcatagcgcgtaatacggcagacctcagcgctagattattgaagcatttat
cagggttattgtctcatgagcggatacatatttgaatgtatttagaaaaataaacaaataggggttccgcgcacatttccccgaaaagtgc
cacctgacgttaactataacggtcctaaggtagcgaaaatgtagtcttatgcaatactcttgtagtcttgcaacatggtaacgatgagttag
caacatgccttacaaggagagaaaaagcaccgtgcatgccgattggtggaagtaaggtggtacgatcgtgccttattaggaaggcaa
cagacgggtctgacatggattggacgaaccactgaattgccgcattgcagagatattgtatttaagtgcctagctcgatacataaacgg
gtctctctggttagaccagatctgagcctgggagctctctggctaactagggaacccactgcttaagcctcaataaagcttgccttgagt
gcttcaagtagtgtgtgcccgtctgttgtgtgactctggtaactagagatccctcagacccttttagtcagtgtggaaaatctctagcagtg
gcgcccgaacagggacttgaaagcgaaagggaaaccagaggagctctctcgacgcaggactcggcttgctgaagcgcgcacggc
aagaggcgaggggcggcgactggtgagtacgccaaaaattttgactagcggaggctagaaggagagagatgggtgcgagagcgt
cagtattaagcgggggagaattagatcgcgatgggaaaaaattcggttaaggccagggggaaagaaaaaatataaattaaaacatata
gtatgggcaagcagggagctagaacgattcgcagttaatcctggcctgttagaaacatcagaaggctgtagacaaatactgggacag
ctacaaccatcccttcagacaggatcagaagaacttagatcattatataatacagtagcaaccctctattgtgtgcatcaaaggatagaga
taaaagacaccaaggaagctttagacaagatagaggaagagcaaaacaaaagtaagaccaccgcacagcaagcccgctgatcttca
gacctggaggaggagatatgagggacattggagaagtgaattatataaatataaagtagtaaaaattgaaccattaggagtagcaccc
accaaggcaaagagaagagtggtgcagagagaaaaaagagcagtgggaataggagctttgttccttgggttcttgggagcagcagg
aagcactatgggcgcagcgtcaatgacgctgacggtacaggccagacaattattgtctggtatagtgcagcagcagaacaatttgctg
agggctattgaggcgcaacagcatctgttgcaactcacagtctggggcatcaagcagctccaggcaagaatcctggctgtggaaaga
tacctaaaggatcaacagctcctggggatttggggttgctctggaaaactcatttgcaccactgctgtgccttggaatgctagttggagta
ataaatctctggaacagatttggaatcacacgacctggatggagtgggacagagaaattaacaattacacaagcttaatacactccttaa
ttgaagaatcgcaaaaccagcaagaaaagaatgaacaagaattattggaattagataaatgggcaagtttgtggaattggtttaacataa
caaattggctgtggtatataaaattattcataatgatagtaggaggcttggtaggtttaagaatagtttttgctgtactttctatagtgaataga
gttaggcagggatattcaccattatcgtttcagacccacctcccaaccccgaggggacccgacaggcccgaaggaatagaagaaga
aggtggagagagagacagagacagatccattcgattagtgaacggatctcgacggtatcgaaagcttgggattcgaatttaaaagaaa
aggggggattggggggtacagtgcaggggaaagaatagtagacataatagcaacagacatacaaactaaagaactacaaaaacaa
attacaaaaattcaaaattttcgggtttttcgaacctagggttccgcgttacataacttacggtaaatggcccgcctggctgaccgcccaa
cgacccccgcccattgacgtcaataatgacgtatgttcccatagtaacgccaatagggactttccattgacgtcaatgggtggagtattta
cggtaaactgcccacttggcagtacatcaagtgtatcatatgccaagtacgccccctattgacgtcaatgacggtaaatggcccgcctg
gcattatgcccagtacatgaccttatgggactttcctacttggcagtacatctacgtttagtcatcgctattaccatggtgatgcggttttggc
agtacatcaatgggcgtggatagcggtttgactcacggggatttccaagtctccaccccattgacgtcaatgggagtttgttttggcacc
aaaatcaacgggactttccaaaatgtcgtaacaactccgccccattgacgcaaatgggcggtaggcgtgtacggtgggaggtctatat
aagcagagctcgtttagtgaaccgtcagatcgcctggagacgccatccacgctgttttgacctccatagaagaaccgagtttaaactcc
ctatcagtgatagagatctccctatcagtgatagagagctagaatctagaggtaccgccaccatggtgagcaagggcgaggagctgtt
caccggggtggtgcccatcctggtcgagctggacggcgacgtaaacggccacaagttcagcgtgtccggcgagggcgagggcga
tgccacctacggcaagctgaccctgaagttcatctgcaccaccggcaagctgcccgtgccctggcccaccctcgtgaccaccctgac
ctacggcgtgcagtgcttcagccgctaccccgaccacatgaagcagcacgacttcttcaagtccgccatgcccgaaggctacgtcca
ggagcgcaccatcttcttcaaggacgacggcaactacaagacccgcgccgaggtgaagttcgagggcgacaccctggtgaaccgc
atcgagctgaagggcatcgacttcaaggaggacggcaacatcctggggcacaagctggagtacaactacaacagccacaacgtcta
tatcatggccgacaagcagaagaacggcatcaaggtgaacttcaagatccgccacaacatcgaggacggcagcgtgcagctcgcc
gaccactaccagcagaacacccccatcggcgacggccccgtgctgctgcccgacaaccactacctgagcacccagtccgccctga
gcaaagaccccaacgagaagcgcgatcacatggtcctgctggagttcgtgaccgccgccgggatcactctcggcatggacgagctg
tacaagtgaggtaccgatatcgaattcatagctagccctgcaggtctagactcgagGTCATACACGGCTCTCCTCTC
Tgcggccgcagtcgagtacccatacgacgtcccagactacgcttgagtttaaacacgcgtggtgtggaaagtccccaggctcccca
gcaggcagaagtatgcaaagcatgcatctcaattagtcagcaaccaggtgtggaaagtccccaggctccccagcaggcagaagtat
gcaaagcatgcatctcaattagtcagcaaccatagtcccgcccctaactccgcccatcccgcccctaactccgcccagttccgcccatt
ctccgccccatggctgactaattttttttatttatgcagaggccgaggccgcctcggcctctgagctattccagaagtagtgaggaggctt
ttttggaggccatgaccgagtacaagcccacggtgcgcctcgccacccgcgacgacgtccctcgggccgtacgcaccctcgccgcc
gcgttcgccgactaccccgccacgcgccacaccgtggacccggaccgccacatcgagcgggtcaccgagctgcaagaactcttcct
cacgcgcgtcgggctcgacatcggcaaggtgtgggtcgcggacgacggcgccgcggtggcggtctggaccacgccggagagcg
tcgaagcgggggcggtgttcgccgagatcggcccgcgcatggccgagttgagcggttcccggctggccgcgcagcaacagatgg
aagggctcctggcgccgcaccggcccaaggagcccgcgtggttcctggccaccgtcggcgtctcgcccgaccaccagggcaagg
gtctgggcagcgccgtcgtgctccccggagtggaggcggccgagcgcgccggggtgcccgccttcctggagacctccgcgcccc
gcaacctccccttctacgagcggctcggcttcaccgtcaccgccgacgtcgaggtgcccgaaggaccgcgcacctggtgcatgacc
cgcaagcccggtgcctgaacgcgttccggaaatcaacctctggattacaaaatttgtgaaagattgactggtattcttaactatgttgctc
cttttacgctatgtggatacgctgctttaatgcctttgtatcatgctattgcttcccgtatggctttcattttctcctccttgtataaatcctg
gttgctgtctctttatgaggagttgtggcccgttgtcaggcaacgtggcgtggtgtgcactgtgtttgctgacgcaacccccactggttggggc
attgccaccacctgtcagctcctttccgggactttcgctttccccctccctattgccacggcggaactcatcgccgcctgccttgcccgct
gctggacaggggctcggctgttgggcactgacaattccgtggtgttgtcggggaagctgacgtcctttccatggctgctcgcctgtgtt
gccacctggattctgcgcgggacgtccttctgctacgtcccttcggccctcaatccagcggaccttccttcccgcggcctgctgccggc
tctgcggcctcttccgcgtctcgccttcgccctcagacgagtcggatctccctttgggccgcctccccgcctgtccggatggaagggct
aattcactcccaacgaatacaagatctgctttttgcttgtactgggtctctctggttagaccagatctgagcctgggagctctctggctaact
agggaacccactgcttaagcctcaataaagcttgccttgagtgcttcaagtagtgtgtgcccgtctgttgtgtgactctggtaactagaga
tccctcagacccttttagtcagtgtggaaaattctagcagtagtagttcatgtcatcttattattcagtatttataacttgcaaagaaatgaata
tcagagagtgagaggaacttgtttattgcagcttataatggttacaaataaagcaatagcatcacaaatttcacaaataaagcatttttttca
ctgcattctagttgtggtttgtccaaactcatcaatgtatcttatcatgtctggcatctatgtcgggtgcggagaaagaggtaatgaaatgg
cattatgggtattatgggtctgcattaatgaatcggccaacgatcccggtgtgaaataccgcacagatgcgtaaggagaaaataccgca
tcaggcgctcttccgcttcctcgctcactgactcgctgcgctcggtcgttcggctgcggcgagcggtatcagctcactcaaaggcggta
atacggttatccacagaatcaggggataacgcaggaaagaacatgtgagcaaaaggccagcaaaaggccaggaaccgtaaaaagg
ccgcgttgctggcgtttttccataggctccgcccccctgacgagcatcacaaaaatcgacgctcaagtcagaggtggcgaaacccga
caggactataaagataccaggcgtttccccctggaagctccctcgtgcgctctcctgttccgaccctgccgcttaccggatacctgtccg
cctttctcccttcgggaagcgtggcgctttctcatagctcacgctgtaggtatctcagttcggtgtaggtcgttcgctccaagctgggctgt
gtgcacgaaccccccgttcagcccgaccgctgcgccttatccggtaactatcgtcttgagtccaacccggtaagacacgacttatcgcc
actggcagcagccactggtaacaggattagcagagcgaggtatgtaggcggtgctacagagttcttgaagtggtggcctaactacgg
ctacactagaaggacagtatttggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctcaagaagatcctttgatcttttcta
cggggtctgacgctcagtggaacgaaaactcacgttaagggattttggtcatgagattatcaaaaaggatcttcacctagatccttttaaa
ttaaaaatgaagttttaaatcaatctaaagtatatatgagtaaacttggtctgacagttaccaatgcttaatcagtgaggcacctatctcagc
gatctgtctatttcgttcatccatagttgcctgactccccgtcgtgtagataactacgatacgggagggcttaccatctggccccagtgctg
caatgataccgcgagacccacgctcaccggctccagatttatcagcaataaaccagccagccggaagggccgagcgcagaagtgg
tcctgcaactttatccgcctccatccagtctattaattgttgccgggaagctagagtaagtagttcgccagttaatagtttgcgcaacgttgt
tgaaaaaggatcttcacctagatccttttcacgtagaaagccagtccgcagaaacggtgctgaccccggatgaatgtcagctactgggc
tatctggacaagggaaaacgcaagcgcaaagagaaagcaggtagcttgcagtgggcttacatggcgatagctagactgggcggtttt
atggacagcaagcgaaccggaattgccagctggggcgccctctggtaaggttgggaagccctgcaaagtaaactggatggctttctc
gccgccaaggatctgatggcgcaggggatcaagctctgatcaagagacaggatgaggatcgtttcgcatgattgaacaagatggatt
gcacgcaggttctccggccgcttgggtggagaggctattcggctatgactgggcacaacagacaatcggctgctctgatgccgccgt
gttccggctgtcagcgcaggggcgcccggttctttttgtcaagaccgacctgtccggtgccctgaatgaactgcaagacgaggcagc
gcggctatcgtggctggccacgacgggcgttccttgcgcagctgtgctcgacgttgtcactgaagcgggaagggactggctgctattg
ggcgaagtgccggggcaggatctcctgtcatctcaccttgctcctgccgagaaagtatccatcatggctgatgcaatgcggcggctgc
atacgcttgatccggctacctgcccattcgaccaccaagcgaaacatcgcatcgagcgagcacgtactcggatggaagccggtcttgt
cgatcaggatgatctggacgaagagcatcaggggctcgcgccagccgaactgttcgccaggctcaaggcgagcatgcccgacggc
gaggatctcgtcgtgacccatggcgatgcctgcttgccgaatatcatggtggaaaatggccgcttttctggattcatcgactgtggccgg
ctgggtgtggcggaccgctatcaggacatagcgttggctacccgtgatattgctgaagagcttggcggcgaatgggctgaccgcttcc
tcgtgctttacggtatcgccgctcccgattcgcagcgcatcgccttctatcgccttcttgacgagttcttctgaattttgttaaaatttttgtta
aatcagctcattttttaaccaataggccgaaatcggcaacatcccttataaatcaaaagaatagaccgcgatagggttgagtgttgttccag
tttggaacaagagtccactattaaagaacgtggactccaacgtcaaagggcgaaaaaccgtctatcagggcgatggcccactacgtg
aaccatcacccaaatcaagttttttgcggtcgaggtgccgtaaagctctaaatcggaaccctaaagggagcccccgatttagagcttga
cggggaaagccggcgaacgtggcgagaaaggaagggaagaaagcgaaaggagcgggcgctagggcgctggcaagtgtagcg
gtcacgctgcgcgtaaccaccacacccgcgcgcttaatgcgccgctacagggcgcgtccattcgccattcaggatcgaattaattctta
attaacatcatcaataatatacctt

### Producing Lentivirus in 293T Cells

HEK 293T cells were plated in a 10 cm tissue culture plate until they reached 70-80% confluency (e.g., 3×10⁶ cells in 10 ml of DMEM complete growth medium). Two hours prior to transfection of viral DNA, the culture medium was removed from the 293T cells and replaced with 5 ml of DMEM growth medium. TransIT®-Lenti Reagent (Minis Bio, Cat#s 6600, 6603, 6604, 6605, 6606, and 6610) was warmed to room temperature and vortexed gently. 1 ml of Opti-MEM reduced-serum medium was placed in a sterile tube. 10 ug of pMDLg/pRRE packaging plasmid (Addgene, plasmid #12251), 5 ug of pRSV-REV packaging plasmid (Addgene, plasmid #12253), 2.5 ug of pMD2.G envelope plasmid (Addgene, plasmid #12259), and 2.5 ug of 485-3p-lenti-mini-7-GFP-F plasmid (discussed above) were combined in a separate sterile tube, mixed thoroughly, and transferred to the tube containing Opti-MEM reduced-serum medium. 35 ul of TransIT®-Lenti Reagent was added to the diluted and incubated for 20 minutes at room temperature to allow transfection complexes to form. TransIT®-Lenti Reagent: DNA complexes (prepared above) were distributed drop-wise to the 10-cm culture plate containing the 293T cells. Transfected cell cultures were incubated at 37° C. in 5% CO2 for 72 hours prior to lentivirus harvest.

Following the 72-hour incubation, cells were centrifuged in a sterile tube at 6000 RPM for 15 minutes. The virus-containing supernatant was ultra-centrifuged at 29000 RPM for 2 hours to obtain the virus-containing pellet. The pellet was diluted 1:1000 in cold PBS and aliquots were stored at −80° C.

### Virus Titration

To establish the titer of viral preparations, 293T cells were plated at 4×10⁵ cells per well in 6-well tissue culture plates in DMEM complete growth medium. Next day, approximately 1 mL of the growth medium was left in each of the wells, and 1 μL of the lentiviral construct (either the control vector or vector comprising miR-485-3p and GFP) and polybrene (8 μg/mL) were added to the individual wells. The lentiviral constructs were serially diluted (1, 0.1, and 0.01) prior to the addition. After six hours of incubation, the media was changed with fresh media. Then, after 48 hours, the cells were washed with warm PBS, trypsinized, and analyzed by flow cytometry (BD Accuri C6 plus) for GFP expression.

### Animals

Mice (wild-type; male; C57BL/6J; 6 weeks old) were purchased from Dae Han Bio Link Co Ltd (Chungju-si, Republic of Korea). Mice undergoing surgery and behavioral experiments were reared in single cages to eliminate physical injuries and psychological anxiety caused by attacks from other males. Water and food were provided ad libitum and in a 12-hour light/12-hour dark cycle environment.

Any physical abnormalities in appearance and body weight were regularly checked after surgery in both mice injected with the lentiviral control vector (without miR-485-3p) and the lenti-mir485-3p vector into the brain. All behavioral experiments were conducted in the light phase, and all mice in each group were tested under the same conditions.

### In Vivo Lentiviral Vector Injections and Tissue Preparation

6 weeks old mice were anesthetized with intraperitoneal injection using an anesthetic (2,2,2-tribromoethanol (250 mg/kg i.p.; Sigma-Aldrich, cat #75-80-9)). Using stereotaxic surgery equipment and Hamilton syringe 700 series, lentiviral vector was injected into the dentate gyrus and CA1 region (AP=−2 mm, L=±1.5 mm, ventral (V)=−2.7 & −2.0 mm) in hippocampus. The virus volume per site was 1.5 ul, the injection flow rate was 0.2 ul/min, and the remaining time after injection was 15 minutes. After surgery, the process of recovering from the anesthesia and the body weight were checked to see if there were any health problems caused by the surgery.

After the behavioral experiment was completed, the mice were anesthetized, sacrificed through cardiac perfusion, and the brains were removed carefully and post-fixed in 4% paraformaldehyde for 4 hours at 4° C. and then cryopreserved in 30% sucrose/0.1 M PBS at 4° C. for about 48 hours. Brains were embedded in OCT compound (Tissue-Tek®, Sakura, Inc., cat #4583) and sectioned sagittally into 40 μm-thick slices at −22° C. using a Leica CM1860 cryostat (Leica Microsystems). In the case of the virus GFP image, the nuclei were stained with DAPI (1:500; Invitrogen, cat #D 3571) and mounted with a hardset anti-fade medium. Images were obtained using a confocal microscope (Leica DMi8).

### Behavioral Tests

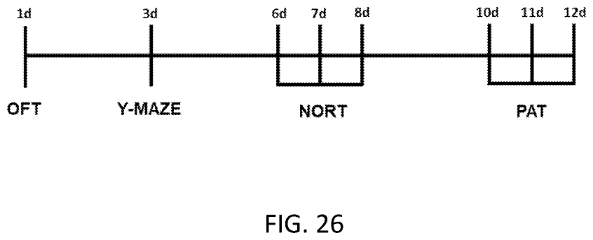

Mouse behavioral experiments were conducted in the following order: (i) open field test, (ii) Y-maze, (iii) novel object recognition test, and (iv) passive avoidance test. (see e.g., FIG. 26). When one behavioral experiment was completed, the next experiment was conducted with a recovery period of 2 days. Behavioral experiments excluding passive avoidance were analyzed using the smart 3.0 video tracking system (Panlab, worldwideweb.harvardapparatus.com/smart-video-tracking-system).

(i) Open Field Test

Mice were placed in the center of a white matte chamber (450 mm×450 mm×450 mm) and allowed to move freely for 30 minutes. Digital video tracking was performed. By analyzing the total distance in cm increments, the basal locomotion for 30 minutes was measured, and center distance (the distance traveled in the center zone) (cm) and the total distance (cm) moved in the entire area were recorded. The center distance divided by the total distance×100 was calculated as the center zone activity (%).The center distance—total distance ratio can be used as an index of anxiety-related responses.

(ii) Y-Maze

The Y-maze consisted of three white matte plastic arms (65 mm×400 mm×130 mm), 120° from each other. The mice were placed in the center and were allowed to move freely for 8 minutes and explore all three arms. The number of arm entries and number of trials (a shift is 10 cm from the center, entries into three separate arms) were recorded to calculate the percentage of alternation. An entry was defined as all three appendages entering a Y-maze arm. Alternation behavior was defined as the number of triads divided by the number of arm entries minus 2 and multiplied by 100.

(iii) Novel Object Recognition Test

Mice were placed in the center of a white matter chamber (450 mm×450 mm×450 mm) and allowed to move freely for 5 minutes on the first day (day 1) to adapt to the space. After 24 hours (day 2), the two same objects were placed on the first and fourth quarters of the chamber, and mice were allowed to move freely for 10 minutes to learn about the two objects (A&A) and space. After 1 hour of measuring short-term memory, one of the two objects was changed to a different shape and color (A&B), and the curiosity about a new object (the number of nose poking) was measured. For a long-term memory measurement, after 24 hours (day 3) and after 3 weeks (day 24) one of the two objects was changed to a different shape and color (e.g., A&C to A&D), and the number of nose poking on a new object was measured and analyzed. The results were compared by analyzing the number of nose poking on the new and familiar objects for each group, and the discrimination index was calculated with the following formula: [(novel-familiar)/(novel+familiar)].

(iv) Passive Avoidance Test

The passive avoidance test was performed as described in Example 1.

### Cell Culture

Mouse Primary Neuronal Cell Culture

The cortex including hippocampus was isolated from the head of the mouse corresponding to 18.5 days old embryo and primary culture was performed, as described in e.g., Seibenhener M L. et al., J Vis Exp., (65): 3634 (2012). On the 8^{th} day of DIV (days in vitro), neuronal cells were transduced with 485-3p-lenti-mini-7-GFP-F lentiviral vector or lentiviral control vector (without the miR-485-3p) using polybrene, and GFP expression was observed 30 hours later. After confirming that the cells were properly infected, immunocytochemistry was performed (30 hours later) after fixation using 4% paraformaldehyde, as described above (see e.g., In Vivo Lentiviral Vector Injections and Tissue Preparation).

Mouse Primary Glial Cell Culture

1-day-old postnatal mice were sacrificed, and mixed glia cells were cultured. Microglia cells were isolated on the 10^{th} day of DIV (days in vitro) and astrocytes were isolated on the 11^{th} day of DIV to prepare glial cell culture, as described in e.g., Lian H., et al., Bio Protoc., 6(21): e1989 (2016). On the 14^{th} day of DIV, glial cells were transduced with 485-3p-lenti-mini-7-GFP-F lentiviral vector or lentiviral control vector (without the miR-485-3p) using polybrene, and GFP expression was observed 30 hours later. After confirming that the cells were properly infected, immunocytochemistry was performed after fixation using 4% paraformaldehyde, as described above (see e.g., In Vivo Lentiviral Vector Injections and Tissue Preparation).

Human Cell Line

Human microglia primary cells derived from the central nervous system (CNS) cortex, immortalized human astrocytes, and fetal SV40 cells were transduced with 485-3p-lenti-mini-7-GFP-F lentiviral vector or lentiviral control vector (without the miR-485-3p) using polybrene, and GFP expression was observed 24 hours later. After confirming that the cells were properly infected, immunocytochemistry was performed after fixation using 4% paraformaldehyde, as described above (see e.g., In Vivo Lentiviral Vector Injections and Tissue Preparation).

### Disease Induction

CA1 is known as a part of the brain that plays an important role in the onset of AD disease. It is believed that the pathology of AD starts from distal CA1, which is the border between CA1 of hippocampus and subiculum, and proceeds to CA2. See e.g., Arjun V. Masurkar, J Alzheimers Dis Parkinsonism, 8(1):412 (2018). Therefore, in order to inhibit neurogenesis in hippocampus dentate gyrus and induce pathology in CA1, both regions were selected as target sites for overexpressing miR-485-3p.

The neural circuit in the rodent hippocampus is composed of the excitatory trisynaptic pathway (entorhinal cortex (EC)-dentate gyrus (DG)-CA3-CA1 -EC). See e.g., Wei D. et al., Nat Rev Neurosci, 11:339-350 (2010). The experiment was designed to be projected to the entire hippocampus region including CA3-CA1 . If the lentiviral vector is injected by selecting only one coordinate, it is thought that there will be a limit to expressing miR-485-3p in the entire hippocampus. Therefore, the injection coordinates were set so that the lentiviral vector was injected into the posterior hippocampus and the virus-infected neurons could be projected to the anterior hippocampus.

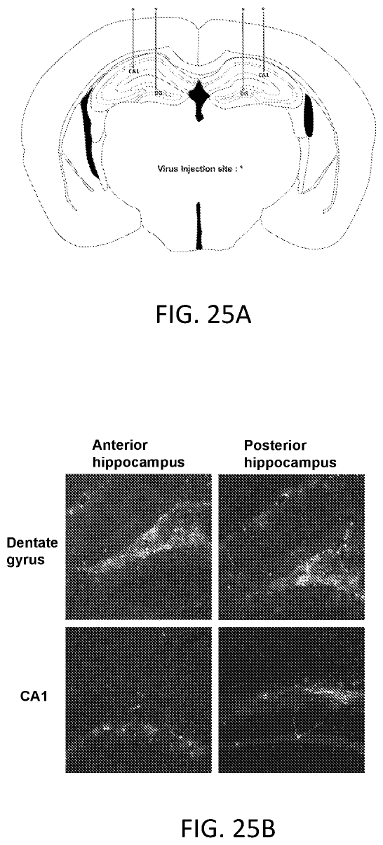

Mouse hippocampus DG and CA1 were subjected to a 2-point injection per hemisphere (total of 4 point) (FIG. 25A) of lenti-miR485-3p GFP-F containing vector. As shown in FIG. 25B, there was significant GFP expression in the dentate gyrus and CA1 of both the anterior hippocampus and posterior hippocampus. This result demonstrates that using the methods described above, miR-485-3p was successfully overexpressed broadly across the mouse hippocampus.

### Example 20

### Observation of Cognition-Related Behavioral Changes by miR485-3p Overexpression

Next, the effect of miR485-3p overexpression on cognition, learning, and memory-related behavioral changes were observed. Briefly, after about a month after miR485-3p overexpression, the different behavioral tests described in Example 19 were performed as described in FIG. 26.

### Open Field Test

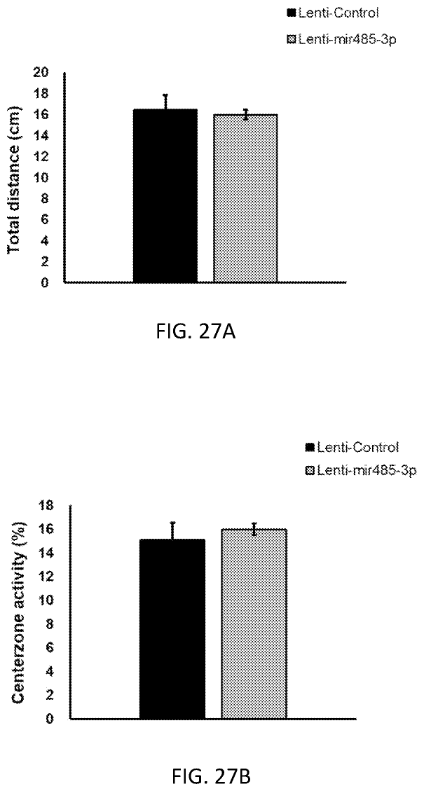

With the open field test (to measure changes in basic locomotion and anxiety-related responses), there was no significant difference in movement (total distance traveled) of the lenti-miR485-3p vector injected mice group compared to the control group (FIG. 27A). Similar results were observed when the centerzone activity (i.e., amount of time spent in the center portion of the open field arena; increase in center time thought to be indicative of anxiety-like activity) was assessed between the two groups (FIG. 27B). Thus, the overexpression of miR485-3p in the mouse hippocampus did not affect locomotion and emotion-related (e.g., anxiety) functions.

### Y-Maze Test

As described herein (e.g., Example 14 and Example 19), the Y-maze test is a behavioral experiment that evaluates spatial working memory. The test is based on the notion that normal rodents like to explore new environments (e.g., normal mice generally prefer to navigate from a previously visited arm to a new arm, rather than returning to one that was previously visited, of a Y-maze apparatus). In order to perform this action, various brain regions, such as the hippocampus, septum, basal forebrain, and prefrontal cortex, are involved. Accordingly, the Y-maze test can be useful in assessing the proper functioning of any of these different brain regions.

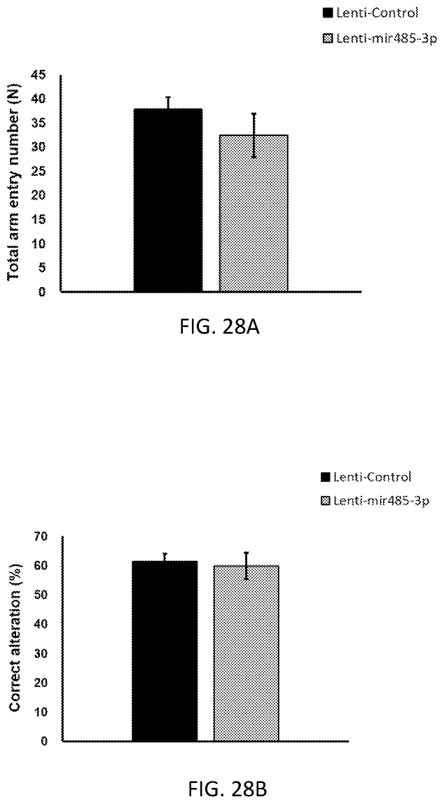

As observed, there was no statistically significant difference in the total arm entry number (FIG. 28A) between the control group and the mir485-3p overexpression group.

Similarly, the alternation behavior (i.e., the notion that normal mice will choose a different arm than the one it arrived from) was also comparable among the different animals (FIG. 28B). These results indicate that levels of general motor and exploratory activity in the Y-maze were not changed after miR-485 overexpression.

### Novel Object Recognition Test

As described herein (e.g., Example 19), novel object recognition test is a behavioral test that is often used in rodent models to assess possible deficits in object recognition memory. This test is based on the characteristics of mice to search for new objects with more curiosity than for familiar objects, and measures both short-term and long-term memory.

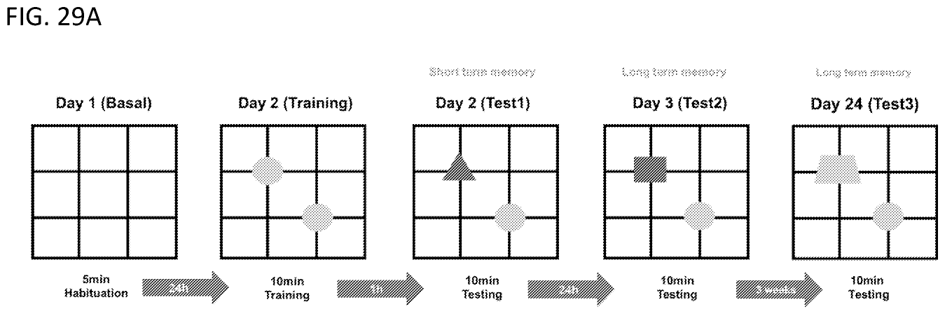

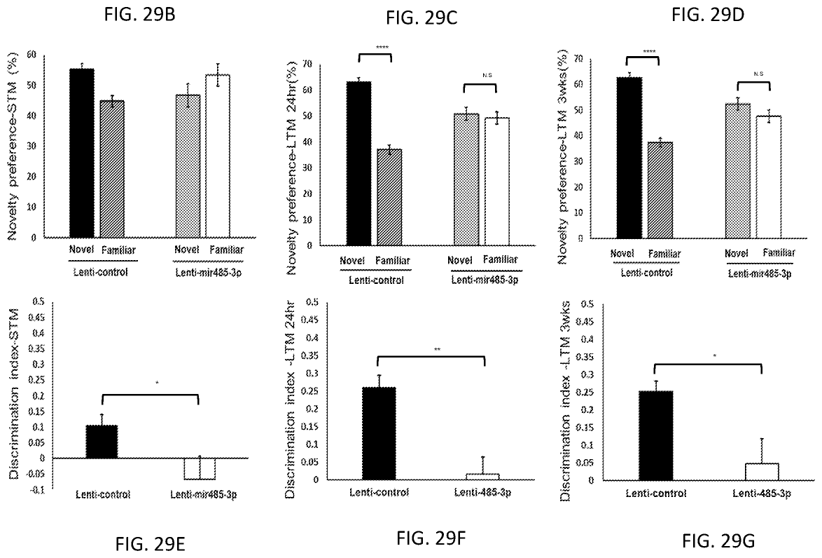

Compared to the control group (i.e., no miR485-3p overexpression in the hippocampus), mice from the experimental group (i.e., miR485-3p overexpression in the hippocampus) did exhibit statistically significant impairment in their ability to recognize objects both short-term (at 1 hour after object recognition training; see FIGS. 29B and 29E) and long-term (at 24 hours (see FIGS. 29C and 29F) and 3 weeks (see FIGS. 29D and 29G) after object recognition training). Animals from the control groups were able to distinguish between old and new objects and showed more interest in exploring the new objects. In contrast, mice with miR485-3p overexpression did not distinguish between the old and new objects. These results suggest that overexpression of the miR485-3p in the hippocampus has deleterious effects on the formation of both short-term and long-term memory.

### Passive Avoidance Test

As described herein, the passive avoidance test is a fear-motivated test. It tests the ability of mice to recognize and learn about the environment in order to avoid an environment where aversive stimulus, such as foot-shock, is given (i.e., associative memory).

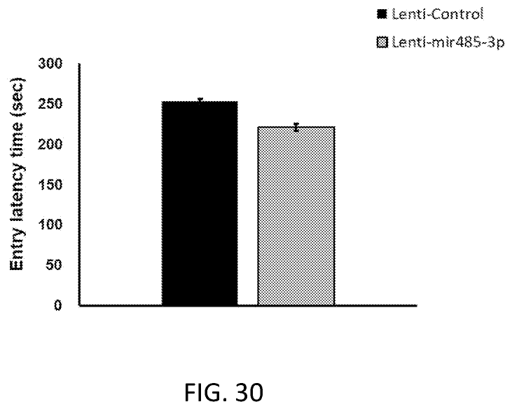

As shown in FIG. 30, there was no significant differences in the entry latency time values of the control group and the miR485-3p overexpression group. This data demonstrates that the overexpression of miR485-3p in the mouse hippocampus did not have any noticeable effect on fear acquisition.

Collectively, the above results demonstrate that the overexpression of miR-485 within the hippocampus impairs both short- and long-term memory, similar to that observed in many AD patients. The above results also suggest that the miR-485 inhibitors disclosed herein could be useful in improving certain cognitive functions (e.g., short- and long-term memory) in AD patients (e.g., by decreasing the expression of miR-485 within different brain regions).

### Example 21

### Effects of miR-485-3p Overexpression on Neural Cells

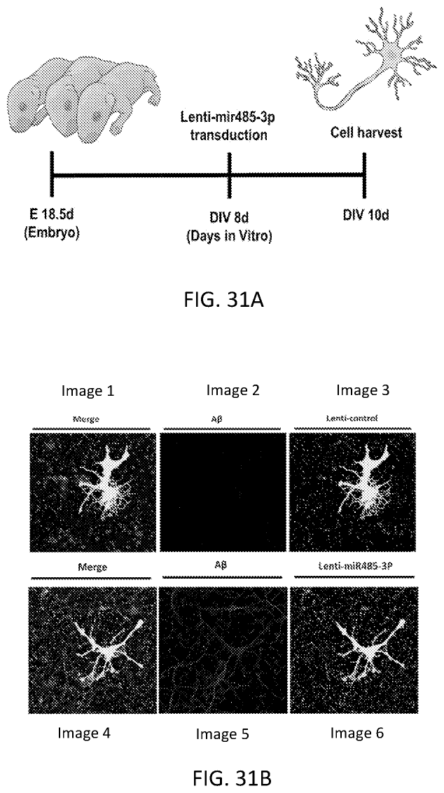

In order to observe whether the cognitive decline induced by the miR485-3p overexpression described above (e.g., Example 20) was caused by cellular changes within the hippocampus, neural cells were transduced with the lenti-miR485-3p (experimental group) or lenti-control vector (control group) as described in Example 19 (see FIG. 31A).

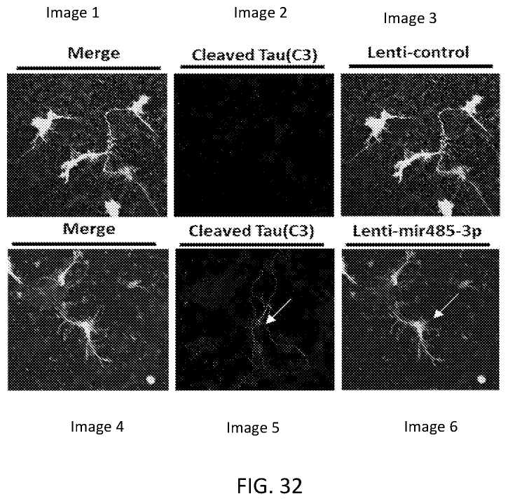

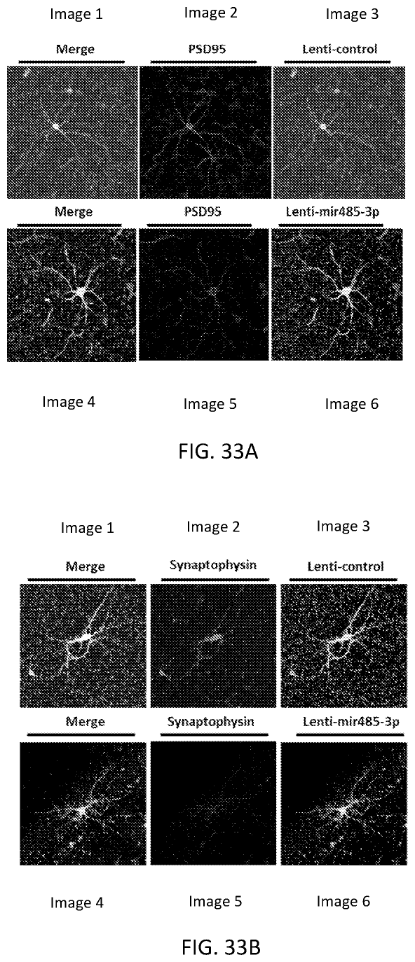

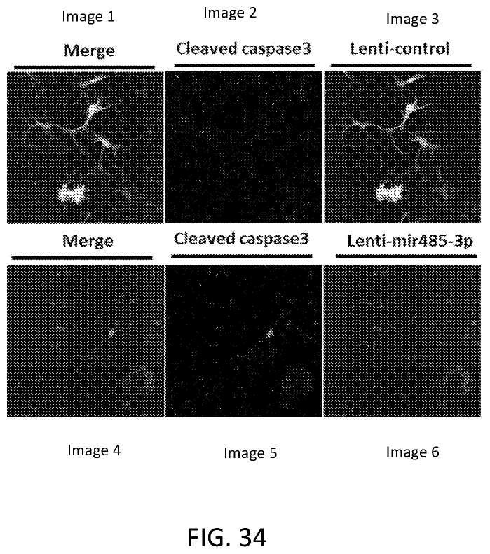

FIG. 31B shows that, in contrast to the lenti-control group, amyloid beta was increased and accumulated in cells overexpressed with miR485-3p. Amyloid beta was also observed in cells that were not infected with the virus in the experimental group, demonstrating the neuron to neuron spreading of amyloid beta. The truncated tau protein, known as a neuropathological hallmark of AD, was also observed to be increased in the miR485-3p overexpression group compared to the lenti-control group (FIG. 32). Additionally, in neurons transduced to overexpress miR485-3p, there was also a noticeable decrease in the expression of both PSD-95 (an important scaffolding protein that regulates synaptic distribution and activity of both NMDA and AMPA receptors; see FIG. 33A) and synaptophysin (thought to play a role in neurotransmitter release from synaptic vesicles by regulating membrane prefusion; see FIG. 33B). Not to be bound by any one theory, in some aspects, it is thought that the miR485-3p overexpression can negatively affect neural cell fate by increasing neuron amyloid beta expression, inducing tauopathy, weakening synaptogenesis, and/or increasing neuronal cell death (see FIG. 34).

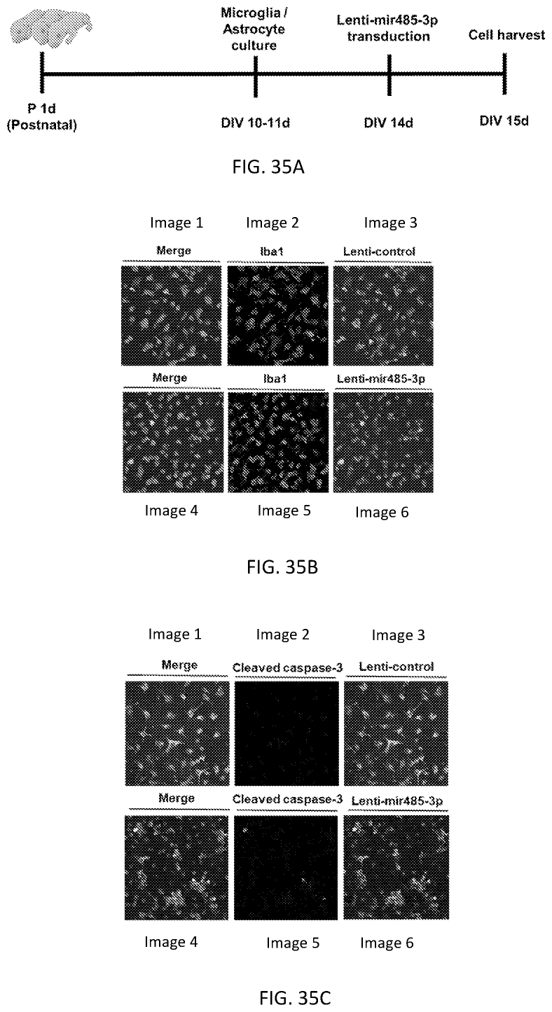

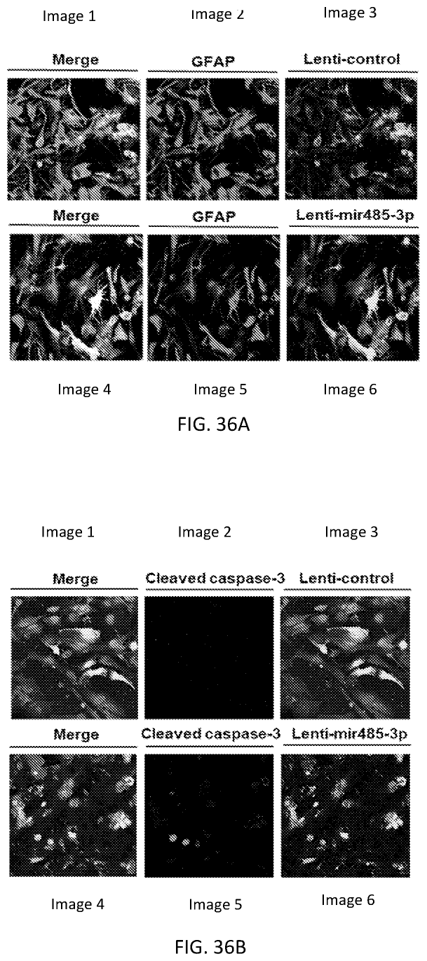

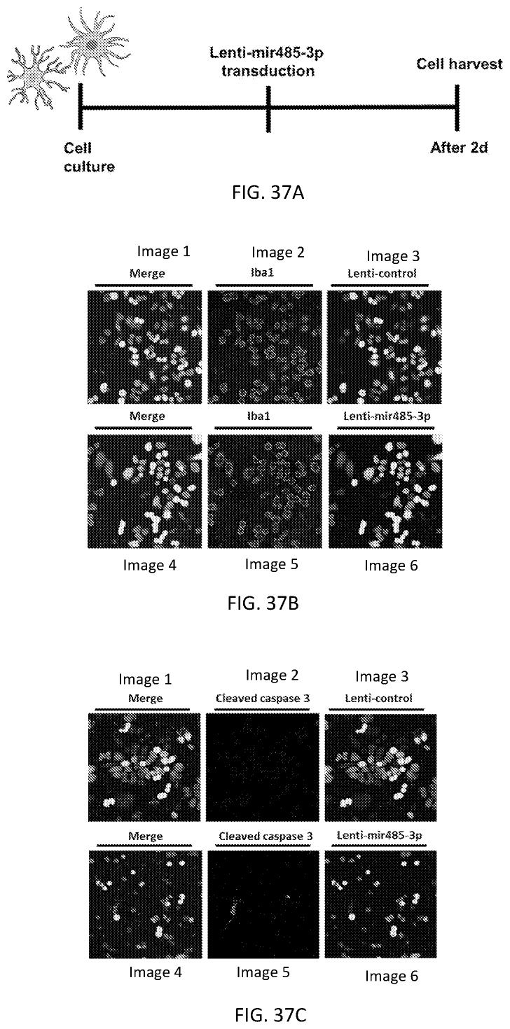

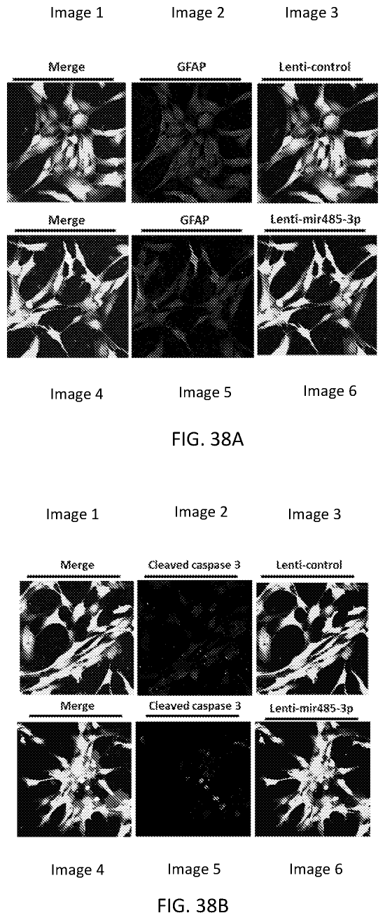

Next, to assess whether the overexpression of miR485-3p can also affect other neural cells, mouse primary astrocytes and microglia cells were transduced with the lenti-miR485-3p or lenti-control vector as described in Example 19 (see FIG. 35A). Lenti-virus infection was confirmed through the expression of Iba-1 (FIG. 35B), a cell-specific marker of microglia isolated from the mouse whole brain, and GFAP (FIG. 36A), a cell-specific marker of astrocytes, and observation of the characteristics of each cell and GFP signal. In both microglia (FIG. 35C and FIG. 37C) and astrocytes (FIG. 36B and FIG. 38B), cleaved caspase 3 was increased as a result of the miR485-3p overexpression. These results demonstrate that miR485-3p not only directly damages the neuron, but also causes gliosis of the glial cells, which play a major role in neuroinflammation and synaptic homeostasis.

The results described above in Examples 19-21 further demonstrate the role that miR485-3p expression can have in Alzheimer's disease induction. Not to be bound by any one theory, by decreasing and/or inhibiting the expression of miR485-3p, the miR-485 inhibitors described herein can be a useful therapeutic for the treatment of various neurodegenerative diseases and disorders, such as Alzheimer's disease.

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The contents of all cited references (including literature references, patents, patent applications, and websites) that can be cited throughout this application are hereby expressly incorporated by reference in their entirety for any purpose, as are the references cited therein.

What is claimed is:

1. A method of increasing a level of a SIRT1 protein and/or a SIRT1 gene in a subject in need thereof comprising administering to the subject a compound that inhibits miR-485 (miRNA inhibitor).

2. The method of claim 1, wherein the subject has a disease or a condition associated with a decreased level of a SIRT1 protein and/or a SIRT1 gene.

3. The method of claim 1 or 2, wherein the miRNA inhibitor induces autophagy and/or treats or prevents inflammation.

4. A method of increasing a level of a CD36 protein and/or a CD36 gene in a subject in need thereof comprising administering to the subject a compound that inhibits miR-485 (miRNA inhibitor).

5. The method of any one of claims 1 to 4, wherein the subject has a disease or a condition associated with a decreased level of a CD36 protein and/or a CD36 gene.

6. A method of increasing a level of a PGC-1α protein and/or a PGC-1α gene in a subject in need thereof comprising administering to the subject a compound that inhibits miR-485 (miRNA inhibitor).

7. The method of any one of claims 1 to 6, wherein the subject has a disease or a condition associated with a decreased level of a PGC-1α protein and/or a PGC-1α gene.

8. A method of increasing a level of a LRRK2 protein and/or a LRRK2 gene in a subject in need thereof comprising administering to the subject a compound that inhibits miR-485 (miRNA inhibitor).

9. The method of any one of claims 1 to 8, wherein the subject has a disease or a condition associated with a decreased level of a LRRK2 protein and/or a LRRK2 gene.

10. A method of increasing a level of a NRG1 protein and/or a NRG1 gene in a subject in need thereof comprising administering to the subject a compound that inhibits miR-485 (miRNA inhibitor).

11. The method of any one of claims 1 to 10, wherein the subject has a disease or a condition associated with a decreased level of a NRG1 protein and/or a NRG1 gene.

12. A method of increasing a level of a STMN2 protein and/or a STMN2 gene in a subject in need thereof comprising administering to the subject a compound that inhibits miR-485 (miRNA inhibitor).

13. The method of any one of claims 1 to 12, wherein the subject has a disease or a condition associated with a decreased level of a STMN2 protein and/or a STMN2 gene.

14. A method of increasing a level of a VLDLR protein and/or a VLDLR gene in a subject in need thereof comprising administering to the subject a compound that inhibits miR-485 (miRNA inhibitor).

15. The method of any one of claims 1 to 14, wherein the subject has a disease or a condition associated with a decreased level of a VLDLR protein and/or a VLDLR gene.

16. A method of increasing a level of a NRXN1 protein and/or a NRXN1 gene in a subject in need thereof comprising administering to the subject a compound that inhibits miR-485 (miRNA inhibitor).

17. The method of any one of claims 1 to 16, wherein the subject has a disease or a condition associated with a decreased level of a NRXN1 protein and/or a NRXN1 gene.

18. A method of increasing a level of a GRIA4 protein and/or a GRIA4 gene in a subject in need thereof comprising administering to the subject a compound that inhibits miR-485 (miRNA inhibitor).

19. The method of any one of claims 1 to 18, wherein the subject has a disease or a condition associated with a decreased level of a GRIA4 protein and/or a GRIA4 gene.

20. A method of increasing a level of a NXPH1 protein and/or a NXPH1 gene in a subject in need thereof comprising administering to the subject a compound that inhibits miR-485 (miRNA inhibitor).

21. The method of any one of claims 1 to 20, wherein the subject has a disease or a condition associated with a decreased level of a NXPH1 protein and/or a NXPH1 gene.

22. A method of increasing a level of a PSD-95 protein and/or a PSD-95 gene in a subject in need thereof comprising administering to the subject a compound that inhibits miR-485 (miRNA inhibitor).

23. The method of any one of claims 1 to 22, wherein the subject has a disease or a condition associated with a decreased level of a PSD-95 protein and/or a PSD-95 gene.

24. A method of increasing a level of a synaptophysin protein and/or a synaptophysin gene in a subject in need thereof comprising administering to the subject a compound that inhibits miR-485 (miRNA inhibitor).

25. The method of any one of claims 1 to 24, wherein the subject has a disease or a condition associated with a decreased level of a synaptophysin protein and/or a synaptophysin gene.

26. A method of decreasing a level of a caspase-3 protein and/or a caspase-3 gene in a subject in need thereof comprising administering to the subject a compound that inhibits miR-485 (miRNA inhibitor).

27. The method of any one of claims 1 to 26, wherein the subject has a disease or a condition associated with an increased level of a caspase-3 protein and/or a caspase-3 gene.

28. The method of any one of claims 1 to 27, wherein the miRNA inhibitor induces neurogenesis.

29. The method of claim 28, wherein inducing neurogenesis comprises an increased proliferation, differentiation, migration, and/or survival of neural stem cells and/or progenitor cells.

30. The method of claim 28 or 29, wherein inducing neurogenesis comprises an increased number of neural stem cells and/or progenitor cells.

31. The method of any one of claims 28 to 30, wherein inducing neurogenesis comprises an increased axon, dendrite, and/or synapse development.

32. The method of any one of claims 1 to 31, wherein the miRNA inhibitor induces phagocytosis.

33. A method of treating a disease or condition associated with an abnormal level of a SIRT1 protein and/or a SIRT1 gene in a subject in need thereof comprising administering to the subject a compound that inhibits miR-485 (miRNA inhibitor), wherein the miRNA inhibitor increases the level of the SIRT1 protein and/or SIRT1 gene.

34. A method of treating a disease or condition associated with an abnormal level of a CD36 protein and/or a CD36 gene in a subject in need thereof comprising administering to the subject a compound that inhibits miR-485 (miRNA inhibitor), wherein the miRNA inhibitor increases the level of the CD36 protein and/or CD36 gene.

35. A method of treating a disease or condition associated with an abnormal level of a PGC-1α protein and/or a PGC-1α gene in a subject in need thereof comprising administering to the subject a compound that inhibits miR-485 (miRNA inhibitor), wherein the miRNA inhibitor increases the level of the PGC-1α protein and/or PGC-1α gene.

36. A method of treating a disease or condition associated with an abnormal level of a LRRK2 protein and/or a LRRK2 gene in a subject in need thereof comprising administering to the subject a compound that inhibits miR-485 (miRNA inhibitor), wherein the miRNA inhibitor increases the level of the LRRK2 protein and/or LRRK2 gene.

37. A method of treating a disease or condition associated with an abnormal level of a NRG1 protein and/or a NRG1 gene in a subject in need thereof comprising administering to the subject a compound that inhibits miR-485 (miRNA inhibitor), wherein the miRNA inhibitor increases the level of the NRG1 protein and/or NRG1 gene.

38. A method of treating a disease or condition associated with an abnormal level of a STMN2 protein and/or a STMN2 gene in a subject in need thereof comprising administering to the subject a compound that inhibits miR-485 (miRNA inhibitor), wherein the miRNA inhibitor increases the level of the STMN2 protein and/or STMN2 gene.

39. A method of treating a disease or condition associated with an abnormal level of a VLDLR protein and/or a VLDLR gene in a subject in need thereof comprising administering to the subject a compound that inhibits miR-485 (miRNA inhibitor), wherein the miRNA inhibitor increases the level of the VLDLR protein and/or VLDLR gene.

40. A method of treating a disease or condition associated with an abnormal level of a NRXN1 protein and/or a NRXN1 gene in a subject in need thereof comprising administering to the subject a compound that inhibits miR-485 (miRNA inhibitor), wherein the miRNA inhibitor increases the level of the NRXN1 protein and/or NRXN1 gene.

41. A method of treating a disease or condition associated with an abnormal level of a GRIA4 protein and/or a GRIA4 gene in a subject in need thereof comprising administering to the subject a compound that inhibits miR-485 (miRNA inhibitor), wherein the miRNA inhibitor increases the level of the GRIA4 protein and/or GRIA4 gene.

42. A method of treating a disease or condition associated with an abnormal level of a NXPH1 protein and/or a NXPH1 gene in a subject in need thereof comprising administering to the subject a compound that inhibits miR-485 (miRNA inhibitor), wherein the miRNA inhibitor increases the level of the NXPH1 protein and/or NXPH1 gene.

43. A method of treating a disease or condition associated with an abnormal level of a PSD-95 protein and/or a PSD-95 gene in a subject in need thereof comprising administering to the subject a compound that inhibits miR-485 (miRNA inhibitor), wherein the miRNA inhibitor increases the level of the PSD-95 protein and/or PSD-95 gene.

44. A method of treating a disease or condition associated with an abnormal level of a synaptophysin protein and/or a synaptophysin gene in a subject in need thereof comprising administering to the subject a compound that inhibits miR-485 (miRNA inhibitor), wherein the miRNA inhibitor increases the level of the synaptophysin protein and/or synaptophysin gene.

45. A method of treating a disease or condition associated with an abnormal level of a caspase-3 protein and/or a caspase-3 gene in a subject in need thereof comprising administering to the subject a compound that inhibits miR-485 (miRNA inhibitor), wherein the miRNA inhibitor decreases the level of the caspase-3 protein and/or caspase-3 gene.

46. The method of any one of claims 1 to 45, wherein the miRNA inhibitor inhibits miR485-3p.

47. The method of claim 46, wherein the miR485-3p comprises 5′-gucauacacggcucuccucucu-3′ (SEQ ID NO: 1).

48. The method of any one of claims 1 to 47, wherein the miRNA inhibitor comprises a nucleotide sequence comprising 5′-UGUAUGA-3′ (SEQ ID NO: 2) and wherein the miRNA inhibitor comprises about 6 to about 30 nucleotides in length.

49. The method of any one of claims 1 to 48, wherein the miRNA inhibitor increases transcription of an SIRT1 gene and/or expression of a SIRT1 protein; increases transcription of a CD36 gene and/or expression of a CD36 protein; increases transcription of a PGC1 gene and/or expression of a PGC1 protein; increases transcription of a LRRK2 gene and/or expression of a LRRK2 protein; increases transcription of a NRG1 gene and/or expression of a NRG1 protein; increases transcription of a STMN2 gene and/or expression of a STMN2 protein; increases transcription of a VLDLR gene and/or expression of a VLDLR protein; increases transcription of a NRXN1 gene and/or expression of a NRXN1 protein; increases transcription of a GRIA4 gene and/or expression of a GRIA4 protein; increases transcription of a NXPH1 gene and/or expression of a NXPH1 protein; increases transcription of a PSD-95 gene and/or expression of a PSD-95 protein; increases transcription of a synaptophysin gene and/or expression of a synaptophysin protein; decreases transcription of a caspase-3 gene and/or expression of a caspase-3 protein; or any combination thereof.

50. The method of any one of claims 1 to 49, wherein the miRNA inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 5′ of the nucleotide sequence.

51. The method of any one of claims 1 to 50, wherein the miRNA inhibitor comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides at the 3′ of the nucleotide sequence.

52. The method of any one of claims 1 to 51, wherein the miRNA inhibitor has a sequence selected from the group consisting of: 5′-UGUAUGA-3′ (SEQ ID NO: 2), 5′-GUGUAUGA-3′ (SEQ ID NO: 3), 5′-CGUGUAUGA-3′ (SEQ ID NO: 4), 5′-CCGUGUAUGA-3′ (SEQ ID NO: 5), 5′-GCCGUGUAUGA-3′ (SEQ ID NO: 6), 5′-AGCCGUGUAUGA-3′ (SEQ ID NO: 7), 5′-GAGCCGUGUAUGA-3′ (SEQ ID NO: 8), 5′-AGAGCCGUGUAUGA-3′ (SEQ ID NO: 9), 5′-GAGAGCCGUGUAUGA-3′ (SEQ ID NO: 10), 5′-GGAGAGCCGUGUAUGA-3′ (SEQ ID NO: 11), 5′-AGGAGAGCCGUGUAUGA-3′ (SEQ ID NO: 12), 5′-GAGGAGAGCCGUGUAUGA-3′ (SEQ ID NO: 13), 5′-AGAGGAGAGCCGUGUAUGA-3′ (SEQ ID NO: 14), 5′-GAGAGGAGAGCCGUGUAUGA-3′ (SEQ ID NO: 15); 5′-UGUAUGAC-3′ (SEQ ID NO: 16), 5′-GUGUAUGAC-3′ (SEQ ID NO: 17), 5′-CGUGUAUGAC-3′ (SEQ ID NO: 18), 5′-CCGUGUAUGAC-3′ (SEQ ID NO: 19), 5′-GCCGUGUAUGAC-3′ (SEQ ID NO: 20), 5′-AGCCGUGUAUGAC-3′ (SEQ ID NO: 21), 5′-GAGCCGUGUAUGAC-3′ (SEQ ID NO: 22), 5′-AGAGCCGUGUAUGAC-3′ (SEQ ID NO: 23), 5′-GAGAGCCGUGUAUGAC-3′ (SEQ ID NO: 24), 5′-GGAGAGCCGUGUAUGAC-3′ (SEQ ID NO: 25), 5′-AGGAGAGCCGUGUAUGAC-3′ (SEQ ID NO: 26), 5′-GAGGAGAGCCGUGUAUGAC-3′ (SEQ ID NO: 27), 5′-AGAGGAGAGCCGUGUAUGAC-3′ (SEQ ID NO: 28), 5′-GAGAGGAGAGCCGUGUAUGAC-3′ (SEQ ID NO: 29), or AGAGAGGAGAGCCGUGUAUGAC (SEQ ID NO: 30).

53. The method of any one of claims 1 to 46 and 49 to 51, wherein the miRNA inhibitor has a sequence selected from the group consisting of: 5′-TGTATGA-3′ (SEQ ID NO: 62), 5′-GTGTATGA-3′ (SEQ ID NO: 63), 5′-CGTGTATGA-3′ (SEQ ID NO: 64), 5′-CCGTGTATGA-3′ (SEQ ID NO: 65), 5′-GCCGTGTATGA-3′ (SEQ ID NO: 66), 5′-AGCCGTGTATGA-3′ (SEQ ID NO: 67), 5′-GAGCCGTGTATGA-3′ (SEQ ID NO: 68), 5′-AGAGCCGTGTATGA-3′ (SEQ ID NO: 69), 5′-GAGAGCCGTGTATGA-3′ (SEQ ID NO: 70), 5′-GGAGAGCCGTGTATGA-3′ (SEQ ID NO: 71), 5′-AGGAGAGCCGTGTATGA-3′ (SEQ ID NO: 72), 5′-GAGGAGAGCCGTGTATGA-3′ (SEQ ID NO: 73), 5′-AGAGGAGAGCCGTGTATGA-3′ (SEQ ID NO: 74), 5′-GAGAGGAGAGCCGTGTATGA-3′ (SEQ ID NO: 75); 5′-TGTATGAC-3′ (SEQ ID NO: 76), 5′-GTGTATGAC-3′ (SEQ ID NO: 77), 5′-CGTGTATGAC-3′ (SEQ ID NO: 78), 5′-CCGTGTATGAC-3′ (SEQ ID NO: 79), 5′-GCCGTGTATGAC-3′ (SEQ ID NO: 80), 5′-AGCCGTGTATGAC-3′ (SEQ ID NO: 81), 5′-GAGCCGTGTATGAC-3′ (SEQ ID NO: 82), 5′-AGAGCCGTGTATGAC-3′ (SEQ ID NO: 83), 5′-GAGAGCCGTGTATGAC-3′ (SEQ ID NO: 84), 5′-GGAGAGCCGTGTATGAC-3′ (SEQ ID NO: 85), 5′-AGGAGAGCCGTGTATGAC-3′ (SEQ ID NO: 86), 5′-GAGGAGAGCCGTGTATGAC-3′ (SEQ ID NO: 87), 5′-AGAGGAGAGCCGTGTATGAC-3′ (SEQ ID NO: 88), 5′-GAGAGGAGAGCCGTGTATGAC-3′ (SEQ ID NO: 89), and 5′-AGAGAGGAGAGCCGTGTATGAC-3′ (SEQ ID NO: 90).

54. The method of any one of claims 1 to 51, wherein the sequence of the miRNA inhibitor is at least about 50%, at least about 55%, at least about 60%, at least about 65%, at least about 70%, at least about 75%, at least about 80%, at least about 85%, at least about 90%, or at least about 95% sequence identity to 5′-AGAGAGGAGAGCCGUGUAUGAC-3′ (SEQ ID NO: 30) or 5′-AGAGAGGAGAGCCGTGTATGAC-3′ (SEQ ID NO: 90).

55. The method of claim 54, wherein the miRNA inhibitor has a sequence that has at least 90% similarity to 5′-AGAGAGGAGAGCCGUGUAUGAC-3′ (SEQ ID NO: 30) or 5′-AGAGAGGAGAGCCGTGTATGAC-3′ (SEQ ID NO: 90).

56. The method of any one of claims 1 to 51, wherein the miRNA inhibitor comprises the nucleotide sequence 5′-AGAGAGGAGAGCCGUGUAUGAC-3′ (SEQ ID NO: 30) or 5′-AGAGAGGAGAGCCGTGTATGAC-3′ (SEQ ID NO: 90) with one substitution or two substitutions.

57. The method of any one of claims 1 to 51, wherein the miRNA inhibitor comprises the nucleotide sequence 5′-AGAGAGGAGAGCCGUGUAUGAC-3′ (SEQ ID NO: 30) or 5′-AGAGAGGAGAGCCGTGTATGAC-3′ (SEQ ID NO: 90).

58. The method of claim 57, wherein the miRNA inhibitor comprises the nucleotide sequence 5′-AGAGAGGAGAGCCGUGUAUGAC-3′ (SEQ ID NO: 30).

59. The method of any one of claims 1 to 58, wherein the miRNA inhibitor comprises at least one modified nucleotide.

60. The method of claim 59, wherein the at least one modified nucleotide is a locked nucleic acid (LNA), an unlocked nucleic acid (UNA), an arabino nucleic acid (ABA), a bridged nucleic acid (BNA), and/or a peptide nucleic acid (PNA).

61. The method of any one of claims 1 to 60, wherein the miRNA inhibitor comprises a backbone modification.

62. The method of claim 61, wherein the backbone modification is a phosphorodiamidate morpholino oligomer (PMO) and/or phosphorothioate (PS) modification.

63. The method of any one of claims 1 to 62, wherein the miRNA inhibitor is delivered in a delivery agent.

64. The method of claim 63, wherein the delivery agent is a micelle, an exosome, a lipid nanoparticle, an extracellular vesicle, or a synthetic vesicle.

65. The method of any one of claims 1 to 64, wherein the miRNA inhibitor is delivered by a viral vector.

66. The method of claim 65, wherein the viral vector is an AAV, an adenovirus, a retrovirus, or a lentivirus.

67. The method of claim 66, wherein the viral vector is an AAV that has a serotype of AAV2, AAV3, AAV4, AAV5, AAV6, AAV7, AAV8, AAV9, AAV10, or any combination thereof.

68. The method of any one claims 1 to 67, wherein the miRNA inhibitor is delivered with a delivery agent.

69. The method of claim 68, wherein the delivery agent comprises a lipidoid, a liposome, a lipoplex, a lipid nanoparticle, a polymeric compound, a peptide, a protein, a cell, a nanoparticle mimic, a nanotube, or a conjugate.

70. The method of claim 68 or 69, wherein the delivery agent comprises a cationic carrier unit comprising
[WP]-L1-[CC]-L2-[AM] (formula I)
or
[WP]-L1-[AM]-L2-[CC] (formula II)
wherein
WP is a water-soluble biopolymer moiety;
CC is a positively charged carrier moiety;
AM is an adjuvant moiety; and,
L1 and L2 are independently optional linkers, and
wherein when mixed with a nucleic acid at an ionic ratio of about 1:1, the cationic carrier unit forms a micelle.

71. The method of claim 70, wherein the miRNA inhibitor interacts with the cationic carrier unit via an ionic bond.

72. The method of claim 70 or 71, wherein the water-soluble polymer comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (“POZ”) poly(N-acryloylmorpholine), or any combinations thereof.

73. The method of claims 70 to 72, wherein the water-soluble polymer comprises polyethylene glycol (“PEG”), polyglycerol, or poly(propylene glycol) (“PPG”).

74. The method of any one of claims 70 to 73, wherein the water-soluble polymer comprises: 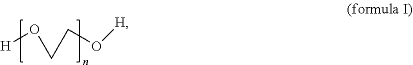
wherein n is 1-1000.

75. The method of claim 74, wherein the n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141.

76. The method of claim 74, wherein then is about 80 to about 90, about 90 to about 100, about 100 to about 110, about 110 to about 120, about 120 to about 130, about 140 to about 150, about 150 to about 160.

77. The method of any one of claims 70 to 76, wherein the water-soluble polymer is linear, branched, or dendritic.

78. The method of any one of claims 70 to 77, wherein the cationic carrier moiety comprises one or more basic amino acids.

79. The method of claim 78, wherein the cationic carrier moiety comprises at least three, at least four, at least five, at least six, at least seven, at least eight, at least nine, at least ten, at least 11, at least 12, at least 13, at least 14, at last 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 basic amino acids.

80. The method of claim 79, wherein the cationic carrier moiety comprises about 30 to about 50 basic amino acids.

81. The method of claim 79 or 80, wherein the basic amino acid comprises arginine, lysine, histidine, or any combination thereof.

82. The method of any one of claims 70 to 81, wherein the cationic carrier moiety comprises about 40 lysine monomers.

83. The method of any one of claims 70 to 82, wherein the adjuvant moiety is capable of modulating an immune response, an inflammatory response, and/or a tissue microenvironment.

84. The method of any one of claims 70 to 82, wherein the adjuvant moiety comprises an imidazole derivative, an amino acid, a vitamin, or any combination thereof.

85. The method of claim 84, wherein the adjuvant moiety comprises: 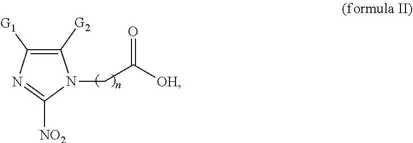
wherein each of G1 and G2 is H, an aromatic ring, or 1-10 alkyl, or G1 and G2 together form an aromatic ring, and wherein n is 1-10.

86. The method of claim 84, wherein the adjuvant moiety comprises nitroimidazole.

87. The method of claim 84, wherein the adjuvant moiety comprises metronidazole, tinidazole, nimorazole, dimetridazole, pretomanid, ornidazole, megazol, azanidazole, benznidazole, or any combination thereof.

88. The method of any one of claims 70 to 84, wherein the adjuvant moiety comprises an amino acid.

89. The method of claim 88, wherein the adjuvant moiety comprises 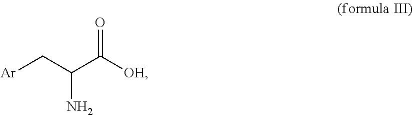
wherein Ar is 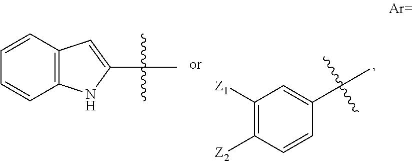
and
wherein each of Z1 and Z2 is H or OH.

90. The method of any one of claims 70 to 84, wherein the adjuvant moiety comprises a vitamin.

91. The method of claim 90, wherein the vitamin comprises a cyclic ring or cyclic hetero atom ring and a carboxyl group or hydroxyl group.

92. The method of claim 90 or 91, wherein the vitamin comprises: 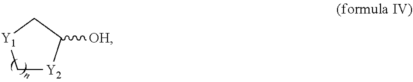
wherein each of Y1 and Y2 is C, N, O, or S, and wherein n is 1 or 2.

93. The method of any one of claims 90 to 92, wherein the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof.

94. The method of any one of claims 90 to 93, wherein the vitamin is vitamin B3.

95. The method of any one of claims 90 to 94, wherein the adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, or at least about 20 vitamin B3.

96. The method of claim 95, wherein the adjuvant moiety comprises about 10 vitamin B3.

97. The method of any one of claims 90 to 96, wherein the delivery agent comprises about a water-soluble biopolymer moiety with about 120 to about 130 PEG units, a cationic carrier moiety comprising a poly-lysine with about 30 to about 40 lysines, and an adjuvant moiety with about 5 to about 10 vitamin B3.

98. The method of any one of claims 90 to 97, wherein the delivery agent is associated with the miRNA inhibitor, thereby forming a micelle.

99. The method of claim 98, wherein the association is a covalent bond, a non-covalent bond, or an ionic bond.

100. The method of claim 98 or 99, wherein the cationic carrier unit and the miRNA inhibitor in the micelle is mixed in a solution so that the ionic ratio of the positive charges of the cationic carrier unit and the negative charges of the miRNA inhibitor is about 1: 1.

101. The method of any one of claims 98 to 100, wherein the cationic carrier unit is capable of protecting the miRNA inhibitor from enzymatic degradation.

102. The method of any one of claims 2, 3, 5, 7, 9, 11, 13, 15, 17, 19, and 21-101, wherein the disease or condition comprises Alzheimer's disease.

103. The method of any one of claims 2, 3, 5, 7, 9, 11, 13, 15, 17, 19, and 21-101, wherein the disease or condition comprises autism spectrum disorder, mental retardation, seizure, stroke, Parkinson's disease, spinal cord injury, or combinations thereof.

104. The method of claim 103, wherein the disease or condition is Parkinson's disease.

105. The method of claim 63, wherein the delivery agent is a micelle.

106. The method of claim 105, wherein the micelle comprises (i) about 100 to about 200 PEG units, (ii) about 30 to about 40 lysines, each with an amine group, (iii) about 15 to about 20 lysines, each with a thiol group, and (iv) about 30 to about 40 lysines, each linked to vitamin B3.

107. The method of claim 105, wherein the micelle comprises (i) about 120 to about 130 PEG units, (ii) about 32 lysines, each with an amine group, (iii) about 16 lysines, each with a thiol group, and (iv) about 32 lysines, each linked to vitamin B3.

108. The method of claim 106 or 107, wherein a targeting moiety is further linked to the PEG units.

109. The method of claim 108, wherein the targeting moiety is a LAT 1 targeting ligand.

110. The method of claim 109, wherein the targeting moiety is pennyl alanine.


SEQUENCE LISTING (122 sequences)

SEQ ID NO: 1 - RNA, 22 nt, Artificial Sequence
  oligonucleotide
gucauacacggcucuccucucu

SEQ ID NO: 2 - RNA, 7 nt, Artificial Sequence
  oligonucleotide
uguauga

SEQ ID NO: 3 - RNA, 8 nt, Artificial Sequence
  oligonucleotide
guguauga

SEQ ID NO: 4 - RNA, 9 nt, Artificial Sequence
  oligonucleotide
cguguauga

SEQ ID NO: 5 - RNA, 10 nt, Artificial Sequence
  oligonucleotide
ccguguauga

SEQ ID NO: 6 - RNA, 11 nt, Artificial Sequence
  oligonucleotide
gccguguauga

SEQ ID NO: 7 - RNA, 12 nt, Artificial Sequence
  oligonucleotide
agccguguauga

SEQ ID NO: 8 - RNA, 13 nt, Artificial Sequence
  oligonucleotide
gagccguguauga

SEQ ID NO: 9 - RNA, 14 nt, Artificial Sequence
  oligonucleotide
agagccguguauga

SEQ ID NO: 10 - RNA, 15 nt, Artificial Sequence
  oligonucleotide
gagagccguguauga

SEQ ID NO: 11 - RNA, 16 nt, Artificial Sequence
  oligonucleotide
ggagagccguguauga

SEQ ID NO: 12 - RNA, 17 nt, Artificial Sequence
  oligonucleotide
aggagagccguguauga

SEQ ID NO: 13 - RNA, 18 nt, Artificial Sequence
  oligonucleotide
gaggagagccguguauga

SEQ ID NO: 14 - RNA, 19 nt, Artificial Sequence
  oligonucleotide
agaggagagccguguauga

SEQ ID NO: 15 - RNA, 20 nt, Artificial Sequence
  oligonucleotide
gagaggagagccguguauga

SEQ ID NO: 16 - RNA, 8 nt, Artificial Sequence
  oligonucleotide
uguaugac

SEQ ID NO: 17 - RNA, 9 nt, Artificial Sequence
  oligonucleotide
guguaugac

SEQ ID NO: 18 - RNA, 10 nt, Artificial Sequence
  oligonucleotide
cguguaugac

SEQ ID NO: 19 - RNA, 11 nt, Artificial Sequence
  oligonucleotide
ccguguaugac

SEQ ID NO: 20 - RNA, 12 nt, Artificial Sequence
  oligonucleotide
gccguguaugac

SEQ ID NO: 21 - RNA, 13 nt, Artificial Sequence
  oligonucleotide
agccguguaugac

SEQ ID NO: 22 - RNA, 14 nt, Artificial Sequence
  oligonucleotide
gagccguguaugac

SEQ ID NO: 23 - RNA, 15 nt, Artificial Sequence
  oligonucleotide
agagccguguaugac

SEQ ID NO: 24 - RNA, 16 nt, Artificial Sequence
  oligonucleotide
gagagccguguaugac

SEQ ID NO: 25 - RNA, 17 nt, Artificial Sequence
  oligonucleotide
ggagagccguguaugac

SEQ ID NO: 26 - RNA, 18 nt, Artificial Sequence
  oligonucleotide
aggagagccguguaugac

SEQ ID NO: 27 - RNA, 19 nt, Artificial Sequence
  oligonucleotide
gaggagagccguguaugac

SEQ ID NO: 28 - RNA, 20 nt, Artificial Sequence
  oligonucleotide
agaggagagccguguaugac

SEQ ID NO: 29 - RNA, 21 nt, Artificial Sequence
  oligonucleotide
gagaggagagccguguaugac

SEQ ID NO: 30 - RNA, 22 nt, Artificial Sequence
  oligonucleotide
agagaggagagccguguaugac

SEQ ID NO: 31 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 32 - protein, 561 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 33 - RNA, 22 nt, Homo sapiens
agaggcuggccgugaugaauuc

SEQ ID NO: 34 - RNA, 22 nt, Artificial Sequence
  miR-485-3p
agucauacacggcucuccucuc

SEQ ID NO: 35 - RNA, 22 nt, Artificial Sequence
  miR-485-5p
agaggcuggccgugaugaauuc

SEQ ID NO: 36 - protein, 472 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 37 - protein, 288 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRETCVHFTSSFSVCKS

SEQ ID NO: 38 - protein, 433 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 39 - protein, 412 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 40 - protein, 798 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 41 - protein, 271 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNLFL

SEQ ID NO: 42 - protein, 803 aa, Homo sapiens
MDETSPRLEEDWKKVLQREAGWQCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 43 - protein, 786 aa, Homo sapiens
MDEGYFCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 44 - protein, 289 aa, Homo sapiens
MDEGYFCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGVKTNLISK

SEQ ID NO: 45 - protein, 276 aa, Homo sapiens
MDETSPRLEEDWKKVLQREAGWQCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNLFL

SEQ ID NO: 46 - protein, 138 aa, Homo sapiens
MDEGYFCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLVRTLPTV

SEQ ID NO: 47 - protein, 301 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGVKTNLISK

SEQ ID NO: 48 - protein, 671 aa, Homo sapiens
MPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 49 - RNA, 7 nt, Artificial Sequence
  oligonucleotide
ucauaca

SEQ ID NO: 50 - RNA, 7 nt, Artificial Sequence
  oligonucleotide
gaggcug

SEQ ID NO: 51 - DNA, 18 bp, Artificial Sequence
  oligonucleotide
cgaggtcgacttcctaga

SEQ ID NO: 52 - DNA, 23 bp, Artificial Sequence
  oligonucleotide
catacacggctctcctctctaaa

SEQ ID NO: 53 - DNA, 20 bp, Artificial Sequence
  oligonucleotide
tcctgtggcatccatgaaac

SEQ ID NO: 54 - DNA, 20 bp, Artificial Sequence
  oligonucleotide
caatgcctgggtacatggtg

SEQ ID NO: 55 - DNA, 18 bp, Artificial Sequence
  oligonucleotide
ccaagtggaggagcagct

SEQ ID NO: 56 - DNA, 20 bp, Artificial Sequence
  oligonucleotide
gacaaggtacaacccatcgg

SEQ ID NO: 57 - DNA, 22 bp, Artificial Sequence
  oligonucleotide
ttcgacacatgggataacgagg

SEQ ID NO: 58 - DNA, 21 bp, Artificial Sequence
  oligonucleotide
tttttgctgtgagtcccggag

SEQ ID NO: 59 - DNA, 20 bp, Artificial Sequence
  oligonucleotide
cagcctagcagcacgtaaat

SEQ ID NO: 60 - DNA, 19 bp, Artificial Sequence
  oligonucleotide
gaatcgagcaccagttacg

SEQ ID NO: 61 - DNA, 22 bp, Artificial Sequence
  oligonucleotide
ccttccctgaaggttcctcctt

SEQ ID NO: 62 - DNA, 7 bp, artificial Sequence
  oligonucleotide
tgtatga

SEQ ID NO: 63 - DNA, 8 bp, artificial Sequence
  oligonucleotide
gtgtatga

SEQ ID NO: 64 - DNA, 9 bp, artificial Sequence
  oligonucleotide
cgtgtatga

SEQ ID NO: 65 - DNA, 10 bp, artificial Sequence
  oligonucleotide
ccgtgtatga

SEQ ID NO: 66 - DNA, 11 bp, artificial Sequence
  oligonucleotide
gccgtgtatga

SEQ ID NO: 67 - DNA, 12 bp, artificial Sequence
  oligonucleotide
agccgtgtatga

SEQ ID NO: 68 - DNA, 13 bp, artificial Sequence
  oligonucleotide
gagccgtgtatga

SEQ ID NO: 69 - DNA, 14 bp, artificial Sequence
  oligonucleotide
agagccgtgtatga

SEQ ID NO: 70 - DNA, 15 bp, artificial Sequence
  oligonucleotide
gagagccgtgtatga

SEQ ID NO: 71 - DNA, 16 bp, artificial Sequence
  oligonucleotide
ggagagccgtgtatga

SEQ ID NO: 72 - DNA, 17 bp, artificial Sequence
  oligonucleotide
aggagagccgtgtatga

SEQ ID NO: 73 - DNA, 18 bp, artificial Sequence
  oligonucleotide
gaggagagccgtgtatga

SEQ ID NO: 74 - DNA, 19 bp, artificial Sequence
  oligonucleotide
agaggagagccgtgtatga

SEQ ID NO: 75 - DNA, 20 bp, artificial Sequence
  oligonucleotide
gagaggagagccgtgtatga

SEQ ID NO: 76 - DNA, 8 bp, artificial Sequence
  oligonucleotide
tgtatgac

SEQ ID NO: 77 - DNA, 9 bp, artificial Sequence
  oligonucleotide
gtgtatgac

SEQ ID NO: 78 - DNA, 10 bp, artificial Sequence
  oligonucleotide
cgtgtatgac

SEQ ID NO: 79 - DNA, 11 bp, artificial Sequence
  oligonucleotide
ccgtgtatgac

SEQ ID NO: 80 - DNA, 12 bp, artificial Sequence
  oligonucleotide
gccgtgtatgac

SEQ ID NO: 81 - DNA, 13 bp, artificial Sequence
  oligonucleotide
agccgtgtatgac

SEQ ID NO: 82 - DNA, 14 bp, artificial Sequence
  oligonucleotide
gagccgtgtatgac

SEQ ID NO: 83 - DNA, 15 bp, artificial Sequence
  oligonucleotide
agagccgtgtatgac

SEQ ID NO: 84 - DNA, 16 bp, artificial Sequence
  oligonucleotide
gagagccgtgtatgac

SEQ ID NO: 85 - DNA, 17 bp, artificial Sequence
  oligonucleotide
ggagagccgtgtatgac

SEQ ID NO: 86 - DNA, 18 bp, artificial Sequence
  oligonucleotide
aggagagccgtgtatgac

SEQ ID NO: 87 - DNA, 19 bp, artificial Sequence
  oligonucleotide
gaggagagccgtgtatgac

SEQ ID NO: 88 - DNA, 20 bp, artificial Sequence
  oligonucleotide
agaggagagccgtgtatgac

SEQ ID NO: 89 - DNA, 21 bp, artificial Sequence
  oligonucleotide
gagaggagagccgtgtatgac

SEQ ID NO: 90 - DNA, 22 bp, artificial Sequence
  oligonucleotide
agagaggagagccgtgtatgac

SEQ ID NO: 91 - protein, 640 aa, artificial Sequence
  NRG1 Isoform 1
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 92 - protein, 648 aa, artificial Sequence
  NRG1 Isoform 2
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKHLGIEFIEAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 93 - protein, 462 aa, artificial Sequence
  NRG1 Isoform 3
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERHNLIAELRRNKAHRSKCMQIQLSATHLRSSSIPHLGFIL

SEQ ID NO: 94 - protein, 247 aa, artificial Sequence
  NRG1 Isoform 4
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQESAQMSLLVIAAKTT

SEQ ID NO: 95 - protein, 645 aa, artificial Sequence
  NRG1 Isoform 6
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYKHLGIEFMEAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 96 - protein, 637 aa, artificial Sequence
  NRG1 Isoform 7
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYKAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 97 - protein, 241 aa, artificial Sequence
  NRG1 Isoform 8
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYSTSTPFLSLPE

SEQ ID NO: 98 - protein, 422 aa, artificial Sequence
  NRG1 Isoform 9
MRWRRAPRRSGRPGPRAQRPGSAARSSPPLPLLPLLLLLGTAALAPGAAAGNEAAPAGASVCYSSPPSVGSVQELAQRAAVVIEGKVHPQRRQQGALDRKAAAAAGEAGAWGGDREPPAAGPRALGPPAEEPLLAANGTVPSWPTAPVPSAGEPGEEAPYLVKVHQVWAVKAGGLKKDSLLTVRLGTWGHPAFPSCGRLKEDSRYIFFMEPDANSTSRAPAAFRASFPPLETGRNLKKEVSRVLCKRCALPPRLKEMKSQESAAGSKLVLRCETSSEYSSLRFKWFKNGNELNRKNKPQNIKIQKKPGKSELRINKASLADSGEYMCKVISKLGNDSASANITIVESNATSTSTTGTSHLVKCAEKEKTFCVNGGECFMVKDLSNPSRYLCKCPNEFTGDRCQNYVMASFYSTSTPFLSLPE

SEQ ID NO: 99 - protein, 296 aa, artificial Sequence
  NRG1 Isoform 10
MEIYSPDMSEVAAERSSSPSTQLSADPSLDGLPAAEDMPEPQTEDGRTPGLVGLAVPCCACLEAERLRGCLNSEKICIVPILACLVSLCLCIAGLKWVFVDKIFEYDSPTHLDPGGLGQDPIISLDATAASAVWVSSEAYTSPVSRAQSESEVQVTVQGDKAVVSFEPSAAPTPKNRIFAFSFLPSTAPSFPSPTRNPEVRTPKSATQPQTTETNLQTAPKLSTSTSTTGTSHLVKCAEKEKTFCVNGGECFMVKDLSNPSRYLCKCPNEFTGDRCQNYVMASFYSTSTPFLSLPE

SEQ ID NO: 100 - protein, 590 aa, artificial Sequence
  NRG1 Isoform 11
MGKGRAGRVGTTALPPRLKEMKSQESAAGSKLVLRCETSSEYSSLRFKWFKNGNELNRKNKPQNIKIQKKPGKSELRINKASLADSGEYMCKVISKLGNDSASANITIVESNATSTSTTGTSHLVKCAEKEKTFCVNGGECFMVKDLSNPSRYLCKCPNEFTGDRCQNYVMASFYKHLGIEFMEAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 101 - protein, 420 aa, artificial Sequence
  NRG1 Isoform 12
MSERKEGRGKGKGKKKERGSGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYKAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSER

SEQ ID NO: 102 - protein, 179 aa, artificial Sequence
  STMN2 Isoform 1
MAKTAMAYKEKMKELSMLSLICSCFYPEPRNINIYTYDDMEVKQINKRASGQAFELILKPPSPISEAPRTLASPKKKDLSLEEIQKKLEAAEERRKSQEAQVLKQLAEKREHEREVLQKALEENNNFSKMAEEKLILKMEQIKENREANLAAIIERLQEKERHAAEVRRNKELQVELSG

SEQ ID NO: 103 - protein, 187 aa, artificial Sequence
  STMN2 Isoform 2
MAKTAMAYKEKMKELSMLSLICSCFYPEPRNINIYTYDDMEVKQINKRASGQAFELILKPPSPISEAPRTLASPKKKDLSLEEIQKKLEAAEERRKSQEAQVLKQLAEKREHEREVLQKALEENNNFSKMAEEKLILKMEQIKENREANLAAIIERLQEKLVKFISSELKESIESQFLELQREGEKQ

SEQ ID NO: 104 - protein, 1477 aa, artificial Sequence
  NRXN1-Alpha Isoform 1a
MGTALLQRGGCFLLCLSLLLLGCWAELGSGLEFPGAEGQWTRFPKWNACCESEMSFQLKTRSARGLVLYFDDEGFCDFLELILTRGGRLQLSFSIFCAEPATLLADTPVNDGAWHSVRIRRQFRNTTLFIDQVEAKWVEVKSKRRDMTVFSGLFVGGLPPELRAAALKLTLASVREREPFKGWIRDVRVNSSQVLPVDSGEVKLDDEPPNSGGGSPCEAGEEGEGGVCLNGGVCSVVDDQAVCDCSRTGFRGKDCSQEDNNVEGLAHLMMGDQGKSKGKEEYIATFKGSEYFCYDLSQNPIQSSSDEITLSFKTLQRNGLMLHTGKSADYVNLALKNGAVSLVINLGSGAFEALVEPVNGKFNDNAWHDVKVTRNLRQHSGIGHAMVTISVDGILTTTGYTQEDYTMLGSDDFFYVGGSPSTADLPGSPVSNNFMGCLKEVVYKNNDVRLELSRLAKQGDPKMKIHGVVAFKCENVATLDPITFETPESFISLPKWNAKKTGSISFDFRTTEPNGLILFSHGKPRHQKDAKHPQMIKVDFFAIEMLDGHLYLLLDMGSGTIKIKALLKKVNDGEWYHVDFQRDGRSGTISVNTLRTPYTAPGESEILDLDDELYLGGLPENKAGLVFPTEVWTALLNYGYVGCIRDLFIDGQSKDIRQMAEVQSTAGVKPSCSKETAKPCLSNPCKNNGMCRDGWNRYVCDCSGTGYLGRSCEREATVLSYDGSMFMKIQLPVVMHTEAEDVSLRFRSQRAYGILMATTSRDSADTLRLELDAGRVKLTVNLDCIRINCNSSKGPETLFAGYNLNDNEWHTVRVVRRGKSLKLTVDDQQAMTGQMAGDHTRLEFHNIETGIITERRYLSSVPSNFIGHLQSLTFNGMAYIDLCKNGDIDYCELNARFGFRNIIADPVTFKTKSSYVALATLQAYTSMHLFFQFKTTSLDGLILYNSGDGNDFIVVELVKGYLHYVFDLGNGANLIKGSSNKPLNDNQWHNVMISRDTSNLHTVKIDTKITTQITAGARNLDLKSDLYIGGVAKETYKSLPKLVHAKEGFQGCLASVDLNGRLPDLISDALFCNGQIERGCEGPSTTCQEDSCSNQGVCLQQWDGFSCDCSMTSFSGPLCNDPGTTYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGRQLTIFNSQATIIIGGKEQGQPFQGQLSGLYYNGLKVLNMAAENDANIAIVGNVRLVGEVPSSMTTESTATAMQSEMSTSIMETTTTLATSTARRGKPPTKEPISQTTDDILVASAECPSDDEDIDPCEPSSGGLANPTRAGGREPYPGSAEVIRESSSTTGMVVGIVAAAALCILILLYAMYKYRNRDEGSYHVDESRNYISNSAQSNGAVVKEKQPSSAKSSNKNKKNKDKEYYV

SEQ ID NO: 105 - protein, 1496 aa, artificial Sequence
  NRXN1-Alpha Isoform 2a
MGTALLQRGGCFLLCLSLLLLGCWAELGSGLEFPGAEGQWTRFPKWNACCESEMSFQLKTRSARGLVLYFDDEGFCDFLELILTRGGRLQLSFSIFCAEPATLLADTPVNDGAWHSVRIRRQFRNTTLFIDQVEAKWVEVKSKRRDMTVFSGLFVGGLPPELRAAALKLTLASVREREPFKGWIRDVRVNSSQVLPVDSGEVKLDDEPPNSGGGSPCEAGEEGEGGVCLNGGVCSVVDDQAVCDCSRTGFRGKDCSQEDNNVEGLAHLMMGDQGKSKGKEEYIATFKGSEYFCYDLSQNPIQSSSDEITLSFKTLQRNGLMLHTGKSADYVNLALKNGAVSLVINLGSGAFEALVEPVNGKFNDNAWHDVKVTRNLRQVTISVDGILTTTGYTQEDYTMLGSDDFFYVGGSPSTADLPGSPVSNNFMGCLKEVVYKNNDVRLELSRLAKQGDPKMKIHGVVAFKCENVATLDPITFETPESFISLPKWNAKKTGSISFDFRTTEPNGLILFSHGKPRHQKDAKHPQMIKVDFFAIEMLDGHLYLLLDMGSGTIKIKALLKKVNDGEWYHVDFQRDGRSGTISVNTLRTPYTAPGESEILDLDDELYLGGLPENKAGLVFPTEVWTALLNYGYVGCIRDLFIDGQSKDIRQMAEVQSTAGVKPSCSKETAKPCLSNPCKNNGMCRDGWNRYVCDCSGTGYLGRSCEREATVLSYDGSMFMKIQLPVVMHTEAEDVSLRFRSQRAYGILMATTSRDSADTLRLELDAGRVKLTVNLDCIRINCNSSKGPETLFAGYNLNDNEWHTVRVVRRGKSLKLTVDDQQAMTGQMAGDHTRLEFHNIETGIITERRYLSSVPSNFIGHLQSLTFNGMAYIDLCKNGDIDYCELNARFGFRNIIADPVTFKTKSSYVALATLQAYTSMHLFFQFKTTSLDGLILYNSGDGNDFIVVELVKGYLHYVFDLGNGANLIKGSSNKPLNDNQWHNVMISRDTSNLHTVKIDTKITTQITAGARNLDLKSDLYIGGVAKETYKSLPKLVHAKEGFQGCLASVDLNGRLPDLISDALFCNGQIERGCEGPSTTCQEDSCSNQGVCLQQWDGFSCDCSMTSFSGPLCNDPGTTYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGNNDNERLAIARQRIPYRLGRVVDEWLLDKGRQLTIFNSQATIIIGGKEQGQPFQGQLSGLYYNGLKVLNMAAENDANIAIVGNVRLVGEVPSSMTTESTATAMQSEMSTSIMETTTTLATSTARRGKPPTKEPISQTTDDILVASAECPSDDEDIDPCEPSSANPTRAGGREPYPGSAEVIRESSSTTGMVVGIVAAAALCILILLYAMYKYRNRDEGSYHVDESRNYISNSAQSNGAVVKEKQPSSAKSSNKNKKNKDKEYYV

SEQ ID NO: 106 - protein, 1547 aa, artificial Sequence
  NRXN1-Alpha Isoform 3a
MGTALLQRGGCFLLCLSLLLLGCWAELGSGLEFPGAEGQWTRFPKWNACCESEMSFQLKTRSARGLVLYFDDEGFCDFLELILTRGGRLQLSFSIFCAEPATLLADTPVNDGAWHSVRIRRQFRNTTLFIDQVEAKWVEVKSKRRDMTVFSGLFVGGLPPELRAAALKLTLASVREREPFKGWIRDVRVNSSQVLPVDSGEVKLDDEPPNSGGGSPCEAGEEGEGGVCLNGGVCSVVDDQAVCDCSRTGFRGKDCSQEIKFGLQCVLPVLLHDNDQGKYCCINTAKPLTEKDNNVEGLAHLMMGDQGKSKGKEEYIATFKGSEYFCYDLSQNPIQSSSDEITLSFKTLQRNGLMLHTGKSADYVNLALKNGAVSLVINLGSGAFEALVEPVNGKFNDNAWHDVKVTRNLRQHSGIGHAMVNKLHCSVTISVDGILTTTGYTQEDYTMLGSDDFFYVGGSPSTADLPGSPVSNNFMGCLKEVVYKNNDVRLELSRLAKQGDPKMKIHGVVAFKCENVATLDPITFETPESFISLPKWNAKKTGSISFDFRTTEPNGLILFSHGKPRHQKDAKHPQMIKVDFFAIEMLDGHLYLLLDMGSGTIKIKALLKKVNDGEWYHVDFQRDGRSGTISVNTLRTPYTAPGESEILDLDDELYLGGLPENKAGLVFPTEVWTALLNYGYVGCIRDLFIDGQSKDIRQMAEVQSTAGVKPSCSKETAKPCLSNPCKNNGMCRDGWNRYVCDCSGTGYLGRSCEREATVLSYDGSMFMKIQLPVVMHTEAEDVSLRFRSQRAYGILMATTSRDSADTLRLELDAGRVKLTVNLDCIRINCNSSKGPETLFAGYNLNDNEWHTVRVVRRGKSLKLTVDDQQAMTGQMAGDHTRLEFHNIETGIITERRYLSSVPSNFIGHLQSLTFNGMAYIDLCKNGDIDYCELNARFGFRNIIADPVTFKTKSSYVALATLQAYTSMHLFFQFKTTSLDGLILYNSGDGNDFIVVELVKGYLHYVFDLGNGANLIKGSSNKPLNDNQWHNVMISRDTSNLHTVKIDTKITTQITAGARNLDLKSDLYIGGVAKETYKSLPKLVHAKEGFQGCLASVDLNGRLPDLISDALFCNGQIERGCEGPSTTCQEDSCSNQGVCLQQWDGFSCDCSMTSFSGPLCNDPGTTYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGNNDNERLAIARQRIPYRLGRVVDEWLLDKGRQLTIFNSQATIIIGGKEQGQPFQGQLSGLYYNGLKVLNMAAENDANIAIVGNVRLVGEVPSSMTTESTATAMQSEMSTSIMETTTTLATSTARRGKPPTKEPISQTTDDILVASAECPSDDEDIDPCEPSSGGLANPTRAGGREPYPGSAEVIRESSSTTGMVVGIVAAAALCILILLYAMYKYRNRDEGSYHVDESRNYISNSAQSNGAVVKEKQPSSAKSSNKNKKNKDKEYYV

SEQ ID NO: 107 - protein, 139 aa, artificial Sequence
  NRXN1-Alpha Isoform 4a
MDMRWHCENSQTTDDILVASAECPSDDEDIDPCEPSSANPTRAGGREPYPGSAEVIRESSSTTGMVVGIVAAAALCILILLYAMYKYRNRDEGSYHVDESRNYISNSAQSNGAVVKEKQPSSAKSSNKNKKNKDKEYYV

SEQ ID NO: 108 - protein, 472 aa, artificial Sequence
  NRXN1-Beta Isoform 1b
MYQRMLRCGAELGSPGGGGGGGGGGGAGGRLALLWIVPLTLSGLLGVAWGASSLGAHHIHHFHGSSKHHSVPIAIYRSPASLRGGHAGTTYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGNNDNERLAIARQRIPYRLGRVVDEWLLDKGRQLTIFNSQATIIIGGKEQGQPFQGQLSGLYYNGLKVLNMAAENDANIAIVGNVRLVGEVPSSMTTESTATAMQSEMSTSIMETTTTLATSTARRGKPPTKEPISQTTDDILVASAECPSDDEDIDPCEPSSGGLANPTRAGGREPYPGSAEVIRESSSTTGMVVGIVAAAALCILILLYAMYKYRNRDEGSYHVDESRNYISNSAQSNGAVVKEKQPSSAKSSNKNKKNKDKEYYV

SEQ ID NO: 109 - protein, 442 aa, artificial Sequence
  NRXN1-Beta Isoform 3b
MYQRMLRCGAELGSPGGGGGGGGGGGAGGRLALLWIVPLTLSGLLGVAWGASSLGAHHIHHFHGSSKHHSVPIAIYRSPASLRGGHAGTTYIFSKGGGQITYKWPPNDRPSTRADRLAIGFSTVQKEAVLVRVDSSSGLGDYLELHIHQGKIGVKFNVGTDDIAIEESNAIINDGKYHVVRFTRSGGNATLQVDSWPVIERYPAGRQLTIFNSQATIIIGGKEQGQPFQGQLSGLYYNGLKVLNMAAENDANIAIVGNVRLVGEVPSSMTTESTATAMQSEMSTSIMETTTTLATSTARRGKPPTKEPISQTTDDILVASAECPSDDEDIDPCEPSSGGLANPTRAGGREPYPGSAEVIRESSSTTGMVVGIVAAAALCILILLYAMYKYRNRDEGSYHVDESRNYISNSAQSNGAVVKEKQPSSAKSSNKNKKNKDKEYYV

SEQ ID NO: 110 - protein, 2527 aa, artificial Sequence
  LRKK2 Protein
MASGSCQGCEEDEETLKKLIVRLNNVQEGKQIETLVQILEDLLVFTYSERASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 111 - protein, 873 aa, artificial Sequence
  VLDLR isoform long
MGTSALWALWLLLALCWAPRESGATGTGRKAKCEPSQFQCTNGRCITLLWKCDGDEDCVDGSDEKNCVKKTCAESDFVCNNGQCVPSRWKCDGDPDCEDGSDESPEQCHMRTCRIHEISCGAHSTQCIPVSWRCDGENDCDSGEDEENCGNITCSPDEFTCSSGRCISRNFVCNGQDDCSDGSDELDCAPPTCGAHEFQCSTSSCIPISWVCDDDADCSDQSDESLEQCGRQPVIHTKCPASEIQCGSGECIHKKWRCDGDPDCKDGSDEVNCPSRTCRPDQFECEDGSCIHGSRQCNGIRDCVDGSDEVNCKNVNQCLGPGKFKCRSGECIDISKVCNQEQDCRDWSDEPLKECHINECLVNNGGCSHICKDLVIGYECDCAAGFELIDRKTCGDIDECQNPGICSQICINLKGGYKCECSRGYQMDLATGVCKAVGKEPSLIFTNRRDIRKIGLERKEYIQLVEQLRNTVALDADIAAQKLFWADLSQKAIFSASIDDKVGRHVKMIDNVYNPAAIAVDWVYKTIYWTDAASKTISVATLDGTKRKFLFNSDLREPASIAVDPLSGFVYWSDWGEPAKIEKAGMNGFDRRPLVTADIQWPNGITLDLIKSRLYWLDSKLHMLSSVDLNGQDRRIVLKSLEFLAHPLALTIFEDRVYWIDGENEAVYGANKFTGSELATLVNNLNDAQDIIVYHELVQPSGKNWCEEDMENGGCEYLCLPAPQINDHSPKYTCSCPSGYNVEENGRDCQSTATTVTYSETKDTNTTEISATSGLVPGGINVTTAVSEVSVPPKGTSAAWAILPLLLLVMAAVGGYLMWRNWQHKNMKSMNFDNPVYLKTTEEDLSIDIGRHSASVGHTYPAISVVSTDDDLA

SEQ ID NO: 112 - protein, 845 aa, artificial Sequence
  VLDLR isoform short
MGTSALWALWLLLALCWAPRESGATGTGRKAKCEPSQFQCTNGRCITLLWKCDGDEDCVDGSDEKNCVKKTCAESDFVCNNGQCVPSRWKCDGDPDCEDGSDESPEQCHMRTCRIHEISCGAHSTQCIPVSWRCDGENDCDSGEDEENCGNITCSPDEFTCSSGRCISRNFVCNGQDDCSDGSDELDCAPPTCGAHEFQCSTSSCIPISWVCDDDADCSDQSDESLEQCGRQPVIHTKCPASEIQCGSGECIHKKWRCDGDPDCKDGSDEVNCPSRTCRPDQFECEDGSCIHGSRQCNGIRDCVDGSDEVNCKNVNQCLGPGKFKCRSGECIDISKVCNQEQDCRDWSDEPLKECHINECLVNNGGCSHICKDLVIGYECDCAAGFELIDRKTCGDIDECQNPGICSQICINLKGGYKCECSRGYQMDLATGVCKAVGKEPSLIFTNRRDIRKIGLERKEYIQLVEQLRNTVALDADIAAQKLFWADLSQKAIFSASIDDKVGRHVKMIDNVYNPAAIAVDWVYKTIYWTDAASKTISVATLDGTKRKFLFNSDLREPASIAVDPLSGFVYWSDWGEPAKIEKAGMNGFDRRPLVTADIQWPNGITLDLIKSRLYWLDSKLHMLSSVDLNGQDRRIVLKSLEFLAHPLALTIFEDRVYWIDGENEAVYGANKFTGSELATLVNNLNDAQDIIVYHELVQPSGKNWCEEDMENGGCEYLCLPAPQINDHSPKYTCSCPSGYNVEENGRDCQRINVTTAVSEVSVPPKGTSAAWAILPLLLLVMAAVGGYLMWRNWQHKNMKSMNFDNPVYLKTTEEDLSIDIGRHSASVGHTYPAISVVSTDDDLA

SEQ ID NO: 113 - protein, 902 aa, artificial Sequence
  GRIA4 isoform 1
MRIISRQIVLLFSGFWGLAMGAFPSSVQIGGLFIRNTDQEYTAFRLAIFLHNTSPNASEAPFNLVPHVDNIETANSFAVTNAFCSQYSRGVFAIFGLYDKRSVHTLTSFCSALHISLITPSFPTEGESQFVLQLRPSLRGALLSLLDHYEWNCFVFLYDTDRGYSILQAIMEKAGQNGWHVSAICVENFNDVSYRQLLEELDRRQEKKFVIDCEIERLQNILEQIVSVGKHVKGYHYIIANLGFKDISLERFIHGGANVTGFQLVDFNTPMVIKLMDRWKKLDQREYPGSETPPKYTSALTYDGVLVMAETFRSLRRQKIDISRRGNAGDCLANPAAPWGQGIDMERTLKQVRIQGLTGNVQFDHYGRRVNYTMDVFELKSTGPRKVGYWNDMDKLVLIQDVPTLGNDTAAIENRTVVVTTIMESPYVMYKKNHEMFEGNDKYEGYCVDLASEIAKHIGIKYKIAIVPDGKYGARDADTKIWNGMVGELVYGKAEIAIAPLTITLVREEVIDFSKPFMSLGISIMIKKPQKSKPGVFSFLDPLAYEIWMCIVFAYIGVSVVLFLVSRFSPYEWHTEEPEDGKEGPSDQPPNEFGIFNSLWFSLGAFMQQGCDISPRSLSGRIVGGVWWFFTLIIISSYTANLAAFLTVERMVSPIESAEDLAKQTEIAYGTLDSGSTKEFFRRSKIAVYEKMWTYMRSAEPSVFTRTTAEGVARVRKSKGKFAFLLESTMNEYIEQRKPCDTMKVGGNLDSKGYGVATPKGSSLRTPVNLAVLKLSEAGVLDKLKNKWWYDKGECGPKDSGSKDKTSALSLSNVAGVFYILVGGLGLAMLVALIEFCYKSRAEAKRMKLTFSEAIRNKARLSITGSVGENGRVLTPDCPKAVHTGTAIRQSSGLAVIASDLP

SEQ ID NO: 114 - protein, 433 aa, artificial Sequence
  GRIA4 isoform 2
MRIISRQIVLLFSGFWGLAMGAFPSSVQIGGLFIRNTDQEYTAFRLAIFLHNTSPNASEAPFNLVPHVDNIETANSFAVTNAFCSQYSRGVFAIFGLYDKRSVHTLTSFCSALHISLITPSFPTEGESQFVLQLRPSLRGALLSLLDHYEWNCFVFLYDTDRGYSILQAIMEKAGQNGWHVSAICVENFNDVSYRQLLEELDRRQEKKFVIDCEIERLQNILEQIVSVGKHVKGYHYIIANLGFKDISLERFIHGGANVTGFQLVDFNTPMVIKLMDRWKKLDQREYPGSETPPKYTSALTYDGVLVMAETFRSLRRQKIDISRRGNAGDCLANPAAPWGQGIDMERTLKQVRIQGLTGNVQFDHYGRRVNYTMDVFELKSTGPRKVGYWNDMDKLVLIQDVPTLGNDTAAIENRTVVVTTIMPLMKNPILRN

SEQ ID NO: 115 - protein, 271 aa, artificial Sequence
  NXPH1
MQAACWYVLFLLQPTVYLVTCANLTNGGKSELLKSGSSKSTLKHIWTESSKDLSISRLLSQTFRGKENDTDLDLRYDTPEPYSEQDLWDWLRNSTDLQEPRPRAKRRPIVKTGKFKKMFGWGDFHSNIKTVKLNLLITGKIVDHGNGTFSVYFRHNSTGQGNVSVSLVPPTKIVEFDLAQQTVIDAKDSKSFNCRIEYEKVDKATKNTLCNYDPSKTCYQEQTQSHVSWLCSKPFKVICIYISFYSTDYKLVQKVCPDYNYHSDTPYFPSG

SEQ ID NO: 116 - protein, 724 aa, artificial Sequence
  PSD-95 isoform 1
MDCLCIVTTKKYRYQDEDTPPLEHSPAHLPNQANSPPVIVNTDTLEAPGYELQVNGTEGEMEYEEITLERGNSGLGFSIAGGTDNPHIGDDPSIFITKIIPGGAAAQDGRLRVNDSILFVNEVDVREVTHSAAVEALKEAGSIVRLYVMRRKPPAEKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNAYLSDSYAPPDITTSYSQHLDNEISHSSYLGTDYPTAMTPTSPRRYSPVAKDLLGEEDIPREPRRIVIHRGSTGLGFNIVGGEDGEGIFISFILAGGPADLSGELRKGDQILSVNGVDLRNASHEQAAIALKNAGQTVTIIAQYKPEEYSRFEAKIHDLREQLMNSSLGSGTASLRSNPKRGFYIRALFDYDKTKDCGFLSQALSFRFGDVLHVIDASDEEWWQARRVHSDSETDDIGFIPSKRRVERREWSRLKAKDWGSSSGSQGREDSVLSYETVTQMEVHYARPIIILGPTKDRANDDLLSEFPDKFGSCVPHTTRPKREYEIDGRDYHFVSSREKMEKDIQAHKFIEAGQYNSHLYGTSVQSVREVAEQGKHCILDVSANAVRRLQAAHLHPIAIFIRPRSLENVLEINKRITEEQARKAFDRATKLEQEFTECFSAIVEGDSFEEIYHKVKRVIEDLSGPYIWVPARERL

SEQ ID NO: 117 - protein, 767 aa, artificial Sequence
  PSD-95 isoform 2
MSQRPRAPRSALWLLAPPLLRWAPPLLTVLHSDLFQALLDILDYYEASLSESQKYRYQDEDTPPLEHSPAHLPNQANSPPVIVNTDTLEAPGYELQVNGTEGEMEYEEITLERGNSGLGFSIAGGTDNPHIGDDPSIFITKIIPGGAAAQDGRLRVNDSILFVNEVDVREVTHSAAVEALKEAGSIVRLYVMRRKPPAEKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNAYLSDSYAPPDITTSYSQHLDNEISHSSYLGTDYPTAMTPTSPRRYSPVAKDLLGEEDIPREPRRIVIHRGSTGLGFNIVGGEDGEGIFISFILAGGPADLSGELRKGDQILSVNGVDLRNASHEQAAIALKNAGQTVTIIAQYKPEEYSRFEAKIHDLREQLMNSSLGSGTASLRSNPKRGFYIRALFDYDKTKDCGFLSQALSFRFGDVLHVIDASDEEWWQARRVHSDSETDDIGFIPSKRRVERREWSRLKAKDWGSSSGSQGREDSVLSYETVTQMEVHYARPIIILGPTKDRANDDLLSEFPDKFGSCVPHTTRPKREYEIDGRDYHFVSSREKMEKDIQAHKFIEAGQYNSHLYGTSVQSVREVAEQGKHCILDVSANAVRRLQAAHLHPIAIFIRPRSLENVLEINKRITEEQARKAFDRATKLEQEFTECFSAIVEGDSFEEIYHKVKRVIEDLSGPYIWVPARERL

SEQ ID NO: 118 - protein, 720 aa, artificial Sequence
  PSD-95 isoform 3
MDCLCIVTTKKYRYQDEDTPPLEHSPAHLPNQANSPPVIVNTDTLEAPGYVNGTEGEMEYEEITLERGNSGLGFSIAGGTDNPHIGDDPSIFITKIIPGGAAAQDGRLRVNDSILFVNEVDVREVTHSAAVEALKEAGSIVRLYVMRRKPPAEKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNAYLSDSYAPPDITTSYSQHLDNEISHSSYLGTDYPTAMTPTSPRRYSPVAKDLLGEEDIPREPRRIVIHRGSTGLGFNIVGGEDGEGIFISFILAGGPADLSGELRKGDQILSVNGVDLRNASHEQAAIALKNAGQTVTIIAQYKPEEYSRFEAKIHDLREQLMNSSLGSGTASLRSNPKRGFYIRALFDYDKTKDCGFLSQALSFRFGDVLHVIDASDEEWWQARRVHSDSETDDIGFIPSKRRVERREWSRLKAKDWGSSSGSQGREDSVLSYETVTQMEVHYARPIIILGPTKDRANDDLLSEFPDKFGSCVPHTTRPKREYEIDGRDYHFVSSREKMEKDIQAHKFIEAGQYNSHLYGTSVQSVREVAEQGKHCILDVSANAVRRLQAAHLHPIAIFIRPRSLENVLEINKRITEEQARKAFDRATKLEQEFTECFSAIVEGDSFEEIYHKVKRVIEDLSGPYIWVPARER

SEQ ID NO: 119 - protein, 313 aa, artificial Sequence
  Synaptophysin isoform 1
MLLLADMDVVNQLVAGGQFRVVKEPLGFVKVLQWVFAIFAFATCGSYSGELQLSVDCANKTESDLSIEVEFEYPFRLHQVYFDAPTCRGGTTKVFLVGDYSSSAEFFVTVAVFAFLYSMGALATYIFLQNKYRENNKGPMLDFLATAVFAFMWLVSSSAWAKGLSDVKMATDPENIIKEMPVCRQTGNTCKELRDPVTSGLNTSVVFGFLNLVLWVGNLWFVFKETGWAAPFLRAPPGAPEKQPAPGDAYGDAGYGQGPGGYGPQDSYGPQGGYQPDYGQPAGSGGSGYGPQGDYGQQGYGPQGAPTSFSNQM

SEQ ID NO: 120 - protein, 193 aa, artificial Sequence
  Synaptophysin isoform 2
ALATYIFLQNKYRENNKGPMLDFLATAVFAFMWLVSSSAWAKGLSDVKMATDPENIIKEMPVCRQTGNTCKELRDPVTSGLNTSVVFGFLNLVLWVGNLWFVFKETGWAAPFLRAPPGAPEKQPAPGDAYGDAGYGQGPGGYGPQDSYGPQGGYQPDYGQPAGSGGSGYGPQGDYGQQGYGPQGAPTSFSNQM

SEQ ID NO: 121 - protein, 277 aa, artificial Sequence
  Caspase-3
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 122 - DNA, 8873 bp, artificial Sequence
  pLenti-III-mir-GFP vector
ttttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatacggcagacctcagcgctagattattgaagcatttatcagggttattgtctcatgagcggatacatatttgaatgtatttagaaaaataaacaaataggggttccgcgcacatttccccgaaaagtgccacctgacgttaactataacggtcctaaggtagcgaaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcccgctgatcttcagacctggaggaggagatatgagggacattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgaaagcttgggattcgaatttaaaagaaaaggggggattggggggtacagtgcaggggaaagaatagtagacataatagcaacagacatacaaactaaagaactacaaaaacaaattacaaaaattcaaaattttcgggtttttcgaacctagggttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtttagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagaaccgagtttaaactccctatcagtgatagagatctccctatcagtgatagagagctagaatctagaggtaccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgaggtaccgatatcgaattcatagctagccctgcaggtctagactcgaggtcatacacggctctcctctctgcggccgcagtcgagtacccatacgacgtcccagactacgcttgagtttaaacacgcgtggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggccatgaccgagtacaagcccacggtgcgcctcgccacccgcgacgacgtccctcgggccgtacgcaccctcgccgccgcgttcgccgactaccccgccacgcgccacaccgtggacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaagggctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttccggaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtctcgccttcgccctcagacgagtcggatctccctttgggccgcctccccgcctgtccggatggaagggctaattcactcccaacgaatacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaattctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggcatctatgtcgggtgcggagaaagaggtaatgaaatggcattatgggtattatgggtctgcattaatgaatcggccaacgatcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttgttaaaatttttgttaaatcagctcattttttaaccaataggccgaaatcggcaacatcccttataaatcaaaagaatagaccgcgatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgcgcgcttaatgcgccgctacagggcgcgtccattcgccattcaggatcgaattaattcttaattaacatcatcaataatatacctt